(19) United States
(12) Patent Application Publication
(10) Pub. No.: US 2011/0236325 A1
(43) Pub. Date: Sep. 29, 2011

(54) METHODS TO TREAT OR PREVENT A SKIN CONDITION USING A NELL1 PEPTIDE

(75) Inventors: Dora P. Mitchell, Langhorne, PA (US); Cymbeline T. Culiat, Oak Ridge, TN (US); Beverly W. Lubit, Kinnelon, NJ (US)

(73) Assignee: NellOne Therapeutics, Inc., Oak Ridge, TN (US)

(21) Appl. No.: 13/011,339

(22) Filed: Jan. 21, 2011

Related U.S. Application Data
(60) Provisional application No. 61/296,965, filed on Jan. 21, 2010.

(51) Int. Cl. A61K 38/17; C12Q 1/02; C12Q 1/37; A61K 31/7088; A61K 48/00; A61P 17/02; A61P 17/00; A61P 35/00; A61P 17/12; A61P 29/00; A61P 17/04; A61P 31/12; A61P 33/02; A61P 31/10; A61Q 17/04; A61P 31/00; A61P 17/10; A61P 23/00; A61P 39/06

(52) U.S. Cl. 424/59; 514/18.6; 514/18.7; 514/18.8; 435/29; 435/23; 514/44 R

(57) ABSTRACT

The described invention provides methods and compositions utilizing a NELL1 peptide or nucleic acid encoding a NELL1 peptide for treating or preventing a skin condition. The methods include treating or preventing manifestations of aging in human skin, repairing damage to skin, and preventing skin scarring. Methods are also provided for assaying a test peptide for NELL1 activity.

### CROSS-REFERENCE TO RELATED APPLICATIONS

This application claims the benefit of U.S. Provisional Application No. 61/296,965, filed on Jan. 21, 2010, the contents of which are hereby incorporated by reference in their entirety.

### REFERENCE TO A SEQUENCE LISTING SUBMITTED AS A TEXT FILE VIA EFS-WEB

The official copy of the sequence listing is submitted electronically via EFS-Web as an ASCII formatted sequence listing with a file named 400589SEQLIST.TXT, created on Jan. 21, 2011, and having a size of 369 kilobytes and is filed concurrently with the specification. The sequence listing contained in this ASCII formatted document is part of the specification and is herein incorporated by reference in its entirety.

### FIELD OF THE INVENTION

The described invention relates to the use of NELL1 peptides and nucleic acids for treating or preventing manifestations of a skin condition in human skin.

### BACKGROUND OF THE INVENTION

The integument is the human body's most massive organ. It is composed of the skin (including the epidermis and dermis) that covers the entire body and accessory organs, which are derivatives of the skin, such as nails, hair, and sweat, sebaceous, and mammary glands.

The skin is regularly subjected to numerous physical, chemical and biological insults, including those of skin aging and defective wound repair, which can result in injuries and diseases that leave unsightly scars or poor skin appearance and function. These skin conditions may result in both physical and emotional distress to a patient.

Photo-aged skin is an example of a common condition that can be treated with various in-office procedures and numerous topical agents, most of which are intended to resurface the epidermis; generally, this means removing the damaged epidermis and, in some cases, the dermis, and replacing the tissue with remodeled skin layers. Results of these resurfacing procedures are variable, and may spur the formation of new collagen.

Clinically, the use of growth factors, cytokines, and telomerase to treat photo-aged skin is not practiced. Prevention of extrinsic aging remains the best approach for treating aged skin. This entails avoiding exposure to the sun, using sunscreen when sun avoidance is impossible, avoiding cigarette smoke and pollution, eating a diet high in fruits and vegetables, and taking oral anti-oxidant supplements or topical anti-oxidant formulations. The regular use of prescription retinoids also may help prevent or treat wrinkles

The formation of rhytides (wrinkles) is considered the most conspicuous and common manifestation of skin aging. Wrinkles appear as a result of changes in the lower, dermal layers of the skin. Many consumers do not realize, given the ubiquity of advertising that touts the newest topical formulations to eliminate wrinkles and the related expenditure of millions of dollars by consumers on these ‘anti-aging’ products, that few skin care product ingredients have the capacity to penetrate far enough into the dermis to ameliorate deep wrinkles

The only known defenses against photo-aging beyond sun avoidance are sunscreens, retinoids and antioxidants. It generally is believed that the effects of aged skin, once apparent, may not be rectified. There is no curative solution to the problem of skin aging.

Wound healing, or wound repair, is the body's natural process of regenerating dermal and epidermal tissue in response to injury. When an individual is wounded, a set of complex biochemical events takes place to repair the damage, including angiogenesis, collagen deposition, and granulation tissue formation. Regulation of the steps that govern epidermal renewal must enable the epidermis to respond to rough usage by becoming thick and callused, and to repair itself when wounded.

A scar (cicatrices) is an area of fibrous tissue that replaces normal skin (or other tissue) after injury. A scar results from the biologic process of wound repair in the skin and other tissues of the body. Skin scars occur when the deep, thick layer of skin (the dermis) is damaged.

Scars arise after almost every dermal injury; rare exceptions include tattoos, superficial scratches, and venipunctures. Scars often are considered trivial, but they can be disfiguring and aesthetically unpleasant and cause severe itching, tenderness, pain, sleep disturbance, anxiety, depression, and disruption of other daily activities. Other psychological sequelae include development of post-traumatic stress reactions, loss of self esteem, and stigmatization, leading to diminished quality of life. Physical deformity as a result of skin scar contractures can be disabling. Many scars take several years to pale and mature.

In spite of media suggestions to the contrary, scars cannot be made to disappear. Many patients arrive at plastic surgery clinics with unrealistic expectations. When considering treatment, clinical judgment, balancing the potential benefits of the various treatments available against the likelihood of a poor response and possible iatrogenic complications, is required. The evidence base for the use of many current treatments is poor, and some may have only placebo benefit.

There is considerable quantitative and qualitative variation in scarring potential between individuals and even within the same individual. Important determinants of scar production include the extent and duration of inflammation at the wound site, the magnitude of mechanical tension acting on the scar, and the genetic phenotype of the individual. Although multiple management options are available for the treatment of scars, no skin scar can be removed completely.

### BRIEF SUMMARY OF THE INVENTION

The described invention provides methods and compositions utilizing a NELL1 peptide or a nucleic acid molecule encoding the same for treating or preventing a skin condition in a subject. In certain embodiments, the NELL1 peptide or a nucleic acid encoding the same treats or prevents the manifestations of aging in human skin, for repair of damage to skin, and for the prevention or treatment of skin scarring. According to one aspect, the described invention provides a method for treating or preventing a skin condition, such as aged skin or a skin scar, by administering a therapeutically effective amount of a NELL1 peptide or a nucleic acid molecule encoding the same to a subject in need thereof, thereby treating or preventing at least one manifestation of the skin condition. In those embodiments wherein the skin condition is a skin scar, the skin scar can be selected from the group consisting of a keloid scar, a widespread scar, and an atrophic scar.

The NELL1 peptide or a nucleic acid molecule encoding the same can be administered along with a carrier in a composition. The composition can be a pharmaceutical, cosmetic, or cosmeceutical composition that in some embodiments, is administered topically to an epithelial surface. In some embodiments wherein the formation of a scar is prevented, the composition is administered topically to a wounded epithelial surface.

The NELL1 peptide can be from any source, but in some embodiments is human NELL1 or an active variant or fragment thereof. Thus, in some embodiments, the NELL1 peptide has an amino acid sequence having at least 70% sequence identity to SEQ ID NO: 2, 3, 4, 5, 6, 7, 8, 9, 11, 12, 13, 14, 15, 16, or 17.

Methods are also provided for assaying a test peptide for NELL1 activity, such as the ability to reduce the levels of inflammatory mediators and matrix metalloproteinases, increase the levels of aquaporins, and the ability to reduce the number of sunburned cells in a skin sample. Such methods can utilize skin equivalents, such as three-dimensional skin models.

### BRIEF DESCRIPTION OF THE FIGURES

FIG. 1 shows the general structure of the nel-like 1 isoform 1 (SEQ ID NO: 2).

FIG. 2 shows a diagrammatic representation of skin epithelial histology.

FIG. 3 shows a diagrammatic representation of the anatomy of the skin.

FIG. 4 shows the number of sunburned cells in hematoxylin and eosin-stained slices of paraffin-embedded EpiDerm-FT™ 400 three-dimensional skin model tissues that have been treated with ultraviolet radiation and/or 50 μM genistein or 100 ng/mL recombinant human NELL1 protein.

FIGS. 5A and 5B show the number of sunburned cells in histological sections of EpiDerm-FT™ 400 three-dimensional skin models that have been treated with ultraviolet radiation and/or 50 μM genistein, 100 ng/mL, or 150 ng/ml recombinant human NELL1 protein in two separate experiments.

FIGS. 6A and 6B show the relative RNA levels of interleukin (IL)-1β, interleukin (IL)-8, matrix metalloproteinase1 (MMP1) (FIG. 6A), and aquaporin 3 (AQP3) (FIG. 6B) in EpiDerm-FT™ 400 three-dimensional skin model tissues that have been treated with ultraviolet radiation and/or 50 μM genistein, 100 ng/mL recombinant human NELL1 protein. The RNA levels have been normalized to housekeeping gene levels and compared to no UV control levels.

### DETAILED DESCRIPTION OF THE INVENTION

Methods and compositions comprising a NELL1 peptide or a nucleic acid molecule encoding the same are provided for the prevention or treatment of skin conditions, such as aging skin or skin scars. Methods comprise administering a therapeutically effective amount of a NELL1 peptide or a nucleic acid molecule encoding the same to a subject in need thereof whereby at least one manifestation of the skin condition is prevented or treated. In some embodiments, the skin condition is aged skin. In other embodiments, the skin condition is skin scarring.

Methods are also provided for assaying a test peptide for NELL1 activity, such as the ability to reduce the expression level of inflammatory mediators and matrix metalloproteinases, the ability to increase the level of aquaporins, and the ability to reduce the number of sunburned cells.

### 1. Structural Components of Human Skin

### 1.1. Skin

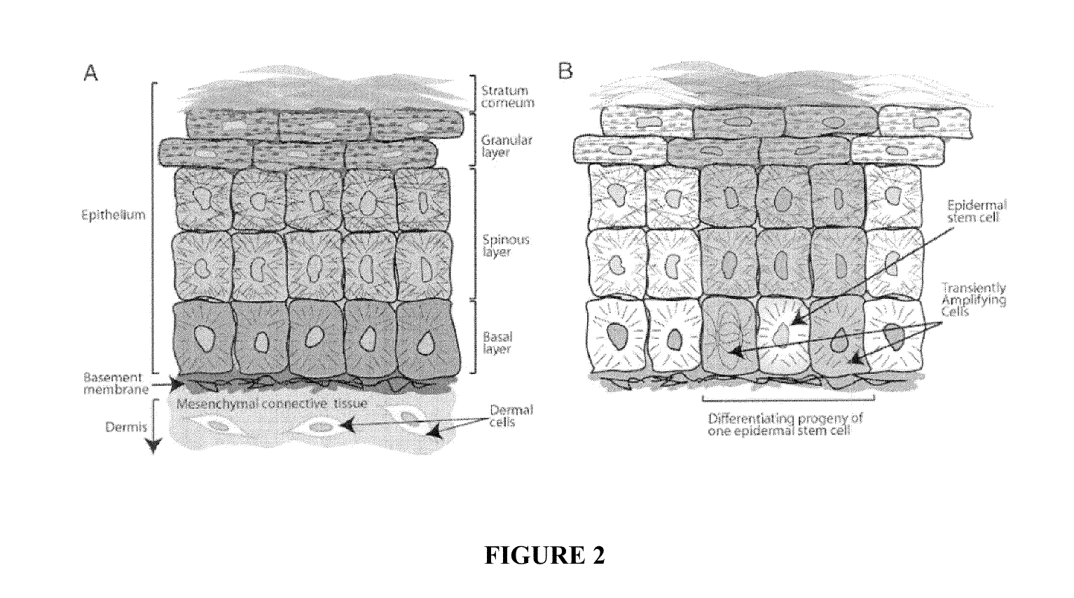

The skin (“cutis”), the outer covering of the body, is comprised of the epidermis (a stratified epithelial layer derived from the ectoderm) and the dermis (“corium”, “cutis veria”) (a connective tissue derivative of the mesoderm). As shown in FIG. 2A, beneath the dermis is the subcutaneous tissue (“superficial fascia”) (a layer of loose connective tissue), which attaches the skin to the underlying organs. Regions of the subcutaneous tissue with high levels of fat are called the “subcutaneous adipose tissue.” The subcutaneous tissue provides mobility for the skin.

The defining component of the skin is the epidermis, which is a multilayered (“stratified”) epithelium composed largely of keratinocytes that synthesize keratins that give the epidermis its toughness. The outer, horny layer of the epidermis is the stratum corneum, which consists of several layers of flat keratinized nonnucleated cells. The cell envelopes of the cells in the stratum corneum tend to be mainly polar lipids, such as ceramides, sterols, and fatty acids, while the cytoplasm of stratum corneum cells remains polar and aqueous. Despite the close packing of the cells, about 15% of the stratum corneum is intercellular and, generally lipid-based. The lipid-rich intercellular space in the stratum corneum comprises lamellar matrices with alternating hydrophilic layers and lipophilic bilayers formed during the process of keratinization.

The dermis comprises a fibrous protein matrix embedded in an amorphous, colloidal, ground substance. It supports and interacts with the epidermis, facilitating its conformation to underlying muscles and bones. Blood vessels, lymphatics, and nerves are found within the dermis.

In humans, the usual thickness of the skin is from 1-2 mm, although there is considerable variation in different parts of the body. The relative proportions of the epidermis and dermis also vary, and a thick skin is found in regions where there is a thickening of either or both layers. For example, on the interscapular (between the shoulder blades) region of the back, where the dermis is particularly thick, the skin may be more than 5 mm thick, whereas on the eyelids it may be less than 0.5 mm. Generally, the skin is thicker on the dorsal or extensor surfaces of the body than on the ventral or flexor surfaces; however, this is not the case for the hands and feet. The skin of the palms and soles is thicker than on any dorsal surface except the intrascapular region. The palms and soles have a characteristically thickened epidermis, in addition to a thick dermis.

The entire skin surface is traversed by numerous fine furrows, which run in definite directions and cross each other to bound small rhomboid or rectangular fields. These furrows correspond to similar ones on the surface of the dermis so that, in section, the boundary line between epidermis and dermis appears wavy. On the thick skin of the palms and soles, the fields form long, narrow ridges separated by parallel coursing furrows, and in the fingertips these ridges are arranged in the complicated loops, whorls (verticil) and spirals that give the fingerprints characteristic for each individual. These ridges are more prominent in those regions where the epidermis is thickest.

Where there is an epidermal ridge externally there is a corresponding narrower projection, called a “rete peg,” on the dermal surface. Dermal papillae on either side of each rete peg project irregularly into the epidermis. In the palms and soles, and other sensitive parts of the skin, the dermal papillae are numerous, tall and often branched, and vary in height (from 0.05 mm to 0.2 mm). Where mechanical demands are slight and the epidermis is thinner, such as on the abdomen and face, the papillae are low and fewer in number.

1.1.1. Epidermis

The stratified squamous epithelium of the epidermis contains four distinct cell types: keratinocytes, melanocytes, Langerhans cells, and Merkel cells. Keratinocytes and Merkel cells develop from embryonic ectoderm while melanocytes and Langerhans cells originate elsewhere and secondarily take up residence in the epidermis.

Keratinocytes (“basal cells” or “basal keratinocytes”) are the major constituent of the epidermis and continually are replaced by mitotic proliferation in the basal layers of the epithelium. When mitosis of a keratinocyte occurs, one daughter cell moves upward and begins to differentiate, while the other remains undifferentiated. FIG. 2B shows a diagram of the epidermal proliferative unit. Differentiation involves the accumulation of keratin, secretion of a membrane-coating material, the loss of nucleus and cytoplasmic organelles, and a change in shape from cuboidal to squamous. This process of transformation gives rise to a series of morphologically identifiable strata representing phases in the turnover of keratinocytes. The turnover time varies from a few weeks to several months, depending upon the region of the body. This process is greatly accelerated after injury.

FIG. 2A shows a diagrammatic representation of skin epithelial histology and the five different strata of the epidermis, moving from deeper layers to more superficial layers: (1) the stratum basale (“basal layer”, “stratum cylindricum”); (2) stratum spinosum (“prickel-cell layer”, “spinous layer”); (3) stratum granulosum (“granular layer”); (4) stratum lucidum; and (5) stratum corneum. The stratum basale and stratum spinosum together compose the stratum Malpighii.

The stratum basale is comprised primarily of columnar or high cuboidal keratinocytes arranged in a single layer, which rests on a well-defined basement membrane. The lamina reticularis portion of this basement membrane is relatively thin in mammals but thicker and more complex in some lower animals. The border of the cell and its underlying basal lamina follow an irregular course. Thin strands of connective tissue penetrate spaces between the infoldings of the cell membrane, and hemidesmosomes (cell junction structures that aid attachment of the basal surface of the epithelial cells to the basal lamina) are frequent along the base of the cells. Adherence between the epithelium and its underlying connective tissue is aided by the irregularity of the boundary between the two tissues and by the hemidesmosomes.

The stratum Malpighii is a layer of the skin composed of both the stratum basale and the stratum spinosum where the nuclei of the keratinocytes are deeply chromatic (meaning that staining of these cells indicates active protein synthesis). The shape of the nuclei varies with the shape of the cells (for example, ovoid nuclei are observed in the stratum basale and round nuclei in the stratum spinosum). The cytoplasm of the cells contains numerous intermediate filaments, and bundles of filaments are distributed throughout the cytoplasm. These bundles consistently are found in the cytoplasm adjacent to the desmosomes, often coursing toward these points of cell adhesion. The epidermal filaments of the deeper cells subsequently mature to become the fibrous elements of the filament-matrix complex of the stratum corneum.

The keratinocytes of the stratum Malpighii also contain variable numbers of melanin pigment granules. These melanin granules are not produced by keratinocytes, but are formed in melanocytes and transferred secondarily to keratinocytes.

Melanocytes (cells capable of producing the pigment melanin) differentiate from melanoblasts, which are of neural crest origin, and migrate to their definitive position at the dermoepidermal junction during embryonic development. Subsequent differentiation involves a change in cell shape (from round to stellate) and the formation of melanin granules. The process of melanin granule formation begins when membrane-bounded, lamellar bodies (“premelanosomes”) form. The premelanosomes accumulate the proenzyme (protyrosinase), which initiates melanin synthesis when activated by tyrosinase. Once tyrosinase is formed and melanin synthesis begins, the membrane-bounded bodies are called “melanosomes.” As melanin pigment accumulates in melanosomes, the melanosomes are transformed into mature melanin granules. The differentiation of melanosomes is accompanied by a change in position in the cell. The premelanosomes appear in the region of the Golgi complex, the melanosomes appear in the basal portions of the long dendritic processes, and the mature melanin granules are mainly in the peripheral portions of the dendritic processes. The melanocyte processes insinuate between the keratinocytes of the stratum Malpighii where transfer of the melanin granules occurs. Transfer occurs by a process of endocytosis involving entire tips of the dendritic processes of melanoncytes.

The cell bodies of melanocytes usually are confined to the basal layer of the epidermis, near their place of origin from primitive melanoblasts; however, melanocyte processes extend for some distance between epidermal cells.

Langerhans cells also are present in the stratum Malpighii. These cells are the dendritic cells (meaning immune cells that process antigen material and present it on the surface to other cells of the immune system) of the epidermis. Upon infection of the skin, local Langerhans cells will process antigens to become fully-functional antigen-presenting cells.

Merkel cells are oval receptor cells that lie basally and have synaptic contacts with somatosensory afferent nerves. Merkel cells may be found in the rete ridges, and often are associated with sensory nerve endings. Merkel cells are associated with the sense of light touch discrimination of shapes and textures.

The stratum spinosum (“prickel-cell layer”, “spinous layer”) is composed of keratinocytes of polygonal shape and is the layer where keratinization begins. These keratinocytes contain small membrane-coating granules. It generally is believed that the contents of these granules are secreted into the intercellular space where they contribute to a thickening of the cell membrane that occurs by the time the cells reach the stratum lucidum.

The stratum granulosum comprises two to five rows of flattened, rhombic cells with their long axes parallel with the surface of the skin. The cytoplasm of the cells contains numerous keratohyalin granules that are in close association with bundles of filaments and serve as precursors of the amorphous portion of the extensive filament-matrix complex of stratum corneum.

The stratum lucidum is a thin zone located between the stratum granulosum and the cornified surface layer. The lucidum is seen readily in the epidermis of the palms and soles but usually is not identifiable in other parts of the body. The nuclei begin to degenerate in the outer cells of the granulosa layer and disappear in the lucidum.

The stratum corneum is the outer layer of the epidermis. It is composed of clear, dead, scale-like cells, which become more and more flattened as the surface is approached. The most peripheral layer contains flat, horny plates which constantly are desquamated. The cells have a thickened membrane and are closely interdigitated. The nuclei have disappeared, but some of the spaces that they occupied can be seen. The former cytosol of these dead cells is significantly shrunken and almost completely occupied by filaments tightly packed in orthogonal arrays that lie parallel to the skin surface and embedded in an opaque interfilamentous material. Modified desmosomes may assist in spatial stabilization. The stratum corneum is very thick in the palms of the hands and soles of the feet.

A number of changes occur during the keratinization process as the cells move outward from the stratum basale to the cornified layer, including aggregation of filaments, formation of keratohyalin granule precursors of the interfilamentous matrix material of keratin, and the loss of cell organelles after the keratohyalin granules have reached their maximal size. The emergence of the keratin complex is characterized by formation of disulfide groups in keratin from sulfhydryl groups in the filaments of the deeper layers.

The thickened membranes that envelop the keratinized cells are resistant to keratinolytic agents and provide integrity for the filament-matrix complex. The filamentous portion of the complex provides flexibility and elastic recovery of the cell content. The amorphous portion of the filament-matrix complex is primarily responsible for the chemical resistance of the keratinized cells.

The cornified layer of the epidermis is composed of “soft keratin” (as contrasted with the “hard keratin” found in the nails and the cortex of hairs). Hard keratin contains relatively more sulfur, is less elastic, and does not desquamate, as does the epidermis.

The term “stratum disjunction” refers to the peripheral region of the stratum corneum which is constantly being desquamated.

1.1.1.1. Epidermis of the General Body Surface

The epidermis of most of the body is considerably thinner than that of the palms, soles and volar surfaces of the digits. The structure of the epidermis varies from region to region of the body, with all layers of the epidermis reduced, and the stratum corneum and stratum Malpighii being the only layers that are constantly present. A thin stratum granulosum, composed of only one or two cell rows, frequently is present, but a definite stratum lucidum generally is absent. For example, on the leg, where the epidermis is thicker than that of abdominal or pubic skin yet much thinner than that of the fingertip, a faint stratum lucidum may be found.

1.1.2. Dermis (“Corium”)

The dermis is of variable thickness (from 0.2 mm to 0.4 mm) and is composed of dense, irregularly arranged connective tissue. It contains three types of connective tissue fibers, plus fibroblasts and macrophages. Two layers can be distinguished, although they blend without distinct demarcation; the deeper layer (reticular layer) is relatively thick while the superficial (subepithelial or papillary) layer is thinner.

The reticular layer is characterized by coarse collagenous fibers and fiber bundles which often unite to form secondary bundles of considerable thickness (nearly 100 μm in diameter). The fibers cross each other to form an extensive feltwork with rhomboid meshes, the direction of the fibers generally being parallel to the surface of the skin. The elastic fibers form complex elastic nets permeating the entire dermis. The course of the main fibers is parallel with the surface, although vertical and oblique fibers are present in substantial number. The elastic fibers form basket-like, capsular condensations around the hair-bulbs, sweat glands, and sebaceous glands.

The papillary layer is similar in structure to the reticular layer, but the fibers are finer and more closely arranged.

Although the connective tissue fibers of the dermis form complex nets and meshes, those bundles which course parallel with the lines of tension of the skin are more numerous and developed than the others. The lines of skin tension (“Langer's lines”) are caused by the direction of the predominant fibers. These lines have different directions in the various parts of the body.

The surface of the dermis is studded with numerous papillae that indent the underside of the epidermis. Some contain loops of capillary blood vessels (vascular papillae) and others contain special nerve terminations (nervous papillae).

Smooth muscle is found in the skin in connection with skin hair and other areas of the skin. Smooth muscle fibers are arranged in a network parallel to the surface, and contraction of the fibers gives the skin of these regions its wrinkled appearance. For example, in the face and neck, skeletal muscle fibers from the mimic musculature (meaning the facial muscles by which emotions and intelligence are expressed) likewise penetrate the dermis. Both smooth and skeletal fibers end in delicate, elastic bands that are continuous with the general elastic network of the dermis.

1.1.3. Glands and Hair of the Skin

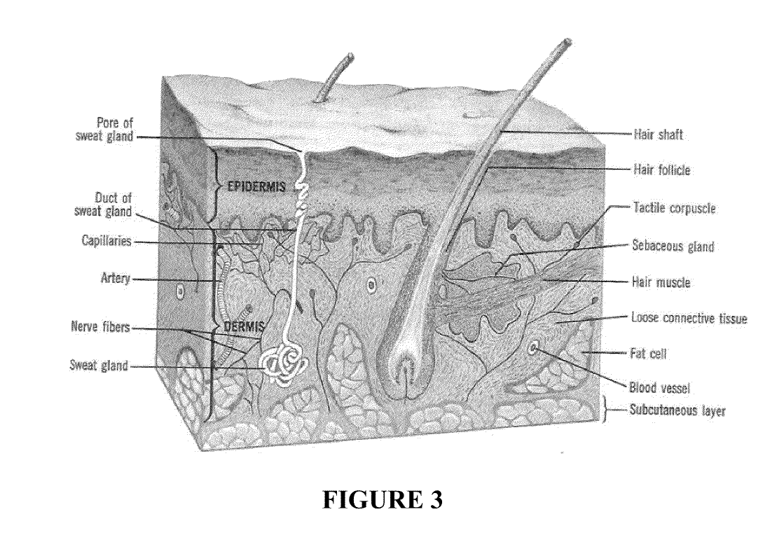

FIG. 3 shows a diagram of the anatomy of the skin.

There are three kinds of glands in the skin: sweat glands, sebaceous glands, and mammary glands. Most of the sweat glands are of the eccrine (merocrine) type, i.e., the product of the cell is secreted by exocytosis. Eccrine sweat glands are found over the entire body surface, excepting the margin of the lips, the eardrum, the inner surface of the prepuce, and the glans penis. The main components of sweat released by the secretory tubule are water, sodium chloride, urea, ammonia, uric acid and proteoglycans. The clear cells apparently function in releasing water, sodium chloride, urea, ammonia, and uric acid while dark cells secrete proteoglycans.

Sebaceous glands usually are associated with hair follicles. Hairs are elastic, cornified threads developed from the epidermis, which grows from hair follicles deep in the dermis and projects from the epidermis of the skin. Each hair comprises two structures: the follicle in the skin and the shaft that projects above the surface.

The hair follicle contains several layers. At the base of the follicle is a projection called a papilla, which contains capillaries, or tiny blood vessels, that feed the cells. The living part of the hair, the area surrounding the papilla called the bulb, is the only part fed by the capillaries. The cells in the bulb divide every 23 to 72 hours, faster than any other cells in the body. The follicle is surrounded by two sheaths: an inner root sheath (“IRS”) and an outer root sheath (“ORS”). These sheaths protect and mold the growing hair shaft. The IRS follows the hair shaft and ends below the opening of a sebaceous (oil) gland, which produces sebum, a natural conditioner and sometimes an apocrine (scent) gland. The ORS continues all the way up to the sebaceous gland.

The rapidly proliferating cells of the bulb differentiate to populate all of the layers of the IRS and the hair shaft itself. The hair follicle bulge resides within the ORS in a small niche just below the sebaceous gland, at or near the site of insertion of the arrector pili muscle. The bulge is below the surface of the skin, protected by a column of cells in the upper portion of the hair follicle, as well as by the heavily keratinized hair shaft itself. Across the basement membrane, it is surrounded by a supportive dermal pocket that is richly vascularized and innervated. Although there are no known specific markers of epidermal stem cells, these putative stem cells can be identified either in vivo (with pulse-chase labeling of slow cycling cells), or in vitro (by clonogenicity). Neither method of identification provides for easy isolation of stem cells for analysis. Accordingly, the transcriptional and functional characteristics of these cells have not yet been determined

An erector pili muscle attaches below the sebaceous gland to a fibrous layer around the outer sheath. When this muscle contracts, it causes the hair to stand up.

1.1.4. Functions of Skin

The skin performs many functions including those consistent with the barrier function of the skin, including, but not limited to, protection, regulation of heat loss, excretion, sensory function, secretory function and nutritional functions.

The barrier function, which protects internal organs from the environment, resides in the stratum corneum of the skin. The homeostatic function of the skin barrier is a self-referential system that constantly monitors its original function, i.e., water impermeability. When the stratum corneum barrier function is damaged, a series of homeostatic processes in the barrier function is immediately accelerated and the barrier recovers to its original level. This process includes lipid synthesis, lipid processing and the acceleration of exocytosis of lamellar bodies. The skin barrier function also has an ability to adapt to the environment. For example, under a low humidity environmnent, the barrier function is enhanced. The content of the intercellular lipid in the stratum corneum increases and consequently, the transepidermal water loss decreases, i.e., the water impermeability increases. In the nucleated layer of the epidermis, the number of lipid-containing lamellar bodies increases and the recovery rate after barrier disruption increases.

The skin protects the underlying tissues against mechanical injury, microbial infection, excessive loss of moisture, chemical insult, extreme temperature changes in the external environment and, overexposure to ultraviolet radiation (sunlight).

The skin also provides for temperature regulation. The skin contains a blood supply far greater than its requirements which allows precise control of energy loss by radiation, convection and conduction. The diameters of the arteries and capillaries in the skin (and therefore the volume of blood flowing through the skin) are controlled by the autonomic nervous system according to the needs of the body. Dilated blood vessels increase perfusion and heat loss while constricted vessels greatly reduce cutaneous blood flow and conserve heat. Increased perspiration by the sweat glands promotes greater heat loss or cooling by evaporation.

The excretory function of the skin supplements the role of the kidneys. Sweat contains a large content of water (about 99%) as well as a variety of other substances including urea, inorganic salts, uric acid, ammonia and creatinine

The skin further serves in a sensory capacity. The skin contains numerous nerve endings or receptors concerned with the senses of touch, pressure, temperature and pain, providing information of the immediate external environment.

The skin also secretes oils to prevent the skin from drying out and cracking, to protect against excessive ultraviolet irradiation from the sun, and to help maintain hair, and provides an acid mantle (a very fine film on the surface of the skin with pH 4.2-5.6) produced from the secretions from the sebaceous and eccrine glands, to inhibit microbial colonization. Further, the mammary glands secrete milk.

The skin also serves a nutritional role. The skin contains the steroid 7-dehydrocholesterol, which is transformed to vitamin D upon exposure to ultraviolet light.

### 1.2. Skin Aging

In some embodiments of the presently disclosed methods, a NELL1 peptide or a nucleic acid molecule encoding the same is administered to a subject in need thereof to treat or prevent skin aging. In these embodiments, at least one manifestation of aged skin is treated or prevented. The term “manifestation” as used herein refers to an outward or perceptible indication. Any of the manifestations of skin aging described hereinbelow can be treated or prevented through the administration of a NELL1 peptide or a nucleic acid molecule encoding the same. In some embodiments, the at least one manifestation of aged skin that is treated or prevented with the NELL1 peptide or nucleic acid molecule is selected from the group consisting of skin dryness, skin roughness, a rhytide, a pigmented lesion, redness, an ephelide, a lentigine, patchy hyperpigmentation, a depigmented lesion, a guttate hypomelanosis, skin fragility, an area of purpura, a benign lesion, an acorchordon, a senile angioma, a seborrheic keratosis, a lentigo, a sebaceous hyperplasia, or a combination thereof. In other embodiments, the manifestation of skin aging that is treated or prevented with a NELL1 peptide or nucleic acid molecule is inflammation and the NELL1 peptide or nucleic acid molecule reduces the levels of inflammatory mediators, including but not limited to, a reduction in inflammatory cytokines (e.g., IL-8, IL-1β), when compared to an untreated control. The NELL1 peptide or nucleic acid molecule can also reduce the levels of matrix metalloproteinases (e.g., MMP1). The term “reduced” or “to reduce” as used herein refers to a diminishing, a decrease in, an attenuation or abatement of the degree, intensity, extent, size, amount, density or number of.

The administration of a NELL1 peptide or nucleic acid molecule can treat or prevent skin dryness through, for example, increasing the levels of aquaporins (AQPs) in the skin.

Skin aging is a process in which both intrinsic and extrinsic determinants lead progressively to a loss of structural integrity and physiological function. Intrinsic aging of the skin occurs as a natural consequence of physiological changes over time at variable genetically determined rates. Extrinsic factors are, to varying degrees, controllable and include exposure to sunlight, pollution, nicotine, repetitive muscle movements, such as squinting or frowning, and miscellaneous lifestyle components, such as diet, sleeping position and overall health.

The synergistic effects of intrinsic and extrinsic aging factors over the human lifespan produce deterioration of the cutaneous barrier with significant associated morbidity. Aging skin is at risk of breakdown and ultimately failure. “Acute skin failure” refers to a state of total dysfunction resulting from both different dermatological conditions as well as internal body responses characterized by a loss of normal temperature control with an inability to maintain core body temperature and failure to prevent percutaneous loss of fluid, electrolytes and protein with a resulting imbalance and failure of the barrier to prevent penetration of foreign substances, infection, peripheral edema and altered immune functioning.

1.2.1. Intrinsic and Extrinsic Skin Aging Factors

Intrinsically aged skin is smooth and unblemished, and characterized by normal geometric patterns, with some exaggerated expression lines. Histologically, such skin manifests epidermal and dermal atrophy, flattening of the epidermal rete ridges, as well as reduced numbers of fibroblasts and mast cells. Collagens are found in the extracellular matrix and basement membranes of nearly all tissues, where their primary role is to provide a supportive extracellular framework for cells. In intrinsically aged skin, increases are seen in the number of collagen fibrils as well as the ratio of collagen III to collagen I.

Intrinsic skin aging also may involve the telomeres (regions of protective repetitive DNA) that are found at the ends of eukaryotic chromosomes. Intact telomeres are integral to extending the lifespan of cells. With age, telomere length shortens. The epidermis is one of the few regenerative tissues to express telomerase (a cellular reverse transcriptase enzyme that stabilizes or lengthens telomeres). Some studies suggest that telomere shortening associated with aging is characterized by tissue-specific loss rates and that the natural, progressive shortening of telomeres may be one of the primary mechanisms of cellular aging in skin.

Extrinsic aging is largely preventable. Factors with clearly exogenous origins include smoking, poor nutrition and solar exposure. Most extrinsically aged skin results from the cumulative effects of life-long ultraviolet radiation exposure, and commonly occurs at exposed areas of the skin, typically the face, chest and extensor surfaces of the arms. Skin damage results from ultraviolet (UV) light exposure through several mechanisms, including the formation of sunburned cells, as well as formation of thymine and pyrimidine dimers, collagenase production, and the induction of an inflammatory response. The clinical presentation of photo-aged skin includes rhytides (wrinkles), pigmented lesions such as ephelides (freckles), lentigines (brown spots), patchy hyperpigmentation, and depigmented lesions, such as guttate hypomelanosis (skin lesions that occur chiefly on the forearms). Losses in tone also are observed in photo-aged skin, along with skin fragility, areas of purpura (red or purple disclorations) due to blood vessel weakness, and benign lesions such as acrochordons (skin tags or polyps), keratoses and telangiectases (small dilated blood vessels).

Additionally, photo-aged skin is characterized by epidermal atrophy, distinct alteration in collagen and elastic fibers, and exhibition of fragmented, thickened and more soluble collagen fibers. Elastic fibers also experience fragmentation and may exhibit progressive cross-linkage and calcification.

Overall, aging skin is marked by increased inelasticity, fragmentation and collagen bundle fragility. Additionally, aged skin accumulates senescence markers (such as the cyclin-dependent kinase 4 and 6 inhibitor p16^{INK4A}). Studies indicate that there is a near-complete loss of dendritic epidermal T cells (DETCs) as skin ages, which may indicate impaired innate immunity.

Multiple aspects of skin health have been associated with the ECM. It is widely believed that loss of ECM integrity (vastly due to the decrease in collagen production) with age contributes to poorer water retention in the skin, loss of elasticity and plumpness, formation of wrinkles and sagging. Photo-aged skin is characterized by epidermal atrophy, disorganized collagen fibers, elastic fiber fragmentation and loss of elasticity. The composition of aged/damaged skin changes with respect to young skin such that in addition to poor organization and ECM destruction, aged skin has a different ratio of ECM components along with deficiency in the expression of certain species.

Hydration of the skin is another aspect of skin health that is compromised in aged or damaged skin (Almond (2007) Cell Mol Life Sci 64(13):1591-1596, which is herein incorporated by reference in its entirety). A major role in maintaining the water retention of skin is played by hyaluronic acid (HA), which binds water molecules and contributes to the plump young look of skin. Another class of molecules implicated in hydration are the aquaporins (AQPs). AQPs are integral membrane proteins that facilitate the transport of water, glycerol and other small uncharged species and contribute to the hydration, biosynthetic functions and wound healing of tissues. Aquaporin proteins are comprised of six transmembrane alpha-helices arranged in a right-handed bundle, with intracellular amino and carboxyl termini (Gonen and Walz (2006) Q Rev Biophys 39(4):361-396; Fu and Lu (2007) Mol Membr Biol (24)5-6:366-374). Although aquaporins associate as tetramers in the cell membrane, each monomer can function as a water channel.

Of interest in skin is AQP3, the deficiency of which causes reduced skin water content and elasticity as a result of impaired glycerol transport in AQP3 knockout mice (Hara et al. (2002) J Biol Chem 277(48):46616-46621; Hara-Chikuma and Verkman (2008) J Invest Dermatol 128:2145-2151; each of which is herein incorporated by reference in its entirety). There are thirteen known aquaporins in mammals, but the most well-studied are AQP1, AQP2, AQP3, and AQP4. In some embodiments of the presently disclosed methods, the administration of a NELL 1 peptide or nucleic acid molecule results in an increase in the level of an aquaporin (e.g., aquaporin 3) by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or greater when compared to an untreated control sample or subject. The nucleotide sequence of human AQP3 is set forth GenBank Acc. No. NM_{—}004925 and in SEQ ID NO: 46 (the amino acid sequence is set forth in SEQ ID NO: 47). The increase in the levels of an aquaporin can be detected using methods known in the art to detect the transcript (e.g., quantitative polymerase chain reaction) or the protein directly (e.g., immunoblot, enyzyme-linked immunoassay) or indirectly by measuring the activity of the protein (e.g., water or glycerol transport).

1.2.2. Characteristics of Aging Skin: Epidermis

The epidermis manifests several age-related changes. In aged skin, the intersection of the epidermis and dermis (the dermal-epidermal junction (“DEJ”) is altered. Aged epidermis manifests a flattened DEJ with a correspondingly diminished connecting surface area. It generally is believed that such a loss of DEJ surface area may contribute to the increased fragility of the skin associated with age and may lead to reduced nutrient transfer between the dermal and epidermal layers.

1.2.2.1. Decreased Cell Turnover

The epidermal turnover rate slows as individuals age. The lengthening of the cell cycle in older adults coincides with a protracted stratum corneum replacement rate, epidermal atrophy, slower wound healing and often less desquamation. Studies have indicated that older adults require nearly double the time to re-epithelialize after dermabrasion in comparison to younger adults. The cascade of changes related to this slowed cell turnover results in the development of heaps of corneocytes that render the skin surface rough and dull in appearance.

1.2.3. Characteristics of Aging Skin: Dermis

As individuals age, the dermis becomes thinner, acellular and avascular. Aged dermis further is characterized by changes in collagen production, and the development of fragmented elastic fibers. Photo-aged dermis exhibits disorganized collagen fibers and the accumulation of abnormal elastin-containing material. Subsequently, the collagen, elastin and glycosaminoglycan components of the dermis may be affected.

1.2.3.1. Collagen

The cutaneous signs of aging appear as the structural proteins and main components of the skin deteriorate over time.

Collagen, the primary structural component of the dermis and the most abundant protein found in humans, accounting for 70% of the dry skin mass, is responsible for conferring strength and support to human skin. The term “collagen” as used herein refers to the main protein of connective tissue in animals. Type I collagen is ubiquitous in all vertebrates, and is among the largest and most complex of all macromolecules. It is synthesized as a soluble, procollagen form containing two α1, and one α2 chains, each of about 1000 amino acids. Upon secretion from the cell the propeptides are cleaved, and the collagen monomer is assembled into the fibril, proposed to consist of aggregates of microfibrils, or 5-mer bundles of overlapping monomers. Type I collagen fibrils contribute to the integrity and function of many tissues via interactions with other collagens, peptidoglycans (PGs), and growth and differentiation factors. Type III collagen is the second most abundant collagen in human tissues and occurs particularly in tissues exhibiting elastic properties, such as skin, blood vessels, and various internal organs. It is a homotrimer composed of three α1 (III) chains and resembles other fibrillar collagens in its structure and function. It is synthesized as a procollagen similarly to Type I collagen, but the N-terminal propeptide remains attached in the mature, fibrillar type III collagen more often than it does in type I.

In aged skin, collagen is characterized by thickened fibrils, organized in rope-like bundles, which appear to be in disarray in comparison to the pattern observed in younger skin. In addition, lower levels of collagen are synthesized by aged fibroblasts. The ratio of collagen types found in skin also changes with age. In young skin, collagen I comprises 80% and collagen III comprises about 15% of total skin collagen; in older skin, the ratio of Type III to Type I collagen has been shown to increase due, significantly, to an appreciable loss of collagen I. Furthermore, the overall collagen content per unit area of skin surface is known to decline approximately 1% per year. In irradiated skin, collagen I levels are reduced by about 60% and shown to be linked to the extent of photodamage. Although collagen I is the most abundant and significant collagen type found in the skin, the effects of aging are seen in other types of collagen in human dermis.

Collagen IV is an integral component of the DEJ that provides a structural framework for other molecules and for maintenance of mechanical stability. Studies have shown no significant differences between collagen IV levels found in sun-exposed skin compared to unexposed skin, but significantly lower levels of collagen IV have been identified at the base of wrinkles in comparison to the flanks of the same wrinkles The mechanical stability of the DEJ may be adversely affected by this loss of collagen IV, thereby contributing to wrinkle formation.

Collagen VII is the primary constituent in anchoring fibrils that attach the basement membrane zone to the underlying papillary dermis. Studies have shown a significantly lower number of anchoring fibrils in those individuals with chronically sun-exposed skin in comparison to those unexposed. Additional studies have shown that there is a marked loss of collagen VII at the base of the wrinkle, similar to that observed with collagen IV.

1.2.3.1.1. Pathogenesis of Ultraviolet Radiation-Induced Collagen Damage

It is known that ultraviolet radiation (“UVR”) exposure significantly upregulates the synthesis of several types of collagen-degrading enzymes (matrix metalloproteinases (“MMPs”)). First, UV exposure leads to an increase in the amount of the transcription factor c-jun; the transcription factor c-fos is abundant without UV exposure. This initiates a cascade effect where (i) the transcription factors c-jun and c-fos then combine to form activator protein-1 (“AP-1”); (ii) AP-1 then activates the MMP genes; which (iii) stimulate the production of collagenase, gelatinase and stromelysin. Subsequently, collagen degradation is mediated by AP-1 activation and by inhibition of transforming growth factor (“TGF”) β signaling. Studies have shown that the MMPs collagenase and gelatinase are produced upon exposure to ultraviolet B (“UVB”) light and that multiple exposures to UVB provide a sustained induction of MMPs. This long-term elevation in the levels of MMPs may contribute to the disorganized and clumped collagen identified in photo-aged skin and may further provide the mechanism by which collagen I levels decline in response to UV exposure. MMP1, or interstitial collagenase, is one of the major degradative enzymes induced by UV radiation in vivo (Brenneisen et al. (2002) Ann NY Acad Sci 973:31-43) In some embodiments of the presently disclosed methods wherein at least one manifestation of a skin condition (e.g., aging skin) is treated or prevented by a NELL1 peptide or a nucleic acid molecule encoding the same, at least one manifestation is elevated MMP levels and administration of the NELL1 peptide or nucleic acid molecule reduces MMP levels. In some of these embodiments, the MMP is matrix metalloproteinase 1 (MMP1). The nucleotide sequence of human MMP1 is set forth in GenBank Acc. No. NM_{—}002421 and in SEQ ID NO: 44 (the amino acid sequence is set forth in SEQ ID NO: 45). In some embodiments, the NELL1 peptide or nucleic acid molecule reduces MMP (e.g., MMP1) levels by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or greater when compared to an untreated control sample or subject. The reduction in the levels of MMP can be detected using methods known in the art to detect the transcript (e.g., quantitative polymerase chain reaction) or the protein directly (e.g., immunoblot, enzyme-linked immunoassay) or indirectly by measuring the activity of the protein (e.g., degradation of extracellular matrix components).

1.2.3.2. Elastin

Elastosis (accumulation of amorphous elastin material) is characteristic of photo-aged skin. UV exposure induces a thickening and coiling of elastic fibers in the papillary dermis and, with chronic UV exposure, in the reticular dermis. UV-exposed skin manifests a reduction in the number of microfibrils and increases in interfibrillar areas.

The initial response of elastic fibers to photodamage is hyperplastic (meaning an abnormal multiplication of cells), resulting in a greater amount of elastic tissue. The level of sun exposure determines the magnitude of the hyperplastic response. In aged elastic fibers, a secondary response to photodamage occurs but is degenerative, with a decrease observed in skin elasticity and resiliency. Subsequently, aged skin is characterized by changes in the normal pattern of immature elastic fibers (oxytalan) located in the papillary dermis. These fibers form a network in young skin that ascends perpendicularly from the uppermost section of the papillary dermis to just beneath the basement membrane; however, this network gradually disappears with age. Consequently, skin elasticity also decreases with age.

1.2.3.3. Glycosaminoglycans

Glycosaminoglycans (“GAGs”) are responsible for conferring the outward appearance of the skin. These polysaccharide chains, with repeating disaccharide units attached to a core protein, have the capacity to bind water up to 1000 times their volume, and render normal skin plump, soft and hydrated, and are believed to assist in maintaning proper salt and water balance. Members of the GAG family include hyaluronic acid (“HA”), dermatan sulphate and chondroitin sulphate. As skin ages intrinsically, the total dermal HA level remains stable; however, epidermal HA diminishes almost completely.

1.2.3.3.1. Hyaluronic Acid

Reduced levels of HA and elevated levels of chondroitin sulfate proteoglycans are characteristics of photo-aged skin. HA is found in young skin at the periphery of collagen and elastin fibers, and at their intersections. In aged skin, such connections with HA disappear. These decreases in HA, which contribute to its disassociation with collagen and elastin, as well as reduced water binding, may be involved in aged skin-related changes such as wrinkling, altered elasticity, reduced turgidity, and diminished capacity to support the microvasculature of the skin.

HA can bind up to 1000 times its weight in water, and as such, may help the skin retain and maintain water. HA is found in all connective tissue and is produced mainly by fibroblasts and keratinocytes in the skin. HA is localized in the dermis and epidermal intercellular spaces (especially the middle spinous layer), but is not localized in the stratum corneum or stratum granulosum. HA does not penetrate the skin upon topical application.

1.2.4. Additional Characteristics of Aging Skin

The number of melanocytes decreases about 8-20% per decade. Accordingly, this loss of melanin (which absorbs UV radiation) may cause the skin to become more susceptible to sun-induced cancers.

Additionally, studies have shown aged skin to be avascular. This leads to a reduced blood flow, depleted nutrient exchange, inhibited thermoregulation, decreased skin surface temperature and skin pallor.

Further, site-specific changes that affect the appearance of the skin occur within the subcutaneous tissue. These changes include the diminishment of subcutaneous fat in the face, dorsal aspects of the hands and shins, and amassment of fat in the waist and abdomen.

1.2.4.1. Changes in Skin Appearance

The degradation or loss of skin barrier function may lead to a dry, scaly skin that frequently is seen in aged skin. Studies have shown that the recovery of damaged barrier function is slower in aged skin, resulting in greater susceptibility to developing dryness. This multifactorial process is due, in part, to lower lipid levels in lamellar bodies and a decrease in epidermal filaggrin (a protein of keratohyalin granules). Increased trans-epidermal water loss also is exhibited by aged skin, leaving the stratum corneum more susceptible to becoming dry in low humidity environments. In addition to dryness, aged skin often is characterized by roughness, wrinkling, skin pallor, hyperpigmentation, hypopigmenation, laxity, fragility, easy bruising and benign neoplasms. These benign neoplasms include acrochordons (skin tags), cherry angiomas (senile angiomas), seborrheic keratoses (senile wart or the “barnacles of old age”), lentigos (sun spots) and sebaceous hyperplasias.

Irregular hyperpigmenation and hypopigmentation, both discrete and limited or diffuse and irregular, may be noted and clinically represented by freckles, solar lentigines (blemishes on the skin that range in color from light brown to red or black) and hypomelanotic macules (white or light patches of skin sometimes in an ash-leaf shape).

Alternating compact and basketweave patterns of the stratum corneum and cellular heterogenetity may lead to an appearance and feel of surface roughness, dryness or scaliness, reflective of abnormalities of keratinocyte production, adhesion and separation.

Wrinkles of various depth, length, and location are a reflection of underlying dermal damage to collagen, elastin, and ground substance and their incomplete repair.

The color of photo-aged skin may be sallow (yellowish) in some instances but otherwise variable due to the irregularity of the surface and of reflected light, the variability of total skin thickness, melanin content and distribution, and the influences of saturated and unsaturated hemoglobin.

Photo-aged skin also may accumulate changes to epidermal cell DNA and result in many benign and malignant neoplasms of the skin. These include benign seborrheic keratosis (round or oval skin growths that originate in keratinocytes that appear in various colors from light tan to black), actinic keratosis (a premalignant condition of thick, scaly or crusty patches of skin) and squamous cell carcinoma.

### 2.3. Skin Aging Prevention and Treatment

In current anti-aging skin care, the prevention of wrinkle development has assumed great prominence. To prevent the formation of wrinkles, it generally is believed necessary to halt the degradation of the skin's three primary structural constituents, collagen, elastin and HA, since all three components are known to decline with age. Consequently, most existing anti-aging procedures and products are designed or formulated with the intention of salvaging at least one of these constituents. However, topical products containing collagen, elastin or HA are unable to serve as adequate replacements for aged skin since it generally is believed that the technology required to deliver these compounds into the skin does not exist. Although some products do promote the natural synthesis of these substances, no products replenish them. For example, collagen production has been shown to be stimulated by the use of retinoids, vitamin C (including oral) and copper peptide. HA levels also are thought to be augmented with glucosamine supplementation. There are no products yet approved for increasing the production of, or enhancement of elastin.

Another approach to preventing wrinkle formation is through the reduction of inflammation.

The term “inflammation” as used herein refers to the physiologic process by which vascularized tissues respond to injury. See, e.g., FUNDAMENTAL IMMUNOLOGY, 4th Ed., William E. Paul, ed. Lippincott-Raven Publishers, Philadelphia (1999) at 1051-1053, incorporated herein by reference. During the inflammatory process, cells involved in detoxification and repair are mobilized to the compromised site by inflammatory mediators. Inflammation is often characterized by a strong infiltration of leukocytes at the site of inflammation, particularly neutrophils (polymorphonuclear cells). These cells promote tissue damage by releasing toxic substances at the vascular wall or in uninjured tissue. Traditionally, inflammation has been divided into acute and chronic responses.

The term “acute inflammation” as used herein refers to the rapid, short-lived (minutes to days), relatively uniform response to acute injury characterized by accumulations of fluid, plasma proteins, and neutrophilic leukocytes. Examples of injurious agents that cause acute inflammation include, but are not limited to, pathogens (e.g., bacteria, viruses, parasites), foreign bodies from exogenous (e.g. asbestos) or endogenous (e.g., urate crystals, immune complexes), sources, and physical (e.g., burns) or chemical (e.g., caustics) agents.

The term “chronic inflammation” as used herein refers to inflammation that is of longer duration and which has a vague and indefinite termination. Chronic inflammation takes over when acute inflammation persists, either through incomplete clearance of the initial inflammatory agent or as a result of multiple acute events occurring in the same location. Chronic inflammation, which includes the influx of lymphocytes and macrophages and fibroblast growth, may result in tissue scarring at sites of prolonged or repeated inflammatory activity.

In some embodiments of the presently disclosed methods wherein a NELL1 peptide or a nucleic acid molecule encoding the same is administered to a subject in need thereof to prevent or treat at least one manifestation of a skin condition (e.g., aging skin), the at least one manifestation is inflammation. In some of these embodiments, NELL1 reduces the level of inflammatory mediators when compared to an untreated control sample or subject. As used herein, the term “inflammatory mediator” refers to immunoregulatory proteins, such as lymphokines, cytokines, interferons, and chemokines that promote inflammation, locally or systemically.

Non-limiting examples of inflammatory mediators include interleukin (IL)-1-alpha, IL-1-beta, IL-2, IL-3, IL-6, IL-7, IL-9, tumor necrosis factor (TNF)-alpha, leukemia inhibitory factor (LIF), interferon (IFN)-alpha, IFN-beta, IFN-gamma, oncostatin M (OSM), ciliary neurotrophic factor (CNTF), transforming growth factor (TGF)-beta, granulocyte macrophage colony-stimulating factor (GM-CSF), IL-11, IL-12, IL-17, IL-18, and IL-8. In particular embodiments, NELL1 administration reduces the levels of IL-1β or IL-8. In certain embodiments, the NELL1 peptide or nucleic acid molecule reduces the levels of at least one inflammatory mediator (e.g., IL-1β or IL-8) by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or greater when compared to an untreated control sample or subject. The human nucleotide sequences for IL-1β and IL-8 are set forth in GenBank Acc. Nos. NM_{—}000576 and NM_{—}000584, respectively, and in SEQ ID NOs: 40 and 42, respectively (the amino acid sequences are set forth in SEQ ID NO: 41 and 43, respectively). The reduction in the levels of an inflammatory mediator can be detected using methods known in the art to detect the transcript (e.g., quantitative polymerase chain reaction) or the protein directly (e.g., immunoblot, enyzyme-linked immunoassay) or indirectly by measuring the activity of the protein (e.g., degradation of extracellular matrix components).

Inflammation is a known contributor to the degradation of collagen, elastin and HA. Skin inflammation is a known sequela of free radicals directly acting on cytokine and growth factor receptors in dermal cells and keratinocytes. Consequently, anti-oxidants (free radical scavengers), each of which has its own unique and various properties and activities, have been used to protect the skin. Studies have shown that free radical activation of MAPK pathways stimulates collagenase production; thus it generally is believed that the use of anti-oxidants to inhibit these pathways may be beneficial in the prevention of the effects of photo-aging. Several anti-oxidants have been used in attempts to prevent and/or treat wrinkles, including coenzyme Q10, Polypodium leucotomos, silymarin, and pycnogenol.

A “free radical” is a highly chemically reactive and usually short-lived molecular fragment with one or more unpaired electrons. Because a free radical needs to extract a second electron from a neighboring molecule to pair its single electron, it often reacts with other molecules, which initiates the formation of additional free radical species in a self-propagating chain reaction. This ability to be self-propagating makes free radicals highly toxic to living organisms. Oxidative injury may lead to widespread biochemical damage within the cell. The molecular mechanisms responsible for this damage are complex. For example, free radicals may damage intracellular macromolecules, such as nucleic acids (e.g., DNA and RNA), proteins, and lipids. Free radical damage to cellular proteins may lead to loss of enzymatic function and cell death. Free radical damage to DNA may cause problems in replication or transcription, leading to cell death or uncontrolled cell growth. Free radical damage to cell membrane lipids may cause the damaged membranes to lose their ability to transport oxygen, nutrients or water to cells.

### 2. Scars

In some embodiments of the presently disclosed methods, a NELL1 peptide or a nucleic acid molecule encoding the same is administered to a subject in need thereof to treat or prevent skin scarring. In these embodiments, at least one manifestation of skin scarring is treated or prevented. Any of the manifestations of skin scarring described herein can be treated or prevented by administering a NELL1 peptide or a nucleic acid molecule encoding the same. Skin scars that can be treated or prevented with a NELL1 peptide or a nucleic acid molecule encoding the same include, but are not limited to, a widespread scar, an atrophic scar, or a raised skin scar (e.g., hypertrophic scar, keloid scar). In some embodiments, the formation of the skin scar is prevented by administering the NELL1 peptide or nucleic acid molecule encoding the same to a wounded epithelial surface.

2.1. Skin Scar Types

Skin tissue repair results in a broad spectrum of scar types, ranging from a “normal” fine line to a variety of abnormal scars, including widespread scars, atrophic scars, scar contractures, hypertrophic scars, and keloid scars.

2.1.1. Widespread (Stretched) Scars

Widespread (stretched) scars appear when the fine lines of surgical scars gradually become stretched and widened, which usually happens in the three weeks after surgery. They typically are flat, pale, soft, symptomless scars. Stretch marks (abdominal striae) after pregnancy are variants of widespread scars in which there has been injury to the dermis and subcutaneous tissues but the epidermis is unbreached. There is no elevation, thickening, or nodularity in mature widespread scars, which distinguishes them from hypertrophic scars.

2.1.2. Atrophic Scars

Atrophic scars are flat and depressed below the surrounding skin. They generally are small and round with an indented or inverted center. Atrophic scars commonly arise, for example, after acne or chickenpox.

2.1.3. Raised Skin Scars

Raised skin scars are described as hypertrophic or keloid scars.

2.1.3.1. Hypertrophic Scars

A hypertrophic scar generally is described as an overgrowth of scar tissue that remains within the boundaries of a wound. The wound boundary grows wider as more scar tissue forms. Currently, no objective diagnositc criteria have been formulated to indicate when a scar can be considered hypertrophic.

Studies have reported several epigenetic causes for hypertrophic scarring. Factors that increase or prolong wound inflammation or wound tension predispose to hypertrophic scar formation. Such factors include, but are not limited to, wound infection, prolonged healing by secondary intention, or the presence of immunologically foreign material in the wound. Hypertrophic scars begin as the result of injury to the deep dermis. The incidence of hypertrophic scars following surgery is about 40% to 70%, wherease it is higher (up to 91%) following burn injury; several reports have concluded that there is a substantially increased risk for hypertrophic scarring in burn wounds that take longer than 21 days to heal.

Hypertrophic scarring also results from dynamic mechanical skin tension acting on the healing wound. As a result of mechanical tension, scars located on certain areas of the body (for example, but not limited to, the sternum, deltoid, and upper back) frequently are hypertrophic. It generally is believed this anatomic dependency is correlated to the patterns of skin tension. Hypertrophic scars regress with time after injury, leaving behind, however, an unsightly wide gap of thinned dermis between wound edges.

Populations with higher skin melanin are known to have a higher incidence of hypertrophic scars. Hormones also influence hypertrophic scarring, particularly at the start of puberty or during pregnancy.

2.1.3.2. Keloid Scars

A keloid is a type of scar that results from an overgrowth of granulation tissue (type III collagen) that later is replaced by type I collagen at the site of a healed skin injury. Keloids appear as firm, rubbery lesions or shiny, fibrous nodules. The most common anatomical sites for keloids include the chest, shoulders, earlobes, upper arms, and cheeks. This condition presents a formidable challenge, since recurrence often is difficult to prevent despite use of multiple therapeutic interventions. Part of the reason for the absence of a definitive treatment is the incomplete understanding of the pathogenesis of keloid formation, which creates a frustrating situation for both physician and patient. Although keloid formation traditionally has been understood to result from indefinite collagen production, no single accepted hypothesis has been accepted to fully explain the pathological mechanism.

Keloid formation commonly is seen after invasive medical procedures, elective cosmesis, and mundane events, such as insect bites and trauma from scratching. Symptoms can extend beyond cosmesis. Some studies have reported pruritus in 27% of patients and pain in 19%. Additional studies have reported that keloids also may ulcerate and develop draining sinus tracts.

2.1.3.2(a) Diagnosis and Differential Diagnosis of Keloid Scars

The most immediate differential diagnosis to consider when evaluating an overgrowth of scar tissue is to distinguish a keloid from a hypertrophic scar. Phenotypically, hypertrophic scars remain within the confines of the original scar border, whereas keloids invade adjacent normal dermis. Moreover, hypertrophic scars generally arise within 4 weeks of the initial scar, grow intensely for several months, and then regress. In contrast, a considerable amount of time may elapse before a keloid and an initial scar appear. Beyond this, the keloid may proliferate indefinitely.

Histologically, both keloids and hypertrophic scars exhibit increased fibroblast density. However, only keloid formation is associated with increased fibroblast proliferation. The collagen fibers in keloids are larger, thicker, wavier, with a random orientiation, whereas those in hypertrophic scars are oriented parallel to the epidermal surface.

Biochemical markers also may be used to distinguish between keloids and hypertrophic scars. Studies have reported that the levels of alanine transferase and adenosine triphosphate are higher in keloids than in normal scar tissue and hypertrophic scars. Additional studies have reported that fibroblasts from keloids have higher levels of type I and type III collagen mRNAs than those fibroblasts isolated from hypertrophic scars.

2.1.3.2(b) Pathogenesis of Keloid Scars

Although the pathogenesis of keloid formation remains unknown, several theories have been proposed.

According to the alteration in milieu theory, it has been hypothesized that keloids result from excess scar tissue secondary to increased growth factor activity and alterations in the extracellular matrix (ECM). Studies have reported that keloid fibroblasts have an increased sensitivity to TGF-β, which normally is produced during the proliferative phase of wound healing. Studies have reported that keloid fibroblasts have a 4-fold to 5-fold increase in the level of platelet-derived growth factor receptor and speculate that this may result in a synergistic growth-stimulator effect with TGF-β. Studies also have reported that keloids often have elevated levels of fibronectin and certain proteoglycans along with decreased levels of HA, and suggest that a dysregulation of fibronectin and HA expression contributes to the fibrotic phenotype seen in keloids.

The collagen turnover theory posits that defects in collagen turnover may be involved in the mechanism of keloid formation.

Collagen is produced by fibroblasts and endothelial cells. The collagen produced by fibroblasts is normally degraded by collagenase synthesized by fibroblasts and inflammatory cells. Keloid fibroblasts have a greater capacity to proliferate because of a lower threshold to enter the S phase of mitosis, resulting in greater autonomous production of collagen. Studies have reported that although this collagen is disorganized with thicker and wavier bundles, the hallmark of the keloid structure is the ‘collagen nodules’ present at the microstructural level. Further studies have reported that the ratio of type I collagen to type III collagen is significantly increased in keloids due to alterations at the pretranscriptional and posttranscriptional levels.

Studies also have reported that collagenase inhibitors, such as α-globulins and plasminogen activator inhibitor-1, consistently are present at elevated levels in both in vitro and in vivo keloid samples, whereas levels of degradative enzymes are frequently decreased.

Based on the observation that keloid lesions are associated with particular human leukocyte antigen subtypes, it has been suggested that an inherited abnormal immune response to dermal injury may be the cause of keloid formation. Studies have reported that patterns in the serum complement, IgG, and IgM levels in patients with keloids suggest a systemic immune state genetically predisposed to keloid formation.

According to the sebum reaction theory, keloids arise from an immune reaction to sebum, i.e., dermal injury exposes the pilosebaceous unit to the systemic circulation, initiating a cell-mediated immune response in persons who retain T lymphocytes sensitive to sebum. Subsequent release of cytokines, including various interleukins and TGF-β, stimulates chemotaxis of mast cells and production of collagen by fibroblasts. The theory further proffers that as the keloid expands, further pilosebaceous units on the advancing border are disrupted, leading to further propagation.

2.2. Structured Scar Assessment

Accurate scar assessment is essential for diagnosis and for beginning, monitoring, and evaluating a therapeutic strategy for scar management. The cause and course of scar development are important. A decision to treat frequently depends upon (1) the site (anatomical location of the scar), (2) symptoms (for example, but not limited to, pain and itching), (3) severity of functional impairment (for example, but not limited to, joint mobility), and (4) stigma. The severity of scars often is judged by eye but can be assessed quantitatively with a scar assessment guide such as, for example, the Vancouver scar scale, or the Manchester scar proforma. The exact anatomical location of scars, number of scars, size per site, and a description of their margins, surface, color, and texture are recorded. From these, a score is compiled, with the lower score indicating a better scar. A standardized color photograph of the scar lesion at each consultation provides a reference to evaluate effectiveness of treatment since changes occur slowly.

The presence of a positive family history, previous abnormal scarring in the same or other anatomical sites, poor response to treatment or recurrence of scarring, specific anatomical locations (e.g., sternum), large size, prolonged inflammation, and severe symptoms are associated with abnormal scarring.

2.3. Management of Problematic Scars

Currently, problematic scars may be treated with several courses of action such as non-invasive treatment, invasive treatment, and leave-alone management.

Non-invasive options include use of compression therapy (such as pressure garments with or without gel sheeting); static and dynamic splints; acrylic casts; masks and clips; application of a variety of oils, lotions and creams; antihistamine drugs; hydrotherapy; and psychological counselling and advice. Other non-invasive options include silicon sheeting, with or without adhesive and massage therapy. However, all of these treatments are empirical, and difficult to quantify objectively, although a placebo benefit may be appreciated by patients.

Invasive treatments include surgical excision and resuture. Generally, revision should be considered only if the surgeon believes that more favorable conditions for wound healing can be provided than on the first occasion (for example, less inflammation and better technique). Intralesional corticosteroid injection is used widely but is prone to complications (for example, fat atrophy, dermal thinning, and pigment changes). Other treatments that have been advocated with variable outcomes include injections of fluoruracil, interferon gamma, and bleomycin; radiotherapy; laser therapy; and cryptosurgery.

Leave it alone management utilizes monitoring (wait and watch) to allow ongoing assessment of appearance, symptoms, and psychological impact. Some scars may be best left alone in the long term. Informed, shared decision-making with patients may help reduce inappropriate demands for treatment.

2.3.2. Management of Keloid Scars

Although multiple management options are currently available for the treatment of keloids, they can be expensive and recurrence rates remain high.

Intralesional steroids are the most effective and widely used treatment for keloids. Intralesional triamcinolone acetonide, a potent anti-inflammatory fluoridated hydrocortisone, is delivered via direct intralesional injection and often is used as the first-line therapy. Studies have reported that intralesional injection of triamcinolone acetonide led to symptomatic improvement in 72% of patients and complete flattening in 64% of lesions; however additional studies reported a 5 year recurrence rate of 50% when triamcinolone acetone was used as monotherapy. Further, adverse effects occur in approximately 50% of patients treated with triamcinolone and include subcutaneous atrophy, telangiectasia, and pigmentary changes, which generally self-resolve.

Surgical excision of keloids generally results in recurrence in lesions, with reported rates ranges ranging from 40% to 100%. Simple excision is generally believed to stimulate additional collagen synthesis, resulting in rapid regrowth and often a larger keloid.

Radiation therapy has been used to reduce the recurrence rate of keloids by directly damaging fibroblasts thereby altering collagen structure and organization. Studies have reported that radiation therapy increases the rate of apoptosis in keloid fibroblasts, returning the cell population to equilibrium. However, studies have reported acute side-effects including erythema, inflammation, edema, desquamation, ulceration, chronic changes such as changes in pigmentation, skin atrophy, and fibrosis.

Silicone gel has been used as an adjunct to keloid excision and as prophylaxis to prevent abnormal scarring following elective incisions. The mechanism of action is not understood. Silicone gel can be administered either as a topical gel or impregnated elastic sheet; however poor patient compliance remains a primary limiting factor. The patient is instructed to cover the entire lesion for at least 12 hours each day and ideally up to 24 hours per day, except when the skin is being cleaned. Studies have reported that if used correctly, silicone gel may induce more rapid healing and can be used in conjunction with excision methods to decrease recurrence rates. Adverse effects of silicone gel include occassional skin maceration, erosion, rash and pruritus.

Pressure therapy following excision also is used to manage keloids. The exact mechanism of action remains unknown. Patient compliance remains a limiting factor as patients are instructed to wear compression dressings 24 hours per day after suture removal.

Studies have reported that laser therapy is not effective in managing keloids. Some studies have combined CO₂ laser therapy with various modalities, including interferon, triamcinolone, and silicone gel, resulting in success rates similar to those observed with scapel excision, however the cost of the laser and the recurrence rate are prohibitive over its use over the scalpel.

There is no ideal therapy for treating keloids. Treatment continues to be various combinations that focus on decreasing recurrence rates.

The described invention addresses these issues and provides compositions and methods utilizing a NELL1 peptide or a nucleic acid molecule encoding the same, for treating and preventing skin aging and for scar management.

### 3. Neural Epidermal Growth-Factor-Like 1 (NELL1)

The presently disclosed methods and compositions utilize a NELL1 peptide or a nucleic acid molecule encoding the same to prevent or treat a skin condition (e.g., skin aging, skin scarring).

The terms “peptide”, “polypeptide”, and “protein” are used interchangeably herein, and refer to a sequence of subunit amino acids, amino acid analogs, or peptidomimetics. The term “peptidomimetic” as used herein refers to a small protein-like chain designed to mimic a peptide. A peptidomimetic typically arises from modification of an existing peptide in order to alter the molecule's properties.

The terms “peptide”, “polypeptide” and “protein” also apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids. The terms “polypeptide”, “peptide” and “protein” also are inclusive of modifications including, but not limited to, glycosylation, lipid attachment, sulfation, gamma-carboxylation of glutamic acid residues, hydroxylation and ADP-ribosylation. It will be appreciated, as is well known and as noted above, that polypeptides may not be entirely linear. For instance, polypeptides may be branched as a result of ubiquitination, and they may be circular, with or without branching, generally as a result of posttranslational events, including natural processing events and events brought about by human manipulation which do not occur naturally. Circular, branched and branched circular polypeptides may be synthesized by non-translation natural process and by entirely synthetic methods, as well.

The terms “amino acid residue” or “amino acid” or “residue” are used interchangeably to refer to an amino acid that is incorporated into a protein, a polypeptide, or a peptide, including, but not limited to, a naturally occurring amino acid and known analogs of natural amino acids that can function in a similar manner as naturally occurring amino acids.

The abbreviations used herein for amino acids are those abbreviations which are conventionally used: A=Ala=Alanine; R=Arg=Arginine; N=Asn=Asparagine; D=Asp=Aspartic acid; C=Cys=Cysteine; Q=Gln=Glutamine; E=Glu=Glutamic acid; G=Gly=Glycine; H=His=Histidine; I=Ile=lsoleucine; L=Leu=Leucine; K=Lys=Lysine; M=Met=Methionine; F=Phe=Phenyalanine; P=Pro=Proline; S=Ser=Serine; T=Thr=Threonine; W=Trp=Tryptophan; Y=Tyr=Tyrosine; V=Val=Valine. The amino acids may be L- or D-amino acids. An amino acid may be replaced by a synthetic amino acid which is altered so as to increase the half-life of the peptide or to increase the potency of the peptide, or to increase the bioavailability of the peptide.

The human neural epidermal growth-factor-like 1 (NEL-like 1, NELL1) gene encodes an 810-amino acid polypeptide, which trimerizes to form a mature protein of about 400 kDa involved in the regulation of cell growth and differentiation. The neural epidermal growth-factor-like (nel) gene was first detected in neural tissue from an embryonic chicken cDNA library, and its human orthologue NELL1 was discovered later in B-cells. Studies have reported the presence of NELL1 in various fetal and adult organs, including, but not limited to, the brain, kidneys, colon, thymus, lung, and small intestine.

3.1. General Structure

Generally, the arrangement of the functional domains of the 810 amino acid NELL1 protein bears resemblance to thrombospondin-1 (“THBS1”) and consists of a thrombospondin N-terminal domain (“TSPN”) and several von Willebrand factor, type C (“VWC”), and epidermal growth-factor (“EGF”) domains. The term “domain” as used herein refers to a region of a protein with a characteristic primary structure and function.

Additional studies have shown that there are at least two transcript variants encoding different isoforms. In humans, the nel-like 1 isoform 1 precursor transcript variant SEQ ID NO: 1 represents the longer transcript (set forth in GenBank Acc. No. NM_{—}006157) and encodes the longer isoform 1 SEQ ID NO: 2.

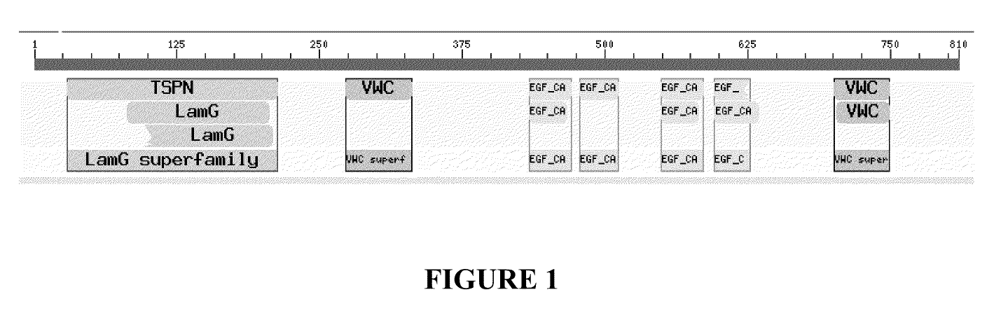

FIG. 1 shows the general structure of human nel-like 1 isoform 1 (SEQ ID NO: 2). The conserved domains reside in seven regions of the isoform 1 peptide and include: (1) a TSPN domain/Laminin G superfamily domain; (2) a VWC domain; (3) an EGF-like domain; (4) an EGF-like domain; (5) an EGF-like domain; (6) an EGF-like domain and (7) a VWC domain. NELL1 also comprises a secretion signal peptide domain (amino acid residues 1-16 of SEQ ID NO: 2) that is generally involved in transport of the protein to cell organelles where it is processed for secretion outside the cell.

The first conserved domain region comprises amino acids (amino acids 29 to 213 of SEQ ID NO: 2; set forth in SEQ ID NO: 3) that are most similar to a thrombospondin N-terminal-like domain. Thrombospondins are a family of related, adhesive glycoproteins, which are synthesized, secreted and incorporated into the extracellular matrix of a variety of cells, including alpha granules of platelets following thrombin activation and endothelial cells. They interact with a number of blood coagulation factors and anticoagulant factors, and are involved in cell adhesion, platelet aggregation, cell proliferation, angiogenesis, tumor metastasis, vascular smooth muscle growth and tissue repair. The first conserved domain also comprises amino acids (amino acids 82 to 206; amino acids 98 to 209) that are similar to a Laminin G-like domain. Laminin G-like (LamG) domains usually are Ca²⁺ mediated receptors that can have binding sites for steroids, β1-integrins, heparin, sulfatides, fibulin-1, and α-dystroglycans. Proteins that contain LamG domains serve a variety of purposes, including signal transduction via cell-surface steroid receptors, adhesion, migration and differentiation through mediation of cell adhesion molecules.

Much of what is known about NELL1 concerns its role in bone development. It is generally believed that during osteogenic differentiation, NELL1 signaling may involve an integrin-related molecule and tyrosine kinases that are triggered by NELL1 binding to a NELL1 specific receptor and a subsequent formation of an extracellular complex. As thus far understood, in human NELL1 (hNELL1), the laminin G domain comprises about 128 amino acid residues that show a high degree of similarity to the laminin G domain of extracellular matrix (“ECM”) proteins; such as human laminin α3 chain (hLAMA3), mouse laminin α3 chain (mLAMA3), human collagen 11 α3 chain (hCOLA1), and human thrombospondin-1 (hTSP1). This complex facilitates either activation of Tyr-kinases, inactivation of Tyr phosphatases, or intracellular recruitment of Tyr-phosphorylated proteins. The ligand bound integrin (cell surface receptors that interact with ECM proteins such as, for example, laminin 5, fibronectin, vitronectin, TSP1/2) transduces the signals through activation of the focal adhesion kinase (FAK) followed by indirect activation of the Ras-MAPK cascade, and then leads to osteogenic differentiation through Runx2; the laminin G domain is believed to play a role in the interaction between integrins and a 67 kDa laminin receptor.

The second conserved domain (amino acids 273 to 331 of SEQ ID NO: 2, set forth in SEQ ID NO: 4) and seventh conserved domain (amino acids 701 to 749 of SEQ ID NO: 2, set forth in SEQ ID NO: 5) are similar to von Willebrand factor type C (VWC) domains, also known as chordin-like repeats. VWC domains occur in numerous proteins of diverse functions and have been associated with facilitating protein oligomerization.

The third conserved domain (amino acids 434 to 466 of SEQ ID NO: 2, set forth in SEQ ID NO: 8), fourth conserved domain (amino acids 478 to 512 of SEQ ID NO: 2, set forth in SEQ ID NO: 7), fifth conserved domain (amino acids 549 to 586 of SEQ ID NO: 2, set forth in SEQ ID NO: 6), and sixth conserved domain (amino acids 596 to 627 of SEQ ID NO: 2, set forth in SEQ ID NO: 9) are similar to a calcium-binding EGF-like domain. Calcium-binding EGF-like domains are present in a large number of membrane-bound and extracellular (mostly animal) proteins. Many of these proteins require calcium for their biological function. Calcium-binding sites have been found to be located at the N-terminus of particular EGF-like domains, suggesting that calcium-binding may be crucial for numerous protein-protein interactions. Six conserved core cysteines form three disulfide bridges as in non-calcium-binding EGF domains whose structures are very similar. The calcium-binding EGF-like domains of NELL1 bind protein kinase C beta, which is typically involved in cell signaling pathways in growth and differentiation.

The nel-like 1 isoform 2 precursor transcript variant (set forth in GenBank Acc. No. NM_{—}201551 and SEQ ID NO: 10) lacks an alternate in-frame exon compared to variant 1. The resulting isoform 2 (set forth in SEQ ID NO: 11), which has the same N- and C-termini as isoform 1 but is shorter compared to isoform 1, has six conserved regions including a TSPN domain/LamG superfamily domain (amino acids 29 to 213 of SEQ ID NO: 11; set forth in SEQ ID NO: 12); VWC domains (amino acids 273 to 331 of SEQ ID NO: 11; set forth in SEQ ID NO: 13; amino acids 654 to 702 of SEQ ID NO: 11; set forth in SEQ ID NO: 14); and calcium-binding EGF-like domains (amino acids 478 to 512 of SEQ ID NO: 11; set forth in SEQ ID NO: 15; amino acids 434 to 466 of SEQ ID NO: 11; set forth in SEQ ID NO: 16; amino acids 549 to 580 of SEQ ID NO: 11; set forth in SEQ ID NO: 17).

NELL1 and its orthologs are found across several species including Homo sapiens (man), Bos taurus (cow; the nucleic acid sequence of which is set forth in GenBank Acc. No. XM_{—}002699102 and in SEQ ID NO: 48; the amino acid sequence of which is set forth in SEQ ID NO: 49), Equus caballus (horse; the nucleic acid sequence of isoforms 1 and 2 are set forth in GenBank Acc. Nos. XM_{—}001504986 and XM_{—}001504987, respectively, and in SEQ ID NO: 50 and 52, respectively; the amino acid sequences are set forth in SEQ ID NO: 51 and 53, respectively), Macaca mulatta (rhesus monkey; the nucleic acid sequence of isoforms 1, 2, 3, and 4 are set forth in GenBank Acc. Nos. XM_{—}002799606, XM_{—}001092428, XM_{—}001092540, and XM_{—}001092655, respectively, and in SEQ ID NO: 54, 56, 58, and 60, respectively; the amino acid sequences are set forth in SEQ ID NO: 55, 57, 59, and 61, respectively), Mus musculus (mouse; the nucleic acid sequence of which is set forth in GenBank Acc. No. NM_{—}001037906 and in SEQ ID NO: 18; the amino acid sequence of which is set forth in SEQ ID NO: 19), Rattus norvegicus (rat; the nucleic acid sequence of which is set forth in GenBank Acc. No. NM_{—}031069 and in SEQ ID NO: 20; the amino acid sequence of which is set forth in SEQ ID NO: 21), Pan troglodytes (chimpanzee; the nucleic acid sequence of which is set forth in GenBank Acc. No. XM_{—}508331.2 and in SEQ ID NO: 22; the amino acid sequence of which is set forth in SEQ ID NO: 23), Xenopus (Silurana) tropicalis (frog; the nucleic acid sequence of which is set forth in GenBank Acc. No. BC121467 and in SEQ ID NO: 24; the amino acid sequence of which is set forth in SEQ ID NO: 25), Canis lupus familiaris (dog; the nucleic acid sequence of which is set forth in GenBank Acc. No. XM_{—}534090 and in SEQ ID NO: 26; the amino acid sequence of which is set forth in SEQ ID NO: 27), Culex quinquefasciatus (mosquito; the nucleic acid sequence of which is set forth in GenBank Acc. No. XM_{—}001862888 and in SEQ ID NO28; the amino acid sequence of which is set forth in SEQ ID NO: 29), Pediculus humanus corporis (head louse; the nucleic acid sequence of which is set forth in GenBank Acc. No. XM_{—}002433079 and in SEQ ID NO: 30; the amino acid sequence of which is set forth in SEQ ID NO: 31), Aedes aegypti (mosquito; the nucleic acid sequence of which is set forth in GenBank Acc. No. XM_{—}001650373 and in SEQ ID NO: 32; the amino acid sequence of which is set forth in SEQ ID NO: 33), Ixodes scapularis (tick; the nucleic acid sequence of which is set forth in GenBank Acc. No. XM_{—}001650373 and in SEQ ID NO: 34; the amino acid sequence of which is set forth in SEQ ID NO: 35), Strongylocentrotus purpuratus (purple sea urchin; the nucleic acid sequence of which is set forth in GenBank Acc. No. XM_{—}789134 and in SEQ ID NO: 36; the amino acid sequence of which is set forth in SEQ ID NO: 37), and Acyrthosiphon pisum (pea aphid; the nucleic acid sequence of which is set forth in GenBank Acc. No. XM_{—}001950685 and in SEQ ID NO: 38; the amino acid sequence of which is set forth in SEQ ID NO: 39).

While the complete genome has not been annotated for some large animal species, including, but not limited to, Oryctolagus cuniculus (rabbit), Capra hircus (goat), Ovis aries (sheep), Macaca mulatta (rhesus monkey), Cavia porcellus (guinea pig), Sus scrofa (pig), and Pongo pygmaeus abelii (orangutan), it should be noted that NELL1 sequences identified from these large animal species and active variants and fragments thereof would also be useful in the presently disclosed methods and compositions.

3.2. NELL1 is Variable

NELL1 comprises several regions susceptible to increased recombination. Studies have indicated that susceptibilities to certain diseases may be associated with genetic variations within these regions, suggesting the existence of more than one causal variant in the NELL1 gene. For example, in patients suffering irritable bowel syndrome (“IBS”), six different single nucleotide polymorphisms (SNPs) within NELL1 have been identified, with most of these SNPs near the 5′ end of the gene and fewer at the 3′ end. These include R136S and A153T (which reside in the TSPN) and R354W (which resides in a VWC domain). Additional studies have identified at least 26 variants comprising some of at least 263 SNPs within the NELL1 region.

3.3. Function

The NELL1 protein is a secreted cytoplasmic heterotrimeric protein. Genetic and molecular characterization of the phenotype of mice with overexpression or deficiency of NELL1 function suggests the critical role of this protein is in stimulating cell proliferation and differentiation (Zhang et al. (2002) J Clin Invest 110(6):861-870; Desai et al. (2006) Hum Mol Genet 15(8):1329-1341, each of which is herein incorporated by reference in its entirety). NELL1 can promote proliferation and differentiation of bone and cartilage in culture (Aghaloo et al. (2007) Mol Ther 15(10):1872-1880; Lee et al. (2010) Tissue Eng Part A 16(5):1791-1800, each of which is herein incorporated by reference in its entirety), repair bone defects in rodents and large animals (Aghaloo et al. (2006) Am J Pathol 169(3):903-915, each of which is herein incorporated by reference in its entirety), and induces spinal fusion in rats (Li et al. (2007) Spine J 7(1):50-60; Li et al. (2010) Tissue Eng Part A June Epub ahead of print; each of which is herein incorporated by reference in its entirety).

Several studies have indicated that NELL1 may have a role in bone and cartilage formation, inflammatory bowel disease, and esophageal adenocarcinoma, among others.

3.3.2. Osteogenesis

It generally is believed that NELL1 induces osteogenic differentiation and bone formation of osteoblastic cells during development. Studies have shown that the NELL1 protein (1) transiently activates the mitogen-activated protein kinase (“MAPK”) signaling cascade (which is involved in various cellular activities such as gene expression, mitosis, differentiation, proliferation and apoptosis); and (2) induces phosphorylation of Runx2 (a transcription factor associated with osteoblast differentiation). Consequently, it generally is believed that upon binding to a specific receptor, NELL1 transduces an osteogenic signal through activation of certain Tyr-kinases associated with the Ras-MAPK cascade, which ultimately leads to osteogenic differentiation. Studies have shown that bone development is severely disturbed in transgenic mice where over-expression of NELL1 has been shown to lead to craniosynotosis (premature ossification of the skull and closure of the sutures) and NELL 1 deficiency manifests in skeletal defects due to reduced chondrogenesis and osteogenesis.

Additional studies have supported a role for NELL-1 as a craniosynostosis-related gene. For example, three regions within the NELL-1 promoter have been identified that are directly bound and transactivated by Runx2. Further, studies in rat skullcaps have indicated that forced expression of Runx2 induces NELL1 expression (which is suggestive that NELL1 is likely a downstream target of Runx2).

3.3.3. Inflammatory Bowel Disease

The term “Inflammatory Bowel Disease” (“IBD”) includes Crohn's disease (“CD”) and ulcerative colitis (“UC”). These chronic gastrointestinal inflammatory disorders have a complex genetic background. Studies have shown that NELL1 is a ubiquitous IBD susceptibility locus, and that SNPs within the nel-like 1 precursor (i.e., NELL1) provide a consistent disease-association in populations suffering from CD. The basis of this association is not understood.

3.3.5. Esophageal Adenocarcinoma

Studies have shown that in human esophageal adenocarcinoma (“EAC”), hypermethylation of the NELL1 promoter is a common, tissue-specific event. During Barrett's-associated esophageal neoplastic progression, hypermethylation of the NELL1 promoter occurs early. Consequently, it generally is believed that NELL1 is a potential biomarker of poor prognosis in early-stage EAC. The basis of this association is not understood.

3.3.6. Other NELL1 Functions

Genetic and genomic studies have revealed that NELL1 activity is essential to the production of key components of the ECM such as tenascins (tenascin b, tenascin C), collagens (collagen VI al, collagen IV a1), and proteoglycans (Desai et al. (2006) Hum Mol Genet 15(8):1329-1341; and U.S. Application Publication No. US2009/0142312, each of which is herein incorporated by reference in its entirety).

NELL1 is also essential for normal cardiovascular development by promoting the proper formation of the heart and blood vessels (U.S. Application Publication No. US2009/0087415, which is herein incorporated by reference in its entirety). These data suggest a more general role for NELL1 in the promotion of angiogenesis.

As demonstrated herein, NELL1 inhibits inflammation, at least partially, through reducing levels of inflammatory mediators, such as IL-1β and IL-8. NELL1 further reduces levels of matrix metalloproteinases (e.g., MMP1) and contributes to improved skin hydration through the upregulation of aquaporins (e.g., AQP3). NELL1 also reduces the number of sunburned cells upon UV exposure.

A general role for NELL 1 in stimulating the differentiation of precursor cells has been implicated (see, for example, U.S. Application Publication No. US2009/0142312. For example, NELL1 can stimulate the differentiaion of skeletal muscle cells to maturity or osteoblast precursors to mature bone cells. NELL1 can promote wound healing and stimulate skeletal muscle regeneration. The term “stimulate” as used herein refers to activate, provoke, or spur. The term “stimulating agent” as used herein refers to a substance that exerts some force or effect.

As used herein, the terms “NELL1 peptide”, “NELL1 polypeptide”, and “NELL1 protein” refer to a naturally-occurring NELL1 protein, or a variant or fragment thereof that retains the ability to prevent or reduce the signs of aging or a scar. In some embodiments, the NELL1 peptide exhibits any one of the activities selected from the group consisting of: stimulation of ECM production (e.g., through the upregulation of at least one of tenascins, proteoglycans, elastin, glycosaminoglycans, including epidermal hyaluronic acid, and collagens), reduction in the levels of inflammatory mediators (e.g., IL-1β and IL-8), reduction in the levels of matrix metalloproteinases (e.g., MMP1), increase in aquaporin (e.g., AQP3) levels, reduction in the number of sunburned cells. In other embodiments, the NELL1 peptide can also exhibit at least one of the activities selected from the group consisting of binding to PKC-beta, stimulation of differentiation of a precursor cell (e.g., skeletal satellite cell or osteoblast precursor) to maturity, stimulation of angiogenesis. To determine whether a peptide exhibits any one of these activities, any method known in the art useful for measuring these activities can be used.

Suitable assays for determining if a given peptide can stimulate ECM production and aquaporin levels and reduce the levels of inflammatory mediators or MMPs include assays that measure transcript levels (e.g., quantitative polymerase chain reaction) or levels of the protein (e.g., enzyme-linked immunoassay) directly or indirectly (by measuring the activity of the protein), including those that are described elsewhere herein.

Suitable assays for assessing the binding of NELL1 to PKC beta is described in e.g., Kuroda et al. (1999) Biochem Biophys Res Comm 265:752-757, which is herein incorporated by reference in its entirety. For example, protein-protein interactions can be analyzed by using the yeast two-hybrid system. Briefly, a modified NELL1 protein can be fused with GAL4 activating domain and the regulatory domain of PKC can be fused with the GAL4 DNA-binding domain.

In other embodiments, the NELL1 peptide stimulates the differentiation of precursor cells, such as skeletal satellite cells or osteoblast precursors, to maturity. The maturity of skeletal muscle cells can be assessed cellularly (histology) and molecularly (expression of skeletal muscle-specific proteins or extracellular matrix materials). Still further, a protein can be tested for its ability to drive osteoblast precursors to mature bone cells, by detecting expression of late molecular bone markers or mineralization (i.e., calcium deposits).

The NELL1 peptide may be a naturally-occurring (i.e., wild-type) NELL1 protein or an active variant or fragment thereof. The term “naturally” as used herein refers to as found in nature; wild-type; innately or inherently. A naturally-occurring NELL1 peptide may be purified from a natural source or may be a peptide that has been recombinantly or synthetically produced that has the same amino acid sequence as a NELL1 peptide found in nature.

The term “variant of NELL1 protein” is used herein to refer to a wild-type NELL-1-like polypeptide sequence in which at least one amino acid residue has been modified by deletion of an amino acid, insertion of an amino acid, or substitution of a second amino acid for a first amino acid at a specific position on the polypeptide.

The terms “variants”, “mutants”, and “derivatives” are used herein to refer to nucleotide sequences with substantial identity to a reference nucleotide sequence. The differences in the sequences may by the result of changes, either naturally or by design, in sequence or structure. Natural changes may arise during the course of normal replication or duplication in nature of the particular nucleic acid sequence. Designed changes may be specifically designed and introduced into the sequence for specific purposes. Such specific changes may be made in vitro using a variety of mutagenesis techniques. Such sequence variants generated specifically may be referred to as “mutants” or “derivatives” of the original sequence.

The term “mutation” refers to a change of the DNA sequence within a gene or chromosome of an organism resulting in the creation of a new character or trait not found in the parental type, or the process by which such a change occurs in a chromosome, either through an alteration in the nucleotide sequence of the DNA coding for a gene or through a change in the physical arrangement of a chromosome. Three mechanisms of mutation include substitution (exchange of one base pair for another), addition (the insertion of one or more bases into a sequence), and deletion (loss of one or more base pairs).

The term “substitution” is used herein to refer to that in which a base or bases are exchanged for another base or bases in DNA. Substitutions may be synonymous substitutions or nonsynonymous substitutions. As used herein, “synonymous substitutions” refer to substitutions of one base for another in an exon of a gene coding for a protein, such that the amino acid sequence produced is not modified. The term “nonsynonymous substitutions” as used herein refer to substitutions of one base for another in an exon of a gene coding for a protein, such that the amino acid sequence produced is modified.

The term “addition,” as used in the context of an amino acid or nucleotide sequence, refers to the insertion of one or more bases, or of one or more amino acids, into the sequence.

The terms “deletion” and “deletion mutation” are used interchangeably herein to refer to that in which a base(s) is lost from DNA.

A skilled artisan likewise can produce polypeptide variants having single or multiple amino acid substitutions, deletions, additions or replacements. These variants may include inter alia: (a) variants in which one or more amino acid residues are substituted with conservative or non-conservative amino acids; (b) variants in which one or more amino acids are added; (c) variants in which at least one amino acid includes a substituent group; (d) variants in which amino acid residues from one species are substituted for the corresponding residue in another species, either at conserved or non-conserved positions; and (d) variants in which a target protein is fused with another peptide or polypeptide such as a fusion partner, a protein tag or other chemical moiety, that may confer useful properties to the target protein, such as, for example, an epitope for an antibody. The techniques for obtaining such variants, including genetic (suppressions, deletions, mutations, etc.), chemical, and enzymatic techniques are known to the skilled artisan.

The following represent groups of amino acids that are conservative substitutions for one another:

1) Alanine (A), Serine (S), Threonine (T);

2) Aspartic Acid (D), Glutamic Acid (E);

3) Asparagine (N), Glutamic Acid (Q);

4) Arginine (R), Lysine (K);

5) Isoleucine (I), Leucine (L), Methionine (M), Valine (V); and

6) Phenylalanine (F), Tyrosine (Y), Tryptophan (W).

Any substitutions, additions, and/or deletions in an amino acid sequence are permitted provided that the NELL1 peptide is functional (e.g., retains at least one of the following NELL1-associated activities: ability to prevent or reduce the signs of aging or a scar, stimulates ECM production (e.g., through the upregulation of at least one of tenascins, proteoglycans, elastin, glycosaminoglycans, including epidermal hyaluronic acid, and collagens), reduces the levels of inflammatory mediators (e.g., IL-1β and IL-8), reduces the levels of matrix metalloproteinases (e.g., MMP1), increases aquaporin (e.g., AQP3) levels, reduces the number of sunburned cells, binding to PKC-beta, stimulates the differentiation of a precursor cell (e.g., skeletal satellite cell or osteoblast precursor) to maturity, stimulation of angiogenesis).

One of skill in the art will appreciate that conserved segments of NELL1 proteins will be less tolerant of non-conservative mutations. The term “conserved segment” is used herein to refer to similar or identical sequences that may occur within nucleic acids, proteins or polymeric carbohydrates within multiple species of organism or within different molecules produced by the same organism. The conserved domains of NELL1 proteins have been highlighted hereinabove and include a secretion signal peptide domain, thrombospondin N-terminal domain (“TSPN”), and several von Willebrand factor, type C (“VWC”), and epidermal growth-factor (“EGF”) domains.

Novel naturally-occurring NELL1 proteins (including those that result from genetic polymorphism) can be identified with the use of well-known molecular biology techniques, such as, for example, with polymerase chain reaction (PCR) and hybridization techniques. Variant proteins also include synthetically derived proteins, such as those generated, for example, by using site-directed mutagenesis. Generally, variants of a particular protein will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular protein as determined by sequence alignment programs and parameters.

Biologically active variants of a naturally occurring NELL1 protein will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence for the naturally occurring protein as determined by sequence alignment programs and parameters. A biologically active variant of a naturally occurring NELL1 protein may differ from that protein by as few as 1-15 amino acid residues, as few as 1-10, such as 6-10, as few as 5, as few as 4, 3, 2, or even 1 amino acid residue.

In some embodiments, the NELL1 peptide has at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence set forth in SEQ ID NO: 2, 11, 19, 21, 23, 25, 27, 29, 31, 33, 35, 37, 39, 49, 51, 53, 55, 57, 59, or 61.

The term “hybridization” refers to the process of combining complementary, single-stranded nucleic acids into a single molecule. Nucleotides will bind to their complement under normal conditions, so two perfectly complementary strands will bind (or ‘anneal’) to each other readily. However, due to the different molecular geometries of the nucleotides, a single inconsistency between the two strands will make binding between them more energetically unfavorable. Measuring the effects of base incompatibility by quantifying the rate at which two strands anneal can provide information as to the similarity in base sequence between the two strands being annealed. The term “specifically hybridizes” as used herein refers to the process whereby a nucleic acid distinctively or definitively forming base pairs with complementary regions of at least one strand of DNA that was not originally paired to the nucleic acid. For example, a nucleic acid that may bind or hybridize to at least a portion of an mRNA of a cell encoding a peptide comprising a NELL1 sequence may be considered a nucleic acid that specifically hybridizes. A nucleic acid that selectively hybridizes undergoes hybridization, under stringent hybridization conditions, of the nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 80% sequence identity, at least 90% sequence identity, or at least 100% sequence identity (i.e., complementary) with each other.

The following terms are used herein to describe the sequence relationships between two or more nucleic acids or polynucleotides: (a) “reference sequence”, (b) “comparison window”, (c) “sequence identity”, (d) “percentage of sequence identity”, and (e) “substantial identity”.

The term “reference sequence” refers to a sequence used as a basis for sequence comparison. A reference sequence may be a subset or the entirety of a specified sequence; for example, as a segment of a full-length cDNA or gene sequence, or the complete cDNA or gene sequence.

The term “comparison window” refers to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be at least 30 contiguous nucleotides in length, at least 40 contiguous nucleotides in length, at least 50 contiguous nucleotides in length, at least 100 contiguous nucleotides in length, or longer. Those of skill in the art understand that to avoid a high similarity to a reference sequence due to inclusion of gaps in the polynucleotide sequence, a gap penalty typically is introduced and is subtracted from the number of matches.

Methods of alignment of sequences for comparison are well-known in the art. Optimal alignment of sequences for comparison may be conducted by the local homology algorithm of Smith and Waterman, Adv. Appl. Math. 2:482 (1981); by the homology alignment algorithm of Needleman and Wunsch, J. Mol. Biol. 48:443 (1970); by the search for similarity method of Pearson and Lipman, Proc. Natl. Acad. Sci. 85:2444 (1988); by computerized implementations of these algorithms, including, but not limited to: CLUSTAL in the PC/Gene program by Intelligenetics, Mountain View, Calif.; GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73:237-244 (1988); Higgins and Sharp, CABIOS 5:151-153 (1989); Corpet, et al., Nucleic Acids Research 16:10881-90 (1988); Huang, et al., Computer Applications in the Biosciences 8:155-65 (1992), and Pearson, et al., Methods in Molecular Biology 24:307-331 (1994). The BLAST family of programs, which can be used for database similarity searches, includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995).

Unless otherwise stated, sequence identity/similarity values provided herein refer to the value obtained using the BLAST 2.0 suite of programs using default parameters. Altschul et al., Nucleic Acids Res. 25:3389-3402 (1997). Software for performing BLAST analyses is publicly available, e.g., through the National Center for Biotechnology-Information (http://www.hcbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits then are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue aligng/kg body weightents; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, a cutoff of 100, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. BLAST searches assume that proteins may be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs may be employed to reduce such low-complexity alignment. For example, the SEG (Wooten and Federhen, Comput. Chem., 17:149-163 (1993)) and XNU (Claverie and States, Comput. Chem., 17:191-201 (1993)) low-complexity filters may be employed alone or in combination.

The term “sequence identity” or “identity” in the context of two nucleic acid or polypeptide sequences is used herein to refer to the residues in the two sequences that are the same when aligned for maximum correspondence over a specified comparison window. When percentage of sequence identity is used in reference to proteins it is recognized that residue positions that are not identical often differ by conservative amino acid substitutions, i.e., where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988) e.g., as implemented in the program PC/GENE (Intelligenetics, Mountain View, Calif., USA).

The term “percentage of sequence identity” is used herein to mean the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “substantial identity” of polynucleotide sequences means that a polynucleotide comprises a sequence that has at least 70% sequence identity, at least 75% sequence identity, at least 80% sequence identity, at least 85% sequence identity, at least 90% sequence identity, at least 95% sequence identity, at least 96% sequence identity, at least 97% sequence identity, at least 98% sequence identity, at least 99% sequence identity, compared to a reference sequence using one of the alignment programs described using standard parameters. One of skill will recognize that these values may be adjusted appropriately to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, or at least 65%, at least 70%, at least 75%, at least 80%, at least 85%, at least 90%, at least 95%, at least 96%, at least 97%, at least 98%, or at least 99%. Another indication that nucleotide sequences are substantially identical is if two molecules hybridize to each other under stringent conditions. However, nucleic acids that do not hybridize to each other under stringent conditions are still substantially identical if the polypeptides that they encode are substantially identical. This may occur, e.g., when a copy of a nucleic acid is created using the maximum codon degeneracy permitted by the genetic code. One indication that two nucleic acid sequences are substantially identical is that the polypeptide that the first nucleic acid encodes is immunologically cross-reactive with the polypeptide encoded by the second nucleic acid.

The term “substantial identity” in the context of a peptide indicates that a peptide comprises a sequence with at least 70% sequence identity to a reference sequence, at least 75%, at least 80%, at least 85%, at least 90%, at least 95%, at least 96%, at least 97%, at least 98%, or at least 99% sequence identity to the reference sequence over a specified comparison window. Optionally, optimal alignment is conducted using the homology alignment algorithm of Needleman and Wunsch, J. Mol. Biol. 48:443 (1970). An indication that two peptide sequences are substantially identical is that one peptide is immunologically reactive with antibodies raised against the second peptide. Thus, a peptide is substantially identical to a second peptide, for example, where the two peptides differ only by a conservative substitution. Peptides which are “substantially similar” share sequences as noted above except that residue positions that are not identical may differ by conservative amino acid changes.

The NELL1 peptide may be an active fragment of a naturally-occurring NELL1 protein that retains at least one activity of the NELL1 protein selected from the group consisting of: ability to prevent or reduce the signs of aging or a scar, stimulates ECM production (e.g., through the upregulation of at least one of tenascins, proteoglycans, elastin, glycosaminoglycans, including epidermal hyaluronic acid, and collagens), reduces the levels of an inflammatory mediator (e.g., IL-1β and IL-8), reduces the levels of a matrix metalloproteinase (e.g., MMP1), increases aquaporin (e.g., AQP3) levels, reduces the number of sunburned cells, binding to PKC-beta, stimulates the differentiation of a precursor cell (e.g., skeletal satellite cell or osteoblast precursor) to maturity, and stimulation of angiogenesis. The term “fragment” or “peptide fragment” as used herein refers to a small part derived, cut off, processed, or broken from a larger peptide, polypeptide or protein, which retains the desired biological activity of the larger peptide, polypeptide or protein. An active fragment of a naturally occurring NELL1 protein can be 15, 25, 30, 50, 100, 150, 200, 250, 300, 350, 400, 450, 500, 550, 600, 650, 700, 750, 800, 810 contiguous amino acids, or up to the total number of amino acids present in the full-length NELL1 protein (e.g., SEQ ID NO: 2, 11, 19, 21, 23, 25, 27, 29, 31, 33, 35, 37, 39, 49, 51, 53, 55, 57, 59, or 61).

In some embodiments, the NELL1 peptide useful in the presently disclosed methods and compositions has at least one of the sequences set forth in SEQ ID NO: 3, 4, 5, 6, 7, 8, 9, 12, 13, 14, 15, 16, or 17. In other embodiments, the NELL1 peptide has at least one of the following amino acid sequences: an amino acid sequence having at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence set forth in SEQ ID NO: 3; an amino acid sequence having at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence set forth in SEQ ID NO: 4; an amino acid sequence having at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence set forth in SEQ ID NO: 5; an amino acid sequence having at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence set forth in SEQ ID NO: 6; an amino acid sequence having at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence set forth in SEQ ID NO: 7; an amino acid sequence having at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence set forth in SEQ ID NO: 8; an amino acid sequence having at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence set forth in SEQ ID NO: 9; an amino acid sequence having at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence set forth in SEQ ID NO: 12; an amino acid sequence having at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence set forth in SEQ ID NO: 13; an amino acid sequence having at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence set forth in SEQ ID NO: 14; an amino acid sequence having at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence set forth in SEQ ID NO: 15; an amino acid sequence having at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence set forth in SEQ ID NO: 16; and an amino acid sequence having at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence set forth in SEQ ID NO: 17.

The NELL1 peptide may be prepared by methods that are well known in the art. One such method includes isolating or synthesizing DNA encoding the NELL1 peptide, and producing the recombinant protein by expressing the DNA, optionally in a recombinant vector, in a suitable host cell. Suitable methods for synthesizing DNA are described by Caruthers et al. (1985) Science 230:281-285; and DNA Structure, Part A: Synthesis and Physical Analysis of DNA, Lilley, D. M. J. and Dahlberg, J. E. (Eds.), Methods Enzymol., 211, Academic Press, Inc., New York (1992).

The NELL1 peptide may also be made synthetically, i.e. from individual amino acids, or semisynthetically, i.e. from oligopeptide units or a combination of oligopeptide units and individual amino acids. Suitable methods for synthesizing proteins are described by Stuart and Young in “Solid Phase Peptide Synthesis,” Second Edition, Pierce Chemical Company (1984), Solid Phase Peptide Synthesis, Methods Enzymol., 289, Academic Press, Inc, New York (1997).

3.4 NELL1 Nucleic Acid Molecules

In some embodiments of the presently disclosed methods, a nucleic acid molecule encoding a NELL1 peptide is administered to a subject in need thereof in order to treat or prevent a skin condition (e.g., skin aging, skin scarring).

The terms “nucleic acid”, “nucleic acid molecule”, and “polynucleotide” are used interchangeably herein to refer to a deoxyribonucleotide or ribonucleotide polymer in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids). A nucleic acid, nucleic acid molecule, or polynucleotide may be full-length or a subsequence of a native or heterologous structural or regulatory gene. Unless otherwise indicated, the terms include reference to the specified sequence as well as the complementary sequence thereof. Thus, DNAs or RNAs with backbones modified for stability or for other reasons are “nucleic acids”, “nucleic acid molecules”, and “polynucleotides” as these terms are intended herein. Moreover, DNAs or RNAs comprising unusual bases, such as, for example, inosine, or modified bases, such as tritylated bases, are nucleic acids, nucleic acid molecules, and polynucleotides as the terms are used herein. It will be appreciated that a great variety of modifications have been made to DNA and RNA that serve many useful purposes known to those of skill in the art. The terms “nucleic acid”, “nucleic acid molecule”, and “polynucleotide” as employed herein embrace such chemically, enzymatically or metabolically modified forms of polynucleotides, as well as the chemical forms of DNA and RNA characteristic of viruses and cells, including among other things, simple and complex cells. The terms “nucleic acid”, “nucleic acid molecule”, and “polynucleotide” refer to both linear and circularized molecules. The term “linear DNA” as used herein refers to non-circularized DNA molecules.

The term “nucleotide” is used herein to refer to a chemical compound that consists of a heterocyclic base, a sugar, and one or more phosphate groups. In the most common nucleotides, the base is a derivative of purine or pyrimidine, and the sugar is the pentose deoxyribose or ribose. Nucleotides are the monomers of nucleic acids, with three or more bonding together in order to form a nucleic acid. Nucleotides are the structural units of RNA, DNA, and several cofactors, including, but not limited to, CoA, FAD, DMN, NAD, and NADP. Purines include adenine (A), and guanine (G); pyrimidines include cytosine (C), thymine (T), and uracil (U).

The term “isolated” is used herein to refer to material, such as, but not limited to, a nucleic acid, peptide, polypeptide, or protein, which is: (1) substantially or essentially free from components that normally accompany or interact with it as found in its naturally occurring environment. The terms “substantially free” or “essentially free” are used herein to refer to considerably or significantly free of, or more than about 95% free of, or more than about 99% free of. The isolated material optionally comprises material not found with the material in its natural environment; or (2) if the material is in its natural environment, the material has been synthetically (non-naturally) altered by deliberate human intervention to a composition and/or placed at a location in the cell (e.g., genome or subcellular organelle) not native to a material found in that environment. The alteration to yield the synthetic material may be performed on the material within, or removed, from its natural state. For example, a naturally occurring nucleic acid becomes an isolated nucleic acid if it is altered, or if it is transcribed from DNA that has been altered, by means of human intervention performed within the cell from which it originates. See, for example, Compounds and Methods for Site Directed Mutagenesis in Eukaryotic Cells, Kmiec, U.S. Pat. No. 5,565,350; In Vivo Homologous Sequence Targeting in Eukaryotic Cells; Zarling et al., PCT/US93/03868, each of which is incorporated by reference herein. Likewise, a naturally occurring nucleic acid (for example, a promoter) becomes isolated if it is introduced by non-naturally occurring means to a locus of the genome not native to that nucleic acid.

In some embodiments of the presently disclosed methods, the NELL1 nucleic acid molecule is operably linked to at least one regulatory element. The term “regulatory element” as used herein refers to a nucleic acid sequence(s) capable of effecting the expression of nucleic acid(s), or the peptide or protein product thereof. Non-limiting examples of regulatory elements include promoters, enhancers, polyadenylation signals, transcription or translation termination signals, ribosome binding sites, or other segments of DNA where regulatory proteins, such as, but not limited to, transcription factors, bind preferentially to control gene expression and thus protein expression.

Regulatory elements may be operably linked to the nucleic acids, peptides, or proteins of the described invention. The term “operably linked” refers to a functional linkage two or more elements. For example, when a promoter and a protein coding sequence are operably linked, the promoter sequence initiates and mediates transcription of the protein coding sequence. The regulatory elements need not be contiguous with the nucleic acids, peptides, or proteins whose expression they control as long as they function to direct the expression thereof. Thus, for example, intervening untranslated yet transcribed sequences may be present between a promoter sequence and a nucleic acid of the described invention and the promoter sequence may still be considered “operably linked” to the coding sequence.

The term “promoter” refers to a region of DNA upstream, downstream, or distal, from the start of transcription and involved in recognition and binding of RNA polymerase and other proteins to initiate transcription. For example, T7, T3 and Sp6 are RNA polymerase promoter sequences. In RNA synthesis, promoters are a means to demarcate which genes should be used for messenger RNA creation and by extension, control which proteins the cell manufactures. Promoters represent critical elements that can work in concert with other regulatory regions (enhancers, silencers, boundary elements/insulators) to direct the level of transcription of a given gene.

Any type of promoter can be used to regulate the expression of a NELL1 nucleic acid molecule, including but not limited to, a constitutive promoter, a tissue-specific promoter (e.g., skin-specific promoter), and inducible promoter.

For example, commonly used promoters are derived from polyoma, Adenovirus 2, cytomegalovirus, and Simian Virus 40. For other suitable expression systems for eukaryotic cells, see Chapters 16 and 17 of Sambrook et al. (1989) Molecular Cloning: A Laboratory Manual (2d ed., Cold Spring Harbor Laboratory Press, Plainview, N.Y). See, Goeddel (1990) in Gene Expression Technology: Methods in Enzymology 185 (Academic Press, San Diego, Calif.).

Various constitutive promoters are known. Promoters which may be used include, but are not limited to, the long terminal repeat as described in Squinto et al. (1991) Cell 65:1 20); the SV40 early promoter region (Bernoist and Chambon (1981) Nature 290:304 310), the CMV promoter, the M-MuLV 5′ terminal repeat the promoter contained in the 3′ long terminal repeat of Rous sarcoma virus (Yamamoto et al. (1980) Cell 22:787 797), and the herpes thymidine kinase promoter (Wagner et al. (1981) Proc. Natl. Acad. Sci. U.S.A. 78:144 1445).

The term “transcription termination signal” refers to a section of genetic sequence that marks the end of gene or operon on genomic DNA for transcription. In prokaryotes, two classes of transcription termination signals are known: 1) intrinsic transcription termination signals where a hairpin structure forms within the nascent transcript that disrupts the mRNA-DNA-RNA polymerase ternary complex; and 2) Rho-dependent transcription termination signal that require Rho factor, an RNA helicase protein complex to disrupt the nascent mRNA-DNA-RNA polymerase ternary complex. In eukaryotes, transcription termination signals are recognized by protein factors that co-transcriptionally cleave the nascent RNA at a polyadenlyation signal (i.e, “poly-A signal” or “poly-A tail”) halting further elongation of the transcript by RNA polymerase. The subsequent addition of the poly-A tail at this site stabilizes the mRNA and allows it to be exported outside the nucleus. Termination sequences are distinct from termination codons that occur in the mRNA and are the stopping signal for translation, which also may be called nonsense codons.

The term “translational stop sequence” refers to a sequence which codes for the translational stop codons. A translational stop sequence may be in one, two, or three reading frames.

The term “internal ribosome entry site” (IRES) refers to an element which permits attachment of a downstream coding region or open reading frame with a cytoplasmic polysomal ribosome for purposes of initiating translation thereof in the absence of any internal promoters. Generally, an IRES is included to initiate translation of selectable marker protein coding sequences. Examples of suitable IRESes that can be used include, but are not limited to, the mammalian IRES of the immunoglobulin heavy-chain-binding protein (BiP), and those from the picomaviruses, such as, but not limited to, those from encephalomyocarditis virus (nucleotide numbers 163-746), poliovirus (nucleotide numbers 28-640) and foot and mouth disease virus (nucleotide numbers 369-804).

In certain embodiments, the NELL1 nucleic acid molecule is a recombinant expression cassette or is part of an expression system. The term “recombinant expression cassette” refers to a nucleic acid construct, generated recombinantly or synthetically, with a series of specified nucleic acid elements which permit transcription of a particular nucleic acid (e.g., protein coding sequence) in a host cell. The recombinant expression cassette can be incorporated into a plasmid, chromosome, mitochondrial DNA, virus, or nucleic acid fragment. Typically, the recombinant expression cassette portion of an expression vector includes, among other sequences, a nucleic acid to be transcribed, a promoter, and a transcription termination signal such as a poly-A signal.

The term “protein coding sequence” refers to a nucleotide sequence encoding a polypeptide, such as the NELL1 peptide, or a selectable marker. The protein coding sequence can comprise introns, exons, and functional splice acceptors. The term “functional splice acceptor” refers to any individual functional splice acceptor or functional splice acceptor consensus sequence that permits a construct of the disclosure to be processed such that it is included in any mature, biologically active mRNA. As used herein, the terms “encoding” or “encoded” when used in the context of a specified nucleic acid mean that the nucleic acid comprises the requisite information to direct translation of the nucleotide sequence into a specified polypeptide. The information by which a polypeptide is encoded is specified by the use of codons.

In some embodiments, the protein coding sequence does not include introns and is referred to as complementary DNA or cDNA. The terms “complementary DNA” and “cDNA” refer to DNA synthesized from a mature mRNA template. cDNA most often is synthesized from mature mRNA using the enzyme reverse transcriptase. The enzyme operates on a single strand of mRNA, generating its complementary DNA based on the pairing of RNA base pairs (A, U, G, C) to their DNA complements (T, A, C, G). There are several methods known for generating cDNA to obtain, for example, eukaryotic cDNA whose introns have been spliced. Generally, these methods incorporate the following steps: a) a eukaryotic cell transcribes the DNA (from genes) into RNA (pre-mRNA); b) the same cell processes the pre-mRNA strands by splicing out introns, and adding a poly-A tail and 5′ Methyl-Guanine cap; c) this mixture of mature mRNA strands are extracted from the cell; d) a poly-T oligonucleotide primer is hybridized onto the poly-A tail of the mature mRNA template. (Reverse transcriptase requires this double-stranded segment as a primer to start its operation.); e) reverse transcriptase is added, along with deoxynucleotide triphosphates (A, T, G, C); f) the reverse transcriptase scans the mature mRNA and synthesizes a sequence of DNA that complements the mRNA template. This strand of DNA is complementary DNA. (see also Current Protocols in Molecular Biology, John Wiley & Sons, incorporated in its entirety herein).

In certain embodiments, the NELL1 nucleic acid molecule is a cloning vector. The term “cloning vector” refers to a DNA molecule such as a plasmid, cosmid, or bacterial phage, or virus, such as, for example, retroviruses, adeno-associated adenoviruses, lentivirus, baculoviruses and adenoviruses, that has the capability of replicating autonomously in a host cell. The term “replication” or “replicating” as used herein refers to making an identical copy of an object.

Cloning vectors typically contain one or a small number of restriction endonuclease recognition sites (e.g., multiple cloning site) at which foreign DNA sequences can be inserted in a determinable fashion without loss of essential biological function(s) of the vector, as well as a reporter (e.g., selectable marker gene) that is suitable for use in the identification and selection of cells transformed with the cloning vector.

In some embodiments, the cloning vector comprises a stuffer fragment. The term “stuffer fragment” as used herein refers to a DNA sequence that is inserted into another DNA sequence in order to increase its size.

The term “multiple cloning site” (“MCS”, “polylinker”) as used herein refers to a short segment of DNA which contains many (usually 20+) sites recognized by restriction enzymes or other endonucleases. The term “restriction enzyme” (or restriction endonuclease) refers to an enzyme that cuts double-stranded DNA. The term “restriction sites” or “restriction recognition sites” refer to particular sequences of nucleotides that are recognized by restriction enzymes as sites to cut a DNA molecule. The sites are generally, but not necessarily, palindromic, (because restriction enzymes usually bind as homodimers) and a particular enzyme may cut between two nucleotides within its recognition site, or somewhere nearby. The term “restriction digestion” refers to a procedure used to prepare DNA for analysis or other processing. Also known as DNA fragmentation, it uses a restriction enzyme to selectively cleave strands of DNA into shorter segments.

The term “assay marker” or “reporter gene” (or “reporter”) refers to a gene that can be detected, or easily identified and measured. The expression of the reporter gene may be measured at either the RNA level, or at the protein level. The gene product, which may be detected in an experimental assay protocol, includes, but is not limited to, marker enzymes, antigens, amino acid sequence markers, cellular phenotypic markers, nucleic acid sequence markers, and the like. Researchers may attach a reporter gene to another gene of interest in cell culture, bacteria, animals, or plants. For example, some reporters are selectable markers, or confer characteristics upon on organisms expressing them allowing the organism to be easily identified and assayed. To introduce a reporter gene into an organism, researchers may place the reporter gene and the gene of interest in the same DNA construct to be inserted into the cell or organism. For bacteria or eukaryotic cells in culture, this may be in the form of a plasmid. Commonly used reporter genes may include, but are not limited to, fluorescent proteins, luciferase, beta-galactosidase, and selectable markers, such as chloramphenicol and kanamycin.

The term “detectable response” refers to any signal or response that may be detected in an assay, which may be performed with or without a detection reagent. Detectable responses include, but are not limited to, radioactive decay and energy (e.g., fluorescent, ultraviolet, infrared, visible) emission, absorption, polarization, fluorescence, phosphorescence, transmission, reflection or resonance transfer. Detectable responses also include chromatographic mobility, turbidity, electrophoretic mobility, mass spectrum, ultraviolet spectrum, infrared spectrum, nuclear magnetic resonance spectrum and x-ray diffraction. Alternatively, a detectable response may be the result of an assay to measure one or more properties of a biologic material, such as melting point, density, conductivity, surface acoustic waves, catalytic activity or elemental composition. A “detection reagent” is any molecule that generates a detectable response indicative of the presence or absence of a substance of interest. Detection reagents include any of a variety of molecules, such as antibodies, nucleic acid sequences and enzymes. To facilitate detection, a detection reagent may comprise a marker.

The term “detectable marker” encompasses both selectable markers and assay markers. The term “selectable markers” refers to a variety of gene products to which cells transformed with an expression construct can be selected or screened, including drug-resistance markers, antigenic markers useful in fluorescence-activated cell sorting, adherence markers such as receptors for adherence ligands allowing selective adherence, and the like.

The term “selectable marker” refers to a gene introduced into a cell, especially a bacterium or to cells in culture that confers a trait suitable for artificial selection. They are a type of reporter gene used in laboratory microbiology, molecular biology, and genetic engineering to indicate the success of a transfection or other procedure meant to introduce foreign DNA into a cell. Selectable markers may include, but are not limited to,: antibiotics (ampicillin) and ‘suicide’ genes (for example ccdB). For example, positive selective markers may utilize: adenosine deaminase (thymidine, hypoxanthine, 9-β-D-xylofuranosyl adenine, 2′-deoxycoformycin), aminoglycoside phosphotransferase (neomycin, G418, gentamycin, kanamycin), Bleomycin (bleomycin, phleomycin, zeocin), cytosine deaminase (N-(phosphonacetyl)-L-aspartate, inosine, cytosine); dehydrofolate reductase (methotrexate, aminopterin); histidinol dehydrogenase (histindol); hygromycin-B-phosphotransferase (hygromycin-B); puromycin-N-acetyl transferase (puromycin); thymidine kinase (hypoxanthine, aminopterin, thymidine, glycine); and xanthine-guanine phosphorriobsyltransferase (xanthine, hypoxanthine, thymidine, aminopterin, mycophenolic acid, L-glutamine). Negative selectable markers may utilize: cytosine deaminase (5-fluorocytosine); diptheria toxin; ccdB, and HSV-TK. Other selectable markers include β-galactosidase, tryptophan synthetase, luciferase, chloramphenicol acetyltransferase, dihydrofolate reductase (DHFR), CD4, and CD8.

In some embodiments, the NELL1 nucleic acid molecule is a plasmid. The term “plasmid” as used herein refers to an extra-chromosomal DNA molecule separate from the chromosomal DNA which is capable of replicating independently of the chromosomal DNA. In many cases, it is circular and double-stranded.

The expression cassette or cloning vector can be generated using molecular biology techniques known in the art and utilizing restriction enzymes, ligases, recombinases, and nucleic acid amplification techniques such as polymerase chain reaction that can be coupled with reverse transcription.

The term “ligase” as used herein refers to an enzyme that can link together DNA strands that have double-strand breaks. Common commercially available DNA ligases include those derived from T4, Escherichia coli or other bacteria.

The term “recombinase” as used herein refers to an enzyme that catalyzes genetic recombination. A recombinase enzyme catalyzes the exchange of short pieces of DNA between two long DNA strands, particularly the exchange of homologous regions between the paired maternal and paternal chromosomes.

The term “amplification” as used herein refers to a replication of genetic material that results in an increase in the number of copies of that genetic material. The term “reverse transcription” or “reverse transcription polymerase chain reaction” (RT-PCR) refers to amplifying a defined piece of a ribonucleic acid (RNA) molecule. The RNA strand is first reverse transcribed into its DNA complement or complementary DNA, followed by amplification of the resulting DNA using polymerase chain reaction.

In some embodiments, the NELL1 nucleic acid molecule is in a host cell that can be used for propagation of the nucleic acid molecule or for expression of the NELL1 peptide and subsequent isolation and/or purification. The term “host cell” encompasses any cell that contains a heterologous nucleic acid molecule. “Heterologous” in reference to a polypeptide or a nucleotide sequence is a polypeptide or a sequence that originates from a different species, or if from the same species, is substantially modified from its native form in composition and/or genomic locus by deliberate human intervention. The host cell typically supports the replication and/or expression of the vector. Host cells may be prokaryotic cells such as, but not limited to, Escherichia coli, or eukaryotic cells such as, but not limited to, yeast, insect, amphibian, or mammalian cells. The term as used herein means any cell which may exist in culture or in vivo as part of a unicellular organism, part of a multicellular organism, or a fused or engineered cell culture. The term “cloning host cell” refers to a host cell that contains a cloning vector.

The term “recombinant” refers to a cell or vector that has been modified by the introduction of a heterologous nucleic acid or the cell that is derived from a cell so modified. Recombinant cells express genes that are not found in identical form within the native (non-recombinant) form of the cell or express native genes that are otherwise abnormally expressed, under-expressed or not expressed at all as a result of deliberate human intervention. The term “recombinant” as used herein does not encompass the alteration of the cell or vector by naturally occurring events (e.g., spontaneous mutation, natural transformation transduction/transposition) such as those occurring without deliberate human intervention.

The NELL1 nucleic acid molecule can be introduced into a host cell for propagation of production of NELL1 using any method known in the art, including transfection, transformation, or transduction, so long as the nucleic acid molecule gains access to the interior of the cell. The term “inserted” or “introduced” in the context of inserting a nucleic acid into a cell, refers to “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, or transiently expressed (e.g., transfected mRNA).

The NELL1 nucleic acid molecule can be introduced to allow for stable transformation or transient transformation. “Stable transformation” is intended to mean that the nucleotide construct introduced into a cell integrates into a genome of the cell. “Transient transformation” is intended to mean that a polynucleotide is introduced into the cell and does not integrate into a genome of the cell.

The term “transfection” refers to the introduction of foreign DNA into eukaryotic or prokaryotic cells. Transfection typically involves opening transient holes in cells to allow the entry of extracellular molecules, typically supercoiled plasmid DNA, but also siRNA, among others. There are various methods of transfecting cells. One method is by calcium phosphate. HEPES-buffered saline solution containing phosphate ions is combined with a calcium chloride solution containing the DNA to be transfected. When the two are combined, a fine precipitate of calcium phosphate will form, binding the DNA to be transfected on its surface. The suspension of the precipitate is then added to the cells to be transfected. The cells take up precipitate and the DNA. Alternatively, MgCl₂ or RbCl can be used. Other methods of transfection include electroporation, heat shock, proprietary transfection agents, dendrimers, and the use of liposomes. Liposomes are small, membrane-bounded bodies that fuse to the cell membrane releasing DNA into the cell. For eukaryotic cells, lipid-cation based transfection is typically used. Other methods of transfection include use of the gene gun and viruses. For stable transfection another gene is co-transfected, which gives the cell some selection advantage, such as resistance towards a certain toxin. If the toxin, towards which the co-transfected gene offers resistance, is then added to the cell culture, only those cells with the foreign genes inserted into their genome will be able to proliferate, while other cells will die. After applying this selection pressure for some time, only the cells with a stable transfection remain and can be cultivated further. A common agent for stable transfection is Geneticin, also known as G418, which is a toxin that can be neutralized by the product of the neomycin resistant gene (see Bacchetti and Graham (1997) Proc Natl Acad Sci USA 74(4):1590-94). Conventional transient transfection assays may incorporate internal controls, such as pRL-SV40 (Promega, Inc.) and may be used in combination with any experimental reporter vector to co-transfect mammalian cells.

The term “transformation” refers to the genetic alteration of a cell resulting from the introduction, uptake, and expression of foreign genetic material (DNA or RNA). In bacteria, transformation refers to a genetic change brought about by taking up and expressing DNA, and “competence” refers to a state of being able to take up DNA. Competent cells may be generated by a laboratory procedure in which cells are passively made permeable to DNA, using conditions that do not normally occur in nature, thus cells that have been manipulated to accept foreign DNA are called “competent cells”. These procedures are comparatively easy and simple, and can be used to genetically engineer bacteria. These procedures may include chilling cells in the presence of divalent cations, such as CaCl₂, which prepares the cell walls to become permeable to plasmid DNA. Cells are incubated with the DNA and then briefly heat shocked (e.g., 42° C. for 30-120 seconds), which causes the DNA to enter the cell. This method works well for circular plasmid DNAs. Electroporation is another way to allow DNA to enter cells and involves briefly shocking cells with an electric field of 100-200 V. Plasmid DNA enters cells via the holes created in the cell membrane by the electric shock; natural membrane-repair mechanisms close these holes afterwards. Yeasts may be transformed, for example, by High Efficiency Transformation (see Gietz and Woods (2002) Methods in Enzymology 350:87-96); the Two-hybrid System Protocol (see Gietz et al. (1997) Mol Cell Biochem 172:67-79); and the Rapid Transformation Protocol (see Gietz and Woods (2002) Methods in Enzymology 350:87-96).

In some embodiments, the nucleic acid molecule encoding a NELL1 peptide encodes an amino acid sequence having at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence set forth in SEQ ID NO: 2, 3, 4, 5, 6, 7, 8, 9, 11, 12, 13, 14, 15, 16, 17, 19, 21, 23, 25, 27, 29, 31, 33, 35, 37, 39, 49, 51, 53, 55, 57, 59, or 61. In certain embodiments, the NELL1 nucleic acid molecule has a nucleotide sequence having at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to the nucleotide sequence set forth in SEQ ID NO: 1, 10, 18, 20, 22, 24, 26, 28, 30, 32, 34, 36, 38, 48, 50, 52, 54, 56, 58, or 60.

According to a particular aspect, the described invention provides a cloning host cell, the cloning host cell comprising a cloning vector, the cloning vector comprising a recombinant expression cassette,
the recombinant expression cassette comprising:
(i) at least one regulatory element;
(ii) a reporter;
(iii) a NELL1 nucleotide sequence;
wherein the NELL1 nucleotide sequence encodes a peptide with substantial identity to a peptide having an amino acid sequence according to SEQ ID NO: 2, 11, 19, 21, 23, 25, 27, 29, 31, 33, 35, 37, 39, 49, 51, 53, 55, 57, 59, or 61;
wherein a composition comprising the NELL1 peptide reduces a manifestation of aged skin.

According to some embodiments, the at least one regulatory element is a promoter. According to some such embodiments, the regulatory element is a polyadenylation signal. According to some such embodiments, the regulatory element is a termination signal. According to some such embodiments, the regulatory element is a ribosome binding site.

According to another embodiment, the reporter is a detectable marker. According to some embodiments, the reporter is a selectable marker.

According to another embodiment, the NELL1 nucleic acid molecule encodes a peptide with substantial identity to a peptide having an amino acid sequence according to SEQ ID NO: 3, 4, 5, 6, 7, 8, 9, 12, 13, 14, 15, 16, or 17, wherein the peptide reduces a manifestation of aged skin. According to some such embodiments, the NELL1 nucleic acid molecule encodes a peptide with at least 95% sequence identity to a peptide having an amino acid sequence according to SEQ ID NO: 3, 4, 5, 6,7 8, 9, 12, 13, 14, 15, 16, or 17, wherein the peptide reduces a manifestation of aged skin.

According to another embodiment, the NELL1 nucleic acid molecule encodes a peptide, wherein the peptide is a fusion protein. The term “fusion protein” as used herein refers to a protein created through the joining of two or more genes which originally coded for separate proteins. Translation of the fusion gene results in a single polypeptide with functional properties derived from each of the original proteins. According to some such embodiments, the fusion protein comprises at least one NELL1 peptide selected from the group consisting of a peptide with at least 95% sequence identity to a peptide having an amino acid sequence according to SEQ ID NO: 3, 4, 5, 6, 7, 8, 9, 12, 13, 14, 15, 16, or 17, or a combination thereof. For example, the fusion protein may comprise at least one fragment, or a plurality of fragments, of the NELL1 peptide having an amino acid sequence with at least 70% sequence identity to a peptide having an amino acid sequence according to SEQ ID NO: 2, 11, 19, 21, 23, 25, 27, 29, 31, 33, 35, 37, 39, 49, 51, 53, 55, 57, 59, or 61, in any combination.

According to another embodiment, the manifestation of aged skin is selected from the group consisting of skin dryness, skin roughness, a rhytide, a pigmented lesion, an ephelide, a lentigine, patchy hyperpigmentation, a depigmented lesion, a guttate hypomelanosis, skin fragility, an area of purpura, a benign lesion, an acorchordon, a senile angioma, a seborrheic keratosis, a lentigo, a sebaceous hyperplasia, or a combination thereof.

According to another embodiment, the cloning vector is a plasmid. According to another embodiment, the cloning vector is a cosmid. According to another embodiment, the cloning vector is a bacterial phage. According to another embodiment, the cloning vector is a virus.

According to another embodiment, the cloning host cell is a prokaryotic cell. According to some such embodiments, the cloning host cell is a cell of strain Escherichia coli. According to another embodiment, the cloning host cell is a eukaryotic cell. According to some such embodiments, the eukaryotic cell is a yeast cell. According to some such embodiments, the eukaryotic cell is an insect cell. According to some such embodiments, the eukaryotic cell is an amphibian cell. According to some such embodiments, the eukaryotic cell is a mammalian. According to some such embodiments, the mammalian cell is a Chinese hamster ovary cell.

According to another embodiment, the described invention provides a recombinant expression cassette comprising an isolated nucleic acid that specifically hybridizes to mRNA encoding a peptide with at least 95% sequence identity to a NELL1 peptide having an amino acid sequence according to SEQ ID:2, 11, 19, 21, 23, 25, 27, 29, 31, 33, 35, 37, 39, 49, 51, 53, 55, 57, 59, or 61.

According to another embodiment, the described invention provides a recombination expression cassette comprising an isolated nucleic acid that specifically hybridizes to mRNA encoding a peptide with at least 95% sequence identity to a NELL1 peptide having an amino acid sequence according to SEQ ID: 3, 4, 5, 6, 7, 8, 9, 12, 13, 14, 15, 16, or 17.

### 4. Methods for Assaying Test Peptides for NELL1 Activity

According to one aspect, the described invention provides a method for assaying a test peptide for NELL1 activity. The method comprises administering the test peptide to a skin sample, irradiating the skin sample with ultraviolet radiation, and assessing the expression level of an inflammatory mediator, a matrix metalloproteinase, or an aquaporin in the skin sample, wherein a reduction in UV-stimulated expression of an inflammatory mediator or matrix metalloproteinase is indicative of NELL1 activity, or wherein an increase in UV-stimulated aquaporin expression is indicative of NELL1 activity.

According to another aspect, the described invention provides a method for assaying a test peptide for NELL1 activity. The method comprises administering the test peptide to a skin sample, irradiating the skin sample with ultraviolet radiation, and assessing the number of sunburned cells in the skin sample, wherein a reduced number of sunburned cells is indicative of NELL1 activity

Such methods find use in assaying a batch of NELL1 protein that has been produced and/or purified for activity for quality control purposes or for identifying active variants or fragments of a naturally-occurring NELL1 protein. Specific examples of assays that can be used to assess a test peptide include those disclosed herein in Experimental Example 3.

As used herein, a “test peptide” refers to the peptide that is being assayed using the presently disclosed methods to determine if the peptide has a NELL1 activity (e.g., reduction of UV-stimulated expression of an inflammatory mediator or MMP, increase in expression of an aquaporin, reduction in the number of sunburned cells). The test peptide can be a naturally-occurring NELL1 protein that has been purified from a natural source, or one that has been recombinantly or synthetically produced. Further, the test peptide can be an active variant or fragment of a wild-type NELL1 protein.

As used herein, a “NELL1 activity” refers to an activity of a naturally-occurring NELL1 protein. The NELL1 activity can be at least one of: stimulation of ECM production (e.g., through the upregulation of at least one of tenascins, proteoglycans, elastin, glycosaminoglycans, including epidermal hyaluronic acid, and collagens), reduction in the levels of inflammatory mediators (e.g., IL-1β and IL-8), reduction in the levels of matrix metalloproteinases (e.g., MMP1), increase in aquaporin (e.g., AQP3) levels, reduction in the number of sunburned cells, binding to PKC-beta, stimulation of differentiation of a precursor cell (e.g., skeletal satellite cell or osteoblast precursor) to maturity, and stimulation of angiogenesis.

As used herein “irradiate” or “irradiating” refers to the process by which a sample is exposed to radiation. In the presently disclosed methods, the skin sample is exposed to ultraviolet radiation. As used herein, “ultraviolet (UV) radiation” refers to electromagnetic radiation within the ultraviolet spectrum, with a wavelength of about 10 nm to about 400 nm, with energies from about 3 eV to about 124 eV. The term “ultraviolet” refers to UV A, UV B, and UV C. In some embodiments, the skin sample is irradiated with UV A and UV B. In some of these embodiments, the skin sample is irradiated with about 200 mJ/cm² UV B and about 29.94mW/cm² UV A.

The presently disclosed methods can be performed in vivo or on a skin tissue ex vivo. Alternatively, the skin sample can be a skin equivalent, such as the three-dimensional EpiDerm-FT™ 400 skin model that is commercially available from MatTek Corporation. Skin equivalents can be produced using any method known in the art. For example, dermal fibroblasts can be cultured in a collagen I gel onto which epidermal keratinocytes are seeded to produce stratified, differentiated full-thickness skin equivalents.

EpiDerm-FT™ is a normal (non-transformed), human cell-derived, metabolically active, three-dimensional organotypic in vitro full thickness skin model. Also known generically as reconstructed human epidermis, EpiDerm-FT™ closely mimics human epidermis, both structurally and biochemically, and does so in a very reproducible manner. EpiDerm-FT is a widely used model for studies ranging from drug delivery and toxicology to phototoxicity and wound healing. The well-documented tissue viability tests and a panel of other assays developed in EpiDerm-FT™, allow researchers to quantitatively measure the dermal responses to various experimental materials (Pratt et al. (2007) The Toxicologist 96, 1, 315; Hayden et al. (2003) Alternative Toxicological Methods, Eds. Salem et al., 229-247; Hayden et al. (2008) The Toxicologist 102,1, 69; MatTek Corporation, Technical Reference #622; and Limardi et al. (1996) J Invest Dermatol 106(4):939, Abstract #803).

Types of materials that have been tested using the EpiDerm-FT™ system include cosmetics and their constituents, household products, pharmaceuticals, even mustard gasses (Zhao et al. (1998) Invest Dermatol 110(4):525 Abstract #319; Hayden et al. (2009) Toxicol In Vitro 23:1396-1405; Karande et al. (2004) Nature Biotech 22(2):192-197; and Stinchcomb et al. (2002) American Association of Pharmaceutical Scientists Meeting). EpiDerm has been used successfully as an in vitro alternative in a number of toxicology tests including dermal phototoxicity. In the 3D model, viability and other parameters of both irradiated and non-irradiated tissues can be determined using MTT assay, cytokine release, histological examination, RNA abundance measurements, etc. (MatTek Corporation In Vitro 3-D Model Basics; Limardi et al. (1996) J Invest Dermatol 106(4):939, Abstract #803; and Last et al. (2002) Society of Investigative Dermatology Meeting).

The test peptide (and in some embodiments, the positive control NELL1 peptide) can be administered to the skin sample using any route of administration, including those listed elsewhere herein. In those embodiments wherein the skin sample is a skin equivalent (e.g., three-dimensional skin model), the test peptide (and in some embodiments, the positive control NELL1 peptide) can be administered topically to the stratum corneum surface of the skin equivalent cultures, can be added to the culture medium, can be injected, or can be administered using any route of administration that allows for penetratin of the NELL1 peptide into the tissue.

The test peptide (and in some embodiments, the positive control NELL1 peptide) can be administered at least one of before, during, and after irradiation. In some embodiments, the test peptide (and optionally, the positive control NELL1 peptide) is administered to the skin sample prior to irradiation and following irradiation. In some of these embodiments, the test peptide (and in some embodiments, the positive control NELL1 peptide) is administered to the skin sample for about 5 minutes, about 10 minutes, about 15 minutes, about 20 minutes, about 30 minutes, about 40 minutes, about 50 minutes, about 1 hour, about 2 hours, about 3 hours, about 4 hours, about 5 hours, about 10 hours, or greater prior to irradiation and about 1 hour, about 2 hours, about 3 hours, about 4 hours, about 5 hours, about 10 hours, about 15 hours, about 20 hours, about 24 hours, about 30 hours, about 36 hours, about 48 hours, or greater following irradiation. In certain embodiments, the NELL1 peptide is administered to a skin equivalent sample (e.g., via addition to the culture medium) for about 30 minutes prior to irradiation and about 24 hours following UV irradiation.

As used herein, the terms “sunburned cell” or “sunburn cell” refer to a skin cell that exhibits an abnormal morphology or is undergoing necrosis or apoptosis following exposure to UV. The number of sunburned skin cells can be quantitated by visual inspection using a cell stain, such as hematoxylin and eosin (H&E). Non-limiting examples of assays that can be used to measure levels of apoptosis include, but are not limited to, measurement of DNA fragmentation, caspase activation assays, TUNEL staining, annexin V staining

As used herein, “assessing the expression level” refers to any quantitative method that allows for the measurement of the amount of transcript or protein. Any method known in the art can be used to assess the expression level of an inflammatory mediator, matrix metalloproteinase, or aquaporin, including but not limited to measuring the transcript level or protein level, directly or indirectly (e.g., measuring activity of the protein).

In order to measure transcript levels, RNA must first be extracted from the skin sample. Methods of extraction of RNA are well-known in the art and are described, for example, in J. Sambrook et al., “Molecular Cloning: A Laboratory Manual” (Cold Spring Harbor Laboratory Press, Cold Spring Harbor, N.Y., 1989), vol. 1, ch. 7, “Extraction, Purification, and Analysis of Messenger RNA from Eukaryotic Cells,” incorporated herein by this reference. Other isolation and extraction methods are also well-known, for example in F. Ausubel et al., “Current Protocols in Molecular Biology”, John Wiley & Sons, 2007). Typically, isolation is performed in the presence of chaotropic agents, such as guanidinium chloride or guanidinium thiocyanate, although other detergents and extraction agents alternatively may be used. Typically, the mRNA is isolated from the total extracted RNA by chromatography over oligo(dT)-cellulose or other chromatographic media that have the capacity to bind the polyadenylated 3′-portion of mRNA molecules. Alternatively, but less preferably, total RNA can be used. However, it is generally preferred to isolate poly(A)+RNA from mammalian sources.

The transcript level can be measured using any method known in the art, including but not limited to, Northern blots, nuclease protection assays, reverse transcription (RT)-PCR, real-time RT-PCR, microarray analysis, and the like. The protein level can be measured using any method known in the art, including but not limited to, Western blots, immunoassays, ELISA, flow cytometry, protein microarrays, and the like.

As used herein, a “reduction in UV-stimulated expression” refers to a measurable decrease in the transcript and/or protein level of a gene whose expression is stimulated by UV radiation. UV radiation increases the expression level of the gene as compared to a control, untreated sample and treatment with the test peptide or positive control NELL1 peptide reduces the UV-induced expression level by at least 5%, 10%, 15%, 20%, 25%, 30%, 35%, 40%, 45%, 50%, 55%, 60%, 65%, 70%, 75%, 80%, 85%, 90%, 95%, or greater. In some embodiments, treatment of the UV-irradiated sample with the test peptide or positive control NELL1 peptide reduces the expression level of a gene to a level that is substantially the same or lower than the expression level of the gene in the absence of UV irradiation.

As used herein, an “increase in UV-stimulated expression” refers to a measurable increase in the transcript and/or protein level of a gene whose expression is stimulated by UV radiation. UV radiation increases the expression level of the gene as compared to a control, untreated sample and treatment with the test peptide or positive control NELL1 peptide further increases the UV-induced expression level by at least 5%, 10%, 15%, 20%, 25%, 30%, 35%, 40%, 45%, 50%, 55%, 60%, 65%, 70%, 75%, 80%, 85%, 90%, 95%, or greater.

In certain embodiments, the inflammatory mediator is an inflammatory cytokine, such as IL-1β or IL-8. In particular embodiments, the matrix metalloproteinase is MMP-1. In some embodiments, the aquaporin is AQP3. In some of these embodiments, the expression levels of IL-1, IL-8, MMP-1, or AQP3 can be assessed using primers that span the region that was amplified in the studies presented elsewhere herein (see Experimental Example 3).

In particular embodiments, the expression level of an inflammatory mediator, MMP, or aquaporin or the number of sunburned cells are compared to the expression level of the gene or number of sunburned cells in a negative control skin sample (i.e., a skin sample that has not been treated with the test peptide). In some of these embodiments, the negative control skin sample has been treated with a buffer or other vehicle or carrier that is administered with the test peptide.

In certain embodiments, the expression level of an inflammatory mediator, MMP, or aquaporin, or the number of sunburned cells is compared to the expression level of the gene or number of sunburned cells in a positive control skin sample. The positive control skin sample can be a skin sample (e g , skin equivalent) that has been treated with a NELL1 peptide that is known to have NELL1 activity. In some of these embodiments, the NELL1 peptide known to have NELL1 activity is human recombinant NELL1 protein (SEQ ID NO: 2), expressed in a wheat germ cell-free translation system, such as the NELL1 protein that is commercially available from Abnova Corporation (cat #H00004745-P01). In certain embodiments, the positive control NELL1 protein is added to the culture medium of a three dimensional skin model at a concentration of about 10 ng/ml, about 20 ng/ml, about 30 ng/ml, about 40 ng/ml, about 50 ng/ml, about 60 ng/ml, about 70 ng/ml, about 80 ng/ml, about 90 ng/ml, about 100 ng/ml, about 110 ng/ml, about 120 ng/ml, about 130 ng/ml, about 140 ng/ml, about 150 ng/ml, about 160 ng/ml, about 170 ng/ml, about 180 ng/ml, about 190 ng/ml, or about 200 ng/ml. In some of these embodiments, the positive control NELL1 protein (e.g., Abnova NELL1 having SEQ ID NO: 2) is added to the culture medium of a three-dimensional skin model (e.g., EpiDerm-FT™ 400) at a concentration of about 100 ng/ml.

### 5. Methods of Treatment

A therapeutically effective amount of a NELL1 peptide or a nucleic acid molecule encoding the same can be administered to a subject in need thereof in order to treat or prevent a skin condition (e.g., aging skin, skin scarring).

Any suitable route of administration may be used to deliver the NELL1 peptide or nucleic acid molecule encoding the same for the purposes of treating or preventing a skin condition. The term “administer” as used herein refers to the act of dispensing, supplying, applying, giving, or contributing to. The terms “administering” or “administration” are used interchangeably and include in vivo administration, as well as administration directly to tissue ex vivo. Generally, NELL1 peptides, nucleic acid molecules encoding the same, or compositions comprising the NELL1 peptide or nucleic acid may be administered systemically either orally, buccally, parenterally, topically, by inhalation or insufflation (i.e., through the mouth or through the nose), or rectally in dosage unit formulations, optionally containing the conventional nontoxic pharmaceutically acceptable carriers, adjuvants, and vehicles as desired, or may be locally administered by means such as, but not limited to, injection, implantation, grafting, or topical application. Additional administration may be performed, for example, intravenously, transmucosally, transdermally, intramuscularly, subcutaneously, intraperitoneally, intrathecally, intralymphatically, intralesionally, or epidurally. Administering can be performed, for example, once, a plurality of times, and/or over one or more extended periods.

In certain embodiments, the NELL1 peptide or nucleic acid molecule, or composition comprising the same is topically applied. The term “topical” as used herein refers to administration of an inventive composition at, or immediately beneath, the point of application. The term “topical administration” and “topically applying” as used herein are used interchangeably to refer to delivering a peptide, a nucleic acid, or a vector comprising the peptide or the nucleic acid, onto one or more surfaces of a tissue or cell, including epithelial surfaces. The term “epithelia” or “epithelial” or “epithelial tissues” as used herein is meant to include skin and mucosal membranes. The composition may be applied by pouring, dropping, or spraying, if a liquid; rubbing on, if an ointment, lotion, cream, gel, or the like; dusting, if a powder; spraying, if a liquid or aerosol composition; or by any other appropriate means. Topical administration generally provides a local rather than a systemic effect. The terms “topical administration” and “transdermal administration” as used herein, unless otherwise stated or implied, are used interchangeably.

Substances generally are applied to the skin to elicit one or more of four general effects: an effect on the skin surface, an effect within the stratum corneum; an effect requiring penetration into the epidermis and dermis; or a systemic effect resulting from delivery of sufficient amounts of a given substance through the epidermis and the dermis to the vasculature to produce therapeutic systemic concentrations. One example of an effect on the skin surface is formation of a film. Film formation may be protective (e.g., sunscreen) and/or occlusive (e.g., to provide a moisturizing effect by diminishing loss of moisture from the skin surface). One example of an effect within the stratum corneum is skin moisturization; which may involve the hydration of dry outer cells by surface films or the intercalation of water in the lipid-rich intercellular laminae; the stratum corneum also may serve as a reservoir phase or depot wherein topically applied substances accumulate due to partitioning into or binding with skin components.

It generally is recognized that short-term penetration occurs through the hair follicles and the sebaceous apparatus of the skin, while long-term penetration occurs across cells. Penetration of a substance into the viable epidermis and dermis may be difficult to achieve, but once it has occurred, the continued diffusion of the substance into the dermis is likely to result in its transfer into the microcirculation of the dermis and then into the general circulation. It is possible, however, to formulate delivery systems that provide substantial localized delivery.

When the NELL1 peptide, NELL1 nucleic acid molecule, or a composition comprising the same, along with a carrier are topically administered, the peptide or nucleic acid molecule is absorbed into the layers of the skin and/or beneath the skin. “Percutaneous absorption” is the absorption of substances from outside the skin to positions beneath the skin, including into the blood stream. The epidermis of human skin is highly relevant to absorption rates. Passage through the stratum corneum marks the rate-limiting step for percutaneous absorption. The major steps involved in percutaneous absorption of, for example, a drug, include the establishment of a concentration gradient, which provides a driving force for drug movement across the skin, the release of drug from the vehicle into the skin-partition coefficient and drug diffusion across the layers of the skin-diffusion coefficient. The relationship of these factors to one another is summarized by the following equation:

J=Cᵥₑₕ×KₘD/× [Formula 1]

where J=rate of absorption; Cᵥₑₕ=concentration of drug in vehicle; Kₘ=partition coefficient; and D=diffusion coefficient.

There are many factors which affect the rate of percutaneous absorption of a substance. Primarily they are as follows: (i) Concentration. The more concentrated the substance, the greater the absorption rate; (ii) Size of skin surface area to which the drug is applied. The wider the contact area of the skin to which the substance is applied, the greater the absorption rate; (iii) Anatomical site of application. Skin varies in thickness in different areas of the body. A thicker and more intact stratum corneum decreases the rate of absorbency of a substance. The stratum corneum of the facial area is much thinner than, for example, the skin of the palms of the hands. The facial skin's construction and the thinness of the stratum corneum provide an area of the body that is optimized for percutaneous absorption to allow delivery of active agents both locally and systemically through the body; (iv) Hydration. Hydration (meaning increasing the water content of the skin) causes the stratum corneum to swell which increases permeability; (v) Increased skin temperature increases permeability; and (vi) The composition of the compound and of the vehicle also determines the absorbency of a substance. Most substances applied topically are incorporated into bases or vehicles. The vehicle chosen for a topical application will greatly influence absorption, and may itself have a beneficial effect on the skin. Ideally, a vehicle having use in the present invention is easy to apply and remove, nonirritating, and cosmetically pleasing. In addition, the homeopathic complex must be stable in the chosen vehicle and must be released readily. Factors that determine the choice of vehicle and the transfer rate across the skin are the substance's partition coefficient, molecular weight and water solubility. The protein portion of the stratum corneum is most permeable to water soluble substances and the liquid portion of the stratum corneum is most permeable to lipid soluble substances. It follows that substances having both liquid and aqueous solubility can traverse the stratum corneum more readily. See Dermal Exposure Assessment: Principles and Applications, EPA/600/8-91/011b, January 1992, Interim Report—Exposure Assessment Group, Office of Health and Environmental Assessment, U.S. Environmental Protection Agency, Washington, D.C. 20460. The term “contacting” as used herein refers to bring or put in contact, to be in or come into contact. The term “contact” as used herein refers to a state or condition of touching or of immediate or local proximity. Contacting a composition to a target destination, such as, but not limited to, an organ, tissue, or cell may occur by any means of administration known to the skilled artisan.

In other embodiments, the NELL1 peptide, NELL1 nucleic acid molecule, or a composition comprising the NELL1 peptide or NELL1 nucleic acid molecule are administered parenterally. The term “parenteral” as used herein refers to introduction into the body by way of an injection (i.e., administration by injection), including, for example, subcutaneously (i.e., an injection beneath the skin beneath the dermis into the subcutaneous tissue or “superficial fascia”), intramuscularly (i.e., an injection into a muscle), intravenously (i.e., an injection into a vein), intrathecally (i.e., an injection into the space around the spinal cord or under the arachnoid membrane of the brain), intrasternal injection or infusion techniques. A parenterally administered composition is delivered using a needle, e.g., a surgical needle. The term “surgical needle” as used herein, refers to any needle adapted for delivery of fluid (i.e., capable of flow) compositions into a selected anatomical structure. Injectable preparations, such as sterile injectable aqueous or oleaginous suspensions, may be formulated according to the known art using suitable dispersing or wetting agents and suspending agents. According to some such embodiments, the NELL1 peptide or nucleic acid molecule encoding the same is administered by injection. According to some such embodiments, the injection is a subcutaneous injection. According to some such embodiments, the NELL1 peptide or nucleic acid molecule encoding the same is administered parenterally by injection to an area of the skin selected from the group consisting of the lateral aspect of forearms, the lateral aspect of legs, an elbow, a foot, a backhand, the back, the scalp, the face, and/or any other skin surfaces. According to another embodiment, the pharmaceutical or cosmetic composition is in the form of an implant.

The NELL1 peptide or nucleic acid molecule encoding the same is administered to a subject in need thereof to treat or prevent a skin condition. The term “subject” or “individual” or “patient” are used interchangeably to refer to a member of an animal species of mammalian origin, including but not limited to, mouse, rat, cat, goat, sheep, horse, hamster, ferret, pig, dog, platypus, guinea pig, rabbit and a primate, such as, for example, a monkey, ape, or human. Subjects in need of treatment with a NELL1 peptide or nucleic acid molecule are those having a skin condition (e.g., having at least one manifestation or symptom thereof) or susceptibility to a skin condition. For example, the NELL1 peptide or nucleic acid molecule encoding the same can be administered to a subject prior to the appearance of a manifestation of aging skin in order to prevent at least one manifestation of aging skin. The term “symptom” as used herein refers to a sign or an indication of a disorder or disease, especially when experienced by an individual as a change from normal function, sensation, or appearance. The term “syndrome,” as used herein, refers to a pattern of symptoms indicative of some disease or condition. The term “condition,” as used herein, refers to a variety of health states and is meant to include disorders or diseases caused by any underlying mechanism or disorder.

The term “disease” or “disorder,” as used herein, refers to an impairment of health or a condition of abnormal functioning. A “skin condition” includes, but is not limited to aging skin, a wound to the skin (e.g., epithelial surface wound), and skin scarring. A “skin condition” also includes a state wherein the appearance of the skin is less than desirable. In some embodiments, the skin condition is an injury or wound (e.g., to the epithelial surface). The term “injury” as used herein refers to damage or harm to a structure or function of the body caused by an outside agent or force, which may be physical or chemical. As used herein, the term “open wound” refers to a physical trauma where the skin is lacerated, cut or punctured. The term “a cut” refers to an injury that results in a break or opening in the skin, the term “a laceration” refers to a jagged, irregular cut, and the term “a puncture” refers to a wound made by a pointed object.

The term “treat” and its various grammatical forms as used herein includes abrogating, substantially inhibiting, slowing or reversing the progression of a disease, condition or disorder, substantially ameliorating clinical or esthetical symptoms of a condition, substantially preventing the appearance of clinical or esthetical symptoms of a disease, condition, or disorder, and protecting from harmful or annoying symptoms. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s).

The term “improve” and its various grammatical forms as used herein refers to a betterment or to bring into a more desirable or excellent condition.

The terms “inhibiting”, “inhibit” or “inhibition” are used herein to refer to reducing the amount or rate of a process, to stopping the process entirely, or to decreasing, limiting, or blocking the action or function thereof. Inhibition may include a reduction or decrease of the amount, rate, action function, or process of a substance by at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, at least 85%, at least 90%, at least 95%, at least 98%, or at least 99%.

The term “prevent” as used herein refers to effectual stoppage of action or progress. The term “renew” as used herein refers to the act of making new or as if new or to bringing back to an original condition. The term “repair” as used herein refers to the act of restoring to a good or sound condition after decay or damage.

A therapeutically effective amount of the NELL1 peptide or nucleic acid molecule encoding the same are administered to a subject in need thereof in order to treat or prevent a skin condition. The term “therapeutically effective amount” or an “amount effective” of one or more of the active agents of the present invention is an amount that is sufficient to provide a therapeutic effect. Generally, an effective amount of the active agents that can be employed according to the invention ranges from about 0.000001 mg/kg body weight to about 100 mg/kg body weight. However, dosage levels are based on a variety of factors, including the type of injury, the age, weight, sex, medical condition of the patient, genetic constitution of the patient, the severity of the condition, the route of administration, and the particular active agent employed. Thus the dosage regimen may vary widely, but can be determined routinely by a physician using standard methods. The term “therapeutic effect” as used herein refers to a consequence of treatment, the results of which are judged to be desirable and beneficial. A therapeutic effect may include, directly or indirectly, the arrest, reduction, or elimination of a disease manifestation. A therapeutic effect also may include, directly or indirectly, the arrest reduction or elimination of the progression of a disease manifestation. A therapeutic effect also may include aesthetic improvements (e.g., improvements in the appearance of skin). The term “appearance” as used herein refers to an outward look, aspect, state, condition, manner or style in which a person or object is seen or is perceived. Thus, treating a skin condition can comprise improving the appearance of the skin or skin condition.

The term “therapeutic agent” as used herein refers to a drug, molecule, nucleic acid, protein, metabolite, peptide, composition or other substance that provides a therapeutic effect. The term “active” as used herein refers to the ingredient, component or constituent of the compositions of the present invention responsible for the intended therapeutic effect. The terms “therapeutic agent” and “active agent” are used interchangeably herein. The term “drug” as used herein refers to a therapeutic agent or any substance, other than food, used in the prevention, diagnosis, alleviation, treatment, or cure of disease.

The term “therapeutic component” as used herein refers to a therapeutically effective dosage (i.e., dose and frequency of administration) that eliminates, reduces, or prevents the progression of a particular disease manifestation in a percentage of a population. An example of a commonly used therapeutic component is the ED₅₀ which describes the dose in a particular dosage that is therapeutically effective for a particular disease manifestation in 50% of a population.

In some embodiments, the therapeutically effective amount of a NELL1 peptide is at least about 0.0001 ng/kg body weight, at least about 0.0005 ng/kg body weight, at least about 0.001 ng/kg body weight, at least about 0.005 ng/kg body weight, at least about 0.01 ng/kg body weight, at least about 0.05 ng/kg body weight, at least about 0.1 ng/kg body weight, at least about 0.5 ng/kg body weight, at least about 1.0 ng/kg body weight, at least about 10 ng/kg body weight, at least about 100 ng/kg body weight, at least about 1 ng/kg body weight, at least about 1 ng/kg body weight, at least about 1 g/kg body weight, at least about 5 g/kg body weight, at least about 10 g/kg body weight, at least about 50 g/kg body weight, or at least about 100 g/kg body weight.

According to another embodiment, the therapeutically effective amount of a NELL1 peptide is at least about 0.01 nM, at least about 0.05 nM, at least about 0.1 nM, at least about 0.2 nM, at least about 0.3 nM, at least about 0.4 nM, at least about 0.5 nM, at least about 0.6 nM, at least about 0.7 nM, at least about 0.8 nM, at least about 0.9 nM, at least about 1.0 nM, at least about 10 nM, at least about 100 nM, at least about 500 nM, at least about 1 μM, or at least about 1 mM.

According to one aspect, the described invention provides a method for preventing or treating a skin condition, the method comprising administering a composition comprising a therapeutically effective amount of a NELL1 peptide or a nucleic acid molecule encoding the same and a carrier to an epithelial surface of a subject in need thereof, wherein the epithelial surface is skin, whereby at least one manifestation of the skin condition is prevented or treated.

According to another aspect, the described invention provides a method for treating at least one manifestation of aged skin, the method comprising administering a composition comprising a therapeutically effective amount of a NELL1 peptide or a nucleic acid molecule encoding the same and a carrier to an epithelial surface of a subject in need thereof, wherein the epithelial surface is aged skin, and improving the appearance of at least one manifestation of aged skin.

According to another aspect, the described invention provides a method for improving the appearance of aged skin, the method comprising administering a composition comprising a therapeutically effective amount of a NELL1 peptide or a nucleic acid molecule encoding the same and a carrier to an epithelial surface of a subject in need thereof, wherein the epithelial surface is aged skin, and repairing or improving the appearance of at least one manifestation of aged skin.

According to another aspect, the described invention provides a method for improving the appearance of aged skin, the method comprising administering a composition comprising a therapeutically effective amount of a NELL1 peptide or a nucleic acid molecule encoding the same and a carrier to an epithelial surface of a subject in need thereof, wherein the epithelial surface is aged skin, and repairing or improving the appearance of at least one manifestation of aged skin.

According to another aspect, the described invention provides a method for treating a wounded epithelial surface to prevent formation of a scar on the epithelial surface, the method comprising topically applying a topical composition comprising a therapeutically effective amount of a NELL1 peptide or a nucleic acid molecule encoding the same and a carrier to an epithelial surface of a subject in need thereof wherein the epithelial surface is wounded skin, whereby at least one manifestation of skin scarring is prevented.

According to one embodiment, the NELL1 peptide is a human NELL1 peptide.

According to another embodiment, the NELL1 peptide has an amino acid sequence of substantial identity to the amino acid sequence according to SEQ ID NO: 2. According to another embodiment, the NELL1 peptide has an amino acid sequence of at least about 70%, at least about 75%, at least about 80%, at least about 85%, at least about 90%, at least about 95%, at least about 99% or greater sequence identity to the amino acid sequence according to SEQ ID NO: 2, 3, 4, 5, 6, 7, 8, 9, 11, 12, 13, 14, 15, 16, 17, 19, 21, 23, 25, 27, 29, 31, 33, 35, 37, 39, 49, 51, 53, 55, 57, 59, or 61.

According to some embodiments, the composition comprises at least one NELL1 peptide selected from the group consisting of a peptide having an amino acid sequence of substantial identity to a peptide having an amino acid sequence according to SEQ ID NO: 3, 4, 5, 6, 7, 8, 9, 12, 13, 14, 15, 16, or 17.

According to another embodiment, the skin condition is aged skin. According to some such embodiments, the manifestation of the skin condition is skin dryness. According to some such embodiments, the manifestation of the skin condition is skin roughness. According to some such embodiments, the manifestation of the skin condition is a rhytide. According to some such embodiments, the manifestation of the skin condition is a pigmented lesion. According to some such embodiments, the pigmented lesion is an ephelide. According to some such embodiments, the pigmented lesion is a lentigine. According to some such embodiments, the manifestation of the skin condition is patchy hyperpigmentation. According to some such embodiments, the manifestation of the skin condition is a depigmented lesion. According to some such embodiments, the depigmented lesion is a guttate hypomelanosis. According to some such embodiments, the manifestation of the skin condition is skin fragility. According to some such embodiments, the manifestation of the skin condition is an area of purpura. According to some such embodiments, the manifestation of the skin condition is a benign lesion. According to some such embodiments, the benign lesion is an acrochordon.

According to some such embodiments, the benign neoplasm is a senile angioma. According to some such embodiments, the benign neoplasm is a seborrheic keratosis. According to some such embodiments, the benign neoplasm is a lentigo. According to some such embodiments, the benign neoplasm is a sebaceous hyperplasia. According to some embodiments, the manifestation of the skin condition, wherein the skin condition is aged skin, is at least one manifestation of aged skin selected from the group consisting of skin dryness, skin roughness, a rhytide, a pigmented lesion, an ephelide, a lentigine, patchy hyperpigmentation, a depigmented lesion, a guttate hypomelanosis, skin fragility, an area of purpura, a benign lesion, an acorchordon, a senile angioma, a seborrheic keratosis, a lentigo, a sebaceous hyperplasia, inflammation, or a combination thereof.

According to another embodiment, the skin condition is a scar. According to some such embodiments, the scar is a widespread scar, an atrophic scar, a raised skin scar, a hypertrophic scar or a keloid scar. According to some embodiments, the at least one manifestation of a skin scar is selected from the group consisting of: an elevation of an area of wounded skin, compared to normal skin; a thickening of an area of wounded skin, compared to normal skin; a nodularity of an area of wounded skin, compared to normal skin; a depression of an area of wounded skin, compared to normal skin; overgrowth of scar tissue that remains within the boundaries of an original wound; overgrowth of scar tissue that exceeds the boundaries of an original wound; an increased proliferation of fibroblasts in wounded skin, compared to normal skin; an increased fibroblast density in wounded skin, compared to normal skin, an increased sensitivity to ultraviolet light, compared to normal skin, a plurality of collagen fibers of random orientation in wounded skin; and a collagen content in wounded skin different from that of normal skin. According to some such embodiments, the manifestation of the skin condition, wherein the skin condition is a keloid scar, is a higher concentration of alanine transferase in the keloid scar compared to the concentration of alanine transferase in normal scar tissue. According to some such embodiments, the manifestation of the skin condition, wherein the skin condition is a keloid scar, is a higher concentration of adenosine triphosphate in the keloid scar compared to the concentration of adenosine triphosphate in normal scar tissue. According to some such embodiments, the manifestation of the skin condition, wherein the skin condition is a keloid scar, is an increased ratio of type I collagen to type III collagen compared to the ratio of type I collagen to type III collagen in normal scar tissue.

In those embodiments wherein a nucleic acid molecule encoding a NELL1 peptide is administered to a subject in need thereof, the nucleic acid is expressed to allow for the production of the NELL1 peptide and subsequent therapeutic effects. Thus, in some embodiments, the NELL1 nucleic acid molecule is operably linked to a promoter and optionally, additional regulatory elements. In particular embodiments, the NELL1 nucleic acid molecule is a recombinant expression vector or a cloning vector.

In particular embodiments, the NELL1 nucleic acid molecule has at least 70%, at least 75%, at least 80%, at least 85%, at least 90%, at least 95%, at least 96%, at least 97%, at least 98%, at least 99%, or greater sequence identity to the nucleotide sequence set forth in SEQ ID NO: 1, 10, 18, 20, 22, 24, 26, 28, 30, 32, 34, 36, 38, 48, 50, 52, 54, 56, 58, or 60. In certain embodiments, the NELL1 nucleic acid molecule encodes a NELL1 peptide having an amino acid sequence having at least 70%, at least 75%, at least 80%, at least 85%, at least 90%, at least 95%, at least 96%, at least 97%, at least 98%, at least 99%, or greater sequence identity to the amino acid sequence set forth in SEQ ID NO: 2, 3, 4, 5, 6, 7, 8, 9, 11, 12, 13, 14, 15, 16, 17, 19, 21, 23, 25, 27, 29, 31, 33, 35, 37, 39, 49, 51, 53, 55, 57, 59, or 61.

### 6. Pharmaceutical and Cosmetic Compositions

The NELL1 peptide or nucleic acid encoding the same can be administered to subjects in need thereof in the form of a composition further comprising a carrier. The term “carrier” as used herein describes a material that does not cause significant irritation to an organism and does not abrogate the biological activity and properties of the composition of the described invention. Carriers must be of sufficiently high purity and of sufficiently low toxicity to render them suitable for administration to a subject being treated. The carrier can be inert, or it can possess pharmaceutical benefits, cosmetic benefits or both.

The compositions comprising a NELL1 peptide or a nucleic acid molecule encoding the same can be a cosmetic or pharmaceutical composition. The term “cosmetic composition' as used herein refers to a composition that is intended to be rubbed, poured, sprinkled, or sprayed on, introduced into, or otherwise applied to a subject or any part thereof for cleansing, beautifying, promoting attractiveness, or altering the appearance, or an article intended for use as a component of any such article, except that such term does not include soap.

The phrase “cosmetically acceptable carrier” as used herein refers to a substantially non-toxic carrier, conventionally useable for the topical administration of cosmetics, with which compounds will remain stable and bioavailable.

The term “pharmaceutical composition” is used herein to refer to a composition that is employed to prevent, reduce in intensity, cure or otherwise treat a target condition or disease.

The term “pharmaceutically acceptable carrier” as used herein refers to one or more compatible solid or liquid filler, diluents or encapsulating substances which are suitable for administration to a human or other vertebrate animal. In some embodiments, the pharmaceutically acceptable carrier” is any substantially non-toxic carrier conventionally useable for topical administration of pharmaceuticals in which the compound will remain stable and bioavailable when applied directly to skin or mucosal surfaces.

Some non-limiting representative examples of carriers include moisturizing agents or humectants, pH adjusting agents, hair conditioning agents, chelating agents, preservatives, emulsifiers, thickners, solubilizing agents, penetration enhancers, anti-irritants, colorants and surfactants.

The term “moisturizing agent” as used herein refers to a substance that adds or restores moisture to the skin. Representative examples of moisturizing or humectant agents that are usable in the described invention include, without limitation, guanidine, glycolic acid and glycolate salts (e.g. ammonium salt and quaternary alkyl ammonium salt), aloe vera in any of its variety of forms (e.g., aloe vera gel), allantoin, urazole, polyhydroxy alcohols such as sorbitol, glycerol, hexanetriol, propylene glycol, butylene glycol, hexylene glycol and the like, polyethylene glycols, sugars and starches, sugar and starch derivatives (e.g., alkoxylated glucose), hyaluronic acid (HA), lactamide monoethanolamine, acetamide monoethanolamine and any combination thereof

As is widely recognized in the art, since the pH of the skin is 5.5, compositions for topical skin application (to avoid irritation) should have a pH value of between pH 4.0 and pH 7.0. In some embodiments, the pH is between pH 5.0 and pH 7.0. In some embodiments, the pH is about pH 5.5. Hence, a pH adjusting composition is typically added to bring the pH of the composition to the desired value. The compositions of the described invention therefore preferably are formulated to have a pH value of about 7.0. Suitable pH adjusting agents include, for example, but are not limited to, one or more adipic acids, glycines, citric acids, calcium hydroxides, magnesium aluminometasilicates, buffers or any combinations thereof.

Suitable hair conditioning agents that can be used in the context of the described invention include, for example, one or more collagens, cationic surfactants, modified silicones, proteins, keratins, dimethicone polyols, quaternary ammonium compounds, halogenated quaternary ammonium compounds, alkoxylated carboxylic acids, alkoxylated alcohols, alkoxylated amides, sorbitan derivatives, esters, polymeric ethers, glyceryl esters, or any combinations thereof

The terms “chelating agent”, “chelant” or “chelator” refers to a chemical that forms soluble complex molecules with certain metal ions thereby inactivating the ions. Chelating agents optionally are added to the compositions of the described invention so as to enhance the preservative or preservative system. In some embodiments, the chelating agents are mild agents, such as, for example, ethylenediaminetetraacetic acid (EDTA), EDTA derivatives, or any combination thereof.

Suitable preservatives for use in the compositions of the described composition include, without limitation, one or more alkanols, disodium EDTA (ethylenediamine tetraacetate), EDTA salts, EDTA fatty acid conjugates, isothiazolinone, parabens such as methylparaben and propylparaben, propylene glycols, sorbates, urea derivatives such as diazolindinyl urea, or botanical agents, such as, but not limited to, leuconostocil or radish root ferment filtrate, or any combinations thereof

In another embodiment, a composition comprising a NELL1 peptide or a nucleic acid molecule encoding the same, of the described invention, a carrier and other, optional ingredients can be dispersed in an emulsion.

In some embodiments, the compositions of the invention may be in the form of an oil-in-water emulsion. The oily phase may be a vegetable oil, for example, olive oil or arachis oil, or a mineral oil, for example a liquid paraffin, or a mixture thereof. Suitable emulsifying agents may be naturally-occurring gums, for example, gum acacia or gum tragacanth, naturally-occurring phosphatides, for example soy bean, lecithin, and esters or partial esters derived from fatty acids and hexitol anhydrides, for example sorbitan monooleate, and condensation products of the partial esters with ethylene oxide, for example, polyoxyethylene sorbitan monooleate.

The term “emulsifiers” as used herein are agents that promote the formation and stabilization of an emulsion. Suitable emulsifiers may be natural materials, finely divided solids, or synthetic materials. Natural emulsifying agents may be derived from either animal or vegetable sources. Those from animal sources include gelatin, egg yolk, casein, wool fat, or cholesterol. Those from vegetable sources include acacia, tragacanth, chondrus, or pectin. Vegetable sources specifically from cellulose derivatives include methyl cellulose and carboxymethyl cellulose to increase the viscosity. Finely divided emulsifiers include bentonite, magnesium hydroxide, aluminum hydroxide, or magnesium trisylicate. Synthetic agents include anionic, cationic or nonionic agents.

Particularly useful are sodium lauryl sulfate, benzalkonium chloride or polyethylene glycol 400 monostearate, or any combinations thereof.

The term “thickeners” as used herein refer to agents that make the composition of the described invention dense or viscous in consistency. Suitable thickeners that can be used in the context of the described composition include, for example, non-ionic water-soluble polymers such as hydroxyethylcellulose (commercially available under the Trademark Natrosol® 250 or 350), cationic water-soluble polymers such as Polyquat 37 (commercially available under the Trademark Synthalen® CN), fatty alcohols, fatty acids, anionic polymers, and their alkali salts and mixtures thereof. The term “polymer” as used herein refers to any of various chemical compounds made of smaller, identical molecules (called monomers) linked together. Polymers generally have high molecular weights. The process by which molecules are linked together to form polymers is called “polymerization.”

The term “solubilizing agents” as used herein refers to those substances that enable solutes to dissolve. Representative examples of solubilizing agents that are usable in the context of the described invention include, without limitation, complex-forming solubilizers such as citric acid, ethylenediamine-tetraacetate, sodium meta-phosphate, succinic acid, urea, cyclodextrin, polyvinylpyrrolidone, diethylammonium-ortho-benzoate, and micelle-forming solubilizers such as TWEEN®, e.g., TWEEN 80®, which is polysorbate 80, and SPANS (for example, SPAN® 20) which is sorbitan monolaurate, available from Croda International PLC. Other solubilizers that are usable for the compositions of the described invention are, for example, polyoxyethylene sorbitan fatty acid ester, polyoxyethylene n-alkyl ethers, n-alkyl amine n-oxides, polyoxamers, organic solvents, such as acetone, phospholipids and cyclodextrins.

The term “penetration enhancer” as used herein refers to an agent known to accelerate the delivery of a substance through the skin. Suitable penetration enhancers usable in the described invention include, but are not limited to, sulphoxides (such as dimethylsulphoxide, DMSO), Azones (e.g. laurocapram), pyrrolidones (for example 2-pyrrolidone, 2P), alcohols and alkanols (ethanol, or decanol), glycols (for example propylene glycol, PG, a common excipient in topically applied dosage forms), surfactants (also common in dosage forms), terpenes, and vegetable oils, including, for example, safflower oil, cottonseed oil and corn oil. Additional penetration enhancers include, but are not limited to, ionic surfactants (such as sodium lauryl sulfate, sodium laurate, polyoxyethylene-20-cetylether, laureth-9, sodium dodecylsulfate,dioctyl sodium sulfosuccinate), nonionic surfactants (such as polyoxyethylene-9-lauryl ether, Tween-80, nonylphenoxypolyoxyethylene, polysorbates), bile salts and derivatives (such as sodium glycocholoate, sodium deoxycholate, sodium taurocholate, sodium taurodihydrofusidate, sodium glycodihydrofusidate), fatty acids and derivatives (such as oleic acid, caprylic acid(s), mono(di)glycerides, lauric acids, acylcholines, acylcarnitines, sodium caprate), chelating agents (such as EDTA, citric acid, salicylates), polyols (such as glycerol, propanediol, polyethylene glycol), and other nonsurfactants (such as urea and its derivatives, unsaturated cyclic ureas, cyclodextrin, enamine derivatives, liposomes).

Additional thickeners, penetration enhancers and other adjuvants may generally be found in Remington's Pharmaceutical Sciences, 18th or 19th editions, published by the Mack Publishing Company of Easton, Pa., which is incorporated herein by reference.

The term “anti-irritant” as used herein refers to an agent that prevents or reduces soreness, roughness, or inflammation of a body part. Suitable anti-irritants that can be used in the context of the described invention include, for example, steroidal and non steroidal anti-inflammatory agents or other materials such as aloe vera, chamomile, alpha-bisabolol, cola nitida extract, green tea extract, tea tree oil, licorice extract, allantoin, caffeine or other xanthines, glycyrrhizic acid and its derivatives. Presently known anti-irritants can be divided into water-soluble anti-irritants and water-insoluble anti-irritants. Representative examples of such compositions are described, for example, in U.S. Pat. No. 5,482,710, which is incorporated herein by reference.

Colorants also may be used in the compositions of the invention. The term “colorant” as used herein refers to substance, dye, pigment, ink or paint that colors or modifies the hue of something. Colorants include pigments or dyes or a combination thereof as the cosmetic benefit requires. Preferred pigments include, but are not limited to, iron oxides, and titanium oxides. Suitable dyes include FD&C approved colorants, D&C approved colorants, and those approved for use in Europe and Japan. See Marmion, D. M., Handbook of US Colorants for Food, Drugs, Cosmetics, and Medical Devices, 3rd ed, 1991 herein incorporated by reference.

The term “surfactants” as used herein refers to surface-active substances, such as a detergent. Suitable surfactants for use with the inventive compositions include, but are not limited to, sarcosinates, glutamates, sodium alkyl sulfates, ammonium alkyl sulfates, sodium alkyleth sulfates, ammonium alkyleth sulfates, ammonium laureth-n-sulfates, sodium laureth-n-sulfates, isothionates, glycerylether sulfonates, sulfosuccinates and combinations thereof. The anionic surfactant may be selected from the group consisting of sodium lauroyl sarcosinate, monosodium lauroyl glutamate, sodium alkyl sulfates, ammonium alkyl sulfates, sodium alkyleth sulfates, ammonium alkyleth sulfates, and combinations thereof.

In another embodiment, the compositions of the described invention include a cosmetically acceptable carrier. It will be understood that cosmetically acceptable carriers and pharmaceutically acceptable carriers are similar, if not often identical, in nature.

Suitable cosmetically acceptable carriers are described in the CTFA International Cosmetic Ingredient Dictionary and Handbook, 8th edition, edited by Wenninger and Canterbery, (The Cosmetic, Toiletry, and Fragrance Association, Inc., Washington, D.C., 2000), which is herein incorporated by reference. Also included are the carriers described hereinabove.

In another embodiment, the compositions of the described invention further include one or more additional compatible active ingredients, which are aimed at providing the composition with another pharmaceutical, cosmeceutical or cosmetic effect, in addition to that provided by a compound of the inventive composition.

In one embodiment, the compound of the inventive compositions is an active ingredient.

In another embodiment, the compound of the inventive composition is a new excipient.

The phrase “new excipient” as used herein means any inactive ingredient that is intentionally added to the composition of the described invention and that is not intended to exert therapeutic effects at the intended dosage, although it may act to improve product delivery. A new excipient is not fully qualified by existing safety data with respect to the currently proposed level of exposure, duration of exposure or route of administration. Additional characteristics of new excipients can be found in the Guidance for Industry Nonclinical Studies for the Safety Evaluation of Pharmaceutical Excipients issued by the US Food and Drug Administration Center for Drug Evaluation and Research, in May, 2005, herein incorporated by reference.

Compositions according to the described invention, which further include one or more additional active ingredients, therefore can be further efficiently used, in addition to their use to treat a skin condition, to treat at least one manifestation of aged skin, to improve the appearance of aged skin, and to treat wounded skin to prevent formation of a scar, or a manifestation of a scar, in the treatment of any medical, cosmetic and/or cosmeceutical condition in which applying the additional active ingredient is beneficial.

The phrase “additional active ingredient” as used herein refers to an agent, other than a compound of the inventive composition, that exerts a pharmacological, dermatological or any other beneficial activity. It is to be understood that “other beneficial activity” may be one that is only perceived as such by the subject using the inventive compositions.

Additional active ingredients included in the compositions according to the described invention used to treat a manifestation of aged skin selected from the group consisting of dryness, roughness, wrinkling, skin pallor, hyperpigmentation, hypopigmenation, laxity, fragility, easy bruising and benign neoplasms, such as, but not limited to, acrochordons (skin tags), cherry angiomas (senile angiomas), seborrheic keratoses (senile wart or the “barnacles of old age”), lentigos (sun spots) and sebaceous hyperplasias, include, without limitation, one or more, in any combination, of a protective agent, an emollient, an astringent, an irritant, a keratolytic, a sun screening agent, a sun tanning agent, an antibiotic agent, a non-imidazole analog antifungal agent, an antifungal agent, an antiviral agent, an antiprotozoal agent, an anti-acne agent, an anesthetic agent, a steroidal anti-inflammatory agent, a non-steroidal anti-inflammatory agent, an antipruritic agent, an anti-oxidant agent, a chemotherapeutic agent, an anti-histamine agent, a peptide, a peptidomimetic, a peptide derivative, a vitamin, a vitamin supplement, a fusion protein, a hormone, an anti-dandruff agent, an anti-wrinkle agent, an anti-skin atrophy agent, a sclerosing agent, a cleansing agent, a caustic agent, and/or a hypo-pigmenting agent.

The term “acne” as used herein, refers to an inflammatory disease of the sebaceous glands, characterized by comedones and pimples. The term “anti-acne” as used herein refers to agents that alleviate the symptoms of acne. Examples of anti-acne agents include, without limitation, keratolytics, such as salicylic acid, sulfur, glycolic, pyruvic acid, resorcinol, and N-acetylcysteine; and retinoids such as retinoic acid and its derivatives (e.g., cis and trans, esters).

The term “anesthetic agents” as used herein refers to agents that result in a reduction or loss of sensation. Non-limiting examples of anesthetic drugs that are suitable for use in the context of the described invention include pharmaceutically acceptable salts of lidocaine, bupivacaine, chlorprocaine, dibucaine, etidocaine, mepivacaine, tetracaine, dyclonine, hexylcaine, procaine, cocaine, ketamine, pramoxine and phenol.

The term “antibiotic agent” as used herein means any of a group of chemical substances having the capacity to inhibit the growth of, or to destroy bacteria, and other microorganisms, used chiefly in the treatment of infectious diseases. Examples of antibiotic agents include, but are not limited to, Penicillin G; Methicillin; Nafcillin; Oxacillin; Cloxacillin; Dicloxacillin; Ampicillin; Amoxicillin; Ticarcillin; Carbenicillin; Mezlocillin; Azlocillin; Piperacillin; Imipenem; Aztreonam; Cephalothin; Cefaclor; Cefoxitin; Cefuroxime; Cefonicid; Cefmetazole; Cefotetan; Cefprozil; Loracarbef; Cefetamet; Cefoperazone; Cefotaxime; Ceftizoxime; Ceftriaxone; Ceftazidime; Cefepime; Cefixime; Cefpodoxime; Cefsulodin; Fleroxacin; Nalidixic acid; Norfloxacin; Ciprofloxacin; Ofloxacin; Enoxacin ; Lomefloxacin; Cinoxacin; Doxycycline; Minocycline; Tetracycline; Amikacin; Gentamicin; Kanamycin; Netilmicin; Tobramycin; Streptomycin; Azithromycin; Clarithromycin; Erythromycin; Erythromycin estolate; Erythromycin ethyl succinate; Erythromycin glucoheptonate; Erythromycin lactobionate; Erythromycin stearate; Vancomycin; Teicoplanin; Chloramphenicol; Clindamycin; Trimethoprim; Sulfamethoxazole; Nitrofurantoin; Rifampin; Mupirocin; Metronidazole; Cephalexin; Roxithromycin; Co-amoxiclavuanate; combinations of Piperacillin and Tazobactam; and their various salts, acids, bases, and other derivatives. Anti-bacterial antibiotic agents include, but are not limited to, penicillins, cephalosporins, carbacephems, cephamycins, carbapenems, monobactams, aminoglycosides, glycopeptides, quinolones, tetracyclines, macrolides, and fluoroquinolones.

The term “anti-fungal agent” as used herein means any of a group of chemical substances having the capacity to inhibit the growth of or to destroy fungi. Anti-fungal agents include, but are not limited to, Amphotericin B, Candicidin, Dermostatin, Filipin, Fungichromin, Hachimycin, Hamycin, Lucensomycin, Mepartricin, Natamycin, Nystatin, Pecilocin, Perimycin, Azaserine, Griseofulvin, Oligomycins, Neomycin, Pyrrolnitrin, Siccanin, Tubercidin, Viridin, Butenafine, Naftifine, Terbinafine, Bifonazole, Butoconazole, Chlordantoin, Chlormidazole, Croconazole, Clotrimazole, Econazole, Enilconazole, Fenticonazole, Flutrimazole, Isoconazole, Ketoconazole, Lanoconazole, Miconazole, Omoconazole, Oxiconazole, Sertaconazole, Sulconazole, Tioconazole, Tolciclate, Tolindate, Tolnaftate, Fluconazole, Itraconazole, Saperconazole, Terconazole, Acrisorcin, Amorolfine, Biphenamine, Bromosalicylchloranilide, Buclosamide, Calcium Propionate, Chlorphenesin, Ciclopirox, Cloxyquin, Coparaffinate, Diamthazole, Exalamide, Flucytosine, Halethazole, Hexetidine, Loflucarban, Nifuratel, Potassium Iodide, Propionic Acid, Pyrithione, Salicylanilide, Sodium Propionate, Sulbentine, Tenonitrozole, Triacetin, Ujothion, Undecylenic Acid, and Zinc Propionate.

The term “anti-dandruff agents” as used herein refers to agents that reduce, eliminate or prevent a scurf from forming on skin, especially of the scalp, that comes off in small white or grayish scales. Exemplary anti-dandruff ingredients usable in context of the described invention include, without limitation, zinc pyrithione, shale oil and derivatives thereof such as sulfonated shale oil, selenium sulfide, sulfur; salicylic acid, coal tar, povidone-iodine, imidazoles such as ketoconazole, dichlorophenyl imidazolodioxalan, clotrimazole, itraconazole, miconazole, climbazole, tioconazole, sulconazole, butoconazole, fluconazole, miconazole nitrate and any possible stereo isomers and derivatives thereof such as anthralin, piroctone olamine (Octopirox), selenium sulfide, and ciclopiroxolamine, and mixtures thereof

The term “antihistamine agent” as used herein refers to any of various compounds that counteract histamine in the body and that are used for treating allergic reactions (such as hay fever) and cold symptoms. Non-limiting examples of antihistamines usable in context of the described invention include chlorpheniramine, brompheniramine, dexchlorpheniramine, tripolidine, clemastine, diphenhydramine, promethazine, piperazines, piperidines, astemizole, loratadine and terfenadine.

The term “anti-protozoal agent” as used herein means any of a group of chemical substances having the capacity to inhibit the growth of or to destroy protozoans used chiefly in the treatment of protozoal diseases. Examples of antiprotozoal agents, without limitation, include pyrimethamine (Daraprim®), sulfadiazine, and Leucovorin.

The term “antipruritic agents” as used herein refers to those substances that reduce, eliminate or prevent itching. Suitable antipruritic agents include, without limitation, pharmaceutically acceptable salts of methdilazine and trimeprazine.

The term “anti-oxidant agent” as used herein refers to a substance that inhibits oxidation or reactions promoted by reactive oxygen species and free radicals.

Reactive oxygen species (“ROS”), such as free radicals and peroxides, represent a class of molecules that are derived from the metabolism of oxygen and exist inherently in all aerobic organisms. The term “oxygen radicals” as used herein refers to any oxygen species that carries an unpaired electron (except free oxygen). The transfer of electrons to oxygen also may lead to the production of toxic free radical species. The best documented of these is the superoxide radical. Oxygen radicals, such as the hydroxyl radical (OH—) and the superoxide ion (O₂⁻) are very powerful oxidizing agents that cause structural damage to proteins, lipids and nucleic acids. The free radical superoxide anion, a product of normal cellular metabolism, is produced mainly in mitochondria because of incomplete reduction of oxygen. The superoxide radical, although unreactive compared with many other radicals, may be converted by biological systems into other more reactive species, such as peroxyl (ROO⁻), alkoxyl (RO⁻) and hydroxyl (OH⁻) radicals.

Free radical scavengers/chemical antioxidants counteract and minimize free radical damage by donating or providing unpaired electrons to a free radical and converting it to a nonradical form. Such reducing compounds may terminate radical chain reactions and reduce hydroperoxides and epoxides to less reactive derivatives. Non-limiting examples of anti-oxidants that are usable in the context of the described invention include ascorbic acid (vitamin C) and its salts, ascorbyl esters of fatty acids, ascorbic acid derivatives (e.g., magnesium ascorbyl phosphate, sodium ascorbyl phosphate, ascorbyl sorbate), tocopherol (vitamin E), tocopherol sorbate, tocopherol acetate, other esters of tocopherol, butylated hydroxy benzoic acids and their salts, 6-hydroxy-2,5,7,8-tetramethylchroman-2-carboxylic acid (commercially available under the tradename Trolox®), gallic acid and its alkyl esters, especially propyl gallate, uric acid and its salts and alkyl esters, sorbic acid and its salts, lipoic acid, amines (e.g., N,N-diethylhydroxylamine, amino-guanidine), sulfhydryl compounds (e.g., glutathione), dihydroxy fumaric acid and its salts, glycine pidolate, arginine pilolate, nordihydroguaiaretic acid, bioflavonoids, curcumin, lysine, methionine, proline, superoxide dismutase, silymarin, tea extracts, grape skin/seed extracts, melanin, and rosemary extracts.

The term “anti-skin atrophy actives” refers to substances effective in replenishing or rejuvenating the epidermal layer by promoting or maintaining the natural process of desquamation. Non-limiting examples of antiwrinkle and antiskin atrophy actives, which can be used in context of the described invention, include retinoic acid, its prodrugs and its derivatives (e.g., cis and trans) and analogues; salicylic acid and derivatives thereof, sulfur-containing D and L amino acids (e.g., cysteine, methionine) and their derivatives (e.g., N-acetylcysteine) and salts; thiols, e.g. ethane thiol; alpha-hydroxy acids, e.g. glycolic acid, and lactic acid; phytic acid, lipoic acid; lysophosphatidic acid, and skin peel agents (e.g., phenol and the like).

The term “anti-viral agent” as used herein means any of a group of chemical substances having the capacity to inhibit the replication of or to destroy viruses used chiefly in the treatment of viral diseases. Anti-viral agents include, but are not limited to, Acyclovir, Cidofovir, Cytarabine, Dideoxyadenosine, Didanosine, Edoxudine, Famciclovir, Floxuridine, Ganciclovir, Idoxuridine, Inosine Pranobex, Lamivudine, MADU, Penciclovir, Sorivudine, Stavudine, Trifluridine, Valacyclovir, Vidarabine, Zalcitabine, Zidovudine, Acemannan, Acetylleucine, Amantadine, Amidinomycin, Delavirdine, Foscamet, Indinavir, Interferons (e.g., IFN-alpha), Kethoxal, Lysozyme, Methisazone, Moroxydine, Nevirapine, Podophyllotoxin, Ribavirin, Rimantadine, Ritonavir2, Saquinavir, Stailimycin, Statolon, Tromantadine, Zidovudine (AZT) and Xenazoic Acid.

The term “astringents” as used herein generally refers to protein precipitants that have such a low cell penetrability that their action essentially is limited to the cell surface and interstitial spaces. Astringents are locally applied. The astringent action is accompanied by contraction and wrinkling of the tissue and by blanching. Astringents are used therapeutically to arrest hemorrhage by coagulating the blood, to promote healing, to toughen the skin or to decrease sweating. Components of astringents include salts of aluminum, zinc, manganese, iron or bismuth.

The term “caustic agents” as used herein refers to substances capable of destroying or eating away epithelial tissue by chemical action. Caustic agents can be used to remove dead skin cells. For example, beta-hydroxy acids, naturally derived acids with a strong kerolytic effect, are useful for problem skin, acne or peeling. The term “chemotherapeutic agent” as used herein refers to chemicals useful in the treatment or control of a disease.

The term “emollients” as used herein generally refers to bland, fatty or oleaginous materials which can be applied locally, particularly to the skin. Emollients increase the tissue moisture content, thereby rendering the skin softer and more pliable. Increased moisture content in the skin can be achieved by preventing water loss with an occlusive water-immiscible barrier, by increasing the water-holding capacity in the skin with humectants, or by altering the desquamation of the outermost skin layer, the stratum corneum. Useful emollients include, but are not limited to, lanolin, spermaceti, mineral oil, paraffin, petrolatum, white ointment, white petroleum, yellow ointment. Also included are vegetable oils, waxes, cetyl alcohol, glycerin, hydrophilic petrolatum, isopropyl myristate, myristyl alcohol, and oleyl alcohol.

The term “hypopigmenting agents” as used herein refers to substances capable of depigmenting the skin. Suitable hypopigmenting agents include, but are not limited to, hydroquinones, mequinol, and various protease inhibitors including serine protease inhibitors, active soy and retinoic acid.

The term “irritant” as used herein refers to a material that acts locally on the skin to induce, based on irritant concentration, hyperemia (meaning an excess of blood in an area or body part, usually indicated by red, flushed color or heat in the area), inflammation, and desiccation. Irritant agents include, but are not limited to, alcohol, aromatic ammonia spirits, benzoin tincture, camphor capsicum, and coal tar extracts.

The term “keratolytic” (desquamating agent) as used herein refers to an agent that acts to remove outer layers of the stratum corneum. Keratolytics are particularly useful in hyperkeratotic areas. The keratolytics include, but are not limited to, benzoyl peroxide, fluorouracil, resorcinol, salicylic acid, tretinoin, and the like.

The term “non-steroidal anti-inflammatory agents” as used herein refers to a large group of agents that are aspirin-like in their action, including, but not limited to, ibuprofen (Advil®), naproxen sodium (Aleve®), and acetaminophen (Tylenol®). Additional examples of non-steroidal anti-inflammatory agents that are usable in the context of the described invention include, without limitation, oxicams, such as piroxicam, isoxicam, tenoxicam, sudoxicam, and CP-14,304; disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Mixtures of these non-steroidal anti-inflammatory agents also may be employed, as well as the dermatologically acceptable salts and esters of these agents. For example, etofenamate, a flufenamic acid derivative, is particularly useful for topical application.

The terms “sclerosant” and “sclerosing agent” as used herein refer to an agent used as a chemical irritant injected into a vein in sclerotherapy. Examples of sclerosants include, but are not limited to, morrhuate sodium, sodium tetradecyl sulfate, laureth 9 and ethanolamine oleate.

The term “steroidal anti-inflammatory agent” as used herein refers to any one of numerous compounds containing a 17-carbon 4-ring system and includes the sterols, various hormones (as anabolic steroids), and glycosides. Representative examples of steroidal anti-inflammatory drugs include, without limitation, corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene(fluprednylidene)acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

The term “vitamin” as used herein, refers to any of various organic substances essential in minute quantities to the nutrition of most animals. Vitamins act especially as coenzymes and precursors of coenzymes in the regulation of metabolic processes. Non-limiting examples of vitamins usable in context of the described invention include vitamin A and its analogs and derivatives: retinol, retinal, retinol palmitate, retinoic acid, tretinoin, iso-tretinoin (known collectively as retinoids), vitamin E (tocopherol and its derivatives), vitamin C (L-ascorbic acid and its esters and other derivatives), vitamin B3 (niacinamide and its derivatives), alpha hydroxy acids (such as glycolic acid, lactic acid, tartaric acid, malic acid, citric acid, etc.) and beta hydroxy acids (such as salicylic acid and the like).

Additional active ingredients included in the compositions according to the described invention for treating wounded skin to prevent formation of a scar or at least one manifestation of a scar include, without limitation, at least one of a protective agent, an emollient, an astringent, an irritant, a keratolytic, a sun screening agent, a sun tanning agent, an antibiotic agent, a non-imidazole analog antifungal agent, an antifungal agent, an antiviral agent, an antiprotozoal agent, an anti-acne agent, an anesthetic agent, a steroidal anti-inflammatory agent, a non-steroidal anti-inflammatory agent, an antipruritic agent, an anti-oxidant agent, a chemotherapeutic agent, an anti-histamine agent, a peptide, a peptidomimetic, a peptide derivative, a vitamin, a vitamin supplement, a fusion protein, a hormone, an anti-dandruff agent, an anti-wrinkle agent, an anti-skin atrophy agent, a sclerosing agent, a cleansing agent, a caustic agent, or a hypo-pigmenting agent.

Protectives as described herein may take the form of dusting powders, adsorbents, mechanical protective agents, and plasters. Dusting powders are relatively inert and insoluble materials that are used to cover and protect epithelial surfaces, ulcers and wounds. Usually, these substances are finely subdivided powders that absorb moisture and can act as a dessicant. The term “drying agent” or “dessicant” as used herein refers to a substance that has an affinity for water such that it will extract the water from other materials.

The absorption of skin moisture decreases friction and also discourages certain bacterial growth. Some of the materials used as protective adsorbents include bentonite, insoluble salts of bismuth, boric acid, calcium carbonate, (precipitated), cellulose, cornstarch, magnesium stearate, talc, titanium dioxide, zinc oxide, and zinc stearate.

Protectives also can be administered to the skin to form an adherent, continuous film that may be flexible or semi-rigid depending on the materials and the formulations as well as the manner in which they are applied. This material may serve several purposes including providing occlusion from the external environment, providing chemical support, and serving as vehicles for other medicaments. Mechanical protectives are generally either collodions or plasters. Examples include aluminum hydroxide gel, collodium, dimethicone, petrolatum gauze, absorbable gelatin film, absorbable gelatin sponge, zinc gelatin, kaolin, lanolin, anhydrous lanolin, mineral oil, mineral oil emulsion, mineral oil light, olive oil, peanut oil, petrolatum, silicones, hydrocolloids and the like.

In some embodiments, protectives included in the composition of the invention are demulcents. The term “demulcents” as used herein refers to protective agents employed primarily to alleviate irritation. They often are applied to the surface in a viscid, sticky preparation that covers the area readily and may be medicated. A number of chemical substances possess demulcent properties. These substances include, but are not limited to, the alginates, mucilages, gums, dextrins, starches, certain sugars, and polymeric polyhydric glycols. Others include acacia, agar, benzoin, carbomer, gelatin, glycerin, hydroxyethyl cellulose, hydroxypropyl cellulose, hydroxypropyl methylcellulose, propylene glycol, sodium alginate, tragacanth, hydrogels and the like.

In some embodiments, the irritant is a rubefacient. The term “rubefacient” as used herein refers to an agent that induces hyperemia, wherein hyperemia means an increased amount of blood in a body part or organ. Rubefaction, which is induced by rubefacients, results from increased circulation to an injured area and is accompanied by a feeling of comfort, warmth, itching and hyperesthesia.

Representative examples of sun screening agents usable in context of the described invention include, without limitation, p-aminobenzoic acid and its salts and derivatives thereof (ethyl, isobutyl, glyceryl esters; p-dimethylaminobenzoic acid); anthranilates (i.e., o-amino-benzoates; methyl, menthyl, phenyl, benzyl, phenylethyl, linalyl, terpinyl, and cyclohexenyl esters); salicylates (amyl, phenyl, octyl, benzyl, menthyl, glyceryl, and di-propylene glycol esters); cinnamic acid derivatives (menthyl and benzyl esters, a-phenyl cinnamonitrile; butyl cinnamoyl pyruvate); dihydroxycinnamic acid derivatives (umbelliferone, methylumbelliferone, methylaceto-umbelliferone); trihydroxy-cinnamic acid derivatives (esculetin, methylesculetin, daphnetin, and the glucosides, esculin and daphnin); hydrocarbons (diphenylbutadiene, stilbene); dibenzylacetone and benzylacetophenone; naphtholsulfonates (sodium salts of 2-naphthol-3,6-disulfonic and of 2-naphthol-6,8-disulfonic acids); di-hydroxynaphthoic acid and its salts; o- and p-hydroxybiphenyldisulfonates; coumarin derivatives (7-hydroxy, 7-methyl, 3-phenyl); diazoles (2-acetyl-3-bromoindazole, phenyl benzoxazole, methyl naphthoxazole, various aryl benzothiazoles); quinine salts (bisulfate, sulfate, chloride, oleate, and tannate); quinoline derivatives (8-hydroxyquinoline salts, 2-phenylquinoline); hydroxy- or methoxy-substituted benzophenones; uric and violuric acids; tannic acid and its derivatives (e.g., hexaethylether); (butyl carbotol) (6-propyl piperonyl)ether; hydroquinone; benzophenones (oxybenzene, sulisobenzone, dioxybenzone, benzoresorcinol, 2,2′,4,4′-tetrahydroxybenzophenone, 2,2′-dihydroxy-4,4′-dimethoxybenzophenone, octabenzone; 4-isopropyldibenzoylmethane; butylmethoxydibenzoylmethane; etocrylene; octocrylene; [3-(4′-methylbenzylidene boman-2-one) and 4-isopropyl-di-benzoylmethane, and any combination thereof.

Representative examples of sunless tanning agents usable in the described invention include, without limitation, dihydroxyacetone, glyceraldehyde, indoles and their derivatives. The sunless tanning agents can be used in combination with the sunscreen agents.

Cleansing agents which may be used in the described invention include surfactant based cleansing agents, examples of which have been listed hereinabove. Other non-surfactant-based cleansing agents known to those of skill in the art also may be employed.

The topical compositions of the described invention can be applied locally to the skin and may be in any form including solutions, oils, creams, ointments, gels, lotions, shampoos, milks, cleansers, moisturizers, sprays, skin patches and the like.

In another embodiment, a NELL1 peptide or nucleic acid molecule encoding the same, of the described invention, a carrier and, optionally, an additional active ingredient(s) are formed into a composition comprising a solution, emulsion or gel suspension.

In some embodiments, a NELL1 peptide or nucleic acid molecule encoding the same of the described invention, a pharmaceutical or cosmetic carrier and, optionally, one or more additional active ingredients are in the form of a solution. A “solution” generally is considered as a homogeneous mixture of two or more substances. It is frequently, though not necessarily, a liquid. In a solution, the molecules of the solute (or dissolved substance) are uniformly distributed among those of the solvent. Solvents that may be useful in the compositions of the present invention include water, as well as organic solvents, such as the alcohols.

A solution can be prepared by mixing a solute or dissolved substance (such as a compound of the invention and, optionally, one or more active ingredient(s) uniformly throughout a solvent carrier such as water or organic solvents, such as the alcohols (e.g. ethanol or isopropanol, acetone).

Emulsifying agent carriers useful in the described invention are described hereinabove.

In yet, another embodiment, a composition containing a NELL1 peptide or nucleic acid molecule encoding the same of the described invention can be mixed with a gel suspension, (a semi-solid carrier) or solid carrier to form a paste, powder, ointment, cream, lotion, hydrogel or the like.

In some embodiments, the compositional form is a paste, meaning a semisolid dosage form that contains one or more substances intended for topical application. Pastes generally are divided into fatty paste and those made from a single-phase aqueous gel.

In some embodiments, the compositional form is a cream. The term “cream” as used herein refers to a viscous liquid or semisolid emulsion of either the oil-in-water or water-in-oil type. As used herein “emulsion” refers to a colloid system in which both the dispersed phase and the dispersion medium are immiscible liquids where the dispersed liquid is distributed in small globules throughout the body of the dispersion medium liquid. A stable basic emulsion contains at least the two liquids and an emulsifying agent. Common types of emulsions are oil-in-water, where oil is the dispersed liquid and an aqueous solution, such as water, is the dispersion medium, and water-in-oil, where, conversely, an aqueous solution is the dispersed phase. It also is possible to prepare emulsions that are nonaqueous. Creams of the oil-in-water type include hand creams and foundation creams. Water-in-oil creams include cold creams and emollient creams. The term “emollient” as used herein refers to fats or oils in a two-phase system (meaning one liquid is dispersed in the form of small droplets throughout another liquid). Emollients soften the skin by forming an occlusive oil film on the stratum corneum, preventing drying from evaporation in the deeper layers of skin. Thus, emollients are employed as protectives and as agents for softening the skin, rendering it more pliable. Emollients also serve as vehicles for delivery of hydrophobic compounds. Common emollients used in the manufacture of cosmetics include, but are not limited to, butters, such as Aloe Butter, Almond Butter, Avocado Butter, Cocoa Butter, Coffee Butter, Hemp Seed Butter, Kokum Butter, Mango Butter, Mowrah Butter, Olive Butter, Sal Butter, Shea Butter, glycerin, and oils, such as Almond Oil, Aloe Vera Oil, Apricot Kernel Oil, Avocado Oil, Babassu Oil, Black Cumin Seed Oil, Borage Seed Oil, Brazil Nut Oil, Camellia Oil, Castor Oil, Coconut Oil, Emu Oil, Evening Primrose Seed Oil, Flaxseed Oil, Grape Seed Oil, Hazelnut Oil, Hemp Seed Oil, Jojoba Oil, Kukui Nut Oil, Macadamia Nut Oil, Meadowfoam Seed Oil, Mineral Oil, Neem Seed Oil, Olive Oil, Palm Oil, Palm Kernel Oil, Peach Kernel Oil, Peanut Oil, Plum Kernel Oil, Pomegranate Seed Oil, Poppy Seed Oil, Pumpkin Seed Oil, Rice Bran Oil, Rosehip Seed Oil, Safflower Oil, Sea Buckthorn Oil, Sesame Seed Oil, Shea Nut Oil, Soybean Oil, Sunflower Oil, Tamanu Oil, Turkey Red Oil, Walnut Oil, Wheatgerm Oil

Creams may be diluted only with suitable diluents specified in the appropriate entries, and diluted creams must be freshly prepared without the application of heat. Creams should be stored in a cool place and supplied in well-closed containers that prevent evaporation and contamination of the contents. When making a natural cream, however, butters first are melted. The vessel is removed from the heat and the oils are added. When the solution is 100 degrees F., the balance of the liquid portion of the formula then is slowly added while continuously stirred.

In some embodiments, the compositional form is an ointment, meaning a semi-solid preparations intended for external application to the epithelium. For example, ointments may be prepared which are in gel-suspension form. Generally, ointment bases are categorized into hydrocarbon bases (oleaginous), which may use white petroleum as a base; adsorption bases (anhydrous), which might use hydrophilic petroleum or anhydrous lanolin; emulsion bases (water and oil type); emulsion bases (oil and water type); and water soluble bases, which often use polyethylene glycol as an ointment base.

In some embodiments, the compositional form is a lotion, meaning a liquid or semi-liquid preparation that contains one or more active ingredients in an appropriate vehicle. A lotion may be a suspension of solids in an aqueous medium, an emulsion, or a solution.

In some embodiments, the compositional form of the invention is a gel. The term “gel” as used herein refers to a sticky, jelly-like semisolid or solid prepared from high molecular weight polymers in an aqueous or alcoholic base. Alcoholic gels are drying and cooling, while non-alcoholic gels are more lubricating and are well suited, for example, to dry scaling lesions. Due to their drying effect, especially from those gels containing alcohol, gels may cause irritation and cracking of the skin. Starches and aloe are commonly used agents in the manufacture of gelled cosmetic preparations.

The term “hydrogel” as used herein refers to a substance resulting in a solid, semisolid, pseudoplastic, or plastic structure containing a necessary aqueous component to produce a gelatinous or jelly-like mass.

Additional compositions of the described invention can be readily prepared using technology which is known in the art such as described in Remington's Pharmaceutical Sciences, 18th or 19th editions, published by the Mack Publishing Company of Easton, Pa.

In some embodiments, the compositions of the described invention include about 0.0001% to about 10.0% w/w of a NELL1 peptide of the described invention, including but not limited to about 0.0001%, about 0.001%, about 0.01%, about 0.1%, about 1.0%, about 2.0%, about 3.0%, about 4.0%, about 5.0%, about 6.0%, about 7.0%, about 8.0%, about 9.0%, and about 10.0% w/w.

According to another aspect of the described invention, there is provided a method of preparing the compositions described hereinabove. The process generally includes admixing a NELL1 peptide or nucleic acid molecule encoding the same, as described hereinabove, and the pharmaceutically, cosmetically or cosmeceutically acceptable carrier. In cases where additional active ingredients, as detailed above, are present in the compositions, the process includes admixing these ingredients together with the active ingredients and the carrier. The mixing technique utilized in the process of the described invention can involve any one of the known techniques for formulating topical compositions. A variety of exemplary formulation techniques that are usable in the process of the described invention is described, for example, in Harry's Cosmeticology, Seventh Edition, Edited by J B Wilkinson and R J Moore, Longmann Scientific & Technical, 1982, incorporated herein by reference in its entirety.

According to another aspect of the described invention, there is provided a method of treating a medical, cosmetic and/or cosmeceutical condition associated with epithelial tissues having at least one manifestation of skin aging. The method is effected by topically applying a pharmaceutically, cosmetically or cosmeceutically effective amount of the composition of the described invention as described above onto an epithelial-related surface.

According to some embodiments of the described invention, the compositions of the described invention are applied topically as needed. According to some embodiments, the inventive compositions are topically applied between one and four times a day, or twice a day (e.g., once in the morning and once in the evening). The topical application of the compositions of the described invention may be carried out daily. Some conditions may require topical application for an indeterminate length of time.

In one embodiment, the inventive compositions are topically administered to an epithelial surface of a subject. Non-limiting examples of an epithelial surface onto which the compositions of the described invention can be applied topically include the lateral aspect of a forearm, the lateral aspect of a leg, an elbow, a foot, a backhand, the back, the scalp, the face, and any other skin surface, and any mucosal membrane described herein.

Alternatively, the compositions may be administered to an epithelial surface as a component of, for example, a bandage, adhesive, or transdermal patch. In these instances, the compositions may be an integral component of the bandage, adhesive, or transdermal patch and are thereby applied to the epithelial surface.

According to some embodiments, the carrier is a pharmaceutically acceptable carrier.

Suitable pharmaceutically acceptable carriers include water, petroleum jelly (Vaseline™), petroleum, mineral oil, vegetable oil, animal oil, organic and inorganic waxes, such as microcrystalline, paraffin and ozocerite wax, natural polymers, such as xanthanes, gelatin, cellulose, collagen, starch, or gum arabic, alcohols, polyols, and the like. Also included are the carriers described hereinabove.

In another embodiment, the pharmaceutically acceptable carrier of the composition of the described invention includes a sustained release or delayed release carrier. The carrier can be any material capable of sustained or delayed release of the compound to provide a more efficient administration resulting in less frequent and/or decreased dosage of the compound, ease of handling, and extended or delayed effects on at least one manifestation of skin aging. Non-limiting examples of such carriers include liposomes, microsponges, microspheres, or microcapsules of natural and synthetic polymers and the like. Liposomes, which may enhance the localized delivery of the compounds of the inventive composition within skin layers, may be formed from a variety of phospholipids, such as cholesterol, stearylamines or phosphatidylcholines. The surface of the liposomes may be labeled with a targeting ligand to specifically deliver the NELL1 peptide or nucleic acid encoding the same to desired tissues.

According to another embodiment, the compositions, wherein it is desirable to deliver them locally, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension also may contain suitable stabilizers or agents which increase the solubility of the compounds to allow for the preparation of highly concentrated solutions. Alternatively, the active compounds may be in powder form for constitution with a suitable vehicle, e.g., sterile pyrogen-free water, before use.

The pharmaceutical compositions also may comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Suitable liquid or solid pharmaceutical preparation forms are, for example, microencapsulated, and if appropriate, with one or more excipients, encochleated, coated onto microscopic gold particles, contained in liposomes, pellets for implantation into the skin, or dried onto an object to be rubbed into the skin. Such pharmaceutical compositions also may be in the form of granules, beads, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, or solubilizers are customarily used as described above. The pharmaceutical compositions are suitable for use in a variety of drug delivery systems. For a brief review of methods for drug delivery, see Langer (1990) Science 249, 1527-1533, which is incorporated herein by reference.

The composition, and optionally other therapeutics, may be administered per se or in the form of a pharmaceutically acceptable salt. When used in medicine, the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts may be prepared as alkaline metal or alkaline earth salts, such as sodium, potassium or calcium salts of the carboxylic acid group. By “pharmaceutically acceptable salt” is meant those salts which are, within the scope of sound medical judgment, suitable for use in contact with the tissues of humans and lower animals without undue toxicity, irritation, allergic response and the like and are commensurate with a reasonable benefit/risk ratio. Pharmaceutically acceptable salts are well-known in the art. For example, P. H. Stahl, et al. describe pharmaceutically acceptable salts in detail in “Handbook of Pharmaceutical Salts: Properties, Selection, and Use” (Wiley VCH, Zurich, Switzerland: 2002). The salts may be prepared in situ during the final isolation and purification of the compounds described or separately by reacting a free base function with a suitable organic acid. Representative acid addition salts include, but are not limited to, acetate, adipate, alginate, citrate, aspartate, benzoate, benzenesulfonate, bisulfate, butyrate, camphorate, camphorsufonate, digluconate, glycerophosphate, hemisulfate, heptanoate, hexanoate, fumarate, hydrochloride, hydrobromide, hydroiodide, 2-hydroxyethansulfonate(isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups may be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which may be employed to form pharmaceutically acceptable acid addition salts include, but are not limited to, such inorganic acids as hydrochloric acid, hydrobromic acid, sulphuric acid and phosphoric acid and such organic acids as oxalic acid, maleic acid, succinic acid and citric acid. Basic addition salts may be prepared in situ during the final isolation and purification of compounds described by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like. Pharmaceutically acceptable salts also may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium or magnesium) salts of carboxylic acids may also be made.

The formulations may be presented conveniently in unit dosage form and may be prepared by any of the methods well known in the art of pharmacy. All methods include the step of bringing into association the described composition, or a pharmaceutically acceptable salt or solvate thereof (“active compound”) with the carrier which constitutes one or more accessory agents. In general, the formulations are prepared by uniformly and intimately bringing into association the active agent with liquid carriers or finely divided solid carriers or both and then, if necessary, shaping the product into the desired formulation.

The pharmaceutical agent or a pharmaceutically acceptable ester, salt, solvate or prodrug thereof may be mixed with other active materials that do not impair the desired action, or with materials that supplement the desired action. Solutions or suspensions used for parenteral, intradermal, subcutaneous, intrathecal, or topical application may include, but are not limited to, for example, the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation may be enclosed in ampoules, disposable syringes or multiple dose vials made of glass or plastic. Administered intravenously, particular carriers are physiological saline or phosphate buffered saline (PBS).

Pharmaceutical compositions for parenteral injection comprise pharmaceutically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity may be maintained, for example, by the use of a coating such as lecithin, by the maintenance of the required particle size in the case of dispersions, and by the use of surfactants.

These compositions also may contain adjuvants including preservative agents, wetting agents, emulsifying agents, and dispersing agents. Prevention of the action of microorganisms may be ensured by various antibacterial and antifungal agents, for example, parabens, chlorobutanol, phenol, sorbic acid, and the like. It also may be desirable to include isotonic agents, for example, sugars, sodium chloride and the like. Prolonged absorption of the injectable pharmaceutical form may be brought about by the use of agents delaying absorption, for example, aluminum monostearate and gelatin.

Suspensions, in addition to the active compounds, may contain suspending agents, as, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

Injectable depot forms are made by forming microencapsulated matrices of the drug in biodegradable polymers such as polylactide-polyglycolide. Depending upon the ratio of drug to polymer and the nature of the particular polymer employed, the rate of drug release may be controlled. Such long acting formulations may be formulated with suitable polymeric or hydrophobic materials (for example as an emulsion in an acceptable oil) or ion exchange resins, or as sparingly soluble derivatives, for example, as a sparingly soluble salt. Examples of other biodegradable polymers include poly(orthoesters) and poly(anhydrides). Depot injectable formulations also are prepared by entrapping the drug in liposomes or microemulsions which are compatible with body tissues.

The locally injectable formulations may be sterilized, for example, by filtration through a bacterial-retaining filter or by incorporating sterilizing agents in the form of sterile solid compositions that may be dissolved or dispersed in sterile water or other sterile injectable medium just prior to use. Injectable preparations, for example, sterile injectable aqueous or oleaginous suspensions, may be formulated according to the known art using suitable dispersing or wetting agents and suspending agents. The sterile injectable preparation also may be a sterile injectable solution, suspension or emulsion in a nontoxic, parenterally acceptable diluent or solvent such as a solution in 1,3-butanediol. Among the acceptable vehicles and solvents that may be employed are water, Ringer's solution, U.S.P. and isotonic sodium chloride solution. In addition, sterile, fixed oils conventionally are employed or as a solvent or suspending medium. For this purpose any bland fixed oil may be employed including synthetic mono- or diglycerides. In addition, fatty acids such as oleic acid are used in the preparation of injectables.

Formulations for parenteral (including but not limited to, subcutaneous, intradermal, intramuscular, intravenous, intrathecal and intraarticular) administration include aqueous and non-aqueous sterile injection solutions that may contain anti-oxidants, buffers, bacteriostats and solutes, which render the formulation isotonic with the blood of the intended recipient; and aqueous and non-aqueous sterile suspensions, which may include suspending agents and thickening agents. The formulations may be presented in unit-dose or multi-dose containers, for example sealed ampules and vials, and may be stored in a freeze-dried (lyophilized) condition requiring only the addition of the sterile liquid carrier, for example, saline, water-for-injection, immediately prior to use. Extemporaneous injection solutions and suspensions may be prepared from sterile powders, granules and tablets of the kind previously described.

Another method of formulation of the compositions described herein involves conjugating the compounds described herein to a polymer that enhances aqueous solubility. Examples of suitable polymers include but are not limited to polyethylene glycol, poly-(d-glutamic acid), poly-(1-glutamic acid), poly-(1-glutamic acid), poly-(d-aspartic acid), poly-(1-aspartic acid), poly-(1-aspartic acid) and copolymers thereof. Polyglutamic acids having molecular weights between about 5,000 to about 100,000, with molecular weights between about 20,000 and about 80,000 may be used and with molecular weights between about 30,000 and about 60,000 also may be used.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

In some embodiments, the composition is a pharmaceutical composition. The pharmaceutical compositions described within the present invention contain a therapeutically effective amount of a NELL1 peptide or a nucleic acid molecule encoding the same, and optionally other therapeutic agents included in a pharmaceutically-acceptable carrier. The active ingredient may be a composition comprising a NELL1 peptide or a nucleic acid molecule encoding the same. The components of the pharmaceutical compositions also are capable of being co-mingled in a manner such that there is no interaction which would substantially impair the desired pharmaceutical efficiency.

The therapeutic agent(s), including the described composition, may be provided in or on particles. The term “particle” as used herein refers to a nano or microparticle (or in some instances larger) that may contain in whole or in part the described composition. The particles may contain the therapeutic agent(s) in a core surrounded by a coating. The therapeutic agent(s) also may be dispersed throughout the particles. The therapeutic agent(s) also may be adsorbed on at least one surface of the particles. The particles may be of any order release kinetics, including zero order release, first order release, second order release, delayed release, sustained release, immediate release, etc., and any combination thereof. The particle may include, in addition to the therapeutic agent(s), any of those materials routinely used in the art of pharmacy and medicine, including, but not limited to, erodible, nonerodible, biodegradable, or nonbiodegradable material or combinations thereof. The particles may be microcapsules that contain the described composition in a solution or in a semi-solid state. The particles may be of virtually any shape.

Both non-biodegradable and biodegradable polymeric materials may be used in the manufacture of particles for delivering the therapeutic agent(s). Such polymers may be natural or synthetic polymers. The polymer is selected based on the period of time over which release is desired. Bioadhesive polymers of particular interest include bioerodible hydrogels as described by Sawhney et al in Macromolecules (1993) 26, 581-587, the teachings of which are incorporated herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The therapeutic agent(s) may be contained in controlled release systems. In order to prolong the effect of a drug, it often is desirable to slow the absorption of the drug from subcutaneous, intrathecal, or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. In some embodiments, the use of a long-term sustained release implant may be particularly suitable for treatment of chronic conditions. Long-term sustained release implants are well-known to those of ordinary skill in the art and include some of the release systems described above.

The term “controlled release” is intended to refer to any drug-containing formulation in which the manner and profile of drug release from the formulation are controlled. This includes immediate as well as non-immediate release formulations, with non-immediate release formulations including, but not limited to, sustained release and delayed release formulations. The term “sustained release” (also referred to as “extended release”) is used herein in its conventional sense to refer to a drug formulation that provides for gradual release of a drug over an extended period of time, and that preferably, although not necessarily, results in substantially constant blood levels of a drug over an extended time period. The term “delayed release” is used herein in its conventional sense to refer to a drug formulation in which there is a time delay between administration of the formulation and the release of the drug there from. “Delayed release” may or may not involve gradual release of drug over an extended period of time, and thus may or may not be “sustained release.” The term “long-term” release, as used herein, means that the implant is constructed and arranged to deliver therapeutic levels of the active ingredient for at least 7 days, and preferably about 30 to about 60 days.

In another embodiment, the described composition further comprises a gel, slow-release solid or semisolid compound, wherein the gel, slow-release solid or semisolid compound comprises a therapeutically effective amount of a NELL1 peptide, or fragment(s) thereof, and a coating. The coating can be of any desired material, preferably a polymer or mixture of different polymers. Optionally, the polymer may be utilized during the granulation stage to form a matrix with the active ingredient so as to obtain a desired release pattern of the active ingredient. The gel, slow-release solid or semisolid compound is capable of releasing the active agent over a desired period of time. The gel, slow-release solid or semisolid compound may be implanted in close proximity to a desired location, whereby the release of the active agent produces a localized pharmacologic effect.

In another embodiment, the described composition further comprises a semisolid delivery system that utilizes a semisolid, biodegradable, biocompatible delivery system or a biodegradable, biocompatible multiparticulate dispersed and suspended in a semisolid, biodegradable, biocompatible biodegradable delivery system for injection, deposition or implantation within or upon the body so as to facilitate local therapeutic effects. The term “biocompatible” as used herein refers to not causing injury, toxicity or an immunologic reaction. The term “biodegradable” as used herein refers to a material that will degrade actively or passively over time by simple chemical processes, by action of body enzymes or by other similar mechanisms in the human body.

In another embodiment, the semisolid delivery system comprises partially or in whole a biocompatible, biodegradable, viscous semisolid wherein the semisolid comprises a hydrogel. The hydrogel incorporates and retains significant amounts of H₂O, which eventually will reach an equilibrium content in the presence of an aqueous environment. In one embodiment, glyceryl monooleate, hereinafter referred to as GMO, is the intended semisolid delivery system or hydrogel. However, many hydrogels, polymers, hydrocarbon compositions and fatty acid derivatives having similar physical/chemical properties with respect to viscosity/rigidity may function as a semisolid delivery system.

In another embodiment, the NELL1 peptide or nucleic acid encoding the same is part of a multiparticulate component. According to some such embodiments, the multiparticulate component is comprised of biocompatible, biodegradable, polymeric or non-polymeric systems utilized to produce solid structures including but not limited to nonpareils, pellets, crystals, agglomerates, microspheres, or nanoparticles.

In another embodiment, the multiparticulate component comprises poly(lactic-co-glycolide) (PLGA's). PLGA's are biodegradable polymer materials used for controlled and extended therapeutic agent delivery within the body. Such delivery systems offer enhanced therapeutic efficacy and reduced overall toxicity as compared to frequent periodic, systemic dosing. According to another embodiment, the PLGA composition is sufficiently pure so as to be biocompatible and remains biocompatible upon biodegradation. According to another embodiment, the PLGA polymer is designed and configured into microspheres having a therapeutic agent or drug entrapped therein, whereby the therapeutic agent is subsequently released therefrom. In some such embodiments, the therapeutic agent is a NELL1 peptide or nucleic acid encoding the same.

In another embodiment, the multiparticulate component is comprised of poly d,1(lactic-co-caprolactone). This provides a biodegradable polymer material used for controlled and extended therapeutic agent delivery within the body with a similar drug release mechanism to that of the PLGA polymers. In another embodiment, the multiparticulate microspheres also are produced using biodegradable and/or biocompatible non-polymeric materials such as GMS.

In another embodiment, the multiparticulate component is further modified by methods used to encapsulate or coat the multiparticulate components using polymers of the same composition with the same or different drug substances, different polymers with the same or different drug substances, or with multiple layering processes containing no drug, the same drug, a different drug, or multiple drug substances. This allows the production of a multi-layered (encapsulated) multiparticulate system with a wide range of drug release profiles for single or multiple drug agents simultaneously. In another embodiment, coating materials which control the rate of physical drug diffusion from the multiparticulate may be utilized alone or in concert with the aforementioned embodiments and envisioned embodiments.

General methods in molecular genetics and genetic engineering useful in the present invention are described in the current editions of Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2nd Ed. (R. I. Freshney. 1987. Liss, Inc. New York, N.Y.), and Gene Transfer and Expression Protocols, pp. 109-128, ed. E. J. Murray, The Humana Press Inc., Clifton, N.J.). Reagents, cloning vectors, and kits for genetic manipulation are available from commercial vendors such as BioRad, Stratagene, Invitrogen, ClonTech and Sigma-Aldrich Co.

Unless defined otherwise, all technical and scientific terms used herein have the same meaning as commonly understood by one of ordinary skill in the art to which this invention belongs. Any methods and materials similar or equivalent to those described herein can also be used in the practice or testing of the present invention.

It must be noted that as used herein and in the appended claims, the singular forms, “a”, “an”, “and”, and “the” include plural references unless the context clearly dictates otherwise. As such, the terms “a” (or “an”), “one or more,” and “at least one” can be used interchangeably herein. All technical and scientific terms used herein have the same meaning Efforts have been made to ensure accuracy with respect to numbers used (e.g. amounts, temperature, etc.) but some experimental errors and deviations should be accounted for.

Throughout this specification and the claims, the words “comprise,” “comprises,” and “comprising” are used in a non-exclusive sense, except where the context requires otherwise.

As used herein, the term “about,” when referring to a value is meant to encompass variations of, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Where a range of values is provided, it is understood that each intervening value, to the tenth of the unit of the lower limit, unless the context clearly dictates otherwise, between the upper and lower limit of the range and any other stated or intervening value in that stated range, is encompassed within the invention. The upper and lower limits of these small ranges which may independently be included in the smaller rangers is also encompassed within the invention, subject to any specifically excluded limit in the stated range. Where the stated range includes one or both of the limits, ranges excluding either or both of those included limits are also included in the invention.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. It is intended that the scope of the invention be construed to include all modifications and alterations that may occur to others upon reading and understanding the preceding detailed description insofar as they come within the scope of the following claims or equivalents thereof. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Production and Purification of Human NELL1 Protein

Human NELL1 (hNELL1) protein is produced and purified. First, the 2448 by hNELL1 cDNA is isolated by PCR from a human brain cDNA library. To produce the hNELL1 protein as a C-terminally V5- and His×6-tagged form (hNELL1-VH), the hNELL1 cDNA fragment is inserted downstream from the OpIE2 promoter of the expression vector pIZT/V5-His contained in the InsectSelect Glow System. Next, the High Five cells are transfected with the resultant plasmid pIZT/V5-His-NELL1 using FuGene 6 (Roche, Manneheim, Germany) and incubated for 48 hour in a serum-free medium, Express Five SFM (Invitrogen). An antibiotic Zeocin (Nakalai Tesque, Kyoto, Japan) is added to the medium at 400 μg/ml and Zeocin-resistant cells are selected by replacing the medium every 3 to 4 days. For monitoring the extracellular production of hNELL1-VH protein, the culture medium (6 ml) is incubated with an anti-V5 tag antibody (1 μg; Nakalai Tesque) and the precipitates are subjected to sodium dodecyl sulfate-polyacrylamide gel electrophoresis (SDS-PAGE) with a 6.0% gel and Western blotting using the same antibody on a PVDF membrane. For affinity purification of hNELL1-VH protein in a large scale, the 3-day culture medium (combined to 500 ml) is applied onto a plastic column (Φ1.5×10 cm) packed with Ni Sepharose 6 Fast Flow (GE Healthcare UK, Buckingham, UK), washed extensively with phosphate buffer saline (PBS), and eluted with 500 mM imidazole in PBS. The eluate (about 10 ml) is dialyzed extensively against PBS at 4° C. overnight and concentrated by ultrafiltration to about 900 μg/ml. The purity of hNELL1-VH1 protein is checked by SDS-PAGE and staining with Coomassie Brilliant Blue R-250, migrating to about 120 kDa.

### Example 2

### Animal Studies

NELL1 will be assayed in an animal model of solar-aged skin.

2.1.1. Animals

Female albino hairless Skh:HR-1 mice are obtained, housed five to a cage, and maintained on Purina Chow. Mice are ten weeks old at the start of experimental work.

2.1.2. Irradiation

Briefly, a bank of four 4-foot fluorescent lamps is used. The bank contains various combinations of Westinghouse FS-40 sunlamps and GE F-40 black lights. The latter lights have no detectable emission below 340 nm. The lamp housing is lined with aluminum foil as a reflecting surface. The distance from the lamps to the animal's back is about 45 cm. The energy output of the lamps at that distance is measured with an International Light (Newburyport, Mass.) model 700 A research radiometer, model 791 photomultiplier, model GMA 201-9 wavelength drive, and model 760 photomultiplier power supply. Output is recorded on a model 4950 strip chart recorder (Bausch & Lomb, Austin, Tex.). Irradiation times are adjusted as necessary to correct for decline in lamp outputs with time. Only the area of uniform irradiation under the lamps is used.

For irradiation, mice are housed individually in 7.5 cm×7.5 cm×7.5 cm stainless steel irradiation chambers. There are fifteen mice per group. Irradiation doses for the bulb arrangements are as follows: 20, 40 or 60 mJ cm⁻² for 4 UVB bulbs; 20, 40 or 60 mJ cm⁻² of UVB and 0.5, 1.0 or 1.5 J cm⁻² of UVA, respectively, for 2 UVB plus 2 UVA bulbs; and 2.5, 5.0 and 7.5 J cm⁻² for 4 UVA bulbs. The high dose UVB and UVA plus UVB exposures listed are slightly less than the mouse 1 MED (minimal erythemal dose) levels for the experimental setup. The end-point for MED determinations is the appearance of any edema, erythema or petechia in the irradiated skin in the 48 hours following UV exposure.

Irradiations are done three times weekly (Monday, Wednesday and Friday). For the UVB and UVB plus UVA groups, irradiation is stopped after 16 weeks. There is then a 17-week recovery period. For the UVA groups, irradiation is stopped after 33 weeks. There is then an 18-week recovery period.

For NELL1 treated groups, a NELL1 topical composition (1 μg/kg body weight) is applied at the rate of 3 μg cm⁻² to the back skin immediately before irradiation. Irradiations are as described for the non-NELL1 groups. In all NELL1 groups, irradiation is stopped after 33 weeks. There is then an 18-week recovery period.

Measurement of skin temperature with a surface temperature probe (Model BAT8, Bailey Instruments, Saddle Brook, N.J.) confirms no change in skin temperature with the irradiations used here.

2.1.3. Physical Measures

All physical measurements are made at four- to six-week intervals on all mice.

Least significant difference (LSD) analysis of the data is done to determine statistically significant differences (95% confidence level).

Skin fold thickness is measured with calipers accurate to 2.5 mm. The calipers are modified with brass rectangular plates (0.6 cm×2 cm) in the pinch area. To make measurements, the midline skin is manually pinched upward at the neck and base of the tail. The skin fold thickness of this skin pinch is then measured mid-way between the shoulder blades and hips.

Transepidermal water loss (TEWL) is measured using a Servomed (Stockholm, Sweden) model EP1 evaporimeter. The instrument is calibrated over standard salt solutions before each measurement session. TEWL readings are taken mid-way between the shoulders and hips. Instrument readings are allowd to reach a steady state (about 10 seconds) before recording the data.

The microtopography of the skin surface is measured in vivo by a modified casting technique. Briefly, a mixture of Water Plus cement and Acryl 60 (both from Thoro System Products, Centerville, Ind.) is made to set in 30 seconds. The mixture is applied to the upper back of unrestrained mice. Casts prepared with restrained or unrestrained mice are not different. The casts are evaluated at 12× magnification under a stereomicroscope and graded by the scale in Table 1(half-grade increments may be used with this scale).

TABLE 1
Grading scale of mouse skin casts.

| Grade | Description of Skin |
|---|---|
| 0 | Abundant fine structure. No specific |
| | orientation to structure. |
| 1 | Fine structure has fiber-like appearance. |
| | Fibers are oriented. |
| 2 | Rod-like, parallel bundles present. Fine |
| | structures and fibers still apparent between |
| | bundles. |
| 3 | Bundles are major structure. Fine structure |
| | nearly absent. Fibers and bundles less well |
| | defined. |
| 4 | Absence of fine structure fibers and |
| | bundles. Smooth appearance. |

2.1.4. Grading of Visible Changes

With UV exposure, mouse skin develops visible wrinkling, sagging and tumors. Wrinkling and sagging on all mice are graded by at least two trained graders with the scales in Table 2 and Table 3, respectively (half-grade increments may be used with these scales). Permanent tumors (1 mm diameter or greater) are counted. These assessments are made weekly.

TABLE 2
Grading scale of mouse skin wrinkling.

| Grade | Description of Skin |
|---|---|
| 0 | Numerous fine striations covering back and |
| | flanks of body. Fine striations run length of |
| | body (head-to-tail direction) and appear and |
| | disappear with motion. |
| 1 | All fine striations on back along spine gone. |
| | A few shallow coarse wrinkles across back |
| | (run perpendicular to head-to-tail direction) |
| | which appear and disappear with motion. |
| 2 | All fine striations gone. Some coarse |
| | wrinkles across back (run perpendicular to |
| | head-to-tail direction) which are permanent. |
| 3 | All fine striations gone. Several deep |
| | coarse wrinkles across back (run |
| | perpendicular to head-to-tail direction) |
| | which are permanent. |

TABLE 3
Grading scale of mouse skin sagging.

| Grade | Description of Skin |
|---|---|
| 0 | Numerous fine striations covering back and |
| | flanks of body. Fine striations run length of |
| | body (head-to-tail direction). Skin has |
| | purple-pink coloration. |
| 1 | Loss of fine striations on neck and |
| | shoulders and along flanks. Some sagging |
| | on neck (sags run perpendicular to head-to- |
| | tail direction). Some nodular wrinkling of |
| | back skin. Loss of skin coloration in neck |
| | and shoulder areas. Slight blanching of skin |
| | on upper back area. |
| 2 | All fine striations lost. Moderate sagging |
| | on neck and along flanks. Moderate |
| | modular wrinkling of back skin. All skin |
| | coloration lost. |
| 3 | All fine striations lost. Severe sagging on |
| | neck and along flanks. Severe modular |
| | wrinkling of back skin. All skin coloration |
| | lost. |

2.1.5. Histology

With UVB- and UVB plus UVA-irradiated mice, histology is done at weeks 6, 12, 16, 28 and 33. With UVA-irradiated mice, histology is done at weeks 6, 12, 31 and 51. For NELL1 treated groups, histology is done at weeks 6, 12, 33 and 51. Three mice from each group are sacrificed at each time point for histology.

Animals are sacrificed 4 days after the last irradiation to permit recovery from acute UV effects. Animals are sacrificed by CO₂ asphyxiation. Strips of dorsal skin are fixed in 10% buffered formalin, embedded in paraffin, and sectioned at 6 to 10 μm. Sections are stained with a variety of stains. These are: hematoxylin and eosin (H&E), Pinkus acid orcein-giemsa for elastin, Van Gieson's for collagen, Mowry's collodial iron for glycosaminoglycans (GAGs), and alcian blue at pH 2.5 for hyaluronic acid (HA) and at pH 1.0 for sulfated GAGs. Luna's mast cell stain with the counter stain methyl orange also is used as a stain for elastin. The number of sunburned cells (e.g., cells having abnormal morphology or undergoing necrosis or apoptosis) in the H&E stained sections will also be assessed.

The visible appearance of the skin will be observed to change with UV exposure. Anticipated changes include at least wrinkling, sagging and/or tumor development. The degree of change is expected to be UV dose dependent.

Skin wrinkling will be induced by UVB and UVB plus UVA. Skin sagging will be induced by high doses of UVA. Tumors are expected to be observable starting at week 13 of irradiation with UVB and UVB plus UVA.

2.1.5.1. No UV (Controls)

Mice that have not been exposed to UV will serve as age-matched controls.

### Example 3

### In Vitro Assay of Skin Biomarker Responses to Topical Compositions

NELL1 will be assayed in an in vitro model of skin.

3.1. Human Dermal Fibroblasts and Epidermal Keratinocytes

Human dermal fibroblasts will be treated for 72 hours with a therapeutically effective amount of a NELL1 peptide, and will be analyzed for dose-response in collagen I, collagen III, collagen IV and fibronectin by ELISA, using commercial kits.

Human epidermal keratinocytes will be treated for 6 hours with a therapeutically effective amount of a NELL1 peptide. Levels of biomarkers related to epidermal differentiation (for example, but not limited to, keratin 10 and desmogleins), hydration (for example, but not limited to, aquaporin) and longevity (for example, but not limited to, sirtuin) will be evaluated. RNA will be extracted and purified; labeled target cDNA will be synthesized and analyzed using Affymetrix HG-U133 Plus 2.0 microarrays. Alternatively, RNA will be extracted and purified and synthesized cDNA will be subjected to quantitative real time PCR using marker-specific probes and/or primers. The data will be subjected to rigorous quality control, statistical and bioinformatic analysis.

3.2. Human Skin Equivalents

Human NELL1 (SEQ ID NO: 2) was evaluated in human skin equivalent cultures (MatTek Human Skin EpiDermFT Skin Model, MatTek Corp., Ashland, Mass.). These skin cultures reproduce key structural aspects of natural skin, including, but not limited to, a differentiated epidermis, a basement membrane zone and a dermal fibroblast-containing dermal matrix.

MatTek's EpiDermFT System consists of normal, human-derived epidermal keratinocytes (NHEK) and normal, human-derived dermal fibroblasts (NHFB) which have been cultured to form a multilayered, highly differentiated model of the human dermis and epidermis. The NHEK and NHFB, which are cultured on specially prepared cell culture inserts using serum free medium, attain levels of differentiation. Ultrastructurally, the EpiDermFT Skin Model closely parallels human skin, thus providing a useful in vitro means to assess dermal irritancy and toxicology.

MatTek uses normal (non-transformed) donated human cells as the basis for all of its 3-D tissue equivalents. These cells are grown (cultured) in standard Millipore Millicell™ Single Well Tissue Culture Plate Inserts at the air liquid interface (ALI). At a specific point in this process, all liquid is removed from the apical (top) surface of the tissue. The 3-D tissues are then fed only through the basolateral (bottom) surface, which remains in contact with MatTek's proprietary culture medium.

The EpiDermFT Full Thickness Skin Model exhibits in vivo-like morphological and growth characteristics which are uniform and highly reproducible. EpiDermFT consists of organized basal, spinous, granular, and cornified epidermal layers analogous to those found in vivo. The dermal compartment is composed of a collagen matrix containing viable normal human dermal fibroblasts (NHDF).

EpiDermFT is mitotically and metabolically active. Markers of mature epidermis-specific differentiation such as pro-filaggrin, the K1/K10 cytokeratin pair, involucrin, and type I epidermal transglutaminase have been localized in the model. Ultrastructural analysis has revealed the presence of keratohyalin granules, tonofilament bundles, desmosomes, and a multi-layered stratum corneum containing intercellular lamellar lipid layers arranged in patterns characteristic of in vivo epidermis.

A well-developed basement membrane is present at the dermal/epidermal junction. Hemidesomosomes, lamina lucida, lamina densa and anchoring fibril structures are evident by transmission electron microscopy. Immunohistochemical analysis shows the presence of basement membrane structural and signaling proteins including collagen IV, Laminin, collagen VII and integrin α6.

Full-length human recombinant NELL1 protein (isoform 1), expressed in a wheat germ cell-free translation system, was obtained from Abnova (cat #H00004745-P01). Genistein, a soy derived photoprotective isoflavone, was obtained from Sigma Chemical (Cat #G6649-25, Lot #098K0735) and used as a positive control (Moore et al. (2006) Carcinogenesis 27(8):1627-1635; Kang et al. (2003) J Invest Dermatol 120:835-841).

EpiDerm-FT™ (EFT-400) tissues were produced and packaged in the MatTek Corporation GMP tissue culture production laboratories following standard procedures. Packaged tissues were stored at 4° C. overnight and then re-equilibrated in EFT-400-ASY medium overnight prior to the start of experiments. The sample size was n=3 for each treatment group in the gene expression experiments; and n=6 (two independent experiments of n=3) for the sunburned cell counts.

NELL1 treated tissues were exposed to 100 or 150 ng/ml final concentrations in the basolateral culture medium for 30 minutes. Treated and untreated tissues were exposed to UV irradiation with 200 mJ/cm²UVB/29.94 mW/cm²UVA simulated solar UV light (Honle-500 solar lamp). Additional tissues were pretreated with 50 μM genistein prior to irradiation. Tissues were transferred to culture plates containing phosphate buffered saline for the UV irradiation period, then returned to the control, NELL1- or genistein-containing medium for an additional 24 hrs.

At the conclusion of the 24 hour post-irradiation period, EFT-400 tissues were fixed in 10% formalin for 2 hours at room temperature (two independent experiments with n=3). Formalin fixed tissues were embedded in paraffin and 6 micron slices were stained with haematoxylin and eosin (H&E).

Total RNA was isolated from the EFT-400 tissues (RNAqueous kit, Ambion) (n=3) and utilized for gene expression analysis by quantitative PCR on an SABioscience custom array containing 42 Human Extracellular Matrix (ECM) and Adhesion Molecules RT² Profiler™ PCR Array, stress and toxicity genes whose expression, based on MatTek data, are known to be dramatically altered by UV-protective compounds on UV damaged skin. 42 test genes and 3 housekeeping genes (beta actin, glyceraldehyde-3-phosphate dehydrogenase, and ribosomal protein L13a) were assayed (see Table 4). Further, there were three internal control spots on the array that corresponded to human genomic DNA contamination (HGDC), positive PCR control (PPC), and reverse transcription control (RTC).

TABLE 4
Genes analyzed with custom array.

| GenBank | | |
|---|---|---|
| Acc. No. | Symbol | Description |
| NM_002026 | FN1 | Fibronectin 1 |
| NM_000216 | KAL1 | Kallmann syndrome 1 sequence |
| NM_007112 | THBS3 | Thrombospondin 3 |
| NM_002160 | TNC | Tenascin C |
| NM_000638 | VTN | Vitronectin |
| NM_005559 | LAMA1 | Laminin, alpha 1 |
| NM_000426 | LAMA2 | Laminin, alpha 2 |
| NM_000227 | LAMA3 | Laminin, alpha 3 |
| NM_005560 | LAMA5 | Laminin, alpha 5 |
| NM_002291 | LAMB1 | Laminin, beta 1 |
| NM_000228 | LAMB3 | Laminin, beta 3 |
| NM_002293 | LAMC1 | Laminin, gamma 1 (formerly LAMB2) |
| NM_000575 | IL1A | Interleukin 1, alpha |
| NM_000576 | IL1B | Interleukin 1, beta |
| NM_000584 | IL8 | Interleukin 8 |
| NM_138578 | BCL2L1 | BCL2-like 1 |
| NM_000773 | CYP2E1 | Cytochrome P450, family 2, subfamily |
| | | E, polypeptide 1 |
| NM_001983/ | ERCC1 | Excision repair cross-complementing |
| NM_202001 | | rodent repair deficiency, |
| | | complementation group 4 (includes |
| | | overlapping antisense sequence) |
| NM_000849 | GSTM3 | Glutathione S-transferase mu 3 (brain) |
| NM_006597 | HSPA8 | Heat shock 70 kDa protein 8 |
| NM_005953 | MT2A | Metalloproteinin 2A |
| NM_182649 | PCNA | Proliferating cell nuclear antigen |
| NM_004864 | GDF15 | Growth differentiation factor 15 |
| NM_000358 | TGFB1 | Transforming growth factor, beta- |
| | | induced, 68 kDa |
| NM_000088 | COL1A1 | Collagen, type I, alpha 1 |
| NM_001846 | COL4A2 | Collagen, type IV, alpha 2 |
| NM_000093 | COL5A1 | Collagen, type V, alpha 1 |
| NM_001848 | COL6A1 | Collagen, type VI, alpha 1 |
| NM_001849 | COL6A2 | Collagen, type VI, alpha 2 |
| NM_000094 | COL7A1 | Collagen, type VII, alpha 1 |
| NM_001850 | COL8A1 | Collagen, type VIII, alpha 1 |
| NM_080629 | COL11A1 | Collagen, type XI, alpha 1 |
| NM_004370 | COL12A1 | Collagen, type XII, alpha 1 |
| NM_021110 | COL14A1 | Collagen, type XIV, alpha 1 |
| NM_001855 | COL15A1 | Collagen, type XV, alpha 1 |
| NM_001856 | COL16A1 | Collagen, type XVI, alpha 1 |
| NM_002421 | MMP1 | Matrix metallopeptidase 1 (interstitial |
| | | collagenase) |
| NM_004530 | MMP2 | Matrix metallopeptidase 2 (gelatinase |
| | | A, 72 kDa gelatinase, 72 kDa type IV |
| | | collagenase) |
| NM_004925 | AQP3 | Aquaporin 3 (Gill blood group) |
| NM_005557 | KRT16 | Keratin 16 |
| NM_012238 | SIRT1 | Sirtuin (silent mating type information |
| | | regulation 2 homolog) 1 (S. cerevisiae) |
| NM_001942 | DSG1 | Desmoglein 1 |
| NM_001101 | ACTB | Actin, beta |
| NM_002046 | GAPDH | Glyceraldehyde-3-phosphate |
| | | dehydrogenase |
| NM_012423 | RPL13A | Ribosomal protein L13a |

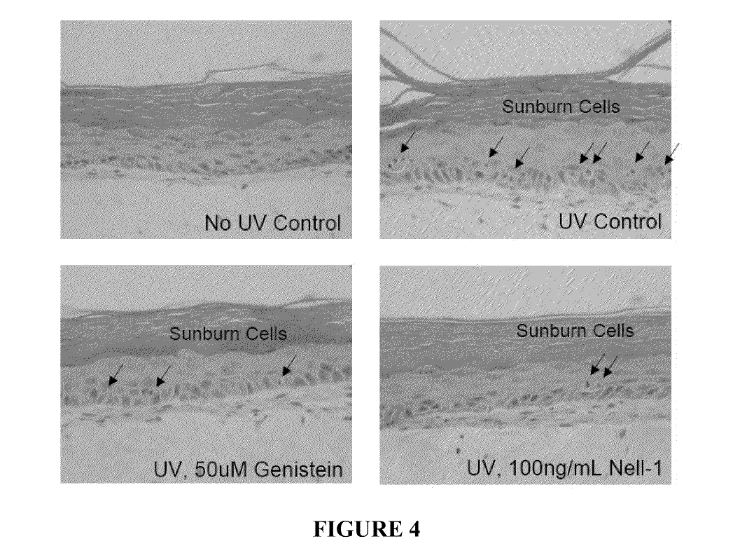

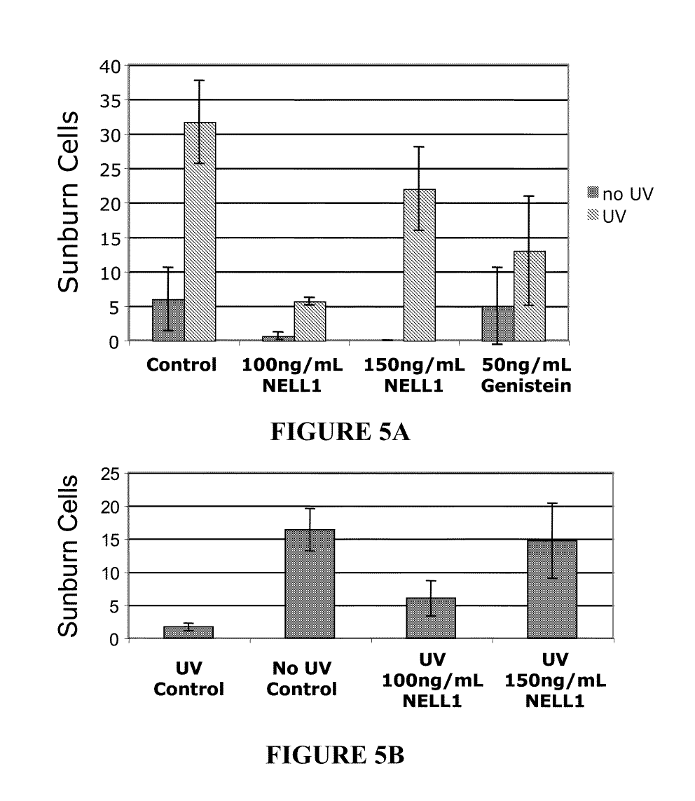

Irradiation of EFT-400 tissues with 200 mJ/cm²UVB/29.94mW/cm²UVA of simulated solar UV radiation produced numerous sunburned cells at 24 hour post irradiation as observed by H&E stained histology. NELL1 protein at 100 ng/ml or 150 ng/ml, or genistein at 50 μM concentrations in the basolateral medium did not produce any noteworthy adverse effects on the EFT-400 tissues. NELL1 protein at 100 ng/ml produced a decrease in sunburned cell formation (FIG. 4 and FIG. 5A). However, NELL1 protein at 150 ng/ml or genistein at 50 μM did not decrease sunburned cell formation to the same extent as 100 ng/ml NELL1 (FIG. 4 and FIG. 5A). To confirm the observation of this experiment, a second experiment was performed on fresh tissues (FIG. 5B), with similar results.

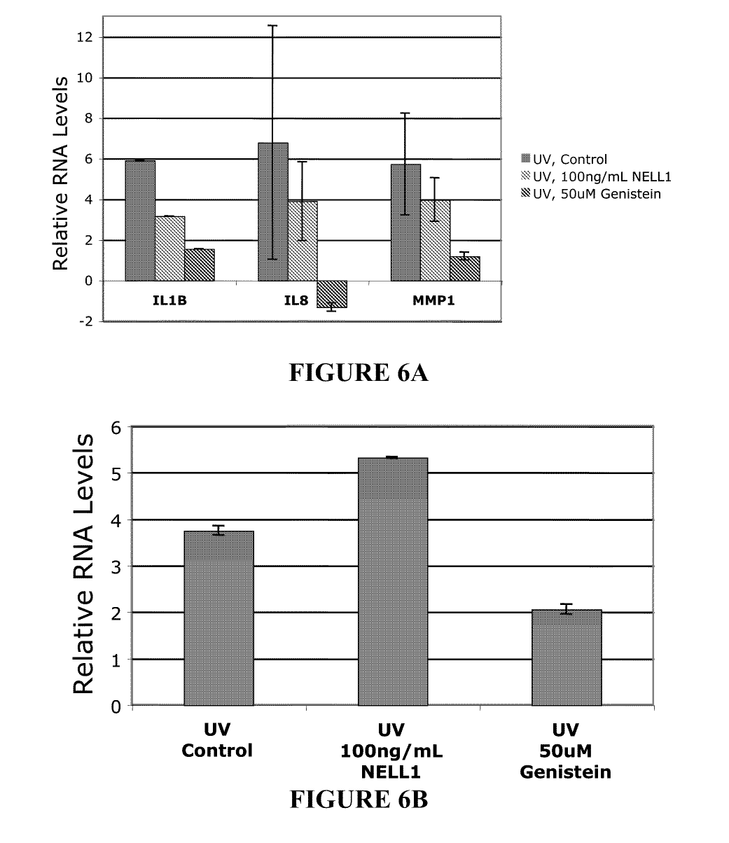

Threshold cycle numbers (Ct), the quantitative measure of gene expression levels, are shown in Table 5. It is evident from the table that many of the genes of interest (e.g. collagens and extracellular matrix proteins) were expressed at high baseline levels in control tissues. The fold-changes in gene expression caused by the UV, NELL1 or genistein treatments are shown in Table 6. NELL1 or genistein treatment caused some decreases in gene expression in the absence of UV irradiation. Genistein caused more changes compared to NELL1. UV-irradiation of the EFT-400 tissues caused an increase in a number of RNA levels compared to no UV controls, including several important inflammatory genes (IL-1β and IL-8), the matrix degrading proteases (MMP-1) and aquaporin 3 (AQP3) (FIGS. 6A and 6B). NELL1 protein at 100 ng/ml slightly modulated the increases in IL-1β, IL-8 and MMP-1, while NELL1 at 150 ng/ml did not. Genistein was the most effective at reversing the UV-induced effects on gene expression in the EFT-400 tissues. NELL1 at 100 ng/ml further increased the UV-elevated levels of AQP3 in the tissues, while NELL1 at 150 ng/ml and genistein did not have the same effect.

TABLE 5
Threshold Cycle (Ct) values for genes evaluated with
quantitative polymerase chain reaction (qPCR) array.

| | AVG Ct | | | | | | | |
|---|---|---|---|---|---|---|---|---|
| | No UV | | | | UVB + UVA | | | |
| | | 100 | 150 | 50 uM | | 100 | 150 | 50 uM |
| | Control | ng/ml | ng/ml | Genis- | | ng/ml | ng/ml | Genis- |
| Symbol | Group | Nell 1 | Nell 1 | tein | Control | Nell 1 | Nell 1 | tein |
| FN1 | 21.22 | 20.04 | 20.71 | 21.06 | 19.77 | 19.79 | 20.13 | 20.79 |
| KAL1 | 31.38 | 31.6 | 33.03 | 31.59 | 32.64 | 31.65 | 32.24 | 31.72 |
| THBS3 | 28.67 | 28.69 | 29.23 | 29.09 | 28.75 | 28.28 | 28.7 | 28.97 |
| TNC | 22.11 | 21.22 | 22.04 | 22.12 | 20.04 | 20.22 | 20.2 | 21.29 |
| VTN | 32.66 | 32.48 | 32.57 | 32.74 | 32.37 | 31.83 | 32.41 | 32.61 |
| LAMA1 | 28.68 | 28.71 | 29.76 | 29.54 | 27.62 | 28.16 | 28.02 | 28.49 |
| LAMA2 | 24.43 | 24.38 | 25.3 | 25.35 | 25.06 | 25.03 | 25.17 | 25.42 |
| LAMA3 | 23.69 | 23.37 | 24.15 | 24.25 | 23.46 | 22.41 | 22.96 | 24.01 |
| LAMA5 | 27.12 | 26.73 | 27.41 | 27.39 | 27.46 | 26.3 | 27.06 | 27.71 |
| LAMB1 | 22.96 | 22.69 | 23.44 | 23.3 | 22.46 | 22.34 | 22.48 | 22.85 |
| LAMB3 | 24.9 | 24.73 | 25.42 | 25.31 | 24.21 | 23.78 | 24.32 | 24.92 |
| LAMC1 | 23.04 | 22.76 | 23.62 | 23.09 | 22.78 | 22.72 | 22.88 | 22.69 |
| IL1A | 25.44 | 25.17 | 25.55 | 25.92 | 24.65 | 24.03 | 23.99 | 25.87 |
| IL1B | 28.61 | 28.25 | 29.2 | 29.03 | 25.91 | 26.48 | 25.98 | 28.03 |
| IL8 | 20.16 | 20.41 | 21.04 | 23.12 | 17.26 | 17.72 | 17.08 | 20.61 |
| BCL2L1 | 24.44 | 24.03 | 24.48 | 24.35 | 23.55 | 23.22 | 23.45 | 23.79 |
| CYP2E1 | 30.66 | 30.79 | 31.34 | 31.05 | 30.26 | 29.93 | 29.88 | 29.69 |
| ERCC1 | 24.24 | 23.72 | 24.07 | 24.94 | 23.56 | 23.26 | 23.41 | 24.08 |
| GSTM3 | 28.95 | 29.11 | 29.74 | 29.01 | 29.27 | 29.13 | 29.32 | 28.37 |
| HSPA8 | 20.46 | 19.83 | 20.36 | 20.53 | 20.16 | 19.74 | 20.04 | 20.14 |
| MT2A | 19.25 | 18.43 | 18.83 | 18.74 | 16.71 | 16.81 | 16.56 | 16.4 |
| PCNA | 24.52 | 23.56 | 24.11 | 23.76 | 23.65 | 23.31 | 23.58 | 23.21 |
| GDF15 | 23.91 | 23.37 | 23.97 | 27.52 | 22.11 | 22.21 | 21.87 | 21.74 |
| TGFBI | 22.41 | 21.73 | 22.16 | 22.04 | 21.93 | 21.97 | 21.91 | 22.03 |
| COL1A1 | 18.06 | 17.82 | 18.8 | 18.66 | 18.74 | 18.36 | 18.78 | 18.54 |
| COL4A2 | 22.68 | 22.01 | 22.92 | 26.06 | 21.5 | 21.55 | 21.59 | 21.82 |
| COL5A1 | 22.76 | 22.36 | 23.36 | 23.19 | 23.32 | 22.98 | 23.33 | 23.29 |
| COL6A1 | 19.25 | 18.82 | 19.6 | 19.41 | 19.21 | 19.15 | 19.34 | 19.47 |
| COL6A2 | 19.66 | 19.33 | 20.05 | 19.82 | 19.34 | 19.19 | 19.56 | 19.68 |
| COL7A1 | 26.28 | 25.75 | 26.73 | 26.3 | 24.79 | 24.38 | 25.28 | 24.97 |
| COL8A1 | 27.9 | 27.28 | 28.22 | 28.27 | 27.03 | 26.96 | 27.46 | 27.28 |
| COL11A1 | 27.23 | 26.77 | 27.51 | 27.54 | 27.13 | 26.86 | 27.23 | 27.6 |
| COL12A1 | 24.87 | 24.14 | 25 | 25.19 | 24.19 | 23.95 | 24.22 | 24.93 |
| COL14A1 | 25.01 | 25.88 | 26.52 | 26.08 | 26.07 | 25.64 | 25.86 | 25.69 |
| COL15A1 | 26 | 24.72 | 25.35 | 25.73 | 25.29 | 25.01 | 25.2 | 25.83 |
| COL16A1 | 23.75 | 23.78 | 24.55 | 24.6 | 23.6 | 23.42 | 23.71 | 23.98 |
| MMP1 | 20.37 | 20.04 | 20.75 | 22.22 | 17.72 | 17.91 | 17.75 | 20.16 |
| MMP2 | 19.1 | 18.98 | 19.62 | 19.2 | 18.76 | 18.77 | 18.67 | 18.5 |
| AQP3 | 24.01 | 23.05 | 23.74 | 23.75 | 21.97 | 21.13 | 21.71 | 23.01 |
| KRT16 | 16.74 | 16.15 | 16.49 | 17.11 | 16.92 | 16.35 | 16.47 | 17.92 |
| SIRT1 | 25.99 | 25.93 | 26.67 | 26.45 | 25.84 | 26 | 26.02 | 25.31 |
| DSG1 | 20.1 | 20.11 | 20.36 | 20.31 | 20.65 | 20.06 | 20.21 | 21.03 |
| ACTB | 18.79 | 18 | 18.54 | 18.95 | 18.19 | 17.67 | 17.99 | 18.85 |
| GAPDH | 18.31 | 17.83 | 18.02 | 17.74 | 18.37 | 18.15 | 18.08 | 18.39 |
| HGDC | 35 | 35 | 35 | 35 | 35 | 35 | 35 | 35 |
| PPC | 19.21 | 19.22 | 19.27 | 19.27 | 19.25 | 19.28 | 19.32 | 19.31 |
| RPL13A | 20.68 | 20.37 | 20.81 | 20.65 | 20.81 | 20.55 | 20.64 | 20.68 |
| RTC | 23.07 | 22.9 | 22.95 | 23.06 | 23 | 23.03 | 23.12 | 23.22 |
| | Standard Deviation | | | | | | | |
| | No UV | | | | UVB + UVA | | | |
| | | 100 | 150 | 50 uM | | 100 | 150 | 50 uM |
| | Control | ng/ml | ng/ml | Genis- | | ng/ml | ng/ml | Genis- |
| Symbol | Group | Nell 1 | Nell 1 | tein | Control | Nell 1 | Nell 1 | tein |
| FN1 | 0.418 | 0.175 | 0.031 | 0.285 | 0.429 | 0.209 | 0.108 | 0.642 |
| KAL1 | 0.040 | 0.276 | 0.155 | 0.289 | 0.364 | 0.344 | 1.094 | 0.512 |
| THBS3 | 0.335 | 0.139 | 0.269 | 0.309 | 0.262 | 0.154 | 0.220 | 0.571 |
| TNC | 0.070 | 0.075 | 0.211 | 0.332 | 0.585 | 0.167 | 0.278 | 0.516 |
| VTN | 0.407 | 0.463 | 0.937 | 0.334 | 0.659 | 0.328 | 0.111 | 0.529 |
| LAMA1 | 0.135 | 0.416 | 0.124 | 0.226 | 0.489 | 0.108 | 0.137 | 0.651 |
| LAMA2 | 0.151 | 0.323 | 0.106 | 0.207 | 0.292 | 0.224 | 0.187 | 0.463 |
| LAMA3 | 0.308 | 0.309 | 0.011 | 0.180 | 0.394 | 0.187 | 0.644 | 0.467 |
| LAMA5 | 0.110 | 0.171 | 0.025 | 0.236 | 0.667 | 0.114 | 0.188 | 0.544 |
| LAMB1 | 0.051 | 0.223 | 0.133 | 0.215 | 0.407 | 0.225 | 0.233 | 0.362 |
| LAMB3 | 0.160 | 0.264 | 0.203 | 0.272 | 0.343 | 0.394 | 1.044 | 0.315 |
| LAMC1 | 0.005 | 0.403 | 0.243 | 0.048 | 0.382 | 0.333 | 0.214 | 0.434 |
| IL1A | 0.215 | 0.046 | 0.123 | 0.225 | 0.752 | 1.068 | 1.138 | 0.405 |
| IL1B | 0.407 | 0.506 | 0.431 | 0.526 | 1.448 | 0.876 | 1.271 | 0.205 |
| IL8 | 1.065 | 0.570 | 0.928 | 1.131 | 0.791 | 0.450 | 0.347 | 0.412 |
| BCL2L1 | 0.012 | 0.169 | 0.129 | 0.241 | 0.480 | 0.085 | 0.003 | 0.259 |
| CYP2E1 | 0.074 | 0.355 | 0.371 | 0.325 | 0.815 | 0.527 | 0.343 | 0.399 |
| ERCC1 | 0.065 | 0.090 | 0.120 | 1.185 | 0.573 | 0.277 | 0.214 | 0.146 |
| GSTM3 | 0.232 | 0.328 | 0.149 | 0.136 | 0.495 | 0.196 | 0.663 | 0.452 |
| HSPA8 | 0.102 | 0.266 | 0.170 | 0.433 | 0.457 | 0.186 | 0.024 | 0.262 |
| MT2A | 0.448 | 0.156 | 0.265 | 0.421 | 0.693 | 0.135 | 0.257 | 0.663 |
| PCNA | 0.001 | 0.213 | 0.016 | 0.119 | 0.417 | 0.218 | 0.052 | 0.453 |
| GDF15 | 0.284 | 0.289 | 0.079 | 6.483 | 0.642 | 0.096 | 0.687 | 1.049 |
| TGFBI | 0.009 | 0.376 | 0.013 | 0.126 | 0.657 | 0.046 | 0.301 | 0.441 |
| COL1A1 | 0.083 | 0.173 | 0.079 | 0.103 | 0.434 | 0.286 | 0.101 | 0.581 |
| COL4A2 | 0.102 | 0.300 | 0.228 | 5.745 | 0.551 | 0.199 | 0.346 | 0.428 |
| COL5A1 | 0.138 | 0.301 | 0.126 | 0.078 | 0.463 | 0.150 | 0.167 | 0.544 |
| COL6A1 | 0.083 | 0.369 | 0.002 | 0.118 | 0.677 | 0.072 | 0.203 | 0.542 |
| COL6A2 | 0.295 | 0.177 | 0.049 | 0.216 | 0.215 | 0.228 | 0.175 | 0.569 |
| COL7A1 | 0.041 | 0.058 | 0.071 | 0.483 | 0.426 | 0.195 | 0.116 | 0.718 |
| COL8A1 | 0.551 | 0.137 | 0.099 | 0.460 | 0.238 | 0.225 | 0.135 | 0.354 |
| COL11A1 | 0.214 | 0.178 | 0.221 | 0.339 | 0.408 | 0.176 | 0.091 | 0.461 |
| COL12A1 | 0.248 | 0.175 | .0369 | 0.204 | 0.470 | 0.130 | 0.252 | 0.444 |
| COL14A1 | 0.112 | 0.232 | 0.080 | 0.174 | 0.311 | 0.291 | 0.344 | 0.250 |
| COL15A1 | 0.289 | 0.264 | 0.065 | 0.405 | 0.330 | 0.212 | 0.108 | 0.604 |
| COL16A1 | 0.122 | 0.254 | 0.115 | 0.121 | 0.538 | 0.133 | 0.366 | 0.519 |
| MMP1 | 0.341 | 0.296 | 0.350 | 0.791 | 0.686 | 0.239 | 0.280 | 0.764 |
| MMP2 | 0.054 | 0.232 | 0.154 | 0.130 | 0.361 | 0.324 | 0.316 | 0.550 |
| AQP3 | 0.182 | 0.076 | 0.214 | 0.411 | 0.338 | 0.077 | 0.042 | 1.284 |
| KRT16 | 0.077 | 0.057 | 0.017 | 0.033 | 0.316 | 0.222 | 0.125 | 0.327 |
| SIRT1 | 0.035 | 0.351 | 0.059 | 0.396 | 0.447 | 0.045 | 0.084 | 0.958 |
| DSG1 | 0.183 | 0.147 | 0.022 | 0.112 | 0.411 | 0.267 | 0.175 | 0.528 |
| ACTB | 0.267 | 0.149 | 0.018 | 0.129 | 0.193 | 0.133 | 0.217 | 0.448 |
| GAPDH | 0.187 | 0.218 | 0.015 | 0.253 | 0.577 | 0.108 | 0.144 | 0.348 |
| HGDC | 0 | 0 | 0 | 0 | 0 | 0 | 0 | 0 |
| PPC | 0.079 | 0.034 | 0.002 | 0.101 | 0.084 | 0.115 | 0.046 | 0.068 |
| RPL13A | 0.074 | 0.085 | 0.105 | 0.115 | 0.285 | 0.072 | 0.307 | 0.324 |
| RTC | 0.051 | 0.140 | 0.148 | 0.029 | 0.062 | 0.090 | 0.087 | 0.141 |

Ct < 25 indicates high expression level; 25 < Ct < 30 indicates moderate expression levels; 30 < Ct < 35 indicates low expression levels.

TABLE 6
Fold change in expression level compared to no UV control for genes evaluated with qPCR array.

| Fold Regulation (compared to No UV Control) | | | | | | | | p-value | | | | | | |
|---|---|---|---|---|---|---|---|---|---|---|---|---|---|---|
| No UV | | | | UVB + UVA | | | | No UV | | | UVB + UVA | | | |
| | 100 | 150 | 50 uM | | 100 | 150 | 50 uM | 100 | 150 | 50 uM | | 100 | 150 | 50 uM |
| | ng/ml | ng/ml | Genis- | | ng/ml | ng/ml | Genis- | ng/ml | ng/ml | Genis- | | ng/ml | ng/ml | Genis- |
| Symbol | Nell 1 | Nell 1 | tein | Control | Nell 1 | Nell 1 | tein | Nell 1 | Nell 1 | tein | Control | Nell 1 | Nell 1 | tein |
| FN1 | 1.57 | 1.30 | 1.01 | 2.48 | 1.94 | 1.66 | 1.40 | 0.037 | 0.183 | 0.977 | 0.002 | 0.024 | 0.028 | 0.256 |
| KAL1 | −1.69 | −3.46 | −1.28 | −2.63 | −1.68 | −2.32 | −1.22 | 0.020 | 0.000004 | 0.010 | 0.0008 | 0.008 | 0.041 | 0.697 |
| THBS3 | −1.47 | −1.62 | −1.48 | −1.16 | −1.06 | −1.31 | −1.19 | 0.074 | 0.061 | 0.077 | 0.612 | 0.651 | 0.286 | 0.552 |
| TNC | 1.28 | −1.04 | −1.11 | 3.85 | 2.69 | 2.94 | 1.84 | 0.004 | 0.647 | 0.656 | 0.001 | 0.002 | 0.001 | 0.010 |
| VTN | −1.28 | −1.03 | −1.17 | 1.11 | 1.28 | −1.07 | 1.07 | 0.507 | 0.860 | 0.408 | 0.663 | 0.308 | 0.630 | 0.778 |
| LAMA1 | −1.48 | −2.33 | −2.01 | 1.90 | 1.03 | 1.23 | 1.18 | 0.053 | 0.0005 | 0.002 | 0.025 | 0.716 | 0.039 | 0.403 |
| LAMA2 | −1.39 | −2.01 | −2.09 | −1.70 | −2.10 | −2.13 | −1.92 | 0.033 | 0.001 | 0.001 | 0.003 | 0.003 | 0.001 | 0.002 |
| LAMA3 | −1.16 | −1.52 | −1.63 | 1.07 | 1.76 | 1.29 | −1.21 | 0.482 | 0.048 | 0.050 | 0.708 | 0.010 | 0.303 | 0.381 |
| LAMA5 | −1.10 | −1.34 | −1.33 | −1.39 | 1.28 | −1.23 | −1.45 | 0.278 | 0.006 | 0.011 | 0.393 | 0.018 | 0.025 | 0.182 |
| LAMB1 | −1.19 | −1.53 | −1.40 | 1.29 | 1.12 | 1.09 | 1.13 | 0.050 | 0.0005 | 0.015 | 0.029 | 0.381 | 0.371 | 0.015 |
| LAMB3 | −1.28 | −1.57 | −1.46 | 1.47 | 1.57 | 1.17 | 1.02 | 0.130 | 0.014 | 0.012 | 0.002 | 0.077 | 0.541 | 0.787 |
| LAMC1 | −1.19 | −1.64 | −1.15 | 1.09 | −1.11 | −1.15 | 1.32 | 0.237 | 0.002 | 0.049 | 0.033 | 0.629 | 0.041 | 0.049 |
| IL1A | −1.19 | −1.18 | −1.54 | 1.58 | 1.92 | 2.13 | −1.30 | 0.172 | 0.154 | 0.033 | 0.099 | 0.273 | 0.208 | 0.283 |
| IL1B | −1.13 | −1.66 | −1.48 | 5.91 | 3.17 | 4.82 | 1.55 | 0.680 | 0.107 | 0.194 | 0.133 | 0.112 | 0.114 | 0.095 |
| IL8 | −1.72 | −2.03 | −8.59 | 6.79 | 3.90 | 6.60 | −1.32 | 0.310 | 0.299 | 0.093 | 0.012 | 0.029 | 0.0007 | 0.432 |
| BCL2L1 | −1.08 | −1.12 | −1.04 | 1.69 | 1.69 | 1.55 | 1.63 | 0.292 | 0.142 | 0.648 | 0.012 | 0.001 | 0.014 | 0.003 |
| CYP2E1 | −1.57 | −1.75 | −1.44 | 1.21 | 1.20 | 1.35 | 2.04 | 0.005 | 0.009 | 0.035 | 0.357 | 0.406 | 0.257 | 0.0003 |
| ERCC1 | −1.01 | 1.02 | −1.80 | 1.46 | 1.43 | 1.39 | 1.15 | 0.874 | 0.688 | 0.154 | 0.022 | 0.027 | 0.017 | 0.176 |
| GSTM3 | −1.61 | −1.90 | −1.15 | −1.37 | −1.57 | −1.65 | 1.55 | 0.024 | 0.007 | 0.245 | 0.063 | 0.032 | 0.134 | 0.040 |
| HSPA8 | 1.08 | −1.02 | −1.16 | 1.12 | 1.19 | 1.05 | 1.30 | 0.521 | 0.782 | 0.507 | 0.356 | 0.102 | 0.600 | 0.062 |
| MT2A | 1.22 | 1.21 | 1.28 | 5.28 | 3.92 | 5.02 | 7.43 | 0.373 | 0.427 | 0.356 | 0.004 | 0.0003 | 0.0001 | 0.012 |
| PCNA | 1.35 | 1.21 | 1.53 | 1.66 | 1.67 | 1.50 | 2.56 | 0.044 | 0.00003 | 0.0000 | 0.026 | 0.009 | 0.018 | 0.0009 |
| GDF15 | 1.01 | −1.14 | −13.46 | 3.17 | 2.36 | 3.22 | 4.69 | 0.997 | 0.305 | 0.331 | 0.004 | 0.001 | 0.023 | 0.111 |
| TGFBI | 1.10 | 1.08 | 1.17 | 1.27 | −1.02 | 1.10 | 1.34 | 0.353 | 0.002 | 0.035 | 0.206 | 0.287 | 0.219 | 0.034 |
| COL1A1 | −1.22 | −1.84 | −1.68 | −1.76 | −1.71 | −2.11 | −1.35 | 0.008 | 0.0002 | 0.0006 | 0.006 | 0.009 | 0.0007 | 0.074 |
| COL4A2 | 1.10 | −1.30 | −11.52 | 2.07 | 1.58 | 1.66 | 1.88 | 0.380 | 0.038 | 0.187 | 0.003 | 0.027 | 0.021 | 0.009 |
| COL5A1 | −1.09 | −1.66 | −1.49 | −1.62 | −1.62 | −1.90 | −1.40 | 0.387 | 0.002 | 0.006 | 0.003 | 0.007 | 0.001 | 0.039 |
| COL6A1 | −1.07 | −1.40 | −1.24 | −1.06 | −1.29 | −1.36 | −1.13 | 0.653 | 0.001 | 0.011 | 0.746 | 0.013 | 0.002 | 0.528 |
| COL6A2 | −1.15 | −1.44 | −1.24 | 1.13 | 1.00 | −1.19 | 1.02 | 0.358 | 0.056 | 0.163 | 0.492 | 0.997 | 0.254 | 0.904 |
| COL7A1 | 1.00 | −1.50 | −1.11 | 2.57 | 2.71 | 1.56 | 2.57 | 0.918 | 0.00005 | 0.669 | 0.004 | 0.003 | 0.002 | 0.041 |
| COL8A1 | 1.06 | −1.37 | −1.43 | 1.67 | 1.38 | 1.06 | 1.59 | 0.944 | 0.234 | 0.241 | 0.069 | 0.259 | 0.962 | 0.079 |
| COL11A1 | −1.04 | −1.33 | −1.37 | −1.02 | −1.06 | −1.27 | −1.24 | 0.659 | 0.070 | 0.061 | 0.832 | 0.639 | 0.144 | 0.132 |
| COL12A1 | 1.15 | −1.20 | −1.37 | 1.46 | 1.37 | 1.23 | −1.01 | 0.272 | 0.361 | 0.090 | 0.022 | 0.054 | 0.118 | 0.931 |
| COL14A1 | −2.63 | −3.12 | −2.33 | −2.29 | −2.14 | −2.30 | −1.54 | 0.0002 | 0.0001 | 0.0003 | 0.003 | 0.003 | 0.0007 | 0.002 |
| COL15A1 | 1.69 | 1.43 | 1.09 | 1.49 | 1.44 | 1.36 | 1.16 | 0.010 | 0.024 | 0.602 | 0.015 | 0.081 | 0.056 | 0.415 |
| COL16A1 | −1.47 | −1.91 | −2.00 | 1.02 | −1.10 | −1.24 | −1.13 | 0.009 | 0.0008 | 0.0008 | 0.852 | 0.342 | 0.050 | 0.585 |
| MMP1 | −1.15 | −1.43 | −3.99 | 5.73 | 3.98 | 4.81 | 1.20 | 0.502 | 0.141 | 0.009 | 0.003 | 0.004 | 0.0001 | 0.487 |
| MMP2 | −1.32 | −1.58 | −1.18 | 1.16 | −1.10 | 1.06 | 1.57 | 0.018 | 0.0007 | 0.156 | 0.176 | 0.669 | 0.549 | 0.022 |
| AQP3 | 1.35 | 1.10 | 1.08 | 3.75 | 5.32 | 3.85 | 2.06 | 0.060 | 0.473 | 0.597 | 0.008 | 0.0000 | 0.002 | 0.186 |
| KRT16 | 1.05 | 1.08 | −1.43 | −1.24 | −1.06 | −1.06 | −2.18 | 0.307 | 0.094 | 0.003 | 0.246 | 0.602 | 0.658 | 0.002 |
| SIRT1 | −1.39 | −1.77 | −1.53 | 1.01 | −1.40 | −1.31 | 1.65 | 0.019 | 0.00001 | 0.010 | 0.877 | 0.001 | 0.011 | 0.258 |
| DSG1 | −1.46 | −1.32 | −1.28 | −1.60 | −1.35 | −1.38 | −1.85 | 0.013 | 0.030 | 0.041 | 0.078 | 0.074 | 0.120 | 0.034 |
| ACTB | 1.20 | 1.08 | −1.24 | 1.38 | 1.57 | 1.36 | −1.01 | 0.177 | 0.546 | 0.147 | 0.054 | 0.009 | 0.032 | 0.968 |
| GAPDH | −1.03 | 1.11 | 1.34 | −1.15 | −1.24 | −1.10 | −1.02 | 0.679 | 0.239 | 0.030 | 0.318 | 0.080 | 0.324 | 0.830 |
| HGDC | −1.44 | −1.10 | −1.11 | −1.10 | −1.38 | −1.28 | 1.034 | 0.001 | 0.002 | 0.165 | 0.637 | 0.0000 | 0.035 | 0.720 |
| PPC | −1.45 | −1.14 | −1.15 | −1.12 | −1.45 | −1.38 | −1.03 | 0.003 | 0.020 | 0.267 | 0.633 | 0.001 | 0.029 | 0.910 |
| RPL13A | −1.16 | −1.21 | −1.09 | −1.20 | −1.27 | −1.24 | 1.03 | 0.042 | 0.011 | 0.073 | 0.018 | 0.005 | 0.009 | 0.530 |
| RTC | −1.28 | −1.01 | −1.10 | −1.05 | −1.35 | −1.32 | −1.07 | 0.0006 | 0.942 | 0.243 | 0.922 | 0.0006 | 0.010 | 0.482 |

The amplicon size for IL-1β (interleukin-1 beta) was 186 and the reference position was nucleotide 1087 of GenBank Accession No. NM_{—}00576 (set forth in SEQ ID NO: 40). The amplicon size for IL-8 (interleukin-8) was 126 and the reference position was nucleotide 274 of GenBank Accession No. NM_{—}000584 (set forth in SEQ ID NO: 42). The amplicon size for MMP-1 (matrix metalloproteinase 1) was 111 and the reference position was nucleotide 1140 of GenBank Accession No. NM_{—}002421 (set forth in SEQ ID NO: 44). The amplicon size for aquaporin 3 (AQP3) was 115 and the reference position was nucleotide 842 of GenBank Accession No. NM_{—}004925 (set forth in SEQ ID NO: 46).

In conclusion, simulated solar UV treatment of EFT-400 in vitro human skin tissues produced sunburned cell formation and gene expression changes in a panel of skin health-related genes. Pre-treatment of the EFT-400 cultures with NELL1 or genistein for 30 minutes prior to UV exposure, and continued treatment with these materials for 24 hours post-irradiation produced a statistically significant reduction in the number of sunburned cells as compared to UV-only controls. Treatment of tissues with NELL1 and genistein also resulted in the decrease in the expression of some inflammatory mediator (IL-1β, IL-8) and matrix degrading protease (MMP-1) genes, which had been elevated after UV exposure. NELL1 protein at 100 ng/ml and genistein at 50 μM were most active in producing the observed effects. The levels of AQP3 RNA were significantly elevated after UV exposure compared to non-UV treated tissues. The addition of NELL1 increased these levels further (highly significant at 100 ng/ml), while genistein did not have an effect on AQP3 levels.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto. Although specific terms are employed herein, they are used in a generic and descriptive sense only and not for purposes of limitation.

That which is claimed:

1. A method for treating or preventing a skin condition, the method comprising administering a NELL1 peptide to a subject in need thereof, whereby at least one manifestation of the skin condition is treated or prevented.

2. The method of claim 1, wherein said NELL1 peptide is a human NELL1 peptide.

3. The method of claim 1, wherein the NELL1 peptide has an amino acid sequence selected from the group consisting of:
a) the amino acid sequence set forth in SEQ ID NO: 2, 3, 4, 5, 6, 7, 8, 9, 11, 12, 13, 14, 15, 16, 17, 19, 21, 23, 25, 27, 29, 31, 33, 35, 37, 39, 49, 51, 53, 55, 57, 59, or 61; and
b) an amino acid sequence having at least 70% sequence identity to SEQ ID NO: 2, 3, 4, 5, 6, 7, 8, 9, 11, 12, 13, 14, 15, 16, 17, 19, 21, 23, 25, 27, 29, 31, 33, 35, 37, 39, 49, 51, 53, 55, 57, 59, or 61.

4. The method of claim 3, wherein the NELL1 peptide has an amino acid sequence having at least 90% sequence identity to SEQ ID NO: 2, 3, 4, 5, 6, 7, 8, 9, 11, 12, 13, 14, 15, 16, 17, 19, 21, 23, 25, 27, 29, 31, 33, 35, 37, 39, 49, 51, 53, 55, 57, 59, or 61.

5. The method of claim 1, wherein the NELL1 peptide has at least one of the following activities: stimulates extracellular matrix production; stimulates expression of an aquaporin; reduces the expression of a matrix metalloproteinase; reduces inflammation; and reduces the expression of an inflammatory mediator.

6. The method of claim 5, wherein the aquaporin is aquaporin 3 (AQP3).

7. The method of claim 5, wherein the matrix metalloproteinase is matrix metalloproteinase 1 (MMP1).

8. The method of claim 5, wherein the inflammatory mediator is interleukin-1-beta (IL-1β) or interleukin-8 (IL-8).

9. The method of claim 1, wherein the NELL1 peptide is administered topically, subcutaneously, intramuscularly, or intravenously.

10. The method of claim 9, wherein the NELL1 peptide is topically administered to an epithelial surface of a subject in need thereof.

11. The method of claim 10, wherein the epithelial surface is skin.

12. The method of claim 1, wherein the skin condition is aged skin.

13. The method of claim 12, wherein the at least one manifestation of the skin condition is at least one manifestation of aged skin selected from the group consisting of skin dryness, skin roughness, a rhytide, a pigmented lesion, an ephelide, patchy hyperpigmentation, a depigmented lesion, a guttate hypomelanosis, skin fragility, an area of purpura, a benign lesion, an acrochordon, a senile angioma, a seborrheic keratosis, a lentigo, inflammation, and a sebaceous hyperplasia.

14. The method of claim 1, wherein the skin condition is a skin scar.

15. The method of claim 14, wherein the skin scar is a keloid scar, a hypertrophic scar, a widespread scar, or an atrophic scar.

16. The method of claim 14, wherein the at least one manifestation of the skin scar is at least one manifestation selected from the group consisting of an elevation of an area of wounded skin, compared to normal skin; a thickening of an area of wounded skin, compared to normal skin; a nodularity of an area of wounded skin, compared to normal skin; a depression of an area of wounded skin, compared to normal skin; overgrowth of scar tissue that remains within the boundaries of an original wound; overgrowth of scar tissue that exceeds the boundaries of an original wound; an increased proliferation of fibroblasts in wounded skin, compared to normal skin; an increased fibroblast density in wounded skin, compared to normal skin, an increased sensitivity to ultraviolet light, compared to normal skin, a plurality of collagen fibers of random orientation in wounded skin; and a collagen content in wounded skin different from that of normal skin.

17. The method of claim 14, wherein the NELL1 peptide is topically administered to a wounded epithelial surface, wherein the wounded epithelial surface is skin, wherein at least one manifestation of the skin scar is prevented.

18. The method of claim 1, wherein treating or preventing at least one manifestation of the skin condition comprises improving the appearance of the skin condition.

19. The method of claim 1, wherein the NELL1 peptide is in a composition further comprising a carrier.

20. The method of claim 19, wherein the composition is a pharmaceutical composition and wherein the carrier is a pharmaceutically acceptable carrier.

21. The method of claim 19, wherein the composition is a cosmetic composition and wherein the carrier is a cosmetically acceptable carrier.

22. The method of claim 19, wherein the composition is in the form selected from the group consisting of a solution, an oil, a cream, a gel, a lotion, a shampoo, a milk, a cleanser, a moisturizer, a spray and a skin patch.

23. The method of claim 19, wherein the composition further comprises at least one additional active ingredient.

24. The method of claim 23, wherein the at least one additional active ingredient is selected from the group consisting of a protective agent, an emollient, an astringent, an irritant, a keratolytic, a sun screening agent, a sun tanning agent, an antibiotic agent, a non-imidazole analog antifungal agent, an antiviral agent, an antiprotozoal agent, an anti-acne agent, an anesthetic agent, a steroidal anti-inflammatory agent, a non-steroidal anti-inflammatory agent, an antipruritic agent, an anti-oxidant agent, a chemotherapeutic agent, an anti-histamine agent, a peptide, a peptidomimetic, a peptide derivative, a vitamin, a vitamin supplement, a fusion protein, a hormone, an anti-dandruff agent, an anti-wrinkle agent, an anti-skin atrophy agent, a sclerosing agent, a cleansing agent, a caustic agent and a hypo-pigmenting agent.

25. A method for assaying a test peptide for NELL1 activity, said method comprising the steps of:
a) administering said test peptide to a skin sample;
b) irradiating said skin sample with ultraviolet light;
c) assessing the expression level of an inflammatory mediator, a matrix metalloproteinase, or an aquaporin in the skin sample;
wherein step a) is performed at least one of before, during, and after step b); and
wherein a reduction in UV-stimulated expression of an inflammatory mediator or matrix metalloproteinase is indicative of NELL1 activity; or
wherein an increase in UV-stimulated aquaporin expression is indicative of NELL1 activity.

26. The method of claim 25, wherein the inflammatory mediator is interleukin-1-beta (IL-1μ) or interleukin-8 (IL-8).

27. The method of claim 25, wherein the matrix metalloproteinase is matrix metalloproteinase 1 (MMP1).

28. The method of claim 25, wherein the aquaporin is aquaporin 3 (AQP3).

29. The method of claim 25, wherein the skin sample is a skin equivalent sample.

30. The method of claim 29, wherein said test peptide is administered to the skin equivalent sample via addition to the culture medium.

31. A method for assaying a test peptide for NELL1 activity, said method comprising the steps of:
a) administering said test peptide to a skin sample;
b) irradiating said skin sample with ultraviolet light;
c) assessing the number of sunburned cells in said skin sample;
wherein step a) is performed at least one of before, during, and after step b); and
wherein a reduction in the number of sunburned cells as compared to a control sample irradiated with ultraviolet light is indicative of NELL1 activity.

32. The method of claim 31, wherein the skin sample is a skin equivalent sample.

33. The method of claim 32, wherein said test peptide is administered to the skin equivalent sample via addition to the culture medium.

34. A method for treating or preventing a skin condition, the method comprising administering a heterologous nucleic acid molecule having a nucleotide sequence encoding a NELL1 peptide to a subject in need thereof, whereby at least one manifestation of the skin condition is treated or prevented.

35. The method of claim 34, wherein said NELL1 peptide is a human NELL1 peptide.

36. The method of claim 34, wherein the NELL1 peptide has an amino acid sequence selected from the group consisting of:
a) the amino acid sequence set forth in SEQ ID NO: 2, 3, 4, 5, 6, 7, 8, 9, 11, 12, 13, 14, 15, 16, 17, 19, 21, 23, 25, 27, 29, 31, 33, 35, 37, 39, 49, 51, 53, 55, 57, 59, or 61; and
b) an amino acid sequence having at least 70% sequence identity to SEQ ID NO: 2, 3, 4, 5, 6, 7, 8, 9, 11, 12, 13, 14, 15, 16, 17, 19, 21, 23, 25, 27, 29, 31, 33, 35, 37, 39, 49, 51, 53, 55, 57, 59, or 61.

37. The method of claim 34, wherein said nucleotide sequence encoding a NELL1 peptide is selected from the group consisting of:
a) the nucleotide sequence set forth in SEQ ID NO: 1, 10, 18, 20, 22, 24, 26, 28, 30, 32, 34, 36, 38, 48, 50, 52, 54, 56, 58, or 60;
b) a nucleotide sequence having at least 70% sequence identity to SEQ ID NO: 1, 10, 18, 20, 22, 24, 26, 28, 30, 32, 34, 36, 38, 48, 50, 52, 54, 56, 58, or 60.

38. The method of claim 34, wherein the NELL1 peptide has at least one of the following activities: stimulates extracellular matrix production; stimulates expression of an aquaporin; reduces the expression of a matrix metalloproteinase; reduces inflammation; and reduces the expression of an inflammatory mediator.

39. The method of claim 34, wherein said heterologous nucleic acid molecule further comprises a promoter operably linked to said nucleotide sequence encoding a NELL1 peptide.

40. The method of claim 34, wherein the skin condition is aged skin or a skin scar.


SEQUENCE LISTING (61 sequences)

SEQ ID NO: 1 - DNA, 3255 bp, Homo sapiens
  CDS (154)...(2586): Coding sequence
atatgcgagcgcagcacccggcgctgccgagccacctcccccgccgcccgctagcaagtttggcggctccaagccaggcgcgcctcaggatccaggctcatttgcttccacctagcttcggtgccccctgctaggcggggaccctcgagagcgatgccgatggatttgattttagttgtgtggttctgtgtgtgcactgccaggacagtggtgggctttgggatggaccctgaccttcagatggatatcgtcaccgagcttgaccttgtgaacaccacccttggagttgctcaggtgtctggaatgcacaatgccagcaaagcatttttatttcaagacatagaaagagagatccatgcagctcctcatgtgagtgagaaattaattcagctgttccggaacaagagtgaattcaccattttggccactgtacagcagaagccatccacttcaggagtgatactgtccattcgagaactggagcacagctattttgaactggagagcagtggcctgagggatgagattcggtatcactacatacacaatgggaagccaaggacagaggcacttccttaccgcatggcagatggacaatggcacaaggttgcactgtcagttagcgcctctcatctcctgctccatgtcgactgtaacaggatttatgagcgtgtgatagaccctccagataccaaccttcccccaggaatcaatttatggcttggccagcgcaaccaaaagcatggcttattcaaagggatcatccaagatgggaagatcatctttatgccgaatggatatataacacagtgtccaaatctaaatcacacttgcccaacctgcagtgatttcttaagcctggtgcaaggaataatggatttacaagagcttttggccaagatgactgcaaaactaaattatgcagagacaagacttagtcaattggaaaactgtcattgtgagaagacttgtcaagtgagtggactgctctatcgagatcaagactcttgggtagatggtgaccattgcaggaactgcacttgcaaaagtggtgccgtggaatgccgaaggatgtcctgtccccctctcaattgctccccagactccctcccagtgcacattgctggccagtgctgtaaggtctgccgaccaaaatgtatctatggaggaaaagttcttgcagaaggccagcggattttaaccaagagctgtcgggaatgccgaggtggagttttagtaaaaattacagaaatgtgtcctcctttgaactgctcagaaaaggatcacattcttcctgagaatcagtgctgccgtgtctgtagaggtcataacttttgtgcagaaggacctaaatgtggtgaaaactcagagtgcaaaaactggaatacaaaagctacttgtgagtgcaagagtggttacatctctgtccagggagactctgcctactgtgaagatattgatgagtgtgcagctaagatgcattactgtcatgccaatactgtgtgtgtcaaccttcctgggttatatcgctgtgactgtgtcccaggatacattcgtgtggatgacttctcttgtacagaacacgatgaatgtggcagcggccagcacaactgtgatgagaatgccatctgcaccaacactgtccagggacacagctgcacctgcaaaccgggctacgtggggaacgggaccatctgcagagctttctgtgaagagggctgcagatacggtggaacgtgtgtggctcccaacaaatgtgtctgtccatctggattcacaggaagccactgcgagaaagatattgatgaatgttcagagggaatcattgagtgccacaaccattcccgctgcgttaacctgccagggtggtaccactgtgagtgcagaagcggtttccatgacgatgggacctattcactgtccggggagtcctgtattgacattgatgaatgtgccttaagaactcacacctgttggaacgattctgcctgcatcaacctggcagggggctttgactgtctctgcccctctgggccctcctgctctggtgactgtcctcatgaaggggggctgaagcacaatggccaggtgtggaccttgaaagaagacaggtgttctgtctgctcctgcaaggatggcaagatattctgccgacggacagcttgtgattgccagaatccaagtgctgacctattctgttgcccagaatgtgacaccagagtcacaagtcaatgtttagaccaaaatggtcacaagctgtatcgaagtggagacaattggacccatagctgtcagcagtgtcggtgtctggaaggagaggtagattgctggccactcacttgccccaacttgagctgtgagtatacagctatcttagaaggggaatgttgtccccgctgtgtcagtgacccctgcctagctgataacatcacctatgacatcagaaaaacttgcctggacagctatggtgtttcacggcttagtggctcagtgtggacgatggctggatctccctgcacaacctgtaaatgcaagaatggaagagtctgttgttctgtggattttgagtgtcttcaaaataattgaagtatttacagtggactcaacgcagaagaatggacgaaatgaccatccaacgtgattaaggataggaatcggtagtttggtttttttgtttgttttgtttttttaaccacagataattgccaaagtttccacctgaggacggtgtttggaggttgccttttggacctaccactttgctcattcttgctaacctagtctaggtgacctacagtgccgtgcatttaagtcaatggttgttaaaagaagtttcccgtgttgtaaatcatgtttcccttatcagatcatttgcaaatacatttaaatgatctcatggtaaatgttgatgtattttttggtttattttgtgtactaacataatagagagagactcagctccttttatttattttgttgatttatggatcaaattctaaaataaagttgcctgttgtgacttttgtcccatctactgcatacttagtgctgagatccctgtaaaatgttttgatgaaaatatgtatgtagagtccagtcgcattatacatacatttcatagtgctgaaccttcttaaatgcctactcattcagcttaaacaggctgaagccaagtatgacaaagaggggaagggccaaaaacataatcaaagaataattttaaagagaattcttgtctctcttgcaaaaaaaaaa

SEQ ID NO: 2 - protein, 810 aa, Homo sapiens
MPMDLILVVWFCVCTARTVVGFGMDPDLQMDIVTELDLVNTTLGVAQVSGMHNASKAFLFQDIEREIHAAPHVSEKLIQLFRNKSEFTILATVQQKPSTSGVILSIRELEHSYFELESSGLRDEIRYHYIHNGKPRTEALPYRMADGQWHKVALSVSASHLLLHVDCNRIYERVIDPPDTNLPPGINLWLGQRNQKHGLFKGIIQDGKIIFMPNGYITQCPNLNHTCPTCSDFLSLVQGIMDLQELLAKMTAKLNYAETRLSQLENCHCEKTCQVSGLLYRDQDSWVDGDHCRNCTCKSGAVECRRMSCPPLNCSPDSLPVHIAGQCCKVCRPKCIYGGKVLAEGQRILTKSCRECRGGVLVKITEMCPPLNCSEKDHILPENQCCRVCRGHNFCAEGPKCGENSECKNWNTKATCECKSGYISVQGDSAYCEDIDECAAKMHYCHANTVCVNLPGLYRCDCVPGYIRVDDFSCTEHDECGSGQHNCDENAICTNTVQGHSCTCKPGYVGNGTICRAFCEEGCRYGGTCVAPNKCVCPSGFTGSHCEKDIDECSEGIIECHNHSRCVNLPGWYHCECRSGFHDDGTYSLSGESCIDIDECALRTHTCWNDSACINLAGGFDCLCPSGPSCSGDCPHEGGLKHNGQVWTLKEDRCSVCSCKDGKIFCRRTACDCQNPSADLFCCPECDTRVTSQCLDQNGHKLYRSGDNWTHSCQQCRCLEGEVDCWPLTCPNLSCEYTAILEGECCPRCVSDPCLADNITYDIRKTCLDSYGVSRLSGSVWTMAGSPCTTCKCKNGRVCCSVDFECLQNN

SEQ ID NO: 3 - protein, 185 aa, Homo sapiens
QMDIVTELDLVNTTLGVAQVSGMHNASKAFLFQDIEREIHAAPHVSEKLIQLFRNKSEFTILATVQQKPSTSGVILSIRELEHSYFELESSGLRDEIRYHYIHNGKPRTEALPYRMADGQWHKVALSVSASHLLLHVDCNRIYERVIDPPDTNLPPGINLWLGQRNQKHGLFKGIIQDGKIIFMP

SEQ ID NO: 4 - protein, 59 aa, Homo sapiens
CQVSGLLYRDQDSWVDGDHCRNCTCKSGAVECRRMSCPPLNCSPDSLPVHIAGQCCKVC

SEQ ID NO: 5 - protein, 49 aa, Homo sapiens
KLYRSGDNWTHSCQQCRCLEGEVDCWPLTCPNLSCEYTAILEGECCPRC

SEQ ID NO: 6 - protein, 38 aa, Homo sapiens
DIDECSEGIIECHNHSRCVNLPGWYHCECRSGFHDDGT

SEQ ID NO: 7 - protein, 35 aa, Homo sapiens
DECGSGQHNCDENAICTNTVQGHSCTCKPGYVGNG

SEQ ID NO: 8 - protein, 33 aa, Homo sapiens
DIDECAAKMHYCHANTVCVNLPGLYRCDCVPGY

SEQ ID NO: 9 - protein, 32 aa, Homo sapiens
DIDECALRTHTCWNDSACINLAGGFDCLCPSG

SEQ ID NO: 10 - DNA, 3114 bp, Homo sapiens
  CDS (154)...(2445): Coding region
atatgcgagcgcagcacccggcgctgccgagccacctcccccgccgcccgctagcaagtttggcggctccaagccaggcgcgcctcaggatccaggctcatttgcttccacctagcttcggtgccccctgctaggcggggaccctcgagagcgatgccgatggatttgattttagttgtgtggttctgtgtgtgcactgccaggacagtggtgggctttgggatggaccctgaccttcagatggatatcgtcaccgagcttgaccttgtgaacaccacccttggagttgctcaggtgtctggaatgcacaatgccagcaaagcatttttatttcaagacatagaaagagagatccatgcagctcctcatgtgagtgagaaattaattcagctgttccggaacaagagtgaattcaccattttggccactgtacagcagaagccatccacttcaggagtgatactgtccattcgagaactggagcacagctattttgaactggagagcagtggcctgagggatgagattcggtatcactacatacacaatgggaagccaaggacagaggcacttccttaccgcatggcagatggacaatggcacaaggttgcactgtcagttagcgcctctcatctcctgctccatgtcgactgtaacaggatttatgagcgtgtgatagaccctccagataccaaccttcccccaggaatcaatttatggcttggccagcgcaaccaaaagcatggcttattcaaagggatcatccaagatgggaagatcatctttatgccgaatggatatataacacagtgtccaaatctaaatcacacttgcccaacctgcagtgatttcttaagcctggtgcaaggaataatggatttacaagagcttttggccaagatgactgcaaaactaaattatgcagagacaagacttagtcaattggaaaactgtcattgtgagaagacttgtcaagtgagtggactgctctatcgagatcaagactcttgggtagatggtgaccattgcaggaactgcacttgcaaaagtggtgccgtggaatgccgaaggatgtcctgtccccctctcaattgctccccagactccctcccagtgcacattgctggccagtgctgtaaggtctgccgaccaaaatgtatctatggaggaaaagttcttgcagaaggccagcggattttaaccaagagctgtcgggaatgccgaggtggagttttagtaaaaattacagaaatgtgtcctcctttgaactgctcagaaaaggatcacattcttcctgagaatcagtgctgccgtgtctgtagaggtcataacttttgtgcagaaggacctaaatgtggtgaaaactcagagtgcaaaaactggaatacaaaagctacttgtgagtgcaagagtggttacatctctgtccagggagactctgcctactgtgaagatattgatgagtgtgcagctaagatgcattactgtcatgccaatactgtgtgtgtcaaccttcctgggttatatcgctgtgactgtgtcccaggatacattcgtgtggatgacttctcttgtacagaacacgatgaatgtggcagcggccagcacaactgtgatgagaatgccatctgcaccaacactgtccagggacacagctgcacctgcaaaccgggctacgtggggaacgggaccatctgcagagctttctgtgaagagggctgcagatacggtggaacgtgtgtggctcccaacaaatgtgtctgtccatctggattcacaggaagccactgcgagaaagacattgatgaatgtgccttaagaactcacacctgttggaacgattctgcctgcatcaacctggcagggggctttgactgtctctgcccctctgggccctcctgctctggtgactgtcctcatgaaggggggctgaagcacaatggccaggtgtggaccttgaaagaagacaggtgttctgtctgctcctgcaaggatggcaagatattctgccgacggacagcttgtgattgccagaatccaagtgctgacctattctgttgcccagaatgtgacaccagagtcacaagtcaatgtttagaccaaaatggtcacaagctgtatcgaagtggagacaattggacccatagctgtcagcagtgtcggtgtctggaaggagaggtagattgctggccactcacttgccccaacttgagctgtgagtatacagctatcttagaaggggaatgttgtccccgctgtgtcagtgacccctgcctagctgataacatcacctatgacatcagaaaaacttgcctggacagctatggtgtttcacggcttagtggctcagtgtggacgatggctggatctccctgcacaacctgtaaatgcaagaatggaagagtctgttgttctgtggattttgagtgtcttcaaaataattgaagtatttacagtggactcaacgcagaagaatggacgaaatgaccatccaacgtgattaaggataggaatcggtagtttggtttttttgtttgttttgtttttttaaccacagataattgccaaagtttccacctgaggacggtgtttggaggttgccttttggacctaccactttgctcattcttgctaacctagtctaggtgacctacagtgccgtgcatttaagtcaatggttgttaaaagaagtttcccgtgttgtaaatcatgtttcccttatcagatcatttgcaaatacatttaaatgatctcatggtaaatgttgatgtattttttggtttattttgtgtactaacataatagagagagactcagctccttttatttattttgttgatttatggatcaaattctaaaataaagttgcctgttgtgacttttgtcccatctactgcatacttagtgctgagatccctgtaaaatgttttgatgaaaatatgtatgtagagtccagtcgcattatacatacatttcatagtgctgaaccttcttaaatgcctactcattcagcttaaacaggctgaagccaagtatgacaaagaggggaagggccaaaaacataatcaaagaataattttaaagagaattcttgtctctcttgcaaaaaaaaaa

SEQ ID NO: 11 - protein, 763 aa, Homo sapiens
MPMDLILVVWFCVCTARTVVGFGMDPDLQMDIVTELDLVNTTLGVAQVSGMHNASKAFLFQDIEREIHAAPHVSEKLIQLFRNKSEFTILATVQQKPSTSGVILSIRELEHSYFELESSGLRDEIRYHYIHNGKPRTEALPYRMADGQWHKVALSVSASHLLLHVDCNRIYERVIDPPDTNLPPGINLWLGQRNQKHGLFKGIIQDGKIIFMPNGYITQCPNLNHTCPTCSDFLSLVQGIMDLQELLAKMTAKLNYAETRLSQLENCHCEKTCQVSGLLYRDQDSWVDGDHCRNCTCKSGAVECRRMSCPPLNCSPDSLPVHIAGQCCKVCRPKCIYGGKVLAEGQRILTKSCRECRGGVLVKITEMCPPLNCSEKDHILPENQCCRVCRGHNFCAEGPKCGENSECKNWNTKATCECKSGYISVQGDSAYCEDIDECAAKMHYCHANTVCVNLPGLYRCDCVPGYIRVDDFSCTEHDECGSGQHNCDENAICTNTVQGHSCTCKPGYVGNGTICRAFCEEGCRYGGTCVAPNKCVCPSGFTGSHCEKDIDECALRTHTCWNDSACINLAGGFDCLCPSGPSCSGDCPHEGGLKHNGQVWTLKEDRCSVCSCKDGKIFCRRTACDCQNPSADLFCCPECDTRVTSQCLDQNGHKLYRSGDNWTHSCQQCRCLEGEVDCWPLTCPNLSCEYTAILEGECCPRCVSDPCLADNITYDIRKTCLDSYGVSRLSGSVWTMAGSPCTTCKCKNGRVCCSVDFECLQNN

SEQ ID NO: 12 - protein, 185 aa, Homo sapiens; identical to SEQ ID NO: 3

SEQ ID NO: 13 - protein, 59 aa, Homo sapiens; identical to SEQ ID NO: 4

SEQ ID NO: 14 - protein, 49 aa, Homo sapiens; identical to SEQ ID NO: 5

SEQ ID NO: 15 - protein, 35 aa, Homo sapiens; identical to SEQ ID NO: 7

SEQ ID NO: 16 - protein, 33 aa, Homo sapiens; identical to SEQ ID NO: 8

SEQ ID NO: 17 - protein, 32 aa, Homo sapiens; identical to SEQ ID NO: 9

SEQ ID NO: 18 - DNA, 2812 bp, Mus musculus
  CDS (40)...(2472): coding region
gcgttggtgcgccctgcttggcggggggcctccggagcgatgccgatggatgtgattttagttttgtggttctgtgtgtgcaccgccaggacagtgctgggctttgggatggaccctgaccttcagatggacatcatcactgaacttgaccttgtgaacaccaccctgggcgtcactcaggtggctggactacacaatgccagtaaggcatttctgtttcaagatgtacagagagagatccactcagcccctcatgtgagtgagaagctgatccagctattccggaataagagtgagtttacctttttggctacagtgcagcagaagccgtccacctcaggggtgatactgtcgatccgggagctggaacacagctattttgaactggagagcagtggcccaagagaagagatacgctatcattacatccatggcggcaagcccaggactgaggcccttccctaccgcatggccgatggacagtggcacaaggtcgcgctgtctgtgagcgcctctcacctcctactccatgtcgactgcaataggatttatgagcgtgtgatagatcctccggagaccaaccttcctccaggaagcaatctatggcttgggcaacgtaatcaaaagcatggctttttcaaaggaatcatccaagatggcaagatcatcttcatgccgaacggcttcatcacacagtgccccaacctaaatcgcacttgcccaacatgcagtgatttcctgagcctggttcaaggaataatggatttgcaagagcttttggccaagatgactgcaaaactgaattatgcagagacgagacttggtcaactggaaaattgccactgtgagaagacctgccaagtgagtgggctgctctacagggaccaagactcctgggtagatggtgacaactgcaggaactgcacatgcaaaagtggtgctgtggagtgccgaaggatgtcctgtcccccactcaactgttccccagactcacttcctgtgcatatttctggccaatgttgtaaagtttgcagaccaaaatgtatctatggaggaaaagttcttgctgagggccagcggattttaaccaagacctgccgggaatgtcgaggtggagtcttggtaaaaatcacagaagcttgccctcctttgaactgctcagagaaggatcatattcttccggagaaccagtgctgcagggtctgccgaggtcataacttctgtgcagaagcacctaagtgtggagaaaactcggaatgcaaaaattggaatacaaaagcgacttgtgagtgcaagaatggatacatctctgtccagggcaactctgcatactgtgaagatatcgatgagtgtgcagcaaagatgcactactgtcatgccaacacggtgtgtgtcaacttgccggggttatatcgctgtgactgcatcccaggatacatccgtgtggatgacttctcttgtacggagcatgatgattgtggcagcggacaacacaactgtgacaaaaatgccatctgtaccaacacagtccagggacacagctgtacctgccagccaggctacgtgggaaatggtactgtctgcaaagcattctgtgaagagggttgcagatacggaggtacctgtgtggcccctaacaaatgtgtctgtccttctggattcacaggaagccactgtgagaaagatattgatgaatgtgcagagggattcgttgagtgccacaaccactcccgctgcgttaaccttccagggtggtaccactgtgagtgcagaagcggtttccatgacgatgggacctattcactgtccggggagtcctgcattgatattgatgaatgtgccttaagaactcacacttgttggaatgactctgcctgcatcaacttagcaggaggatttgactgcctgtgtccctctgggccctcctgctctggtgactgtccccacgaaggggggctgaagcataatgggcaggtgtggattctgagagaagacaggtgttcagtctgttcctgtaaggatgggaagatattctgccggcggacagcttgtgattgccagaatccaaatgttgaccttttctgctgcccagagtgtgacaccagggtcactagccaatgtttagatcaaagcggacagaagctctatcgaagtggagacaactggacccacagctgccagcagtgccgatgtctggaaggagaggcagactgctggcctctagcttgccctagtttgagctgtgaatacacagccatctttgaaggagagtgttgtccccgctgtgtcagtgacccctgcctggctgataatattgcctatgacatcagaaaaacttgcctggacagctctggtatttcgaggctgagcggcgcagtgtggacaatggctggatctccctgtacaacctgtcaatgcaagaatgggagagtctgctgctctgtggatctggtgtgtcttgagaataactgaagattttaaatggactcatcacatgagaaaatggacaaaatgaccatccaacctgaggaagaggaggggctgatttctttttctttttaaccacagtcaattaccaaagtctccatcagaggaaggcgtttgggttgcctttaccactttgctcatccttgctgacctagtctagatgcctgcagtaccgtgtatttcggtcgatggttgttgagtctccgtgctgtaaatcacatttcccttgtcagatcatttacagatacatttaaaggattccatgataaatgttaaagtaccttttgtttattttgtgtaccaacataatagagacttggcacca

SEQ ID NO: 19 - protein, 810 aa, Mus musculus
MPMDVILVLWFCVCTARTVLGFGMDPDLQMDIITELDLVNTTLGVTQVAGLHNASKAFLFQDVQREIHSAPHVSEKLIQLFRNKSEFTFLATVQQKPSTSGVILSIRELEHSYFELESSGPREEIRYHYIHGGKPRTEALPYRMADGQWHKVALSVSASHLLLHVDCNRIYERVIDPPETNLPPGSNLWLGQRNQKHGFFKGIIQDGKIIFMPNGFITQCPNLNRTCPTCSDFLSLVQGIMDLQELLAKMTAKLNYAETRLGQLENCHCEKTCQVSGLLYRDQDSWVDGDNCRNCTCKSGAVECRRMSCPPLNCSPDSLPVHISGQCCKVCRPKCIYGGKVLAEGQRILTKTCRECRGGVLVKITEACPPLNCSEKDHILPENQCCRVCRGHNFCAEAPKCGENSECKNWNTKATCECKNGYISVQGNSAYCEDIDECAAKMHYCHANTVCVNLPGLYRCDCIPGYIRVDDFSCTEHDDCGSGQHNCDKNAICTNTVQGHSCTCQPGYVGNGTVCKAFCEEGCRYGGTCVAPNKCVCPSGFTGSHCEKDIDECAEGFVECHNHSRCVNLPGWYHCECRSGFHDDGTYSLSGESCIDIDECALRTHTCWNDSACINLAGGFDCLCPSGPSCSGDCPHEGGLKHNGQVWILREDRCSVCSCKDGKIFCRRTACDCQNPNVDLFCCPECDTRVTSQCLDQSGQKLYRSGDNWTHSCQQCRCLEGEADCWPLACPSLSCEYTAIFEGECCPRCVSDPCLADNIAYDIRKTCLDSSGISRLSGAVWTMAGSPCTTCQCKNGRVCCSVDLVCLENN

SEQ ID NO: 20 - DNA, 2915 bp, Rattus norvegicus
  CDS (59)...(2491): Coding region
aagcactggtttcttgttagcgttggtgcgccctgcttggcgggggttctccggagcgatgccgatggatgtgattttagttttgtggttctgtgtatgcaccgccaggacagtgttgggctttgggatggaccctgaccttcagctggacatcatctcagagctcgacctggtgaacaccaccctgggagtcacgcaggtggctggactgcacaacgccagtaaagcatttctatttcaagatgtacagagagagatccattcggcccctcacgtgagtgagaagctgatccagctattccggaataagagcgagttcacctttttggctacagtgcagcagaaaccatccacctcaggggtgatactgtccatccgggagctggagcacagctattttgaactggagagcagtggcccaagagaagagatacgctaccattacatacatggtggaaagcccaggactgaggcccttccctaccgcatggcagacggacaatggcacaaggtcgcgctgtcagtgagcgcctctcacctcctgctccacatcgactgcaataggatttacgagcgtgtgatagaccctccggagaccaaccttcctccaggaagcaatctgtggcttgggcaacgtaaccaaaagcatggctttttcaaaggaatcatccaagatggtaagatcatcttcatgccgaatggtttcatcacacagtgtcccaacctcaatcgcacttgcccaacatgcagtgacttcctgagcctggttcaaggaataatggatttgcaagagcttttggccaagatgactgcaaaactgaattatgcagagacgagacttggtcaactggaaaattgccactgtgagaagacctgccaagtgagtgggctgctctacagggaccaagactcctgggtggatggtgacaactgtgggaactgcacgtgcaaaagtggtgccgtggagtgccgcaggatgtcctgtcccccgctcaactgttccccggactcacttcctgtgcacatttccggccagtgttgtaaagtttgcagaccaaaatgtatctatggaggaaaagttcttgctgagggccagcggattttaaccaagacctgccgggaatgtcgaggtggagtcttggtaaaaatcacagaagcttgccctcctttgaactgctcagcaaaggatcatattcttccagagaatcagtgctgcagggtctgcccaggtcataacttctgtgcagaagcacctaagtgcggagaaaactcggaatgcaaaaattggaatacaaaagcaacctgtgagtgcaagaatggatacatctctgtccagggcaactctgcatactgtgaagatattgatgagtgtgcagctaaaatgcactattgtcatgccaacaccgtgtgtgtcaacttgccggggttgtatcgctgtgactgcgtcccagggtacatccgtgtggatgacttctcttgtacggagcatgatgattgtggcagcggacaacacaactgcgacaaaaatgccatctgtaccaacacagtccagggacacagctgcacctgccagccgggttacgtgggaaatggcaccatctgcaaagcattctgtgaagagggttgcagatacggaggtacctgtgtggctcctaacaagtgtgtctgtccttctggattcacgggaagccactgtgagaaagatattgatgaatgcgcagagggattcgttgaatgccacaactactcccgctgtgttaacctgccagggtggtaccactgtgagtgcagaagcggtttccatgacgatgggacctactcactgtccggggagtcctgcattgatatcgatgaatgtgccttaagaactcacacttgttggaatgactctgcctgcatcaacttagcaggaggatttgactgcctgtgtccctctgggccctcctgctctggtgactgtccccacgaaggagggctgaagcataatgggcaggtgtggattctgagagaagacaggtgttcagtctgttcctgcaaggatgggaagatattctgccggcggacagcttgtgattgccagaatccaaatgttgaccttttttgctgcccagagtgcgataccagggtcaccagccaatgtttagatcaaagtggacagaagctctatcgaagtggagacaactggacccacagctgccagcagtgccgatgtctggaaggagaggcagactgctggcctctggcttgccctagtttgggctgtgaatacacagccatgtttgaaggggagtgttgtccccgatgtgtcagtgacccctgcctggctggtaatattgcctatgacatcagaaaaacttgcctggacagctttggtgtttcgaggctgagcggagccgtgtggacaatggctggatctccttgtacaacctgcaaatgcaagaatgggagagtctgctgctctgtggatctggagtgtattgagaataactgaagattttaaatggactcgtcacgtgagaaaatgggcaaaatgatcatcccacctgaggaagaagaggggctgatttctttttctttttaaccacagtcaattaccaaagtctccatctgaggaaggcgtttggattgcctttgccactttgctcatccttgctgacctagtctagatgcctgcagtaccgtgcatttcggtcgatggttgttgagtctcagtgttgtaaatcgcatttccctcgtcagatcatttacagatacatttaaaggggttccatgataaatgttaatgtaacttttgtttattttgtgtactgacataatagagacttggcaccatttatttatttttcttgatttttggatcaaattctaaaaataaagttgcctgttgcgaaaaaaaaaaaaaaaaaaaaaaaaaa

SEQ ID NO: 21 - protein, 810 aa, Rattus norvegicus
MPMDVILVLWFCVCTARTVLGFGMDPDLQLDIISELDLVNTTLGVTQVAGLHNASKAFLFQDVQREIHSAPHVSEKLIQLFRNKSEFTFLATVQQKPSTSGVILSIRELEHSYFELESSGPREEIRYHYIHGGKPRTEALPYRMADGQWHKVALSVSASHLLLHIDCNRIYERVIDPPETNLPPGSNLWLGQRNQKHGFFKGIIQDGKIIFMPNGFITQCPNLNRTCPTCSDFLSLVQGIMDLQELLAKMTAKLNYAETRLGQLENCHCEKTCQVSGLLYRDQDSWVDGDNCGNCTCKSGAVECRRMSCPPLNCSPDSLPVHISGQCCKVCRPKCIYGGKVLAEGQRILTKTCRECRGGVLVKITEACPPLNCSAKDHILPENQCCRVCPGHNFCAEAPKCGENSECKNWNTKATCECKNGYISVQGNSAYCEDIDECAAKMHYCHANTVCVNLPGLYRCDCVPGYIRVDDFSCTEHDDCGSGQHNCDKNAICTNTVQGHSCTCQPGYVGNGTICKAFCEEGCRYGGTCVAPNKCVCPSGFTGSHCEKDIDECAEGFVECHNYSRCVNLPGWYHCECRSGFHDDGTYSLSGESCIDIDECALRTHTCWNDSACINLAGGFDCLCPSGPSCSGDCPHEGGLKHNGQVWILREDRCSVCSCKDGKIFCRRTACDCQNPNVDLFCCPECDTRVTSQCLDQSGQKLYRSGDNWTHSCQQCRCLEGEADCWPLACPSLGCEYTAMFEGECCPRCVSDPCLAGNIAYDIRKTCLDSFGVSRLSGAVWTMAGSPCTTCKCKNGRVCCSVDLECIENN

SEQ ID NO: 22 - DNA, 3200 bp, Pan troglodytes
  CDS (121)...(2556): Coding region
acctcccccgccgccggttagcaagtttggcggctccaagccaggcgcgcctcaggatccaggttcatttgcttccacctagcttcggtggcccctgctaggcggggaccctcgagagcgatgccgatggatttgattttagttgtgtggttctgtgtgtgcactgccaggacagtggtgggctttgggatggaccctgaccttcagatggatatcatcaccgagcttgaccttgtgaacaccactcttggagttgctcaggtgtctggaatgcacaatgccagcaaagcatttttatttcaagacatagaaagagagatccatgcagctcctcatgtgagtgagaaattaattcagctgttccagaacaagagtgaattcacctttttggccactgtacagcagaagccatccacttcaggagtgatactgtccattcgagaactggagcacagctattttgaactggagagcagtggcctgagggatgagattcggtatcactacatacacaatgggaagccaaggacagaggcacttccttaccgcatggcagatggacgatggcacaaggttgcactgtcagttagcgcctctcatctcctgctccatgtcgactgtaacaggatttatgagcgtgtgatagaccctccagataccaaccttcccccaggaatcaatttatggcttggccagcgcaaccaaaagcatggcttattcaaagggatcatccaagatgggaagatcatctttatgccgaatggatatataacacagtgtccaaatctaaatcgcacttgcccaacctgcagtgatttcttaagcctggtgcaaggaataatggatttacaagagcttttggccaagatgactgcaaaactaaattatgcagagacaagacttagtcaattggaaaactgtcattgtgagaagacttgtcaagtgagtggactgctctatcgagatcaagactcttgggtagatggtgaccattgcaggagctgcacttgcaaaagtggtgccgtggaatgccgaaggatgtcctgtccccctctcaattgctccccagactccctcccagtgcacattgctggccagtgctgtaaggtctgccgaccaaaatgtatctatggaggaaaagttcttgcagaaggccagcggattttaaccaagagctgtcgggaatgccgaggtggagttttagtaaaaattacagaaatgtgtcctgccttgaactgctcagaaaaggatcatattcttcctgagaatcagtgctgccgtgtctgtagaggtcataacttctgtgcagaaggacctaaatgtggtgaaaactcagagtgcaaaaactggaatacaaaagctacttgtgagtgcaagagtggttacatctctgtccagggagactctgcctactgtgaagatattgatgagtgtgcagctaagatgcattactgtcatgccaatactgtgtgtgtcaaccttcctgggttatatcgctgtgactgtgtcccaggatacattcgtgtggatgacttctcttgtacagaacacgatgaatgtggcagcggccagcacaactgtgacgagaatgccatctgcaccaacactgtccagggacacagctgcacctgcaaaccgggctacgtggggaacgggaccatctgcagagctttctgtgaagagggctgcagatacggtggaacgtgtgtggctcctaacaaatgtgtctgtccatctggattcacaggaagccactgcgagaaagatattgatgaatgttcagagggaatcattgagtgccacaaccattcccgctgcgttaacctgccagggtggtaccactgtgagtgcagaagcggtttccatgacgatgggacctattcactgtccggggagtcctgtattgacattgatgaatgtgccttaagaactcacacctgttggaacgattctgcctgcatcaacctggcagggggctttgactgtctctgcccctctgggccctcctgctctggtgactgtcctcatgaaggggggctgaagcacaatggccaggtgtggaccttgaaagaagacaggtgttctgtctgctcctgcaaggatggcaagatattctgccgacggacagcttgtgattgccagaatccaagtgctgatctattctgttgcccagaatgtgacaccagagtcacaagtcaatgtttagaccaaaatggtcacaagctgtatcgaagtggagacaattggacccatagctgtcagcagtgtcggtgtctggaaggagaggtagattgctggccactcacttgccccaacttgagctgtgagtatacagctatcttagaaggggaatgttgtccccgctgtgtcagtgacccctgcctagctgataacatcatctatgacatcagaaaaacttgcctggacagctatggtgtttcaggncttagtggctcagtgtggacgatggctggatctccctgcacatgtttctttgatgtacagaatggaagagtctgttgttctgtggattttgagtgtcttcaaaataattgaagtatttacaatggactcaatgcagaagaatggacaaaatgaccatccaacgtgattaaggataggaattggtggtttggtttttttgtttgttttgtttttttaaccacagataattgccaaagtttccatctgaggacggtgtttggaggttgccttttggacctaccactttgctcattcttgctaacctagtctaggtgacctacagtgccgtgcatttaagtcaatggttgttaaaagaagtttcccgtgttgtaaatcaatcatgtttcccttatcagatcatttgcaaatacatttaaatgatctcatggtaaatgttgatgtattttttggtttattttgtgtactaacgtaatagagactcagctccttttatttattttgttgatttatggatcaaattctaaaataaagttgcctgttgtgacttttgtcccatctactgcatacttagtgctgagatccctgtaaaatgttttgatgaaaatatgtatgtagagtcaatcgcattatacatacatttcaaagtgctgaaccttcttaatgcctactcatcagcttaaacaggctgaagccagtgtgacaagagggaaggacaaaacataatcaaagaatatttaagagactcttgtctctgc

SEQ ID NO: 23 - protein, 811 aa, Pan troglodytes
MPMDLILVVWFCVCTARTVVGFGMDPDLQMDIITELDLVNTTLGVAQVSGMHNASKAFLFQDIEREIHAAPHVSEKLIQLFQNKSEFTFLATVQQKPSTSGVILSIRELEHSYFELESSGLRDEIRYHYIHNGKPRTEALPYRMADGRWHKVALSVSASHLLLHVDCNRIYERVIDPPDTNLPPGINLWLGQRNQKHGLFKGIIQDGKIIFMPNGYITQCPNLNRTCPTCSDFLSLVQGIMDLQELLAKMTAKLNYAETRLSQLENCHCEKTCQVSGLLYRDQDSWVDGDHCRSCTCKSGAVECRRMSCPPLNCSPDSLPVHIAGQCCKVCRPKCIYGGKVLAEGQRILTKSCRECRGGVLVKITEMCPALNCSEKDHILPENQCCRVCRGHNFCAEGPKCGENSECKNWNTKATCECKSGYISVQGDSAYCEDIDECAAKMHYCHANTVCVNLPGLYRCDCVPGYIRVDDFSCTEHDECGSGQHNCDENAICTNTVQGHSCTCKPGYVGNGTICRAFCEEGCRYGGTCVAPNKCVCPSGFTGSHCEKDIDECSEGIIECHNHSRCVNLPGWYHCECRSGFHDDGTYSLSGESCIDIDECALRTHTCWNDSACINLAGGFDCLCPSGPSCSGDCPHEGGLKHNGQVWTLKEDRCSVCSCKDGKIFCRRTACDCQNPSADLFCCPECDTRVTSQCLDQNGHKLYRSGDNWTHSCQQCRCLEGEVDCWPLTCPNLSCEYTAILEGECCPRCVSDPCLADNIIYDIRKTCLDSYGVSGLSGSVWTMAGSPCTCFFDVQNGRVCCSVDFECLQNN

SEQ ID NO: 24 - DNA, 3511 bp, Xenopus tropicalis
  CDS (1)...(2487): Coding region
cactttatgtttagcggctgccgggggattattggggctccttctccgctcactgcgccaatggatggaatgctggctgtgtgggcttgggtactgactgccagagcagtgctcggattggggacagaccccgacctacagatcgacgtaataacggaactggaactggtcaattcaacgagaggcgtgacgcaagtattcggggtgcacaatgggagcaaagcgtttttattccaagacggggcgcgagagatccacgcggcgccgcacatgagcgagaaagtcatccagttgttcaggaacaaaagcgaattcactttcttggcctccatccagcaacgctcctccacgtcgggggtcatcctctccatccgcgagatggagcacagtttcttcgagctggagagcagcggcctgcgggatgaaatccgatatcattaccgcttcaacgggaagtcgaggacggaggcctttccctaccgccttgccgacgggcagtggcacaaaatcgccctcaccgtcagcgcctcccacgttctgctgcatgtggactgcaataggatttacgaacgtgtaatagacccgccggacgccaatctctcgccgggaagcagcctgtggctcggacagcgcaaccgcaagcacggattctttaagggagtcattcaagatgtaaaaatgatcttcatgcccaatggatacatcactcagtgcccgaacttaaatcgcacttgcccaacctgcagcgatttcctgagtctcgtgcaaggaattatggatctacaggagctgctggcgaaaatgacagcaaaattgaactatgcagaaacccggctcagccaactggagaactgccattgtgaaaagacgtgtaacgtccgggaggtggtctatagggacagggactcgtgggtggatgacgatcactgccggaactgtacttgtaagaatggggcagttgagtgccggcggatgctgtgcccccctcttaattgttcctccgattctctcccggtccatgtcgcggggcagtgctgcaaagtctgcaggcccaagtgtatctatggagggaaagtgctggcggaaggagaccggatcctcaccaaaagctgccgagaatgcaaaaatggatctttacaaaagctgatggacccttgcccgcccctcaactgctccgaagccgaccgggttttgccagaaaatcagtgctgcagtgtttgcagagggcataacttctgtgccgacggacacaggtgcggtgagaactcggaatgtaggaaccataacaacaaagctacgtgtgagtgtctcagtggctttcaacccatccaaggggatccggcctactgtgaggatattgatgaatgtgcagccaagatgcattactgtcacgccaatacggtctgcgtcaatttaccgggatcttaccggtgtgattgtgtgccaggatacatccgagtcgatgatttctcctgcacagaacatgatgaatgcggcgaaggtgagagcagctgtgacgagaatgccatctgcgccaactccgtgcgagggcacagctgcacctgtaagccggggtatgtcgggaacggcaccgtctgccgggctttttgcgaggaagggtgtcggtacggcgggacctgtgtggcccccaacaaatgtctctgcccctccggattcaccgggagccactgtgaaaaagatattgatgagtgtgcagaagggatcattgagtgtcacaaccattctcgctgtgttaacctcccgggctggttccactgtgagtgccggagcggtttccatgacaatgggacatactccatttctggggagtcctgtgttgacattgatgaatgcgccctaagaactcacacttgttggaacgactcggcttgcgtcaatctcgagggcggcttcgattgcctttgcccttccggaccctcctgcacaggggactgcccccacgaggggggactcaagcgcaatgggcaggtctggacactgaaagaggatcggtgctcagtctgttcatgcaaggatgggaaaatattctgccgaagaaccgcctgcgactgccagaatcccagcgtcgatctgttctgctgccccgagtgcgacacgagagtcactagccaatgcctggatcagactggccacaaggtgtatcatagcggggacaactggacctacagctgccagcagtgccgctgcctggagggagaagtcgactgttggccgctgacctgccccattctgacctgcgagtacacgaccatctcagaaggcgagtgctgccctcactgcgtcgatgacccctgtatagcagacggggacccctatgacatcaggaaaacctgtcaggaccctcaaggaattacacggctggggggctcagtatggacgatggtcgggtcgccgtgtaccacctgtaaatgcaagaacggaagtgtttgctgctcagtggatttggactgtcttcacaataactaaagttctaacccagtggactctctaatggagaggaagtctaatgcagtcccccatctacagcagagtctccgtccgcagctctgtttcctacaacagacaattaccaaagtctttaccaagaggaagatgtttgggagtttcttgtggaccttcgtctatgatcacaccctcggccctcgcctgggcgccacacatacaaggccgacggctcgttctgagcaacctggggggccgccatgcatctctagctccctccaatggcgattgtttgctcattctcagtgttgtgaaccacttttattctcgttacacttttattttctttattgtagacctttgaatttttcccccaacccgtccaccaccaccaccattttttgtatggcatccaccaccatttttttaaaacttgggttccagaatctaccgaatagtcggatctcatccattgcaatctactaaggccccccctgtctgatgtatttatgaaaatatgtatgtatgtacagtctaattgcatagtaattatatttcatagctatggaacattgtacaattcagcccaaacctgtctctcgagaagaaacatcacgagctgcttcttcatcccaaagggaaccttattggacaatttccggatcacatggtccacattgctttgcttcaggaccttcatggatcatgttgggaaatgataaagtccttcctcctttccccacaatagaacaatagagactttgaaggacttacctggtgcacgtggccgagacgtccagagcgtcgctgcgagacctccggggatgcaacagagatacgacatgctgtgctattgcccatcgttatttatccatttattgaggaaaattttcacgggggttgttatttttaaacaaaaaaatagagagtggaaaagtgatgactgtaagtacggaagcgccagagcggcattactggcgggattattttcagtttactttttaatcgatcacaataaaagccttttaccatcaaaaaaaaaaaaaaa

SEQ ID NO: 25 - protein, 828 aa, Xenopus tropicalis
HFMFSGCRGIIGAPSPLTAPMDGMLAVWAWVLTARAVLGLGTDPDLQIDVITELELVNSTRGVTQVFGVHNGSKAFLFQDGAREIHAAPHMSEKVIQLFRNKSEFTFLASIQQRSSTSGVILSIREMEHSFFELESSGLRDEIRYHYRFNGKSRTEAFPYRLADGQWHKIALTVSASHVLLHVDCNRIYERVIDPPDANLSPGSSLWLGQRNRKHGFFKGVIQDVKMIFMPNGYITQCPNLNRTCPTCSDFLSLVQGIMDLQELLAKMTAKLNYAETRLSQLENCHCEKTCNVREVVYRDRDSWVDDDHCRNCTCKNGAVECRRMLCPPLNCSSDSLPVHVAGQCCKVCRPKCIYGGKVLAEGDRILTKSCRECKNGSLQKLMDPCPPLNCSEADRVLPENQCCSVCRGHNFCADGHRCGENSECRNHNNKATCECLSGFQPIQGDPAYCEDIDECAAKMHYCHANTVCVNLPGSYRCDCVPGYIRVDDFSCTEHDECGEGESSCDENAICANSVRGHSCTCKPGYVGNGTVCRAFCEEGCRYGGTCVAPNKCLCPSGFTGSHCEKDIDECAEGIIECHNHSRCVNLPGWFHCECRSGFHDNGTYSISGESCVDIDECALRTHTCWNDSACVNLEGGFDCLCPSGPSCTGDCPHEGGLKRNGQVWTLKEDRCSVCSCKDGKIFCRRTACDCQNPSVDLFCCPECDTRVTSQCLDQTGHKVYHSGDNWTYSCQQCRCLEGEVDCWPLTCPILTCEYTTISEGECCPHCVDDPCIADGDPYDIRKTCQDPQGITRLGGSVWTMVGSPCTTCKCKNGSVCCSVDLDCLHNN

SEQ ID NO: 26 - DNA, 2931 bp, Canis lupus familiaris
  CDS (1)...(2496): Coding region
atgcatgctctacgaggacatctccagggccagcaatgtgtgctacactgtaagcactcagtaatacctgttgaagagtctgtgtttgaagccaagacagctgtttgtttctttccagtggtgggctttgggacggaccctgacctgcagatggatattatcactgagctcgacctcgtgaacatctcccttggagtcactcaagtgtctggactgcacaatgccagcaaagcatacgtgtttcaagatacagcaagagagatccatgcagcccctcatgtgagtgagaaattaatccaactgttccggaataagagtgacttcacctttttggccacggtacagcaaaagccatcgacttcaggagtgatactctccattcgagagctggagcacagctattttgaactggagagcagtggtctgagggatgagatccgataccactatatgcataatgggaagcctaggaccgaggcccttccgtaccggctggcagatggacagtggcataaggtcgcgctgtctgtcagtgcctctcatctcctgctccacatcgactgcaacagaatttatgaacgcgtaatagaccctcctgaaaccaaccttcccccaggaagcaacttatggcttggtcaacgcaaccaaaaacatggctttttcaaaggaatcatccaagatgggaaaatcatctttatgccaaatggatacataactcaatgtccaaacctgaatcgcacttgtccaacttgcagtgatttcttaagcctggtgcaaggaataatggatttgcaagagcttttggccaagatgactgcaaaattaaattatgcagagacgagacttagtcagttggaaaactgtcattgcgagaagacctgtcaagtgagtggacttctctatagagatcaagactcctgggttgatggggatcactgccggaactgcacttgcaaaggtggtgctgtcgaatgtcggaggatgtcctgtccccctctcaattgctccccagactccctccccgtgcacattgctggccagtgctgtaaggtctgccgaccaaaatgtatctatggaggcagagtccttgcagaaggccagaggattttaactaagagctgccgggaatgcagaggtggagttttagtaaaaattacagatgcatgccctcctttgaactgctcggaaaaggatcacattctcccggagaatcagtgctgcagtgtctgtagaggtcacaacttttgtgcagaaggacctaaatgtggtgaaaactcggagtgtaaaaactggaacacaaaagctacttgtgagtgcaagaatggttacatatctgtccagggagactctgcctactgtgaagatattgatgaatgtgcagctaagatgcattactgtcatgccaatactgtgtgtgtcaacctgcctgggttatatcgctgtgactgtgtcccagggtacattcgtgtggatgacttctcttgtacagagcatgatgagtgtggcagcgggcaacacaattgtgatgagaatgccatctgcaccaacaccgtccggggacacagctgcacctgcaaaccaggctacgtggggaatggaaccatctgcagagcattctgtcaagagggctgcaggtatggtggaagctgtgtgtctcctaacaagtgcgtctgtccctctggattcacaggaagccactgtgagaaagatattgatgaatgcacagagggaatcattgagtgccacaaccattcccgctgcgttaacctgccagggtggtaccactgtgagtgcagaagcggtttccatgacgatgggacctattcactgtccggggagtcctgtattgacattgatgaatgtgccttaagaacgcatacctgctggaatgattctgcttgcatcaacctggcagggggctttgactgcctctgtccctcggggccctcctgctctggtgactgcccccatgagggaggactgaagcgcaatggacaggtgtggaccctgaaagaagacaggtgttctgtgtgctcctgcaaggatggaaagatactctgccgacggacagcttgtgattgccagaacccaagcgttgaccttttctgttgcccagagtgtgacaccagggtcacaagtcaatgtttagatcaaaatggacacaagctctatcgaagcggagacaattggactcatagctgccagcagtgccggtgtctggaaggagaggtagactgctggcccctcacttgccccaatttgagctgtgagtatacagccatcttggaaggggagtgttgtccccgctgcgtgagtgacccctgcctagctgataacatcgcctacgacatcagaaaaacctgcctggacagctatggcatttcaaggcttagcggctcagtgtggacaatggctggatctccctgcacaacctgcaaatgcaagaatggaagcgtctgctgttctgtggatttggagtgtcttcataataactgaagtacttaaaatagactcatcgttgcgggagaaaaatggacgaaatgaccatccagcatggtgaagaacaggagttggtgtttcttttgtttttgtttttgttttttaccacaattaccaaagtcccgtctgaggaaggtgtttgggggttacctcttggacctatcactttgttcattcttgctaacctagtctaggtgacctacagtgcactgcatttaagtctatggttgttaaaagaagtttctcgtgttgtaaatcatgtttcccttatcaaatcatttgcgaatacatttaaatgatctcatggtaaacgttgatgtattttttgttttattttgtgtactaacatgatagcgaatccgctcttttaatttattttttttcttgagttttggatcaaattctacaaataaagttgcctgttgtgatttt

SEQ ID NO: 27 - protein, 831 aa, Canis lupus familiaris
MHALRGHLQGQQCVLHCKHSVIPVEESVFEAKTAVCFFPVVGFGTDPDLQMDIITELDLVNISLGVTQVSGLHNASKAYVFQDTAREIHAAPHVSEKLIQLFRNKSDFTFLATVQQKPSTSGVILSIRELEHSYFELESSGLRDEIRYHYMHNGKPRTEALPYRLADGQWHKVALSVSASHLLLHIDCNRIYERVIDPPETNLPPGSNLWLGQRNQKHGFFKGIIQDGKIIFMPNGYITQCPNLNRTCPTCSDFLSLVQGIMDLQELLAKMTAKLNYAETRLSQLENCHCEKTCQVSGLLYRDQDSWVDGDHCRNCTCKGGAVECRRMSCPPLNCSPDSLPVHIAGQCCKVCRPKCIYGGRVLAEGQRILTKSCRECRGGVLVKITDACPPLNCSEKDHILPENQCCSVCRGHNFCAEGPKCGENSECKNWNTKATCECKNGYISVQGDSAYCEDIDECAAKMHYCHANTVCVNLPGLYRCDCVPGYIRVDDFSCTEHDECGSGQHNCDENAICTNTVRGHSCTCKPGYVGNGTICRAFCQEGCRYGGSCVSPNKCVCPSGFTGSHCEKDIDECTEGIIECHNHSRCVNLPGWYHCECRSGFHDDGTYSLSGESCIDIDECALRTHTCWNDSACINLAGGFDCLCPSGPSCSGDCPHEGGLKRNGQVWTLKEDRCSVCSCKDGKILCRRTACDCQNPSVDLFCCPECDTRVTSQCLDQNGHKLYRSGDNWTHSCQQCRCLEGEVDCWPLTCPNLSCEYTAILEGECCPRCVSDPCLADNIAYDIRKTCLDSYGISRLSGSVWTMAGSPCTTCKCKNGSVCCSVDLECLHNN

SEQ ID NO: 28 - DNA, 2205 bp, Culex quinquefasciatus
  CDS (1)...(2205): Coding region
atgaagcggacggccgatttcacgtttgccgtcgtcctgaagcaggagcaagctaactctgggaccatcgtttccttttcaaacggaaacaataggtatttagaattacaatcaagtggaagacgtgacgaaatacgttttcattacacacacataacagcagcgggcgagccgctgatgcgctacgagtccttcccgtatcggcttgcggacgacgccgagcacaaggtggcgctaacggtcagcggcaccgaggtgcagctctacatcgactgccatccactgtataagcgagtgatgcattttttaccagatagaaattttagtgcttccaatatgcaattattcgtcggacaaagaaactcaaatagtcattatttatttaagggtgatttaaaagatttacgtattatcaccggcccgtacggttatttatcacaatgtgaactaatggatgcacaatgccccacctgtgggcagtttttagagtacgaaaaaacgttagcaaaatttcaacaatccttactaagtctacaggttcagctagtggctgcagagaaccgtatcacacacttagagaaatgtgattgtaaaaaatcatgcctaattgacggtacaagcaaagacgacggtgatatatgggatataggttgtagtcagtgtaaatgcgagagtggcgtcgtcacgtgtggtccaagaccatgtccagaggttaagtgtaaacatccagtattagagaagggtgcatgttgtcctaagtgtttaaaaaattgttatattaataagaaagactatgaacacggcgataagcagatcctaggctgtagaaactgctcgtgcatagacggcaatatgttgtgtgatatgctgcagtgtccggagctcaagtgccccccggagcagcaactgtcggtgacggacgagtgctgtaaattttgtcaaggagtggactattgctcaaagggtcacgcatgtcatactaacgctacgtgtcttaatcttaacaccaagtacacgtgtacctgtcgctcgggcttccacggagatgggtatgaatgctctgacattgacgagtgcgctcagcaaggtggtcttaatggtaatcactgccatctgaacacgcggtgcgtcaacacgttcggctcgtacgtgtgcgagtgcgtggccggctaccggcggctggacaagttcaactgcgtcgaggtggacgagtgcaagtccggcgagcacagctgccacgagcacgcggactgcagcaacacggccggctcgtaccactgtcgctgccagccgggctacgagggcgacgggtacgactgcaagccggtctgcaatcaaacctgtctgaacgggggtgagtgccgggcgcctggggtgtgtacgtgccgggccggctacgtcggcgagtcctgcgagaaggacctggacgagtgtgcgaccggggtgcaccggtgtaaggagacaacgaactgtgttaacatgccaggatggtactattgcaaatgcaagcctggatttgaaaccaaaggaaaagattgtgtagatatagacgagtgctatctgaacacgcacagctgtcacccgacggcccggtgcgtcaacacgcagggacactttgagttggtggcgacggttgcggcaaccgtccggtccggtgccgcgtccaactgtaggttaagttgtatgttcgaggacagcgagatcccggacggcggccagatctcgccccggaaccagccgtgcaaggtgtgcacctgttcgcggggggtcatcagctgcgtggagccaccgtgcaactgctcgcggtggcaggagcggggcagcggagcgcgggacctctgctgtccgcagtgcgatcccaaagagtcctgccagcaccaggagctgaagcacgtgacgttccgcagcggcgagcagtggatttaccagtgccagacgtgcgagtgtctgtacggtgaattcgactgctggaggctcgagtgtccgccgctgacttgtgataacccgctgccgatggggcggggcgactgctgcccgcggtgtccggacgacatgtgcggcctggagaacgcaacggcgaccatcggtagcggcggcagtggcgcgagcttgccgtgcctgtacgagcagcacatctacgcctctggacagacgttcaagtacccgtccagagagtgcgccacctgctcctgcaaggtgagttga

SEQ ID NO: 29 - protein, 734 aa, Culex quinquefasciatus
MKRTADFTFAVVLKQEQANSGTIVSFSNGNNRYLELQSSGRRDEIRFHYTHITAAGEPLMRYESFPYRLADDAEHKVALTVSGTEVQLYIDCHPLYKRVMHFLPDRNFSASNMQLFVGQRNSNSHYLFKGDLKDLRIITGPYGYLSQCELMDAQCPTCGQFLEYEKTLAKFQQSLLSLQVQLVAAENRITHLEKCDCKKSCLIDGTSKDDGDIWDIGCSQCKCESGVVTCGPRPCPEVKCKHPVLEKGACCPKCLKNCYINKKDYEHGDKQILGCRNCSCIDGNMLCDMLQCPELKCPPEQQLSVTDECCKFCQGVDYCSKGHACHTNATCLNLNTKYTCTCRSGFHGDGYECSDIDECAQQGGLNGNHCHLNTRCVNTFGSYVCECVAGYRRLDKFNCVEVDECKSGEHSCHEHADCSNTAGSYHCRCQPGYEGDGYDCKPVCNQTCLNGGECRAPGVCTCRAGYVGESCEKDLDECATGVHRCKETTNCVNMPGWYYCKCKPGFETKGKDCVDIDECYLNTHSCHPTARCVNTQGHFELVATVAATVRSGAASNCRLSCMFEDSEIPDGGQISPRNQPCKVCTCSRGVISCVEPPCNCSRWQERGSGARDLCCPQCDPKESCQHQELKHVTFRSGEQWIYQCQTCECLYGEFDCWRLECPPLTCDNPLPMGRGDCCPRCPDDMCGLENATATIGSGGSGASLPCLYEQHIYASGQTFKYPSRECATCSCKVS

SEQ ID NO: 30 - DNA, 2415 bp, Pediculus humanus corporis
  CDS (1)...(2415): Coding region
atgacacgatcaaattgttctggaggagtagattcaggaatagatctcttagaatccttacagcttcacaatacaagtcggcaaggagtgtctgctattccaggaccgcaaaaattacggcctgccttttatttacaaggagactacagagatctgaggctacctcaatcagtatttcaaaaagctgtacagcttttaagacataaccctgaatttacgcttagcgcatctcttaggcaagaagtagctaattctggaagtataatttcattttcttatggttttaataggtatttggaactacaaagtagcggccggaaagatgaaatccggttgcactacacatcgcaattagacgatgtggttcacgtggaggccttcccatacagactggctgacaacagctggcacaagctggcggtcaccgtgagcggatcccaagtagaattactagtggattgtcatcctttgtacagaagactgttgcagagaattccagatactaattttactttgcctcagctcacattatgggtcggccaacgcaacaatcgacactctttgtttaagggtgttttacaagatgtaagactgatatccgggccccatgggtaccttaatcagtgtccacaattggatgcaggttgtcctacttgtggacagtttatgactttgcaacacactgtcgaacaactcactaaaagtttgcatctattaacagacatgttactggttgcagaaggtcgtattacaaaattagaagaatgcgattgtcgaaaatcatgtaccgtaaatggtacagaatatcaagatagttcttcttggcagataggctgtgacatatgtaggtgtaagaagggagaagtaaaatgccaacccgttcaatgtcctaaaacgacgtgtaaacatccagtgataaaacctaaagaatgctgccctacttgtttaaaacaatgttatcacatgaagactttatacgatcatggagaaagaactagtccgaaggcatgtttcgaatgcaagtgtgaagatggatccatggagtgcaagcaaaaagacccaaatttaatttgtcctccccttccgtgtccttctagtcaacaatttagtgttcctggtgaatgctgtaaattttgcccaggtgtagactactgtggtaagggtcatgtgtgtcatgttaatgcaacctgtattaatttaacaactacttatgcttgtcattgcaataatggctatcatggaaatggccacgagtgtcttgatgtagatgaatgcaaagtaaaaggggggctggaagggaatcattgtcacatgaatactcggtgtgtaaatactcccggctcatatacatgcgaatgcctaccgggttatcgaagagtagataaatttaactgtgccgaactcaacgaatgcgtaacgaatcagcacaaatgtgatcctcatgcagactgcataaataccttaggaagttatcattgcgtatgtcaaaatggttattccggagatggatattcgtgcagtcccatttgtgagtcaccttgcttgaatggaggtaaatgcgtgagtccgggtgtttgttcgtgtagaaaaggttaccaaggtgcaaattgcgaacaggatttagacgaatgtgccagtaacttacatgggtgtcatcatacttccatttgcgtcaatatgccaggatggtattactgtaggtgtaaaccgggttacagaagtatacttcacgagggcaccctgggtacaacttgtcaagatttagatgaatgtaaagaaggtttgcattcgtgtcatccgactgctaaatgtgtaaatattgaaggaggatttgaatgcacgtgtcctagaattgatgatacgatagaagaaacttgttcgttaaattgtatttttgagggatccgaaattgccaataatgccatatttatacccactggtcagccttgtcgtacgtgtaaatgtacgaatggtgtcattacttgtcgtgacactaactgtgattgctcaaaacgaggatcctccagagatagatgttgtccgcagtgtgatcccagagcggcttgcacacatcaagaacttcgacacgtggtattcagcagcggtgaaagatggatatatcaatgtcaaacctgcgaatgtttgtttggcgaaatagattgttggtcattggaatgtcctcccgtttcatgttcttctcccgtactcactccaggagattgttgtccaagatgcgaagatgatccttgcggtttaagtacttttgataacacttccaccagtctcgttacgacaactcgggatgaaccttgtttcgtttccggtagactttatcaaagcggaagttcttggggagatccggaaaatagatgcacgacatgtagctgcaagaatttaatcgtcgataacgtttaa

SEQ ID NO: 31 - protein, 804 aa, Pediculus humanus corporis
MTRSNCSGGVDSGIDLLESLQLHNTSRQGVSAIPGPQKLRPAFYLQGDYRDLRLPQSVFQKAVQLLRHNPEFTLSASLRQEVANSGSIISFSYGFNRYLELQSSGRKDEIRLHYTSQLDDVVHVEAFPYRLADNSWHKLAVTVSGSQVELLVDCHPLYRRLLQRIPDTNFTLPQLTLWVGQRNNRHSLFKGVLQDVRLISGPHGYLNQCPQLDAGCPTCGQFMTLQHTVEQLTKSLHLLTDMLLVAEGRITKLEECDCRKSCTVNGTEYQDSSSWQIGCDICRCKKGEVKCQPVQCPKTTCKHPVIKPKECCPTCLKQCYHMKTLYDHGERTSPKACFECKCEDGSMECKQKDPNLICPPLPCPSSQQFSVPGECCKFCPGVDYCGKGHVCHVNATCINLTTTYACHCNNGYHGNGHECLDVDECKVKGGLEGNHCHMNTRCVNTPGSYTCECLPGYRRVDKFNCAELNECVTNQHKCDPHADCINTLGSYHCVCQNGYSGDGYSCSPICESPCLNGGKCVSPGVCSCRKGYQGANCEQDLDECASNLHGCHHTSICVNMPGWYYCRCKPGYRSILHEGTLGTTCQDLDECKEGLHSCHPTAKCVNIEGGFECTCPRIDDTIEETCSLNCIFEGSEIANNAIFIPTGQPCRTCKCTNGVITCRDTNCDCSKRGSSRDRCCPQCDPRAACTHQELRHVVFSSGERWIYQCQTCECLFGEIDCWSLECPPVSCSSPVLTPGDCCPRCEDDPCGLSTFDNTSTSLVTTTRDEPCFVSGRLYQSGSSWGDPENRCTTCSCKNLIVDNV

SEQ ID NO: 32 - DNA, 2220 bp, Aedes aegypti
  CDS (1)...(2220): Coding region
cgaaatcttgcccttccgacgtcggtttttcaccgggccgtggaccagatgaagcggacgtcagattttacgtttgccgtcgtcctgaagcaggagcaagctaactctgggaccatcgtttccttttcaaacggaaacaataggtatttagaattacaatcaagtggaagacgtgacgaaatacgttttcattacacacacataacagcagcgggcgcgacgcagattcacaccgagtcctttccgtatcgactcgcagacgacgccgagcacaaggtggcgctaacggtcagcggcaccgaggtgcagctgtacatcgactgccatccactgtataaacgagtgacacattttttaccagatagaaattttagtgcttccaatatgcaattattcgtcggacaaagaaactcaaatagtcattatttatttaagggtgatttaaaagatttacgtattatcaccggaccgtacggttatttatcacaatgtgaactaatggatgcacaatgccccacatgtgggcagttcttagagttagagaatgctttactaggattaaaagaaatgattacgcatttatcaagaaggctagtggctgcagagcaacgcgtttcgagtatagaggaatgtgattgtaaaaaatcatgcctaattaatggtacaagtaaaaacgatggtgatatatgggatataggttgcagtcagtgtaaatgtgagcgtggtgaagtcacgtgtggtccaagaccatgtccagaggttaagtgtaaacatccagtattagaggagggtgaatgttgtcctaagtgtttaaaaaattgttatattaataagaaagactatgaacacggcgagaagcagatcctaggttgtagaaactgctcgtgcataaacggcaatatgatgtgtgatatgctgcagtgtccggagctcaagtgccccccggagcagcaaatgtcggtgacggacgagtgctgtaaattttgtcaaggagtggactattgctcaaagggtcacgcatgtcatactaacgctacgtgtcttaatcttaacaccaagtacacgtgtacctgtcgctcgggcttccacggagatgggtatgattgctctgacatcgacgagtgcgctcagcaaggtggtcttaatggtaatcactgccatttgaacacgcgctgcgtcaacacgttcggttcgtacgtgtgcgagtgtctgcccggctaccggcagctggacaagttcaactgcatcgaggtggacgagtgcaagtccggggagcacagctgtcaccagcatgcggactgcatcaacacggccggctcgtatcactgccgctgcaaggccggctacgaaggcgacgggtacgactgtaaaccggtgtgcaatcaaacttgtctgaatgggggcgaatgccggacgcccggtgtgtgtacgtgccgagccggctatgtgggtgaatcctgcgagaaggatctggatgagtgtgcgaccggggtgcaccggtgtaaggagacaacgaactgtgttaacatgccaggatggtatttctgcaaatgcaagcctggatttgaaaccaaaggaaaagattgtgtagatatagatgagtgctatctgaacacgcacagctgtcacgcgacggcccggtgcgtgaacacgcagggacatttcgagtgtgttatcggtgcatcccgcgactgtaggttaagttgtgtgttcgaggacagtgagattccggacggtgggcagatctcgccccggaatcaaccctgtaaggtgtgcacctgctccaagggagtcatcagctgcgtggaaccaccgtgcaactgttcgacctggcggaggggcagcggacgtgacctgtgctgtccgcagtgcgatcccaaagagtcctgtcagcaccaggagctgaagcatgtgacgtttcgcagcggagagcagtggatttaccagtgtcagacgtgcgaatgtctgtacggtgaattcgactgctggaagctggagtgtccaccgctgacttgtgataaccctctcccgcttgggccgggtgactgctgtccgcggtgtccggatgacatgtgcgggctcgacaatactacggccgccaacggaataaccggaggctccgggagctcgtgcctgtacgaccagcacatctacgcctctggacagtcattcaagtactcgtccagagagtgtgccacctgttcctgcaaggtgagttga

SEQ ID NO: 33 - protein, 739 aa, Aedes aegypti
RNLALPTSVFHRAVDQMKRTSDFTFAVVLKQEQANSGTIVSFSNGNNRYLELQSSGRRDEIRFHYTHITAAGATQIHTESFPYRLADDAEHKVALTVSGTEVQLYIDCHPLYKRVTHFLPDRNFSASNMQLFVGQRNSNSHYLFKGDLKDLRIITGPYGYLSQCELMDAQCPTCGQFLELENALLGLKEMITHLSRRLVAAEQRVSSIEECDCKKSCLINGTSKNDGDIWDIGCSQCKCERGEVTCGPRPCPEVKCKHPVLEEGECCPKCLKNCYINKKDYEHGEKQILGCRNCSCINGNMMCDMLQCPELKCPPEQQMSVTDECCKFCQGVDYCSKGHACHTNATCLNLNTKYTCTCRSGFHGDGYDCSDIDECAQQGGLNGNHCHLNTRCVNTFGSYVCECLPGYRQLDKFNCIEVDECKSGEHSCHQHADCINTAGSYHCRCKAGYEGDGYDCKPVCNQTCLNGGECRTPGVCTCRAGYVGESCEKDLDECATGVHRCKETTNCVNMPGWYFCKCKPGFETKGKDCVDIDECYLNTHSCHATARCVNTQGHFECVIGASRDCRLSCVFEDSEIPDGGQISPRNQPCKVCTCSKGVISCVEPPCNCSTWRRGSGRDLCCPQCDPKESCQHQELKHVTFRSGEQWIYQCQTCECLYGEFDCWKLECPPLTCDNPLPLGPGDCCPRCPDDMCGLDNTTAANGITGGSGSSCLYDQHIYASGQSFKYSSRECATCSCKVS

SEQ ID NO: 34 - DNA, 2085 bp, Ixodes scapularis
  CDS (1)...(2085): Coding region
aggttttttgaacttcaaagtagcggccgcaaggacgagatccggctgcactacagccacgcgaacggcgagctggtggagacgttcccctaccggttggcggacaacacgtggcacaggctggcggtggcactcagcggtgacagtgtcgaggtgttcgtggactgcaaccgcatctattctcgggtgataaagaagctagacctgaacactgcaaacaggaacctgtccctctggttgggacagaggaacctgaagcactactattttaacggtatattacaagatgtcaaagttgtgttccgacctcacgggtttcttctccagtgtcctcaattagatacagagtgcccgacctgtggccagtttcaggagctccagcagtctgtgaatcaactaaagacgtacattataaatttgacgcagaggttaattgaggctgaagagaggatagcttcggttgaacaatgcgaatgccaaaagagctgccgagtcaacggtacggtccggcccgatggctctatttggaagcaggactgtgatatttgcacctgtgctcaaggaaaagtggagtgccggccaatcagctgccccacgccgccctgcaagcaccccgttctccagccaggagaatgctgccccagctgcctcaagcgctgttttctgaaacaggttctctatgaccacggagagcaagtgacacaaaaacttcacatgattctccaaatcttgcaggacggacaaatgacttgccgtaggattgaccagaagacatgcccagctttgacatgccccgaatctcaaaggttcagcgtgccaggggaatgctgcatgttttgtccaggcgttgactactgtggtcggggccacgattgtgacgtgaatgcaacctgcatcaacctccagaccaagtatgcctgccagtgcaacactggctacaaggggaatggcaggtcctgtgaggacgtggacgaatgcctgaaagagggaggatttgacggccaccactgccaagagagtactcgctgcgtgaatacgcctggtgcctacacctgcgaatgcctgccaggatttcgcagggtcgatgcctacaactgcgaagaacatgacgagtgcgctagcaatgaacacaactgccataaggatgcaaggtgcatcaacacaatgggaagctacaagtgtgaatgtctcccaggctaccgtggcgacggtatcacctgtgaacccatatgcaatcagacctgcctaaatggtggaaaatgcgctgcccccaacgtttgcaattgccgtcgcggttacaagggaagcagctgtgaaattgacgtaaatgaatgctcggagaacgttcaccagtgtcacaaaaactccaactgcatcaacatgcccggctggtactactgcgagagtgacgcaatggtaagtttcagctgtgctcactttttggttgcagacattgatgaatgcagtctgggaactcatacctgccacagcactgcgacctgcatcaacctggaaggcagctttcagtgcatctgtgcagacaacgctacctgttcattcaactgtgatcacctggggatggagcggatcaacggcgaggtgtggcccatcggaccggggtcttgcacgacgtgcgcctgtcgaaacggcgtcgtggactgccgcaaggcgcgctgcgactgctccgacccacggacggacctggagtgctgtccgcagtgcgaccggtccagcttctgcgagcatcaggaggcgcccatcaagttcgccaatggagagcggtggatctaccagtgccagatttgcgagtgtctgtttggcgaggtggactgctggagcatggagtgcccccacgtggcatgcagcaaccctgtgcagcgagccggcgactgctgcccgtactgcgaggacgacccctgcgacactggggccaatgggacgaggtcgccccggggctgcacctacatggggcggctgtaccagaccggggagagggtccccctcgcgcaggacccttgcaccagctgcaagtgctcggacgggcagctctgctgcatgtactctacaaattgccttggcaatgct

SEQ ID NO: 35 - protein, 695 aa, Ixodes scapularis
RFFELQSSGRKDEIRLHYSHANGELVETFPYRLADNTWHRLAVALSGDSVEVFVDCNRIYSRVIKKLDLNTANRNLSLWLGQRNLKHYYFNGILQDVKVVFRPHGFLLQCPQLDTECPTCGQFQELQQSVNQLKTYIINLTQRLIEAEERIASVEQCECQKSCRVNGTVRPDGSIWKQDCDICTCAQGKVECRPISCPTPPCKHPVLQPGECCPSCLKRCFLKQVLYDHGEQVTQKLHMILQILQDGQMTCRRIDQKTCPALTCPESQRFSVPGECCMFCPGVDYCGRGHDCDVNATCINLQTKYACQCNTGYKGNGRSCEDVDECLKEGGFDGHHCQESTRCVNTPGAYTCECLPGFRRVDAYNCEEHDECASNEHNCHKDARCINTMGSYKCECLPGYRGDGITCEPICNQTCLNGGKCAAPNVCNCRRGYKGSSCEIDVNECSENVHQCHKNSNCINMPGWYYCESDAMVSFSCAHFLVADIDECSLGTHTCHSTATCINLEGSFQCICADNATCSFNCDHLGMERINGEVWPIGPGSCTTCACRNGVVDCRKARCDCSDPRTDLECCPQCDRSSFCEHQEAPIKFANGERWIYQCQICECLFGEVDCWSMECPHVACSNPVQRAGDCCPYCEDDPCDTGANGTRSPRGCTYMGRLYQTGERVPLAQDPCTSCKCSDGQLCCMYSTNCLGNA

SEQ ID NO: 36 - DNA, 691 bp, Strongylocentrotus purpuratus
  CDS (1)...(691): Coding region
tcgggtcatgtgagttgtaacccagtagcttgtgcacccgtctactgtaagaatcctatctacccacctggcaactgctgcccaatttgtaaagctaactgcacagtatctggagtgacgtatcaccatggcgacgtgttccaacagaagacttctagaggtgctctacagatttgcagcgtaaaagaatgcaataatggtggaacagtgacccagagacaggacagtgtgagtaacatgtgtcctacgccctcctgcgctgtatcagagaggttcaccgtaccgggtagctgctgtgaatactgtcaaggcactgactactgtgcccagggtcacagctgtgtgtctcccaaggtatgtgtgaacacgcctacatccagcgaatgcctgtgtccgacgggttacctggaggctaatgggaactgcaccgatatcaacgaatgcgtggtgagtgagacgcagcctgatccacactgctacgaaggtactctgtgtgtgaacacaccgggaggttaccagtgtggatgcaagccaggatacgagacggtcacagacacacaatgcagacagattgatgaatgcactgctggaacagacaactgtgatgagaatgctgactgcacagatactgatggtagcttcacctgtacatgccagcatagatatcatggagatgggacatactgcttcc

SEQ ID NO: 37 - protein, 230 aa, Strongylocentrotus purpuratus
SGHVSCNPVACAPVYCKNPIYPPGNCCPICKANCTVSGVTYHHGDVFQQKTSRGALQICSVKECNNGGTVTQRQDSVSNMCPTPSCAVSERFTVPGSCCEYCQGTDYCAQGHSCVSPKVCVNTPTSSECLCPTGYLEANGNCTDINECVVSETQPDPHCYEGTLCVNTPGGYQCGCKPGYETVTDTQCRQIDECTAGTDNCDENADCTDTDGSFTCTCQHRYHGDGTYCF

SEQ ID NO: 38 - DNA, 5330 bp, Acyrthosiphon pisum
  CDS (1)...(5330): Coding region
atgtttaagtacaaccagggcagagatcgattcgtgcccgatcgacatcggagcccgtattcgttttattctgtacaccacaccgccgaaaatcaccagtacgattgtgtctccaacaacagcttcgatgaacccggaagtaccgatttcctggagccgttcgaatgcgaacggcagcaagagcagcaacacagatctttccgccaacttcagtcgtacatgtcactggatttcattggcggacacctgttgcgggacgtattgaacaagcgcaaacatggtacagacgaggcagacctaatgaccatataccgtgacgccaacatgacgtggcccatcagaatggtcagccagagacgtttggcccggaagttcaatcatgccaccagcaaatcactggtagcagatctgctgttggatatgccgaatctgtccaacgactggatgaccaagatactggattggagttcgacgggctacttgacggcggccattcaatcgtcaatacacttgtggtcgagccagacacagagcgtccagtacacgatcgccgcggtggacgcgcacgacccgacgcagaccgaaggtggcgcgacgaagaccactatcggttgtctgaaatgggacgtgcagggcgaaaaactgggatattcgttcactgtgggtaggggtgaagactcgtcattcgattcatcgggtgattcatcgggtcccggcgactcatcgctcttagaacgaattttgttgggtgacggaggtctcggcgactcgtcgctcttagaacgaatgaacgcgtacgcggcacgtgatcagcgactcttatcagagcaggacgacccggacgtgacgtatagatctccggccataaatgcgtctgcagacgatacttaccagcagataccgcggcgaatcaataacgcgatcaagaacactggacacgtaaaggtttggttattgaatgcaccgaatgacaaaacaaataatgaacaaaaaacatcgagttgtggttgtgtaactgatgacacttgtcaatggatttacggatgcagaattattgtcatggattggttggccaacggtcatgttttagtgacgggttgcacgagggccacagtgacatttttccggtacgatttcgcccaaaagaaattgatcactacgcgcattatacgcaggatagaaaaatattcggtaattagctgcctgcaagtgtcacaggacaaatggagtcgtcacttggccgtggccactcattcgcaacagtcatgcgtgttgaaagtttggcgcatcgaagacccgctggagttgtgcccacactacgatcagcgcttagattcgagtatcaacgtgaccgaaaccttgaccgaccgttacgacagtaaacagaatggccgcgctaaatggctgataaaggccttcgcttggcacccgtggaaacacgctctgatgtgttacgctgtcacagcggtcccacaggacggtgcccagatgtacatcaatcgcaagtgtccgtcgtggtttgtgttggcgaacgcttgcaaaggacaagtgttgaagcgcgtcgaacagaccgcaagcaactgctgtccgacggtggacataatgcacgtcaacacgcactcgatgacgttcagccgactgaccggcgaacttctgttgtcggccacgaccgtgtacaatttaccaatttacaaagaaactcttgtgtctattagaaattcaattggtgatggacccatatgggcatcaatcgatgaaacaaccgatgtagatggtagatttattgcaaatattattattggaaaattaaatgacagtaaatcccagccttttttgttaaactgtgaacaattagacaaatgcaaccaccaaactgttgcaagattttttaataattctatgagtatattatggcccgatatagttatgcacgagaatgtattattatttgtatctgatgccgcgccatacatgataaaagccggtaaagcattaaatgcttttttcccaaaaatgatccacttaacgtgtttagcacacgcatttcaccgaatagcagaaactataagattacaatttacaaaagttgacgagttaatatcttcagtcaaaaagatatttctaaaagctcctagccgtgtcgaaatatttaaaaatatgtacccagatctaagccttcctccacaaccaattgtaactagatggggcacttggttaaatgccgctaattattattgccaaaacttccaagaaatcaaaaatgttgttgccgaactcgatgcgactacttcaattggtagtaataaaagtgatactactggttttacattgggtgtcttactcgtggctgtaacagaagatgaattttctcatcccgatgtttttcctcccaaccggttaagaccagcatacagcatgcaaggtgcgttgagacaattaaaagttcctgacattatttacaaacgtgcaattcaattattacgaggtgcggacgagttaactatagcggcagtagtacggcaagaacgtgctaacactggatctatagtatcattttccctcgactccaacagatacttggaattagaatcaagtggacgtaaagacgaggtgaggttgcactatgtgtcacagagtagccgtcgtgttcataccgaagtgtttgcgtactccctggctgatggccattggcatgctgtggccgtaacactgagcggtacccaagcacgcctgttcatagactgccgtctagtatacacgcgaactttggtcgctggaccggctgacactagttttctaggatcgccggatctgtacgtaggccaacgaaacagaggagtacattcaattttcaaggggcttttacaagacgtccggttgatcagaggggcagacggacaacgtgtgcagtgccctaatctggacacgtcatgtccaacgtgcggtcaattcatcgccatgcaaaaaacgatggaaaacctgaacaaaattgtgcatctgttgagtatcaagctcgaggcagccgaaatgagaatcgccgaattagaaacttgtgaatgtcgcaaaccatgtgtacagtttgatgaacgaagtaataacacagtaatgaaagtagatggatcagtgtggcaacatgaatgccaaacgtgtacatgtttgcgcggtcaagttgaatgttcaccggttagatgtcccctagtcgattgcaagtttccaatacgaaaacctggtgaatgttgcgacacttgtcaaaaaccatgccgagccaaaggtgatattgtattcgaacacggtgacacaataaatttggaaccatgcttaacctgcatttgtcttaatggagttgttaagtgtgacaaagtagatgcacaaactcaatgtccagtattagaatgtgacatatctgaacagttttccgtaccagatcagtgttgtaaattttgtccaggcgtcgattattgtgcacgaggtggacgtgactcgtgtcatccgaacgctacttgtatcaatctaaaaaccacacacaagtgtgcgtgcgctgttgggtacgctggtaccggtttacagtgtgatgatgtggacgaatgcttgagcgtcggcggaccaaatggacaccactgcgacgtgaacacgactatttgcgtgaacacgcctggatcgtactcgtgcaagtgcaacgcagggtacgcgcgtctcaacaggttcgaatgtgtggacgtcgacgagtgcagcagcgcggaaccaccgtgtcacgtacacgccgcttgtgccaactcgccgggcagttacagctgcaagtgcgccgacggctacgaaggggacggatatacttgcagacctgtgtgccagcaaaagtgtttgaacggaggcgtgtgcgcacggccagatgtgtgctcgtgcccgaacgcgttcgaggggcctgcttgcgagctggacgtggatgagtgtaccacaggggcgcacggatgtctaggcccctcgtcccggtgcgtcaatatggctggatggtattactgtgcgtgtagcccggggtaccaaaacgtcatcatgtatccggcttcggccgacatcgccaccacagctactagtgacttgaacgcaatggaatcgacctggtcgccttcgttgggcacaatgtgtcaagaatgtttagatatcgacgaatgcgcgactaataaccatacgtgtcatccaacggctacgtgtgtgaacaccgtgggatcgtatgaatgcaaatgcccaacgacccatgccaatgccggtaacatctcttcgaagtgttgtttgtataaggacggcagtaacgtagtgacggaagtgaatgacggcgcctggttctcaaaagattgcaccaactgcacgtgcacaagtgggcgtttacgttgctcacgaattgaatgcgattgcacaaacaatgaccatgtacaatcgatttgttgcgctcattgtaacgcacaagcgtcatgcgttcatcaggaattgacgaacgtgatgttccgcagtggccaacaatggatataccagtgtcagacatgcgagtgtttaatgggccaagtggattgctggccaatgcagtgtccaccgatgctgcccgcgggcgtgggcagttgtagcgactcgacggcagccacgtttgctgaatataacaacccagtggccgattgttgcgcaccgagatgcgattccaaagccgtcgcgtcgggttcatcggcagcggcgaccacgacagcgatgaccgctgctgatccgtgtctacagcagcagctacagctacagccacctgatgattaccggcaccgcggcggtgaagtaaacgacgaagacgacgacgacgatgacgacgacggcgacagcaaccacagcaaaaacaccgacgacgacaacaggaataacaccacgccgtcgccccggggccaaccgtgtttttacggcggacgcgcctatcagtcgggagcccgttgggacgatccggtggatatgtgcacgtcgtgcgattgcaaggacggagtactatgttgttcttacaacgtgcaccactgcaataatagtgctattgccggtgcatcaaatcaatcatcaactacagacgtcaactataatgtgtctactgatcagcctcatcacatttctaacgaaaataccagtagtcaatctaccgatacgaaattacagccagatgttgttaaacggagtcttcgacgtcgaagacaagcaaaaatggttaacaatgatgacactttaacaaaaaagactggtggtggaggaaaacaagacggcgaaaaagaagaaggactcaaaatacgagtagccgtcggaaccaaattaatagt

SEQ ID NO: 39 - protein, 1776 aa, Acyrthosiphon pisum
MFKYNQGRDRFVPDRHRSPYSFYSVHHTAENHQYDCVSNNSFDEPGSTDFLEPFECERQQEQQHRSFRQLQSYMSLDFIGGHLLRDVLNKRKHGTDEADLMTIYRDANMTWPIRMVSQRRLARKFNHATSKSLVADLLLDMPNLSNDWMTKILDWSSTGYLTAAIQSSIHLWSSQTQSVQYTIAAVDAHDPTQTEGGATKTTIGCLKWDVQGEKLGYSFTVGRGEDSSFDSSGDSSGPGDSSLLERILLGDGGLGDSSLLERMNAYAARDQRLLSEQDDPDVTYRSPAINASADDTYQQIPRRINNAIKNTGHVKVWLLNAPNDKTNNEQKTSSCGCVTDDTCQWIYGCRIIVMDWLANGHVLVTGCTRATVTFFRYDFAQKKLITTRIIRRIEKYSVISCLQVSQDKWSRHLAVATHSQQSCVLKVWRIEDPLELCPHYDQRLDSSINVTETLTDRYDSKQNGRAKWLIKAFAWHPWKHALMCYAVTAVPQDGAQMYINRKCPSWFVLANACKGQVLKRVEQTASNCCPTVDIMHVNTHSMTFSRLTGELLLSATTVYNLPIYKETLVSIRNSIGDGPIWASIDETTDVDGRFIANIIIGKLNDSKSQPFLLNCEQLDKCNHQTVARFFNNSMSILWPDIVMHENVLLFVSDAAPYMIKAGKALNAFFPKMIHLTCLAHAFHRIAETIRLQFTKVDELISSVKKIFLKAPSRVEIFKNMYPDLSLPPQPIVTRWGTWLNAANYYCQNFQEIKNVVAELDATTSIGSNKSDTTGFTLGVLLVAVTEDEFSHPDVFPPNRLRPAYSMQGALRQLKVPDIIYKRAIQLLRGADELTIAAVVRQERANTGSIVSFSLDSNRYLELESSGRKDEVRLHYVSQSSRRVHTEVFAYSLADGHWHAVAVTLSGTQARLFIDCRLVYTRTLVAGPADTSFLGSPDLYVGQRNRGVHSIFKGLLQDVRLIRGADGQRVQCPNLDTSCPTCGQFIAMQKTMENLNKIVHLLSIKLEAAEMRIAELETCECRKPCVQFDERSNNTVMKVDGSVWQHECQTCTCLRGQVECSPVRCPLVDCKFPIRKPGECCDTCQKPCRAKGDIVFEHGDTINLEPCLTCICLNGVVKCDKVDAQTQCPVLECDISEQFSVPDQCCKFCPGVDYCARGGRDSCHPNATCINLKTTHKCACAVGYAGTGLQCDDVDECLSVGGPNGHHCDVNTTICVNTPGSYSCKCNAGYARLNRFECVDVDECSSAEPPCHVHAACANSPGSYSCKCADGYEGDGYTCRPVCQQKCLNGGVCARPDVCSCPNAFEGPACELDVDECTTGAHGCLGPSSRCVNMAGWYYCACSPGYQNVIMYPASADIATTATSDLNAMESTWSPSLGTMCQECLDIDECATNNHTCHPTATCVNTVGSYECKCPTTHANAGNISSKCCLYKDGSNVVTEVNDGAWFSKDCTNCTCTSGRLRCSRIECDCTNNDHVQSICCAHCNAQASCVHQELTNVMFRSGQQWIYQCQTCECLMGQVDCWPMQCPPMLPAGVGSCSDSTAATFAEYNNPVADCCAPRCDSKAVASGSSAAATTTAMTAADPCLQQQLQLQPPDDYRHRGGEVNDEDDDDDDDDGDSNHSKNTDDDNRNNTTPSPRGQPCFYGGRAYQSGARWDDPVDMCTSCDCKDGVLCCSYNVHHCNNSAIAGASNQSSTTDVNYNVSTDQPHHISNENTSSQSTDTKLQPDVVKRSLRRRRQAKMVNNDDTLTKKTGGGGKQDGEKEEGLKIRVAVGTKLI

SEQ ID NO: 40 - DNA, 1498 bp, Homo sapiens
  CDS (88)...(897): Coding region
accaaacctcttcgaggcacaaggcacaacaggctgctctgggattctcttcagccaatcttcattgctcaagtgtctgaagcagccatggcagaagtacctgagctcgccagtgaaatgatggcttattacagtggcaatgaggatgacttgttctttgaagctgatggccctaaacagatgaagtgctccttccaggacctggacctctgccctctggatggcggcatccagctacgaatctccgaccaccactacagcaagggcttcaggcaggccgcgtcagttgttgtggccatggacaagctgaggaagatgctggttccctgcccacagaccttccaggagaatgacctgagcaccttctttcccttcatctttgaagaagaacctatcttcttcgacacatgggataacgaggcttatgtgcacgatgcacctgtacgatcactgaactgcacgctccgggactcacagcaaaaaagcttggtgatgtctggtccatatgaactgaaagctctccacctccagggacaggatatggagcaacaagtggtgttctccatgtcctttgtacaaggagaagaaagtaatgacaaaatacctgtggccttgggcctcaaggaaaagaatctgtacctgtcctgcgtgttgaaagatgataagcccactctacagctggagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcctaaagagagctgtacccagagagtcctgtgctgaatgtggactcaatccctagggctggcagaaagggaacagaaaggtttttgagtacggctatagcctggactttcctgttgtctacaccaatgcccaactgcctgccttagggtagtgctaagaggatctcctgtccatcagccaggacagtcagctctctcctttcagggccaatccccagcccttttgttgagccaggcctctctcacctctcctactcacttaaagcccgcctgacagaaaccacggccacatttggttctaagaaaccctctgtcattcgctcccacattctgatgagcaaccgcttccctatttatttatttatttgtttgtttgttttattcattggtctaatttattcaaagggggcaagaagtagcagtgtctgtaaaagagcctagtttttaatagctatggaatcaattcaatttggactggtgtgctctctttaaatcaagtcctttaattaagactgaaaatatataagctcagattatttaaatgggaatatttataaatgagcaaatatcatactgttcaatggttctgaaataaacttcactgaag

SEQ ID NO: 41 - protein, 181 aa, Homo sapiens
MAGVGASGMMATTSGAGAAGAAGGMCSGAACAGGGASAHHTSGAGAASVVVAMAAMVCGTGGAASTGGGATTAAGATVHAAVASACTAASGGSVMSGTGAHGGGAMGGGVVSMSVGGGGSAAVAGGATSCVAATGGSVAATMGAVAGAAGGSAGATTSTSGAGAMVGGTGGGATATMGVSS

SEQ ID NO: 42 - DNA, 1666 bp, Homo sapiens
  CDS (102)...(401): Coding region
ctccataaggcacaaactttcagagacagcagagcacacaagcttctaggacaagagccaggaagaaaccaccggaaggaaccatctcactgtgtgtaaacatgacttccaagctggccgtggctctcttggcagccttcctgatttctgcagctctgtgtgaaggtgcagttttgccaaggagtgctaaagaacttagatgtcagtgcataaagacatactccaaacctttccaccccaaatttatcaaagaactgagagtgattgagagtggaccacactgcgccaacacagaaattattgtaaagctttctgatggaagagagctctgtctggaccccaaggaaaactgggtgcagagggttgtggagaagtttttgaagagggctgagaattcataaaaaaattcattctctgtggtatccaagaatcagtgaagatgccagtgaaacttcaagcaaatctacttcaacacttcatgtattgtgtgggtctgttgtagggttgccagatgcaatacaagattcctggttaaatttgaatttcagtaaacaatgaatagtttttcattgtaccatgaaatatccagaacatacttatatgtaaagtattatttatttgaatctacaaaaaacaacaaataatttttaaatataaggattttcctagatattgcacgggagaatatacaaatagcaaaattgaggccaagggccaagagaatatccgaactttaatttcaggaattgaatgggtttgctagaatgtgatatttgaagcatcacataaaaatgatgggacaataaattttgccataaagtcaaatttagctggaaatcctggatttttttctgttaaatctggcaaccctagtctgctagccaggatccacaagtccttgttccactgtgccttggtttctcctttatttctaagtggaaaaagtattagccaccatcttacctcacagtgatgttgtgaggacatgtggaagcactttaagttttttcatcataacataaattattttcaagtgtaacttattaacctatttattatttatgtatttatttaagcatcaaatatttgtgcaagaatttggaaaaatagaagatgaatcattgattgaatagttataaagatgttatagtaaatttattttattttagatattaaatgatgttttattagataaatttcaatcagggtttttagattaaacaaacaaacaattgggtacccagttaaattttcatttcagataaacaacaaataattttttagtataagtacattattgtttatctgaaattttaattgaactaacaatcctagtttgatactcccagtcttgtcattgccagctgtgttggtagtgctgtgttgaattacggaataatgagttagaactattaaaacagccaaaactccacagtcaatattagtaatttcttgctggttgaaacttgtttattatgtacaaatagattcttataatattatttaaatgactgcatttttaaatacaaggctttatatttttaactttaagatgtttttatgtgctctccaaattttttttactgtttctgattgtatggaaatataaaagtaaatatgaaacatttaaaatataatttgttgtcaaagtaaaaaaaaaaaaaaa

SEQ ID NO: 43 - protein, 99 aa, Homo sapiens
MTSKLAVALLAAFLISAALCEGAVLPRSAKELRCQCIKTYSKPFHPKFIKELRVIESGPHCANTEIIVKLSDGRELCLDPKENWVQRVVEKFLKRAENS

SEQ ID NO: 44 - DNA, 2081 bp, Homo sapiens
  CDS (144)...(1553): Coding region
agcatgagtcagacagcctctggctttctggaagggcaaggactctatatatacagagggagcttcctagctgggatattggagcagcaagaggctgggaagccatcacttaccttgcactgagaaagaagacaaaggccagtatgcacagctttcctccactgctgctgctgctgttctggggtgtggtgtctcacagcttcccagcgactctagaaacacaagagcaagatgtggacttagtccagaaatacctggaaaaatactacaacctgaagaatgatgggaggcaagttgaaaagcggagaaatagtggcccagtggttgaaaaattgaagcaaatgcaggaattctttgggctgaaagtgactgggaaaccagatgctgaaaccctgaaggtgatgaagcagcccagatgtggagtgcctgatgtggctcagtttgtcctcactgaggggaaccctcgctgggagcaaacacatctgacctacaggattgaaaattacacgccagatttgccaagagcagatgtggaccatgccattgagaaagccttccaactctggagtaatgtcacacctctgacattcaccaaggtctctgagggtcaagcagacatcatgatatcttttgtcaggggagatcatcgggacaactctccttttgatggacctggaggaaatcttgctcatgcttttcaaccaggcccaggtattggaggggatgctcattttgatgaagatgaaaggtggaccaacaatttcagagagtacaacttacatcgtgttgcagctcatgaactcggccattctcttggactctcccattctactgatatcggggctttgatgtaccctagctacaccttcagtggtgatgttcagctagctcaggatgacattgatggcatccaagccatatatggacgttcccaaaatcctgtccagcccatcggcccacaaaccccaaaagcgtgtgacagtaagctaacctttgatgctataactacgattcggggagaagtgatgttctttaaagacagattctacatgcgcacaaatcccttctacccggaagttgagctcaatttcatttctgttttctggccacaactgccaaatgggcttgaagctgcttacgaatttgccgacagagatgaagtccggtttttcaaagggaataagtactgggctgttcagggacagaatgtgctacacggataccccaaggacatctacagctcctttggcttccctagaactgtgaagcatatcgatgctgctctttctgaggaaaacactggaaaaacctacttctttgttgctaacaaatactggaggtatgatgaatataaacgatctatggatccaggttatcccaaaatgatagcacatgactttcctggaattggccacaaagttgatgcagttttcatgaaagatggatttttctatttctttcatggaacaagacaatacaaatttgatcctaaaacgaagagaattttgactctccagaaagctaatagctggttcaactgcaggaaaaattgaacattactaatttgaatggaaaacacatggtgtgagtccaaagaaggtgttttcctgaagaactgtctattttctcagtcatttttaacctctagagtcactgatacacagaatataatcttatttatacctcagtttgcatatttttttactatttagaatgtagccctttttgtactgatataatttagttccacaaatggtgggtacaaaaagtcaagtttgtggcttatggattcatataggccagagttgcaaagatcttttccagagtatgcaactctgacgttgatcccagagagcagcttcagtgacaaacatatcctttcaagacagaaagagacaggagacatgagtctttgccggaggaaaagcagctcaagaacacatgtgcagtcactggtgtcaccctggataggcaagggataactcttctaacacaaaataagtgttttatgtttggaataaagtcaaccttgtttctactgttttatacactttcaaaaaaaaaaaaaaaaaaaaaaaaaa

SEQ ID NO: 45 - protein, 469 aa, Homo sapiens
MHSFPPLLLLLFWGVVSHSFPATLETQEQDVDLVQKYLEKYYNLKNDGRQVEKRRNSGPVVEKLKQMQEFFGLKVTGKPDAETLKVMKQPRCGVPDVAQFVLTEGNPRWEQTHLTYRIENYTPDLPRADVDHAIEKAFQLWSNVTPLTFTKVSEGQADIMISFVRGDHRDNSPFDGPGGNLAHAFQPGPGIGGDAHFDEDERWTNNFREYNLHRVAAHELGHSLGLSHSTDIGALMYPSYTFSGDVQLAQDDIDGIQAIYGRSQNPVQPIGPQTPKACDSKLTFDAITTIRGEVMFFKDRFYMRTNPFYPEVELNFISVFWPQLPNGLEAAYEFADRDEVRFFKGNKYWAVQGQNVLHGYPKDIYSSFGFPRTVKHIDAALSEENTGKTYFFVANKYWRYDEYKRSMDPGYPKMIAHDFPGIGHKVDAVFMKDGFFYFFHGTRQYKFDPKTKRILTLQKANSWFNCRKN

SEQ ID NO: 46 - DNA, 1835 bp, Homo sapiens
  CDS (63)...(941): Coding region
ctcgctgcgccaccgcctcccgccacccctgcccgcccgacagcgccgccgcctgccccgccatgggtcgacagaaggagctggtgtcccgctgcggggagatgctccacatccgctaccggctgctccgacaggcgctggccgagtgcctggggaccctcatcctggtgatgtttggctgtggctccgtggcccaggttgtgctcagccggggcacccacggtggtttcctcaccatcaacctggcctttggctttgctgtcactctgggcatcctcatcgctggccaggtctctggggcccacctgaaccctgccgtgacctttgccatgtgcttcctggctcgtgagccctggatcaagctgcccatctacaccctggcacagacgctgggagccttcttgggtgctggaatagtttttgggctgtattatgatgcaatctggcacttcgccgacaaccagctttttgtttcgggccccaatggcacagccggcatctttgctacctacccctctggacacttggatatgatcaatggcttctttgaccagttcataggcacagcctcccttatcgtgtgtgtgctggccattgttgacccctacaacaaccccgtcccccgaggcctggaggccttcaccgtgggcctggtggtcctggtcattggcacctccatgggcttcaactccggctatgccgtcaaccctgcccgggactttggcccccgcctttttacagcccttgcgggctggggctctgcagtcttcacgaccggccagcattggtggtgggtgcccatcgtgtccccactcctgggctccattgcgggtgtcttcgtgtaccagctgatgatcggctgccacctggagcagcccccaccctccaacgaggaagagaatgtgaagctggcccatgtgaagcacaaggagcagatctgagtgggcaggggccatctccccactccgctgccctggccttgagcatccactgactgtccaagggccactcccaagaagcccccttcacgatccaccctttcaggctaaggagctccctatctaccctcaccccacgagacagccccttcaggatttccactggaccttgcccaaatagcaccttaggccactgcccctaagctggggtggaaccggaatttgggtcaatacatccttttgtctcccaagggaagagaatgggcagcaggtatgtgtgtgtgtgcatgtgtgtgcatgtgtgtgcatgtgtgtgcaggggtgtgtgtgtgtggggggggttcccagatattcagggcaagggaccagtcggaagggattctggctattgggggagcccagagacaggggaaggcagcctgtccatctgtgcataaggagaggaaagttccagggtgtgtatgtttcaggggcttcacatggaggagctgcagatagatatgtgtttctgtgtatgtgtatgtctgcctttttttctaagtgggggcttctacaggcttttgggaagtagggtggatgtgggtagggctgggaggagggggccacagcttaggtttggagctctggatgtacatacataagtaggagcagtgggacgtgtttctgtcataatgcaggcatgaagggtggagtgaagtcaggtcataagtttcatgtttgcttttgttttgttttgtttttaatgtatgtagcagatgttacagtcttagggatccgggatgggagaccccactttagaaagggtcgtcactcctttaatcctctactcaacaatgtactcttttacttttatattaaaaaaaataaaataaatatgtgcctaaaaaaaaaaaaaaa

SEQ ID NO: 47 - protein, 292 aa, Homo sapiens
MGRQKELVSRCGEMLHIRYRLLRQALAECLGTLILVMFGCGSVAQVVLSRGTHGGFLTINLAFGFAVTLGILIAGQVSGAHLNPAVTFAMCFLAREPWIKLPIYTLAQTLGAFLGAGIVFGLYYDAIWHFADNQLFVSGPNGTAGIFATYPSGHLDMINGFFDQFIGTASLIVCVLAIVDPYNNPVPRGLEAFTVGLVVLVIGTSMGFNSGYAVNPARDFGPRLFTALAGWGSAVFTTGQHWWWVPIVSPLLGSIAGVFVYQLMIGCHLEQPPPSNEEENVKLAHVKHKEQI

SEQ ID NO: 48 - DNA, 1800 bp, Bos taurus
  CDS (1)...(1800): Coding region
atggctctctgttccttttcagtggtgggctttgggctggatccggatcttcagttggatatcatcactgagctcgaccttgtgaacaccacccttggggtcacgcaggtgtccggactgcacaacaccagcaaagcatttttatttcaagatgcagaaagagagatccacgcagcccctcatgtgagtgagaaattaattcagctgttccggaataagagtgagttcaccttcctagccactgtgcagcagaagccatccacgtcaggagtgatcctgtccattcgagaactggaacacagctattttgaactggagagcagtggtctgagggatgagattcgctatcactacgtacacagtgggaggccccggacagaggcgcttccgtaccggctggctgacgggcagtggcacagggttgcactctcggtcagtgcctcccatctcctgctccacatcgactgcaacaggatttatgagcgtgtgatagaccctcctgagaccaaccttccccctggaagcaatttatggcttggtcagcgcaaccaaaagcatggtttattcaaagggattatccaagatgggaaaatcatcttcatgccaaatggatatataacacagtgtccaaacctgaatcgcacttgcccaacttgcagtgatttcttaagcctggtgcaaggaataatggatttacaagagcttttggccaagatgactgcaaaactgaactatgcagaaacaagacttagtcagttggaaaactgtcattgtgagaagacctgtcaagtgagtggactgctctatagagaccaagactcctgggtcgacggtgatcactgcaggaactgcacgtgcaaaagcggggctgtggaatgccgaaggatgtcctgtccccccctcaactgctctccagactccctccccgtgcacattgccggcgagtgctgcaaagtctgccgaccaaaatgtatctatggaggaaaagtccttgcagaaggccagcggattttaagcaagagttgtcaggaatgcagaggtggagttttagtaaaaattacggaagcatgccctcttttgaactgctcagaaaaggatcacattctcccggagaatcagtgctgcagtgtctgcagaggtcataacttttgtgctgaaggacctaaatgtggtgaaaactcagagtgcaaaaactggaataccaaagctacttgtgagtgcaagaatggttacatttctgtccagggagactctgcctactgtgaagatattgatgagtgtgcagctaagatgcattactgtcatgccaacactgtatgtgtcaacctgcccggattatatcgctgtgactgtgtcccagggtacattcgtgtggatgacttctcttgtacagagcacgacgactgcggcagcgggcaacacaattgtgatgagaatgccatctgcaccaacactgtccaggggcacagctgcacctgcaaaccaggctacgtgggcaacgggaccatctgcagaggcatgcctgaagtgggaccaccccgagcgttgcttaattctcttgatctgggttttctgtcattttctaaggaagcactggctgttggtatgataaccttggagggaaatattgttgccaagtcttttacagatgatgaaactttagttgaaagagggagagagaaagtaattgccttactcttttcttggctacacaaagagaaactctccttagaaaacctgcgggatatatattgtaaggcaaatagtcttgtcggcttggatcatctacctcaaagataa

SEQ ID NO: 49 - protein, 599 aa, Bos taurus
MALCSFSVVGFGLDPDLQLDIITELDLVNTTLGVTQVSGLHNTSKAFLFQDAEREIHAAPHVSEKLIQLFRNKSEFTFLATVQQKPSTSGVILSIRELEHSYFELESSGLRDEIRYHYVHSGRPRTEALPYRLADGQWHRVALSVSASHLLLHIDCNRIYERVIDPPETNLPPGSNLWLGQRNQKHGLFKGIIQDGKIIFMPNGYITQCPNLNRTCPTCSDFLSLVQGIMDLQELLAKMTAKLNYAETRLSQLENCHCEKTCQVSGLLYRDQDSWVDGDHCRNCTCKSGAVECRRMSCPPLNCSPDSLPVHIAGECCKVCRPKCIYGGKVLAEGQRILSKSCQECRGGVLVKITEACPLLNCSEKDHILPENQCCSVCRGHNFCAEGPKCGENSECKNWNTKATCECKNGYISVQGDSAYCEDIDECAAKMHYCHANTVCVNLPGLYRCDCVPGYIRVDDFSCTEHDDCGSGQHNCDENAICTNTVQGHSCTCKPGYVGNGTICRGMPEVGPPRALLNSLDLGFLSFSKEALAVGMITLEGNIVAKSFTDDETLVERGREKVIALLFSWLHKEKLSLENLRDIYCKANSLVGLDHLPQR

SEQ ID NO: 50 - DNA, 2791 bp, Equus caballus
  CDS (1)...(2376): Coding region
atgggctttgggatggaccccgaccttcaaatggatattatcaccgagctcgacctcgtgaacaccacccttggagtcactcaggtgtccggactgcacaatgccagcaaagcatttttatttcaagatgtagagagagagatccatgcagccccacacgtgagtgagaaattaattcagctgttccggaataagagtgaattcacctttttggccactgtgcagcagaagccgtcaacttcaggagtgatactgtccattcgagaactggaaaacagttattttgaactggagagcagtggcctgagagatgagattcgatatcactacacacacaaggggaagcccaggacagaggcacttccctaccggatggcggacggacggtggcacaaggtggcgctgtcagttagcgcctctcatctcctgctccacatcgactgcaacaggatttatgaacgtgtgatagacactcctgagaccaacctccccccaggaagcaatttgtggctgggtcagcgaaaccaaaagcacggcttattcaaaggaatcatccaagatggaaaaatcatcttcatgccgaatggatacataacacagtgtccgaacctgaatcgcacttgcccaacgtgcagtgatttcttaagcctggtgcaaggaatcatggatttacaagagcttctggccaagatgactgcgaaactaaattatgcagagacacgacttagtcaattggaaaactgccactgcgagaagacctgtcaagtgagtggactgctctatagagaccaggactcctgggttgatggcgatcactgtaggaactgcacgtgcaaaagcggcgctgtggaatgtcggaggatgtcttgtccccctctcaattgctccccagactccctccctgtgcacgttgccggccagtgctgtaaggtctgccgaccaaaatgtatctacggagggaaagtccttgcagaaggccagcggattttaaccaagagctgtcgggaatgccgaggtggagttttagtgaaaattacagaagcgtgccctcctttgaactgctcagacaaggatcacattctcccagagaatcagtgctgcagcgtctgcagaggtcataacttttgtgcggaaggacctaaatgtggtgaaaattcagagtgcaaaaactggaatacaaaagctacttgcgagtgcaagaatggttatatctctgtccagggggactccgcctactgtgaagatatcgatgagtgtgctgctaagatgcattactgtcgtgccaatactgtgtgtgtcaacctgcctgggttatatcggtgtgactgtgtcccgggatacattcgcgtggatgatttctcttgtacagaacatgacgaatgtggcagcgggcagcacaactgtgatgagaatgccatctgcaccaacactgtccagggacacagctgcacctgcaaaccgggctacgtggggaatgggaccagctgcagagcgttctgcgaagagggctgcagatatggcgggacatgcgtggctcctaacaaatgtgtctgtccttctggattcacaggaagccactgtgagaaagatattgatgaatgtacagagggaatcattgagtgccacaaccattcccgctgcgttaacctgccagggtggtaccactgtgagtgcagaagcggtttccatgacgatgggacctattcactgtccggggagtcctgtattgacattgatgaatgtgccttaagaactcacacctgttggaatgattctgcctgcatcaacttggcagggggcttcgactgcctgtgtccctcagggccatcctgctctggtgactgcccccacgaaggaggactgaagcgcaacgggcaggtgtggaccctgaaagaagacaggtgttctgtgtgttcctgcaaggatgggaagatattctgccgacggacagcttgtgattgccagaatccaagcgttgaccttttctgttgcccagagtgtgacaccagggtcacaagtcaatgtttagaccaaaatggacacaagctctatcgaagtggagacaattggactcacagctgtcagcagtgccggtgtctggaaggagaggtagattgctggccactcacttgccccagattgagctgtgagtacacagccatcttggaaggggagtgttgtccacgctgtgtcagcgacccctgcctggcggataacatcgtctatgacatcagagaaacttgcctggacagctatggagtttcaaggcttagtggctcagtgtggacattggctggatctccctgcacgacctgcaaatgcaagaatggaagtgtctgctgttctgtggatttggagtgtcttcataataattgaaggatttaaaatggactcatgatcgccagagaaaaatggacaaatgaccatccatgatgatgaaagaacaggagttggtgttttttttaccacagacaattaccaaagtctccgtctgaggaaggtgtttgcaggttgccttttggacctcccactctgctcattcttgctaacctagtctaggtgacctacagtgcatttcagtctatggttgttaaaagaagttttccgtgttgtaaatcacgtttcccttaccaggtcattgcaaatacatttaaatgatttcatggtaaatgttgatgtattttttgggtttattttgtgtactaacataatagagattcagctgcttttatttatttttttcttgacttttggatcaaattcaacaaataaagttgcctgttgtgatttt

SEQ ID NO: 51 - protein, 791 aa, Equus caballus
MGFGMDPDLQMDIITELDLVNTTLGVTQVSGLHNASKAFLFQDVEREIHAAPHVSEKLIQLFRNKSEFTFLATVQQKPSTSGVILSIRELENSYFELESSGLRDEIRYHYTHKGKPRTEALPYRMADGRWHKVALSVSASHLLLHIDCNRIYERVIDTPETNLPPGSNLWLGQRNQKHGLFKGIIQDGKIIFMPNGYITQCPNLNRTCPTCSDFLSLVQGIMDLQELLAKMTAKLNYAETRLSQLENCHCEKTCQVSGLLYRDQDSWVDGDHCRNCTCKSGAVECRRMSCPPLNCSPDSLPVHVAGQCCKVCRPKCIYGGKVLAEGQRILTKSCRECRGGVLVKITEACPPLNCSDKDHILPENQCCSVCRGHNFCAEGPKCGENSECKNWNTKATCECKNGYISVQGDSAYCEDIDECAAKMHYCRANTVCVNLPGLYRCDCVPGYIRVDDFSCTEHDECGSGQHNCDENAICTNTVQGHSCTCKPGYVGNGTSCRAFCEEGCRYGGTCVAPNKCVCPSGFTGSHCEKDIDECTEGIIECHNHSRCVNLPGWYHCECRSGFHDDGTYSLSGESCIDIDECALRTHTCWNDSACINLAGGFDCLCPSGPSCSGDCPHEGGLKRNGQVWTLKEDRCSVCSCKDGKIFCRRTACDCQNPSVDLFCCPECDTRVTSQCLDQNGHKLYRSGDNWTHSCQQCRCLEGEVDCWPLTCPRLSCEYTAILEGECCPRCVSDPCLADNIVYDIRETCLDSYGVSRLSGSVWTLAGSPCTTCKCKNGSVCCSVDLECLHNN

SEQ ID NO: 52 - DNA, 2285 bp, Equus caballus
  CDS (1)...(2235): Coding region
atgggctttgggatggaccccgaccttcaaatggatattatcaccgagctcgacctcgtgaacaccacccttggagtcactcaggtgtccggactgcacaatgccagcaaagcatttttatttcaagatgtagagagagagatccatgcagccccacacgtgagtgagaaattaattcagctgttccggaataagagtgaattcacctttttggccactgtgcagcagaagccgtcaacttcaggagtgatactgtccattcgagaactggaaaacagttattttgaactggagagcagtggcctgagagatgagattcgatatcactacacacacaaggggaagcccaggacagaggcacttccctaccggatggcggacggacggtggcacaaggtggcgctgtcagttagcgcctctcatctcctgctccacatcgactgcaacaggatttatgaacgtgtgatagacactcctgagaccaacctccccccaggaagcaatttgtggctgggtcagcgaaaccaaaagcacggcttattcaaaggaatcatccaagatggaaaaatcatcttcatgccgaatggatacataacacagtgtccgaacctgaatcgcacttgcccaacgtgcagtgatttcttaagcctggtgcaaggaatcatggatttacaagagcttctggccaagatgactgcgaaactaaattatgcagagacacgacttagtcaattggaaaactgccactgcgagaagacctgtcaagtgagtggactgctctatagagaccaggactcctgggttgatggcgatcactgtaggaactgcacgtgcaaaagcggcgctgtggaatgtcggaggatgtcttgtccccctctcaattgctccccagactccctccctgtgcacgttgccggccagtgctgtaaggtctgccgaccaaaatgtatctacggagggaaagtccttgcagaaggccagcggattttaaccaagagctgtcgggaatgccgaggtggagttttagtgaaaattacagaagcgtgccctcctttgaactgctcagacaaggatcacattctcccagagaatcagtgctgcagcgtctgcagaggtcataacttttgtgcggaaggacctaaatgtggtgaaaattcagagtgcaaaaactggaatacaaaagctacttgcgagtgcaagaatggttatatctctgtccagggggactccgcctactgtgaagatatcgatgagtgtgctgctaagatgcattactgtcgtgccaatactgtgtgtgtcaacctgcctgggttatatcggtgtgactgtgtcccgggatacattcgcgtggatgatttctcttgtacagaacatgacgaatgtggcagcgggcagcacaactgtgatgagaatgccatctgcaccaacactgtccagggacacagctgcacctgcaaaccgggctacgtggggaatgggaccagctgcagagcgttctgcgaagagggctgcagatatggcgggacatgcgtggctcctaacaaatgtgtctgtccttctggattcacaggaagccactgtgagaaagacattgatgaatgtgccttaagaactcacacctgttggaatgattctgcctgcatcaacttggcagggggcttcgactgcctgtgtccctcagggccatcctgctctggtgactgcccccacgaaggaggactgaagcgcaacgggcaggtgtggaccctgaaagaagacaggtgttctgtgtgttcctgcaaggatgggaagatattctgccgacggacagcttgtgattgccagaatccaagcgttgaccttttctgttgcccagagtgtgacaccagggtcacaagtcaatgtttagaccaaaatggacacaagctctatcgaagtggagacaattggactcacagctgtcagcagtgccggtgtctggaaggagaggtagattgctggccactcacttgccccagattgagctgtgagtacacagccatcttggaaggggagtgttgtccacgctgtgtcagcgacccctgcctggcggataacatcgtctatgacatcagagaaacttgcctggacagctatggagtttcaaggcttagtggctcagtgtggacattggctggatctccctgcacgacctgcaaatgcaagaatggaagtgtctgctgttctgtggatttggagtgtcttcataataattgaaggatttaaaatggactcatgatcgccagagaaaaatggacaaatgacca

SEQ ID NO: 53 - protein, 744 aa, Equus caballus
MGFGMDPDLQMDIITELDLVNTTLGVTQVSGLHNASKAFLFQDVEREIHAAPHVSEKLIQLFRNKSEFTFLATVQQKPSTSGVILSIRELENSYFELESSGLRDEIRYHYTHKGKPRTEALPYRMADGRWHKVALSVSASHLLLHIDCNRIYERVIDTPETNLPPGSNLWLGQRNQKHGLFKGIIQDGKIIFMPNGYITQCPNLNRTCPTCSDFLSLVQGIMDLQELLAKMTAKLNYAETRLSQLENCHCEKTCQVSGLLYRDQDSWVDGDHCRNCTCKSGAVECRRMSCPPLNCSPDSLPVHVAGQCCKVCRPKCIYGGKVLAEGQRILTKSCRECRGGVLVKITEACPPLNCSDKDHILPENQCCSVCRGHNFCAEGPKCGENSECKNWNTKATCECKNGYISVQGDSAYCEDIDECAAKMHYCRANTVCVNLPGLYRCDCVPGYIRVDDFSCTEHDECGSGQHNCDENAICTNTVQGHSCTCKPGYVGNGTSCRAFCEEGCRYGGTCVAPNKCVCPSGFTGSHCEKDIDECALRTHTCWNDSACINLAGGFDCLCPSGPSCSGDCPHEGGLKRNGQVWTLKEDRCSVCSCKDGKIFCRRTACDCQNPSVDLFCCPECDTRVTSQCLDQNGHKLYRSGDNWTHSCQQCRCLEGEVDCWPLTCPRLSCEYTAILEGECCPRCVSDPCLADNIVYDIRETCLDSYGVSRLSGSVWTLAGSPCTTCKCKNGSVCCSVDLECLHNN

SEQ ID NO: 54 - DNA, 2856 bp, Macaca mulatta
  CDS (148)...(2409): Coding region
cgagcgcagcacaggcgctgccgagccacctcccccgccgcccgctagcaagtttggcggctccaagccaggcgcgcctcaggatccaggctcatttgtttccacctagcttcggtgcgccctgctaggcggggaccctcgggagcgatgccggtggatgtgattttagttgtgtggttctgtgtgtgcactgccaggacagtggtgggctttgggatggaccctgaccttcagatggatatcatcaccgagcttgaccttgtgaacaccacccttggagtggctcaggtgtctggaatgcataatgccagcaaagcatttttatttcaagacatagaaagagagatccatgcagctcctcatgtgagtgagaaattaattcagctgttccggaacaagagtgaattcacctttttggccactgtacagcagaagccatccacttcaggagtaatactgtccattcgagaactggagcacaggatttatgagcgtgtgatagaccctccagataccaaccttcccccaggaatcaatttatggcttggccagcgcaaccaaaagcatggcttattcaaagggatcatccaagatgggaagatcatctttatgccgaatggatatataacacagtgtccaaatctaaatcgcacttgcccaacctgcagtgatttcttaagcctggtacaaggaataatggatttacaagagcttttggccaagatgactgcaaaactaaattatgcagagacaagacttagtcaattggaaaactgtcattgtgagaagacttgtcaagtgagtggactgctctatcgagaccaagactcttgggtagatggtgaccattgcaggaactgcacttgcaaaagtggtgcagtggaatgccgaaggatgtcctgtccccctctcaattgctccccagactccctcccggtgcacattgctggccagtgctgtaaggtctgccgaccaaaatgtatctatggaggaaaagttcttgcagaaggccagcggattttaaccaagagctgtcgggaatgccgaggtggagttttagtaaaaattacggaaatgtgtcctcctttgaactgctcagaaaaggatcacattcttcctgagaatcagtgctgccgtgtctgtagaggtcataacttttgtgcagaaggacctaaatgtggtgaaaactcagagtgcaaaaactggaatacaaaagctacttgtgagtgcaagagtggttacatctctgtccagggagactctgcctactgtgaagatattgatgagtgtgcagctaagatgcattactgtcatgccaatactgtgtgtgtcaaccttcctgggttatatcgctgtgactgtgtcccaggatacattcgtgtggatgacttctcttgtacagaacacgatgaatgtggcagcggccagcacaactgtgacgagaatgccatctgcaccaacactgtccagggacacagctgcacctgcaaaccgggctacgtggggaatgggaccatctgcagagctttctgtgaagagggctgcagatacggtgggacgtgtgtggctcctaacaaatgtgtctgtccatctggattcacaggaagtcactgcgagaaagatattgatgaatgttcagagggaatcattgagtgccacaaccattcccgctgcgttaacctgccagggtggtaccactgtgagtgcagaagcggtttccatgacgatgggacctattcactgtccggggagtcctgtattgacatcgatgaatgtgccttaagaactcacacctgttggaacgattctgcctgcatcaacctggcagggggctttgactgtctctgcccctctgggccctcctgctctggtgactgtcctcatgaaggggggctgaagcgcaatggccaggtgtggaccttgaaagaagacaggtgttctgtctgctcctgcaaggatggcaagatattctgccgacggacggcctgtgattgccagaatccaagtgctgaccttttctgttgcccagaatgtgacaccagggtcacaagtcaatgtttagaccaaaatggtcacaagctgtatcgaagtggagacaattggacccatagctgtcagcaatgtcggtgtctggaaggagaggtagattgctggccactcacttgccccaacttgagctgtgagtacacagctatcttagaaggggagtgttgtccccgctgtgtcagtgacccctgcctagctgataacatcgcctatgacatcagaaaaacttgcctggacagctatggtgtttcaaggcttagtggctcagtgtggacgatggctggatctccctgcacaacctgtaaatgcaagaatggaagagtctgttgttctgtggatttggagtgtcttcaaaataactgaagtatttaaaatggactcatcgcagaagaatggacaaaatgaccatccaacgtgattaagaataggaattggtggtttgggttttttgtttgttttgttttttttaaccacagacaattgccaaagtttccatctgaggaaggtgtttggaggttgccttttggacctaccactttgctcattcttgctaacctagtctaggtgacctacagtgcagtccatttaagtcaatggttgttaaaagaagtttcccgtgttgtaaatcatgtttcccttatcagatcatttgcaaacacatttaaatgatctcatggtaaatgttgatgtattttttggcttattttgtgtacgaacataatagagactcagctccttttatttattttgttgatttatggatcaaattctaaaataaagttgcctgttgtgatttttgtcccatctact

SEQ ID NO: 55 - protein, 753 aa, Macaca mulatta
MPVDVILVVWFCVCTARTVVGFGMDPDLQMDIITELDLVNTTLGVAQVSGMHNASKAFLFQDIEREIHAAPHVSEKLIQLFRNKSEFTFLATVQQKPSTSGVILSIRELEHRIYERVIDPPDTNLPPGINLWLGQRNQKHGLFKGIIQDGKIIFMPNGYITQCPNLNRTCPTCSDFLSLVQGIMDLQELLAKMTAKLNYAETRLSQLENCHCEKTCQVSGLLYRDQDSWVDGDHCRNCTCKSGAVECRRMSCPPLNCSPDSLPVHIAGQCCKVCRPKCIYGGKVLAEGQRILTKSCRECRGGVLVKITEMCPPLNCSEKDHILPENQCCRVCRGHNFCAEGPKCGENSECKNWNTKATCECKSGYISVQGDSAYCEDIDECAAKMHYCHANTVCVNLPGLYRCDCVPGYIRVDDFSCTEHDECGSGQHNCDENAICTNTVQGHSCTCKPGYVGNGTICRAFCEEGCRYGGTCVAPNKCVCPSGFTGSHCEKDIDECSEGIIECHNHSRCVNLPGWYHCECRSGFHDDGTYSLSGESCIDIDECALRTHTCWNDSACINLAGGFDCLCPSGPSCSGDCPHEGGLKRNGQVWTLKEDRCSVCSCKDGKIFCRRTACDCQNPSADLFCCPECDTRVTSQCLDQNGHKLYRSGDNWTHSCQQCRCLEGEVDCWPLTCPNLSCEYTAILEGECCPRCVSDPCLADNIAYDIRKTCLDSYGVSRLSGSVWTMAGSPCTTCKCKNGRVCCSVDLECLQNN

SEQ ID NO: 56 - DNA, 3314 bp, Macaca mulatta
  CDS (148)...(2664): Coding region
cgagcgcagcacaggcgctgccgagccacctcccccgccgcccgctagcaagtttggcggctccaagccaggcgcgcctcaggatccaggctcatttgtttccacctagcttcggtgcgccctgctaggcggggaccctcgggagcgatgccggtggatgtgattttagttgtgtggttctgtgtgtgcactgccaggacagcaggagatatgaagccacccgtgttatcgctgatgtggagtactagagttctgaagtctctgcttcagcaattccttcagggagtggtgggctttgggatggaccctgaccttcagatggatatcatcaccgagcttgaccttgtgaacaccacccttggagtggctcaggtgtctggaatgcataatgccagcaaagcatttttatttcaagacatagaaagagagatccatgcagctcctcatgtgagtgagaaattaattcagctgttccggaacaagagtgaattcacctttttggccactgtacagcagaagccatccacttcaggagtaatactgtccattcgagaactggagcacagctattttgaactggagagcagtggcctgagggatgagattcggtatcactacatacacagtgggaagccaaggacagaggcacttccttaccggatggcagatggacgatggcacaagattgcactgtcagttagtgcctctcatcttctgctccacgtcgactgtaacaggatttatgagcgtgtgatagaccctccagataccaaccttcccccaggaatcaatttatggcttggccagcgcaaccaaaagcatggcttattcaaagggatcatccaagatgggaagatcatctttatgccgaatggatatataacacagtgtccaaatctaaatcgcacttgcccaacctgcagtgatttcttaagcctggtacaaggaataatggatttacaagagcttttggccaagatgactgcaaaactaaattatgcagagacaagacttagtcaattggaaaactgtcattgtgagaagacttgtcaagtgagtggactgctctatcgagaccaagactcttgggtagatggtgaccattgcaggaactgcacttgcaaaagtggtgcagtggaatgccgaaggatgtcctgtccccctctcaattgctccccagactccctcccggtgcacattgctggccagtgctgtaaggtctgccgaccaaaatgtatctatggaggaaaagttcttgcagaaggccagcggattttaaccaagagctgtcgggaatgccgaggtggagttttagtaaaaattacggaaatgtgtcctcctttgaactgctcagaaaaggatcacattcttcctgagaatcagtgctgccgtgtctgtagaggtcataacttttgtgcagaaggacctaaatgtggtgaaaactcagagtgcaaaaactggaatacaaaagctacttgtgagtgcaagagtggttacatctctgtccagggagactctgcctactgtgaagatattgatgagtgtgcagctaagatgcattactgtcatgccaatactgtgtgtgtcaaccttcctgggttatatcgctgtgactgtgtcccaggatacattcgtgtggatgacttctcttgtacagaacacgatgaatgtggcagcggccagcacaactgtgacgagaatgccatctgcaccaacactgtccagggacacagctgcacctgcaaaccgggctacgtggggaatgggaccatctgcagagctttctgtgaagagggctgcagatacggtgggacgtgtgtggctcctaacaaatgtgtctgtccatctggattcacaggaagtcactgcgagaaagatattgatgaatgttcagagggaatcattgagtgccacaaccattcccgctgcgttaacctgccagggtggtaccactgtgagtgcagaagcggtttccatgacgatgggacctattcactgtccggggagtcctgtattgacatcgatgaatgtgccttaagaactcacacctgttggaacgattctgcctgcatcaacctggcagggggctttgactgtctctgcccctctgggccctcctgctctggtgactgtcctcatgaaggggggctgaagcgcaatggccaggtgtggaccttgaaagaagacaggtgttctgtctgctcctgcaaggatggcaagatattctgccgacggacggcctgtgattgccagaatccaagtgctgaccttttctgttgcccagaatgtgacaccagggtcacaagtcaatgtttagaccaaaatggtcacaagctgtatcgaagtggagacaattggacccatagctgtcagcaatgtcggtgtctggaaggagaggtagattgctggccactcacttgccccaacttgagctgtgagtacacagctatcttagaaggggagtgttgtccccgctgtgtcagtgacccctgcctagctgataacatcgcctatgacatcagaaaaacttgcctggacagctatggtgtttcaaggcttagtggctcagtgtggacgatggctggatctccctgcacaacctgtaaatgcaagaatggaagagtctgttgttctgtggatttggagtgtcttcaaaataactgaagtatttaaaatggactcatcgcagaagaatggacaaaatgaccatccaacgtgattaagaataggaattggtggtttgggttttttgtttgttttgttttttttaaccacagacaattgccaaagtttccatctgaggaaggtgtttggaggttgccttttggacctaccactttgctcattcttgctaacctagtctaggtgacctacagtgcagtccatttaagtcaatggttgttaaaagaagtttcccgtgttgtaaatcatgtttcccttatcagatcatttgcaaacacatttaaatgatctcatggtaaatgttgatgtattttttggcttattttgtgtacgaacataatagagactcagctccttttatttattttgttgatttatggatcaaattctaaaataaagttgcctgttgtgatttttgtcccatctactatatacttagtgctgagatccttgtaaaatgttttgatgaaaatatatatgtagggtccaattgcattatacatacatttcaaagtgttgaaccttcttaaatgcctactcattcagcttacacaggctgaagccaagtatgacaaagaagggaaggaccaaaaacaaagaattattttaaagagaactcttgtctcttttgc

SEQ ID NO: 57 - protein, 838 aa, Macaca mulatta
MPVDVILVVWFCVCTARTAGDMKPPVLSLMWSTRVLKSLLQQFLQGVVGFGMDPDLQMDIITELDLVNTTLGVAQVSGMHNASKAFLFQDIEREIHAAPHVSEKLIQLFRNKSEFTFLATVQQKPSTSGVILSIRELEHSYFELESSGLRDEIRYHYIHSGKPRTEALPYRMADGRWHKIALSVSASHLLLHVDCNRIYERVIDPPDTNLPPGINLWLGQRNQKHGLFKGIIQDGKIIFMPNGYITQCPNLNRTCPTCSDFLSLVQGIMDLQELLAKMTAKLNYAETRLSQLENCHCEKTCQVSGLLYRDQDSWVDGDHCRNCTCKSGAVECRRMSCPPLNCSPDSLPVHIAGQCCKVCRPKCIYGGKVLAEGQRILTKSCRECRGGVLVKITEMCPPLNCSEKDHILPENQCCRVCRGHNFCAEGPKCGENSECKNWNTKATCECKSGYISVQGDSAYCEDIDECAAKMHYCHANTVCVNLPGLYRCDCVPGYIRVDDFSCTEHDECGSGQHNCDENAICTNTVQGHSCTCKPGYVGNGTICRAFCEEGCRYGGTCVAPNKCVCPSGFTGSHCEKDIDECSEGIIECHNHSRCVNLPGWYHCECRSGFHDDGTYSLSGESCIDIDECALRTHTCWNDSACINLAGGFDCLCPSGPSCSGDCPHEGGLKRNGQVWTLKEDRCSVCSCKDGKIFCRRTACDCQNPSADLFCCPECDTRVTSQCLDQNGHKLYRSGDNWTHSCQQCRCLEGEVDCWPLTCPNLSCEYTAILEGECCPRCVSDPCLADNIAYDIRKTCLDSYGVSRLSGSVWTMAGSPCTTCKCKNGRVCCSVDLECLQNN

SEQ ID NO: 58 - DNA, 3102 bp, Macaca mulatta
  CDS (151)...(2442): Coding region
ccccgagcgcagcacaggcgctgccgagccacctcccccgccgcccgctagcaagtttggcggctccaagccaggcgcgcctcaggatccaggctcatttgtttccacctagcttcggtgcgccctgctaggcggggaccctcgggagcgatgccggtggatgtgattttagttgtgtggttctgtgtgtgcactgccaggacagtggtgggctttgggatggaccctgaccttcagatggatatcatcaccgagcttgaccttgtgaacaccacccttggagtggctcaggtgtctggaatgcataatgccagcaaagcatttttatttcaagacatagaaagagagatccatgcagctcctcatgtgagtgagaaattaattcagctgttccggaacaagagtgaattcacctttttggccactgtacagcagaagccatccacttcaggagtaatactgtccattcgagaactggagcacagctattttgaactggagagcagtggcctgagggatgagattcggtatcactacatacacagtgggaagccaaggacagaggcacttccttaccggatggcagatggacgatggcacaagattgcactgtcagttagtgcctctcatcttctgctccacgtcgactgtaacaggatttatgagcgtgtgatagaccctccagataccaaccttcccccaggaatcaatttatggcttggccagcgcaaccaaaagcatggcttattcaaagggatcatccaagatgggaagatcatctttatgccgaatggatatataacacagtgtccaaatctaaatcgcacttgcccaacctgcagtgatttcttaagcctggtacaaggaataatggatttacaagagcttttggccaagatgactgcaaaactaaattatgcagagacaagacttagtcaattggaaaactgtcattgtgagaagacttgtcaagtgagtggactgctctatcgagaccaagactcttgggtagatggtgaccattgcaggaactgcacttgcaaaagtggtgcagtggaatgccgaaggatgtcctgtccccctctcaattgctccccagactccctcccggtgcacattgctggccagtgctgtaaggtctgccgaccaaaatgtatctatggaggaaaagttcttgcagaaggccagcggattttaaccaagagctgtcgggaatgccgaggtggagttttagtaaaaattacggaaatgtgtcctcctttgaactgctcagaaaaggatcacattcttcctgagaatcagtgctgccgtgtctgtagaggtcataacttttgtgcagaaggacctaaatgtggtgaaaactcagagtgcaaaaactggaatacaaaagctacttgtgagtgcaagagtggttacatctctgtccagggagactctgcctactgtgaagatattgatgagtgtgcagctaagatgcattactgtcatgccaatactgtgtgtgtcaaccttcctgggttatatcgctgtgactgtgtcccaggatacattcgtgtggatgacttctcttgtacagaacacgatgaatgtggcagcggccagcacaactgtgacgagaatgccatctgcaccaacactgtccagggacacagctgcacctgcaaaccgggctacgtggggaatgggaccatctgcagagctttctgtgaagagggctgcagatacggtgggacgtgtgtggctcctaacaaatgtgtctgtccatctggattcacaggaagtcactgcgagaaagacatcgatgaatgtgccttaagaactcacacctgttggaacgattctgcctgcatcaacctggcagggggctttgactgtctctgcccctctgggccctcctgctctggtgactgtcctcatgaaggggggctgaagcgcaatggccaggtgtggaccttgaaagaagacaggtgttctgtctgctcctgcaaggatggcaagatattctgccgacggacggcctgtgattgccagaatccaagtgctgaccttttctgttgcccagaatgtgacaccagggtcacaagtcaatgtttagaccaaaatggtcacaagctgtatcgaagtggagacaattggacccatagctgtcagcaatgtcggtgtctggaaggagaggtagattgctggccactcacttgccccaacttgagctgtgagtacacagctatcttagaaggggagtgttgtccccgctgtgtcagtgacccctgcctagctgataacatcgcctatgacatcagaaaaacttgcctggacagctatggtgtttcaaggcttagtggctcagtgtggacgatggctggatctccctgcacaacctgtaaatgcaagaatggaagagtctgttgttctgtggatttggagtgtcttcaaaataactgaagtatttaaaatggactcatcgcagaagaatggacaaaatgaccatccaacgtgattaagaataggaattggtggtttgggttttttgtttgttttgttttttttaaccacagacaattgccaaagtttccatctgaggaaggtgtttggaggttgccttttggacctaccactttgctcattcttgctaacctagtctaggtgacctacagtgcagtccatttaagtcaatggttgttaaaagaagtttcccgtgttgtaaatcatgtttcccttatcagatcatttgcaaacacatttaaatgatctcatggtaaatgttgatgtattttttggcttattttgtgtacgaacataatagagactcagctccttttatttattttgttgatttatggatcaaattctaaaataaagttgcctgttgtgatttttgtcccatctactatatacttagtgctgagatccttgtaaaatgttttgatgaaaatatatatgtagggtccaattgcattatacatacatttcaaagtgttgaaccttcttaaatgcctactcattcagcttacacaggctgaagccaagtatgacaaagaagggaaggaccaaaaacaaagaattattttaaagagaactcttgtctcttttgcaaaaaaaaaa

SEQ ID NO: 59 - protein, 763 aa, Macaca mulatta
MPVDVILVVWFCVCTARTVVGFGMDPDLQMDIITELDLVNTTLGVAQVSGMHNASKAFLFQDIEREIHAAPHVSEKLIQLFRNKSEFTFLATVQQKPSTSGVILSIRELEHSYFELESSGLRDEIRYHYIHSGKPRTEALPYRMADGRWHKIALSVSASHLLLHVDCNRIYERVIDPPDTNLPPGINLWLGQRNQKHGLFKGIIQDGKIIFMPNGYITQCPNLNRTCPTCSDFLSLVQGIMDLQELLAKMTAKLNYAETRLSQLENCHCEKTCQVSGLLYRDQDSWVDGDHCRNCTCKSGAVECRRMSCPPLNCSPDSLPVHIAGQCCKVCRPKCIYGGKVLAEGQRILTKSCRECRGGVLVKITEMCPPLNCSEKDHILPENQCCRVCRGHNFCAEGPKCGENSECKNWNTKATCECKSGYISVQGDSAYCEDIDECAAKMHYCHANTVCVNLPGLYRCDCVPGYIRVDDFSCTEHDECGSGQHNCDENAICTNTVQGHSCTCKPGYVGNGTICRAFCEEGCRYGGTCVAPNKCVCPSGFTGSHCEKDIDECALRTHTCWNDSACINLAGGFDCLCPSGPSCSGDCPHEGGLKRNGQVWTLKEDRCSVCSCKDGKIFCRRTACDCQNPSADLFCCPECDTRVTSQCLDQNGHKLYRSGDNWTHSCQQCRCLEGEVDCWPLTCPNLSCEYTAILEGECCPRCVSDPCLADNIAYDIRKTCLDSYGVSRLSGSVWTMAGSPCTTCKCKNGRVCCSVDLECLQNN

SEQ ID NO: 60 - DNA, 3243 bp, Macaca mulatta
  CDS (151)...(2583): Coding region
ccccgagcgcagcacaggcgctgccgagccacctcccccgccgcccgctagcaagtttggcggctccaagccaggcgcgcctcaggatccaggctcatttgtttccacctagcttcggtgcgccctgctaggcggggaccctcgggagcgatgccggtggatgtgattttagttgtgtggttctgtgtgtgcactgccaggacagtggtgggctttgggatggaccctgaccttcagatggatatcatcaccgagcttgaccttgtgaacaccacccttggagtggctcaggtgtctggaatgcataatgccagcaaagcatttttatttcaagacatagaaagagagatccatgcagctcctcatgtgagtgagaaattaattcagctgttccggaacaagagtgaattcacctttttggccactgtacagcagaagccatccacttcaggagtaatactgtccattcgagaactggagcacagctattttgaactggagagcagtggcctgagggatgagattcggtatcactacatacacagtgggaagccaaggacagaggcacttccttaccggatggcagatggacgatggcacaagattgcactgtcagttagtgcctctcatcttctgctccacgtcgactgtaacaggatttatgagcgtgtgatagaccctccagataccaaccttcccccaggaatcaatttatggcttggccagcgcaaccaaaagcatggcttattcaaagggatcatccaagatgggaagatcatctttatgccgaatggatatataacacagtgtccaaatctaaatcgcacttgcccaacctgcagtgatttcttaagcctggtacaaggaataatggatttacaagagcttttggccaagatgactgcaaaactaaattatgcagagacaagacttagtcaattggaaaactgtcattgtgagaagacttgtcaagtgagtggactgctctatcgagaccaagactcttgggtagatggtgaccattgcaggaactgcacttgcaaaagtggtgcagtggaatgccgaaggatgtcctgtccccctctcaattgctccccagactccctcccggtgcacattgctggccagtgctgtaaggtctgccgaccaaaatgtatctatggaggaaaagttcttgcagaaggccagcggattttaaccaagagctgtcgggaatgccgaggtggagttttagtaaaaattacggaaatgtgtcctcctttgaactgctcagaaaaggatcacattcttcctgagaatcagtgctgccgtgtctgtagaggtcataacttttgtgcagaaggacctaaatgtggtgaaaactcagagtgcaaaaactggaatacaaaagctacttgtgagtgcaagagtggttacatctctgtccagggagactctgcctactgtgaagatattgatgagtgtgcagctaagatgcattactgtcatgccaatactgtgtgtgtcaaccttcctgggttatatcgctgtgactgtgtcccaggatacattcgtgtggatgacttctcttgtacagaacacgatgaatgtggcagcggccagcacaactgtgacgagaatgccatctgcaccaacactgtccagggacacagctgcacctgcaaaccgggctacgtggggaatgggaccatctgcagagctttctgtgaagagggctgcagatacggtgggacgtgtgtggctcctaacaaatgtgtctgtccatctggattcacaggaagtcactgcgagaaagatattgatgaatgttcagagggaatcattgagtgccacaaccattcccgctgcgttaacctgccagggtggtaccactgtgagtgcagaagcggtttccatgacgatgggacctattcactgtccggggagtcctgtattgacatcgatgaatgtgccttaagaactcacacctgttggaacgattctgcctgcatcaacctggcagggggctttgactgtctctgcccctctgggccctcctgctctggtgactgtcctcatgaaggggggctgaagcgcaatggccaggtgtggaccttgaaagaagacaggtgttctgtctgctcctgcaaggatggcaagatattctgccgacggacggcctgtgattgccagaatccaagtgctgaccttttctgttgcccagaatgtgacaccagggtcacaagtcaatgtttagaccaaaatggtcacaagctgtatcgaagtggagacaattggacccatagctgtcagcaatgtcggtgtctggaaggagaggtagattgctggccactcacttgccccaacttgagctgtgagtacacagctatcttagaaggggagtgttgtccccgctgtgtcagtgacccctgcctagctgataacatcgcctatgacatcagaaaaacttgcctggacagctatggtgtttcaaggcttagtggctcagtgtggacgatggctggatctccctgcacaacctgtaaatgcaagaatggaagagtctgttgttctgtggatttggagtgtcttcaaaataactgaagtatttaaaatggactcatcgcagaagaatggacaaaatgaccatccaacgtgattaagaataggaattggtggtttgggttttttgtttgttttgttttttttaaccacagacaattgccaaagtttccatctgaggaaggtgtttggaggttgccttttggacctaccactttgctcattcttgctaacctagtctaggtgacctacagtgcagtccatttaagtcaatggttgttaaaagaagtttcccgtgttgtaaatcatgtttcccttatcagatcatttgcaaacacatttaaatgatctcatggtaaatgttgatgtattttttggcttattttgtgtacgaacataatagagactcagctccttttatttattttgttgatttatggatcaaattctaaaataaagttgcctgttgtgatttttgtcccatctactatatacttagtgctgagatccttgtaaaatgttttgatgaaaatatatatgtagggtccaattgcattatacatacatttcaaagtgttgaaccttcttaaatgcctactcattcagcttacacaggctgaagccaagtatgacaaagaagggaaggaccaaaaacaaagaattattttaaagagaactcttgtctcttttgcaaaaaaaaaa

SEQ ID NO: 61 - protein, 810 aa, Macaca mulatta
MPVDVILVVWFCVCTARTVVGFGMDPDLQMDIITELDLVNTTLGVAQVSGMHNASKAFLFQDIEREIHAAPHVSEKLIQLFRNKSEFTFLATVQQKPSTSGVILSIRELEHSYFELESSGLRDEIRYHYIHSGKPRTEALPYRMADGRWHKIALSVSASHLLLHVDCNRIYERVIDPPDTNLPPGINLWLGQRNQKHGLFKGIIQDGKIIFMPNGYITQCPNLNRTCPTCSDFLSLVQGIMDLQELLAKMTAKLNYAETRLSQLENCHCEKTCQVSGLLYRDQDSWVDGDHCRNCTCKSGAVECRRMSCPPLNCSPDSLPVHIAGQCCKVCRPKCIYGGKVLAEGQRILTKSCRECRGGVLVKITEMCPPLNCSEKDHILPENQCCRVCRGHNFCAEGPKCGENSECKNWNTKATCECKSGYISVQGDSAYCEDIDECAAKMHYCHANTVCVNLPGLYRCDCVPGYIRVDDFSCTEHDECGSGQHNCDENAICTNTVQGHSCTCKPGYVGNGTICRAFCEEGCRYGGTCVAPNKCVCPSGFTGSHCEKDIDECSEGIIECHNHSRCVNLPGWYHCECRSGFHDDGTYSLSGESCIDIDECALRTHTCWNDSACINLAGGFDCLCPSGPSCSGDCPHEGGLKRNGQVWTLKEDRCSVCSCKDGKIFCRRTACDCQNPSADLFCCPECDTRVTSQCLDQNGHKLYRSGDNWTHSCQQCRCLEGEVDCWPLTCPNLSCEYTAILEGECCPRCVSDPCLADNIAYDIRKTCLDSYGVSRLSGSVWTMAGSPCTTCKCKNGRVCCSVDLECLQNN